(19) United States
(12) Patent Application Publication
(10) Pub. No.: US 2025/0257365 A1
(43) Pub. Date: Aug. 14, 2025

(54) Targeted DNA Integration in Plants by CRISPR-Associated Transposases (CASTs)

(71) Applicant: California Institute of Technology, Pasadena, CA (US)

(72) Inventors: Gozde Demirer, Altadena, CA (US); Yunqing Wang, Pasadena, CA (US); Kimberley T. Muchenje, Pasadena, CA (US)

(21) Appl. No.: 19/048,891

(22) Filed: Feb. 8, 2025

Related U.S. Application Data
(60) Provisional application No. 63/551,713, filed on Feb. 9, 2024.

(51) Int. Cl. C12N 15/82; C12N 9/12; C12N 9/22; C12N 15/10; C12N 15/11; C12Q 1/6869; C12Q 1/6895

(52) U.S. Cl. CPC C12N 15/8213; C12N 9/1241; C12N 9/22; C12N 15/1086; C12N 15/11; C12N 15/8205; C12N 15/8242; C12N 15/8261; C12Q 1/6869; C12Q 1/6895; C07K 2319/09; C07K 2319/40; C12N 2310/20; C12Q 2600/13

(57) ABSTRACT

Disclosed herein include methods, compositions, and kits suitable for use in integration of a nucleic acid sequence into a double-stranded target sequence of a genome of a plant cell or a target plasmid in a plant cell. In some embodiments, the methods, systems and nucleic acid compositions comprise polynucleotides encoding and RNA-guided DNA binding complex, a transposition complex, for insertion of cargo sequence using transposition.

### RELATED APPLICATIONS

The present application claims priority to U.S. Provisional Application No. 63/551,713, filed Feb. 9, 2024. The entire contents of this application is hereby expressly incorporated by reference in their entireties.

### REFERENCE TO SEQUENCE LISTING

The present application is being filed along with a Sequence Listing in electronic format. The Sequence Listing is provided as a file entitled 30KJ-810004-US_SequenceListing, created Feb. 6, 2025, which is 332,753 bytes in size. The information in the electronic format of the Sequence Listing is incorporated herein by reference in its entirety.

### BACKGROUND

### Field

The present disclosure relates generally to the field of genetic engineering.

### Description of the Related Art

Climate change poses unprecedented challenges to food safety. The world's population is projected to reach 9.6 billion by 2050, resulting in a 60% surge in demand for crops, while detrimental environmental conditions continue to limit plant production. Given the projected reductions in crop yields, it becomes imperative to explore novel strategies to address the food security issue.

Plant genetic engineering has emerged as a promising avenue for imparting new traits, including enhanced resilience to environmental conditions, heightened crop yields, and improved quality. Nevertheless, engineering of plants faces multiple hurdles, ranging from the need for better understanding of plant genomes and expression profiles to the development of advanced DNA editing tools. Several challenges persist in the current genome editing toolbox that rely on targeted nucleases such as zinc finger nucleases (ZFNs), transcription activator-like effector nucleases (TALENs), and CRISPR/Cas system. One critical consideration is that double-strand breaks (DSBs) can give rise to undesirable byproducts, including small insertions and deletions (indels), large-scale genomic deletions, and chromosomal translocations. Additionally, homology-directed repair (HDR)-mediated DNA insertion in plants is challenging because HDR is only initiated in the S and G2 phase of the cell cycle and necessitates the supply of enough repair template. There is a need for improved gene editing techniques in plants.

### SUMMARY

Disclosed herein include systems for integration of a nucleic acid sequence into a double-stranded target sequence of a genome of a plant cell or a target plasmid in a plant cell. In some embodiments, the system comprises: i) an RNA-guided DNA binding complex or one or more first helper polynucleotides each comprising a sequence encoding a component of the RNA-guided DNA binding complex, wherein the RNA-guided DNA binding complex comprises one or more Cas proteins, a transposase, a crRNA, or any combination thereof, optionally the system comprises one or more helper accessory proteins or one or more helper accessory polynucleotides each comprising a sequence encoding at least one of the one or more helper accessory proteins, wherein the one or more helper accessory proteins comprise ClpX and ClpP; ii) a transposition complex or one or more second helper polynucleotides each comprising a sequence encoding a component of the transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

Disclosed herein include nucleic acid compositions for integration of a nucleic acid sequence into a double-stranded target sequence of a genome of a plant cell or a target plasmid in a plant cell. In some embodiments, the nucleic acid composition comprises: i) one or more first helper polynucleotides each comprising a sequence encoding a component of an RNA-guided DNA binding complex, wherein the RNA-guided DNA binding complex comprises one or more Cas proteins, a transposase, a crRNA, or any combination thereof, optionally the nucleic acid composition comprises one or more helper accessory polynucleotides each comprising a sequence encoding at least one of the one or more helper accessory proteins, wherein the one or more helper accessory proteins comprise ClpX and ClpP; ii) one or more second helper polynucleotides each comprising a sequence encoding a component of a transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

In some embodiments, at least one of the one or more first helper polynucleotides and/or at least one of the one or more second helper polynucleotides are comprised within a second autonomous replicon. In some embodiments, the first autonomous replicon, the second autonomous replicon, or both is derived from a geminivirus. In some embodiments, the geminivirus comprises cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, or tomato pseudo-curly top virus. In some embodiments, the donor polynucleotide comprised within the first autonomous replicon comprises, from 5′ to 3′: a first long intergenic region (LIR), the RE, the cargo sequence, the LE, a short intergenic region (SIR), a sequence encoding RepA, and a second LTR. In some embodiments, the at least one of the one or more first helper polynucleotides and/or the at least one of the one or more second helper polynucleotides comprised within the second autonomous replicon comprises, from 5′ to 3′: a first long intergenic region (LIR), the first helper polynucleotide or the second helper polynucleotide, a short intergenic region (SIR), a sequence encoding RepA, and a second LIR. In some embodiments, the first and/or second LIR comprise the sequence of SEQ ID NO: 1 or a sequence having one, two, or three mismatches relative to the sequence of SEQ ID NO: 1; the SIR comprises the sequence of SEQ ID NO: 2 or a sequence having one, two, or three mismatches relative to the sequence of SEQ ID NO: 2; and the sequence encoding RepA comprises the sequence of SEQ ID NO: 3 or a sequence that is at least 80%, 85%, 90%, 95%, 98%, 99%, or 100% identical to SEQ ID NO: 3. In some embodiments, the amount of the donor polynucleotide in the plant cell is capable of increasing by at least 2-fold following the onset of autonomous replication. In some embodiments, the amount of the donor polynucleotide in the plant cell is capable of increasing by at least 10-fold following the onset of autonomous replication. In some embodiments, the amount of a gene product encoded by the donor polynucleotide in the plant cell is capable of increasing by at least 2-fold following the onset of autonomous replication. In some embodiments, the amount of the gene product encoded by the donor polynucleotide in the plant cell is capable of increasing by at least 10-fold following the onset of autonomous replication.

In some embodiments, the LE comprises the sequence of any one of SEQ ID NOs: 4-5 or a sequence having one, two or three mismatches relative to the sequence of any one of SEQ ID NOs: 4-5 and wherein the RE comprises the sequence of any one of SEQ ID NOs: 6-7 or a sequence having one, two or three mismatches relative to the sequence of any one of SEQ ID NOs: 6-7. In some embodiments, the cargo sequence is 0.2 to 1000 kb in length. In some embodiments, the cargo sequence is 200 to 1200 bp in length.

In some embodiments, the cargo sequence comprises one or more exogenous sequences each selected from the group comprising: a nitrogen fixation gene, a plant stress-induced gene, a nutrient utilization gene, a gene that affects plant pigmentation, a gene that encodes an antisense or ribozyme molecule, a gene encoding an antigen capable of being secreted, a toxin gene, a receptor gene, a ligand gene, a seed storage gene, a hormone gene, an enzyme gene, an interleukin gene, a cytokine gene, a growth factor gene, a transcription factor gene, a transcriptional repressor gene, a DNA-binding protein gene, a recombination gene, a DNA replication gene, a programmed cell death gene, a kinase gene, a phosphatase gene, a G protein gene, a cyclin gene, a cell cycle control gene, a gene involved in transcription, a gene involved in translation, a gene involved in RNA processing, a gene involved in RNAi, an organellar gene, a intracellular trafficking gene, an integral membrane protein gene, a transporter gene, a membrane channel protein gene, a cell wall gene, a gene involved in protein processing, a gene involved in protein modification, a gene involved in protein degradation, a gene involved in metabolism, a gene involved in biosynthesis, a gene involved in assimilation of nitrogen or other elements or nutrients, a gene involved in controlling carbon flux, gene involved in respiration, a gene involved in photosynthesis, a gene involved in light sensing, a gene involved in organogenesis, a gene involved in embryogenesis, a gene involved in differentiation, a gene involved in meiotic drive, a gene involved in self incompatibility, a gene involved in development, a gene involved in nutrient, metabolite or mineral transport, a gene involved in nutrient, metabolite or mineral storage, a calcium-binding protein gene, and a lipid-binding protein gene. In some embodiments, the cargo sequence comprises one or more exogenous sequences each selected from the group comprising: a gene encoding an enzyme involved in metabolizing biochemical wastes for use in bioremediation, a gene that encodes an enzyme for modifying pathways that produce secondary plant metabolites, a gene that encodes an enzyme that produces a pharmaceutical, a gene that encodes an enzyme that improves or changes the nutritional content of a plant, a gene that encodes an enzyme involved in vitamin synthesis, a gene that encodes an enzyme involved in carbohydrate, polysaccharide or starch synthesis, a gene that encodes an enzyme involved in mineral accumulation or availability, a gene that encodes a phytase, a gene that encodes an enzyme involved in fatty acid, fat or oil synthesis, a gene that encodes an enzyme involved in synthesis of chemicals or plastics, a gene that encodes an enzyme involved in synthesis of a fuel, a gene that encodes an enzyme involved in synthesis of a fragrance, a gene that encodes an enzyme involved in synthesis of a flavor, a gene that encodes an enzyme involved in synthesis of a pigment or dye, a gene that encodes an enzyme involved in synthesis of a hydrocarbon, a gene that encodes an enzyme involved in synthesis of a structural or fibrous compound, a gene that encodes an enzyme involved in synthesis of a food additive, a gene that encodes an enzyme involved in synthesis of a chemical insecticide, a gene that encodes an enzyme involved in synthesis of an insect repellent, and a gene controlling carbon flux in a plant. In some embodiments, the cargo sequence comprises an exogenous sequence encoding a fluorescent protein. In some embodiments, the fluorescent protein comprises mPlum, mCherry, DsRed, FASTred, mOrange, EYFP, VENUS, YPet, GFP, EGFP, EmGFP, mCFP, Cerulean, CyPet, Kaede, or any combination thereof.

In some embodiments, the one or more Cas proteins comprise a Cas6 protein, a Cas7 protein, and a Cas8 protein. In some embodiments, the Cas6 protein comprises an amino acid sequence that is at least 80%, 85%, 90%, 95%, 98%, 99%, or 100% identical to any one of SEQ ID NOs: 8-9; the Cas7 protein comprises an amino acid sequence that is at least 80%, 85%, 90%, 95%, 98%, 99%, or 100% identical to any one of SEQ ID NOs: 10-11; and/or the Cas8 protein comprises an amino acid sequence that is at least 80%, 85%, 90%, 95%, 98%, 99%, or 100% identical to any one of SEQ ID NOs: 12-13. In some embodiments, the transposase of the RNA-guided DNA binding complex comprises a TniQ protein. In some embodiments, the TniQ protein comprises an amino acid sequence that is at least 80%, 85%, 90%, 95%, 98%, 99%, or 100% identical to any one of SEQ ID NOs: 14-15. In some embodiments, the one or more transposases of the transposition complex comprise a TnsA transposase, a TnsB transposase, and a TnsC protein. In some embodiments, the one or more transposases of the transposition complex comprise a TnsAB fusion protein and a TnsC protein. In some embodiments, the TnsAB fusion protein comprises an amino acid sequence that is at least 80%, 85%, 90%, 95%, 98%, 99%, or 100% identical to any one of SEQ ID NOs: 16-17; and/or the TnsC protein comprises an amino acid sequence that is at least 80%, 85%, 90%, 95%, 98%, 99%, or 100% identical to any one of SEQ ID NOs: 18-19. In some embodiments, the system, the RNA-guided DNA binding complex and/or the transposition complex is derived from a Type I-B, Type I-D, TypeI-F, or Type V-K Crispr-associated transposase system of a bacteria. In some embodiments, the bacteria comprise Vibrio cholera (Vch), Pseudoalteromonas (Pse), or Scytonema hofmanni (Sho). In some embodiments, the ClpX comprises an amino acid sequence that is at least 80%, 85%, 90%, 95%, 98%, 99%, or 100% identical to SEQ ID NO: 21; and/or the ClpP comprises an amino acid sequence that is at least 80%, 85%, 90%, 95%, 98%, 99%, or 100% identical to SEQ ID NO: 20. In some embodiments, at least one of the one or more Cas proteins and/or the transposase of the RNA-guided DNA binding complex comprise a nuclear localization signal (NLS). In some embodiments, the NLS is an N-terminal NLS or a C-terminal NLS; at least one of the one or more transposases of the transposition complex comprises an NLS. In some embodiments, the NLS is an N-terminal NLS or a C-terminal NLS; and/or at least one of the one or more helper accessory proteins comprises an NLS. In some embodiments, the NLS is an N-terminal NLS or a C-terminal NLS. In some embodiments, the TnsAB fusion protein comprises an NLS between the TnsA amino acid sequence and the TnsB amino acid sequence. In some embodiments, the TnsAB fusion protein comprises, from N-terminus to C-terminus: TnsA, the NLS, and TnsB. In some embodiments, the NLS comprises an amino acid sequence encoded by a nucleotide sequence of any one of SEQ ID NOs: 22-23 or a sequence have one, two, or three mismatches relative to any one of SEQ ID NOs: 22-23.

In some embodiments, the crRNA comprises a spacer that is complementary to a search target sequence on a first strand of the double stranded target sequence. In some embodiments, the crRNA comprises a [repeat scaffold]-[spacer]-[repeat scaffold] structure. In some embodiments, the first strand of the double stranded target sequence is the sense strand. In some embodiments, the cargo sequence is capable of being integrated at an integration site following binding of the RNA-guided DNA binding complex to the search target sequence, wherein the integration site is about 48 to 52 base pairs downstream of the double stranded target sequence. In some embodiments, the double stranded target sequence is situated within a selectable marker gene of the genome of the plant cell. In some embodiments, the selectable marker gene comprises a fluorescent protein coding gene, a phytoene desaturase (PDS) gene, a codA gene, a diphtheria toxin a subunits (DT-A) gene, an exotoxin A gene, a ricin toxin A gene, a cytochrome P-450 gene, an RNase T1 gene, or a barnase gene. In some embodiments, the double stranded target sequence is situated within a safe harbor locus of the genome of the plant cell.

In some embodiments, each of the one or more first helper polynucleotides comprises a first promoter operably linked to the sequence encoding the component of the RNA-guided DNA binding complex; each of the one or more second helper polynucleotides comprises a second promoter operably linked to the sequence encoding the component of the transposition complex; and/or each of the one or more helper accessory polynucleotides comprises a third promoter operably linked to the sequence encoding at least one of the one or more helper accessory proteins. In some embodiments, the first, second, and/or third promoters are the same or different. In some embodiments, the first, second, and/or third promoters comprise a ubiquitous promoter, a constitutive promoter, a cell-type specific promoter, a tissue-specific promoter, an inducible promoter, or any combination thereof. In some embodiments, the constitutive promoter is selected from the group comprising: pCmYLCV911 (pCmY), pU6, pU3, pU6, pAct2, pAct-1, pUBQ10, pUBQ4, pUbi1, and PUbi2; the tissue-specific promoter is selected from the group comprising: pSIREO, pNAC10, pPAT21, phspr, pPFn2, pPEPC, PLhcb, pTA29, pLat52, pZml3, pOleosin, pGlutenin, pD-hordein, and pE8; the inducible promoter is selected from the group comprising: pAdh-1, pwunl, pGBSS, pHSP18.2, pRd29, pSR2, pCCA1, pUGT71C5, pGSE, pwin3.12, pR2329, pBs3, pCaPrx, p4xM1.1, p4xM2.3, pIFS2, pSAG12, pSEOF1, pEm, pRd29, pSAUR15A, and pChn48. In some embodiments, each of the one or more first helper polynucleotides comprises a first transcription terminator operably linked to the sequence encoding the component of the RNA-guided DNA binding complex; each of the one or more second helper polynucleotides comprises a second transcription terminator operably linked to the sequence encoding the component of the transposition complex; and/or each of the one or more helper accessory polynucleotides comprises a third transcription terminator operably linked to the sequence encoding at least one of the one or more helper accessory proteins. In some embodiments, the first, second, and/or third transcription terminators are the same or different. In some embodiments, the first, second, and/or third transcription terminators comprise AtHSP18.2 (tHSP), tU6, tACT3, tACT3-tRb7MAR, tACT3-tTM6MAR, tEU, tEU-tTM6MAR, tEU (intronless), tEU (intronless)-tACT3-tRB7MAR, tHSP18-tEU-tRb7MAR, tHSP18-tACT3, tHSP18-tACT3-tRb7, tHSP18-tPINII-tRb7MAR, tHSP18-tPINII-tTM6MAR, tHSP18-tRb7MAR, tProteinase inhibitor II (tPINII), trbcS, or any combination thereof.

The system or the nucleic acid composition can comprise: at least three first helper polynucleotides each comprising a sequence encoding a Cas protein, wherein the sequence FIG. 3 helper polynucleotide comprising a sequence encoding a transposase protein, wherein the sequence encoding the transposase protein is operably linked to a pCmY promoter and a tHSP terminator; a first helper polynucleotide comprising a sequence encoding a crRNA, wherein the sequence encoding the crRNA is operably linked to a pU6 promoter and a tU6 terminator; and at least two second helper polynucleotides each comprising a sequence encoding a transposase, wherein the sequence encoding a transposase is operably linked to a pCmY promoter and a tHSP terminator. The system or the nucleic acid composition can comprise at least two helper accessory polynucleotides, wherein the sequence encoding at least one of the one or more helper accessory proteins is operably linked to a pCmY promoter and a tHSP terminator.

In some embodiments, the sequence encoding the component of the RNA-guided DNA binding complex, the sequence encoding the component of the transposition complex, the sequence encoding at least one of the one or more helper accessory proteins, or any combination thereof, is codon optimized for expression in the plant cell. In some embodiments, the sequence encoding the component of the RNA-guided DNA binding complex encodes a Cas6 protein. In some embodiments, the sequence encoding the Cas6 protein comprises the nucleotide sequence of any one of SEQ ID NOs: 24-25 or a sequence that is at least 80%, 85%, 90%, 95%, 98%, 99%, or 100% identical to any one of SEQ ID NOs: 24-25. In some embodiments, the sequence encoding the component of the RNA-guided DNA binding complex encodes a Cas7 protein. In some embodiments, the sequence encoding the Cas7 protein comprises the nucleotide sequence of any one of SEQ ID NOs: 26-27 or a sequence that is at least 80%, 85%, 90%, 95%, 98%, 99%, or 100% identical to any one of SEQ ID NOs: 26-27. In some embodiments, the sequence encoding the component of the RNA-guided DNA binding complex encodes a Cas8 protein. In some embodiments, the sequence encoding the Cas8 protein comprises the nucleotide sequence of any one of SEQ ID NOs: 28-29 or a sequence that is at least 80%, 85%, 90%, 95%, 98%, 99%, or 100% identical to any one of SEQ ID NOs: 28-29. In some embodiments, the sequence encoding the component of the RNA-guided DNA binding complex encodes a TniQ protein. In some embodiments, the sequence encoding the TniQ protein comprises the nucleotide sequence of any one of SEQ ID NOs: 30-31 or a sequence that is at least 80%, 85%, 90%, 95%, 98%, 99%, or 100% identical to any one of SEQ ID NOs: 30-31. In some embodiments, the sequence encoding the component of the transposition complex encodes a TnsAB fusion protein. In some embodiments, the sequence encoding the TnsAB fusion protein comprises the nucleotide sequence of any one of SEQ ID NOs: 32-33 or a sequence that is at least 80%, 85%, 90%, 95%, 98%, 99%, or 100% identical to any one of SEQ ID NOs: 32-33. In some embodiments, the sequence encoding the component of the transposition complex encodes a TnsC protein. In some embodiments, the sequence encoding the TnsC protein comprises the nucleotide sequence of any one of SEQ ID NOs: 34-35 or a sequence that is at least 80%, 85%, 90%, 95%, 98%, 99%, or 100% identical to any one of SEQ ID NOs: 34-35. In some embodiments, the sequence encoding ClpX comprises the nucleotide sequence of SEQ ID NO: 37 or a sequence that is at least 80%, 85%, 90%, 95%, 98%, 99%, or 100% identical to SEQ ID NO: 37 and the sequence encoding ClpP comprises the nucleotide sequence of SEQ ID NO: 36 or a sequence that is at least 80%, 85%, 90%, 95%, 98%, 99%, or 100% identical to SEQ ID NO: 36.

In some embodiments, the component of the RNA-guided DNA binding complex, the component of the transposition complex, or both, comprises an N-terminal or a C-terminal tag. In some embodiments, the tag is an epitope tag. In some embodiments, the epitope tag comprises a myc tag, a FLAG tag, a polyHistidine tag, a HiBiT tag, HA tag, S-peptide tag, calmodulin-binding peptide (CBP), glutathione S-transferase (GST), maltose binding protein (MBP), or any combination thereof. In some embodiments, the tag comprises a fluorescent protein. In some embodiments, the fluorescent protein comprises mPlum, mCherry, DsRed, FASTred, mOrange, EYFP, VENUS, YPet, GFP, EGFP, EmGFP, mCFP, Cerulean, CyPet, Kaede, or any combination thereof.

In some embodiments, the one or more first helper polynucleotides the one or more second helper polynucleotides, and/or the donor polynucleotide are situated on the same nucleic acid or different nucleic acids. In some embodiments, the one or more first helper polynucleotides, the one or more second helper polynucleotides, and/or the donor polynucleotide are comprised within one or more vectors. In some embodiments, the one or more vectors comprise an RNA viral vector, a DNA viral vector, a plasmid vector, an artificial chromosome, or any combination thereof. In some embodiments, the one or more vectors comprise an Agrobacterium tumefaciens Ti vector. In some embodiments, the one or more first helper polynucleotides, the one or more second helper polynucleotides, and/or the donor polynucleotide are comprised within a T-DNA region of the Agrobacterium tumefaciens Ti vector. In some embodiments, the T-DNA region comprising the one or more first helper polynucleotides, the one or more second helper polynucleotides, and/or the donor polynucleotide comprises the sequence of any one of SEQ ID NOs: 38-46.

Disclosed herein include methods for integration of a nucleic acid sequence into double-stranded target sequence of a genome of a plant cell or a target plasmid in a plant cell. In some embodiments, the method comprises: contacting the plant cell with a system or the nucleic acid composition of the disclosure, wherein the cargo sequence is integrated at an integration site in the genome of the plant cell or at a target site of the target plasmid upon expression of the RNA-guided DNA binding complex and the transposition complex in the plant cell.

In some embodiments, the integration site is about 48 to 52 base pairs downstream of the double stranded target sequence. In some embodiments, the one or more Cas proteins and the transposase of the RNA-guided DNA binding complex are pre-complexed with the crRNA prior to the contacting. In some embodiments, the plant cell is comprised within a plant. In some embodiments, the plant cell is comprised within a flower, a leaf, a stem, a root, terminal bud, a seed, or any other tissue of the plant. In some embodiments, the plant cell is a monocot plant cell or a eudicot plant cell. In some embodiments, the integration of the cargo sequence confers i) a change in one or more of the following traits to the plant: grain number, grain size, grain weight, panicle size, tiller number, fragrance, nutritional value, shelf life, lycopene content, starch content and/or ii) lower gluten content, reduced levels of a toxin, reduced levels of steroidal glycoalkaloids, a substitution of mitosis for meiosis, asexual propagation, improved haploid breeding, and/or shortened growth time. In some embodiments, the integration of the cargo sequence confers one or more of the following traits to the plant cell and/or the plant: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease. In some embodiments, the system or the nucleic acid composition is introduced into the plant cell by a technique comprising: pollen tube pathway, polyethylene glycol (PEG)-mediated gene transfer, electroporation, microinjection, microparticle bombardment, nanomaterial-mediated delivery, Agrobacterium tumefaciens-mediated transformation, or any combination thereof. In some embodiments, the nanomaterial-mediated delivery comprises: clay nanosheets, carbon nanotubes, carbon nanodots, self-assembled protein nanoparticles, peptides, DNA nanostructures, quantum dots, or any combination thereof. In some embodiments, the one or more vectors are introduced into the plant cell via Agrobacterium tumefaciens-mediated transformation of the plant cell.

Disclosed herein include methods for screening for safe harbor loci in plants. In some embodiments, the method comprises: (a) generating a genome-wide crRNA library; (b) contacting a plant cell comprised within a plant with a system or the nucleic acid composition of disclosure, wherein: the system comprises pooled single or combinatorial crRNAs generated in step (a); or the one or more first helper polynucleotides comprise pooled single or combinatorial crRNAs generated in step a), wherein the cargo sequence is integrated into one or more double-stranded targets sites in the genome of the plant cell upon expression of the RNA-guided DNA binding complex and the transposition complex in the plant cell; (c) identifying integrants by expression of a gene product encoded by the cargo sequence; (d) subjecting the integrants to next-generation sequencing; and (e) performing bioinformatics analysis, a high-throughput phenotypic assay, or both to identify a safe harbor locus.

In some embodiments, the plant is a monocot plant or a eudicot plant. In some embodiments, integration of the cargo sequence at the identified safe harbor locus does not affect the growth, lifespan, health, gene expression profile, or any combination thereof, of the plant. In some embodiments, the system or the nucleic acid composition is introduced into the plant cell by a technique comprising: pollen tube pathway, polyethylene glycol (PEG)-mediated gene transfer, electroporation, microinjection, microparticle bombardment, nanomaterial-mediated delivery, Agrobacterium tumefaciens-mediated transformation, or any combination thereof. In some embodiments, the nanomaterial-mediated delivery comprises: clay nanosheets, carbon nanotubes, carbon nanodots, self-assembled protein nanoparticles, peptides, DNA nanostructures, quantum dots, or any combination thereof. In some embodiments, the one or more vectors are introduced into the plant cell via Agrobacterium tumefaciens-mediated transformation of the plant cell. In some embodiments, the T-DNA region of the Ti vector comprises a bi-directional selection marker comprising a positive selection marker and a negative selection marker, wherein the identifying of step c) comprises: (i) generating a first filial generation (F1) plant comprising the T-DNA of the Ti vector by positive selection; and (ii) generating a second filial generation (F2) plant that does not comprise the T-DNA of the Ti vector from the first filial generation plant comprising the T-DNA of the Ti vector, by negative selection.

Disclosed herein include kits comprising a system or nucleic acid composition described herein, and a set of instructions for use.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 displays a construct design for helper, donor, and target plasmids.

FIG. 2A-FIG. 2D display exemplary data related to quantification of luminescence signal from HiBiT-tagged CAST proteins. FIG. 2A displays a schematic of construct design. The upper panel was used to express the VchCAST and PseCAST proteins with the exception of the TnsA-B construct in which the Bipartite (BP) NLS was inserted in between the two proteins. The lower panel was used to express the ShoCAST proteins. FIG. 2B-FIG. 2D display exemplary expression data. Data is presented as luminescence values of three replicates normalized to total protein concentration per sample for the VchCAST, PseCAST and ShoCAST nuclear and cytoplasmic fractions respectively. One way ANOVA test (p<0.05).

FIG. 3A-FIG. 3B display schematics of CAST-mediated episomal DNA integration in protoplast cells. FIG. 3A shows an exemplary construct design: Upper panel illustrates the generic Helper, Donor, and Target plasmid designs. Lower panel shows the target loci before and after integration. FIG. 3B displays a schematic of an experimental procedure of CAST-mediated episomal DNA integration: PEG transfection of genetic materials into A. thaliana protoplasts and downstream phenotypic and molecular analysis.

FIG. 4 displays representative images showing DsRed- and mTurq-positive protoplasts confirmed the co-transfection of plasmids and YFP-positive protoplasts and the successful integration events.

FIG. 5A-FIG. 5C display exemplary data related to the successful integration junctions detected via nested PCR. FIG. 5A shows primer design for OUT and IN nested PCR reactions for detecting the junctions in favored orientation of Target-RE-Cargo-LE. Nested PCR results of the (FIG. 5B) RE and (FIG. 5C) LE junctions of Vch and Pse-mediated integration in protoplasts. Squares indicate the expected bands with the correct integration product size.

FIG. 6A-FIG. 6D display data and schematics showing successful integration junctions detected via Sanger sequencing. Integration junctions from (FIG. 6A) Vch T2 (FIG. 6B) Vch T3 (FIG. 6C) Pse T2 and (FIG. 6D) Pse T3 RE and LE amplification products are shown.

FIG. 7A-FIG. 7B display representative schematics of CAST-mediated DNA integration into the plant genome. FIG. 7A displays a representative construct design. Upper panel illustrates the generic Helper and Donor plasmids design. Lower panel illustrates the genomic target loci before and after integration. FIG. 7B displays a schematic of an experimental procedure of CAST-mediated chromosomal DNA integration, including Agrobacterium-mediated transformation of genetic materials into N. benthamiana leaves, and downstream phenotypic and molecular analysis.

FIG. 8A-FIG. 8C display exemplary data related to the successful integration junctions detected via nested PCR and Sanger sequencing. FIG. 8A shows primer design for OUT and IN nested PCR reactions for detecting the RE junction in favored orientation Target-RE-Cargo-LE. FIG. 8B shows exemplary nested PCR results of the RE junctions of Vch and Pse mediated integration in protoplasts. Square indicates the expected band with the correct integration product size. Shown in FIG. 8C displays a diagram of the integration site from Pse T2 RE.

FIG. 9A-FIG. 9B display schematics of engineering a stable transgenic Arabidopsis with CAST. FIG. 9A shows the construct design. Upper panel illustrates the generic Helper and Donor plasmids design. Lower panel illustrates the genomic target loci PDS or codA before and after integration. Shown in FIG. 9B is an experimental procedure of CAST mediated stable chromosomal DNA integration, including Agrobacterium-mediated transformation of genetic materials into A. thaliana flower, and downstream phenotypic and molecular analysis.

FIG. 10 displays a schematic of an exemplary protocol for application of CAST to screen for genomic safe harbor loci.

FIG. 11A-FIG. 11D display exemplary data related to confocal imaging and HiBiT lytic detection of CAST protein expression. FIG. 11A displays schematics of construct design. FIG. 11B displays a schematic of protein expression and detection workflow. FIG. 11C displays exemplary images of confocal microscopy detection of HiBiT tagged YPet-VchCAST fusion proteins with an N-terminus BP NLS. FIG. 11D displays quantification of HiBiT tagged YPet-VchCAST fusion proteins detected as luminescence signal. n=1 biological replicate.

FIG. 12A-FIG. 12F display exemplary data related to quantification of luminescence signal from HiBiT-tagged CAST proteins. FIG. 12A displays a schematic of the HiBiT Lytic Assay. FIG. 12B displays a schematic of the construct design used to express the VchCAST and PseCAST proteins tagged with an N-terminal HiBiT peptide tag. Shown in FIG. 12C-FIG. 12D are luminescence values for the VchCAST and PseCAST proteins in nuclear and cytoplasmic fractions, respectively. Data are normalized to total protein concentration per sample and represent a minimum of three replicates. Ordinary one-way ANOVA test, *p<0.05, **p<0.01, ***p<0.001, and ****p<0.0001. FIG. 12E displays a schematic of the construct design for CAST transposases (TnsA-B fusion) flanked by a Geminiviral replicon. FIG. 12F shows a graph of luminescence values reflecting transposase protein expression for the VchCAST and PseCAST proteins. Data are normalized to total protein concentration per sample and represent a minimum of three replicates. Ordinary one-way ANOVA test, *p<0.05, **p<0.01, ***p<0.001, and ****p<0.0001. For all graphs, data are shown as aligned dot plots with horizontal lines indicating the mean.

FIG. 13A-FIG. 13C display exemplary data showing mTurq- and YPET-positive protoplasts confirmed the co-transfection at different timepoints. The time points shown are 12 hr (FIG. 13A), 24 hr (FIG. 13B), and 48 hr (FIG. 13C). mTurq- and YPET-positive protoplasts confirmed the co-transfection at different timepoints. mCherry signal was detected only in experimental groups at 12 and 24 hours, but detected in both negative and experiment groups at 48 hours.

FIG. 14A-FIG. 14C display exemplary data showing successful integration junctions detected via nested PCR. Primer design for OUT and IN nested PCR reactions for detecting the junctions in favored orientation of Target-RE-Cargo-LE is shown in FIG. 14A. Also shown are nested PCR results of the RE (FIG. 14B) and LE (FIG. 14C) junctions of PseCAST-mediated integration in protoplasts.

FIG. 15A-FIG. 15B display data showing quantification of integration events via nested TaqMan probe-based qPCR. TaqMan probes and primers targeting Cas8 gene, RE junction, and LE junction are shown in FIG. 15A. FIG. 15B shows qPCR amplification curve and Ct values.

FIG. 16A-FIG. 16B show exemplary amplicon sequencing to determine the integration site distribution. FIG. 16A displays gel images of the first and second round of PCR, showing the expected bands. Shown in FIG. 16B are graphs of integration distance profiles of different samples.

FIG. 17A-FIG. 17B display molecular analysis of CAST function in leaf genome. Primer design for OUT and IN nested PCR reactions for detecting the RE and LE junctions in favored orientation Target-RE-Cargo-LE is shown in FIG. 17B. Shown in FIG. 17B are nested PCR results of the RE and LE junctions from PseCAST-mediated integration in 16c N. benthamiana leaves. Square indicates the bands with the expected integrated product size.

FIG. 18A-FIG. 18B display construct design and confocal images of CAST integration in protoplast genome. FIG. 18A displays a diagram of the constructs used for CAST integration: pTNP-CAS and pDonor-TnsAB-Clp. FIG. 18B shows exemplary confocal images of YPET- and DsRed-positive protoplasts, confirming the successful co-transfection of both plasmids.

FIG. 19 displays exemplary data related to the successful integration junctions detected via nested PCR. Nested PCR results of the RE and LE junctions from Pse mediated integration in 16c N. benthamiana protoplasts. Also shown is a diagram of a simulated integrated sequence.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include systems for integration of a nucleic acid sequence into a double-stranded target sequence of a genome of a plant cell or a target plasmid in a plant cell. In some embodiments, the system comprises: i) an RNA-guided DNA binding complex or one or more first helper polynucleotides each comprising a sequence encoding a component of the RNA-guided DNA binding complex, wherein the RNA-guided DNA binding complex comprises one or more Cas proteins, a transposase, a crRNA, or any combination thereof, optionally the system comprises one or more helper accessory proteins or one or more helper accessory polynucleotides each comprising a sequence encoding at least one of the one or more helper accessory proteins, wherein the one or more helper accessory proteins comprise ClpX and ClpP; ii) a transposition complex or one or more second helper polynucleotides each comprising a sequence encoding a component of the transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

Disclosed herein include nucleic acid compositions for integration of a nucleic acid sequence into a double-stranded target sequence of a genome of a plant cell or a target plasmid in a plant cell. In some embodiments, the nucleic acid composition comprises: i) one or more first helper polynucleotides each comprising a sequence encoding a component of an RNA-guided DNA binding complex, wherein the RNA-guided DNA binding complex comprises one or more Cas proteins, a transposase, a crRNA, or any combination thereof, optionally the nucleic acid composition comprises one or more helper accessory polynucleotides each comprising a sequence encoding at least one of the one or more helper accessory proteins, wherein the one or more helper accessory proteins comprise ClpX and ClpP; ii) one or more second helper polynucleotides each comprising a sequence encoding a component of a transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

Disclosed herein include methods for integration of a nucleic acid sequence into double-stranded target sequence of a genome of a plant cell or a target plasmid in a plant cell. In some embodiments, the method comprises: contacting the plant cell with a system or the nucleic acid composition of the disclosure, wherein the cargo sequence is integrated at an integration site in the genome of the plant cell or at a target site of the target plasmid upon expression of the RNA-guided DNA binding complex and the transposition complex in the plant cell.

Disclosed herein include methods for screening for safe harbor loci in plants. In some embodiments, the method comprises: (a) generating a genome-wide crRNA library; (b) contacting a plant cell comprised within a plant with a system or the nucleic acid composition of disclosure, wherein: the system comprises pooled single or combinatorial crRNAs generated in step (a); or the one or more first helper polynucleotides comprise pooled single or combinatorial crRNAs generated in step a), wherein the cargo sequence is integrated into one or more double-stranded targets sites in the genome of the plant cell upon expression of the RNA-guided DNA binding complex and the transposition complex in the plant cell; (c) identifying integrants by expression of a gene product encoded by the cargo sequence; (d) subjecting the integrants to next-generation sequencing; and (e) performing bioinformatics analysis, a high-throughput phenotypic assay, or both to identify a safe harbor locus.

Disclosed herein include kits comprising a system or nucleic acid composition described herein, and a set of instructions for use.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” means plus or minus 5% of the provided value.

As used herein, the term “double-stranded target DNA” refers to a DNA that includes a “target site” or “target sequence.” The term “target sequence” is used herein to refer to a nucleic acid sequence present in a double-stranded target DNA to which a DNA-targeting sequence or segment (also referred to herein as a “spacer”) of a crRNA can hybridize, provided sufficient conditions for hybridization exist. For example, the target sequence 5′-GAGCATATC-3′ within a target DNA is targeted by (or is capable of hybridizing with, or is complementary to) the RNA sequence 5′-GAUAUGCUC-3′. Hybridization between the DNA-targeting sequence or segment of a crRNA and the target sequence can, for example, be based on Watson-Crick base pairing rules, which enables programmability in the DNA-targeting sequence or segment. The DNA-targeting sequence or segment of a crRNA can be designed, for instance, to hybridize with any target sequence.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases. In some embodiments, a polynucleotide comprises a nucleotide sequence encoding a gene product operably linked to one or more expression control elements (e.g., a promoter), as an expression cassette.

As used herein, the term “binding” refers to a non-covalent interaction between macromolecules (e.g., between a protein and a nucleic acid). While in a state of non-covalent interaction, the macromolecules are said to be “associated” or “interacting” or “binding” (e g., when a molecule X is said to interact with a molecule Y, it means that the molecule X binds to molecule Y in a non-covalent manner). Binding interactions can be characterized by a dissociation constant (Kd), for example a Kd of, or a Kd less than, 10′6 M, 10″ 7 M, 10′8 M, 10′9M, 10′10 M, 10′11 M, 10′12M, 10′13 M, IO′14 M, 10′15M, or a number or a range between any two of these values. Kd can be dependent on environmental conditions, e.g., pH and temperature. “Affinity” refers to the strength of binding, and increased binding affinity is correlated with a lower Kd.

The terms “complementarity” and “complementary” mean that a nucleic acid can form hydrogen bond(s) with another nucleic acid based on traditional Watson-Crick base paring rule, that is, adenine (A) pairs with thymine (U) and guanine (G) pairs with cytosine (C). Complementarity can be perfect (e.g. complete complementarity) or imperfect (e.g. partial complementarity). Perfect or complete complementarity indicates that each and every nucleic acid base of one strand is capable of forming hydrogen bonds according to Watson-Crick canonical base pairing with a corresponding base in another, antiparallel nucleic acid sequence. Partial complementarity indicates that only a percentage of the contiguous residues of a nucleic acid sequence can form Watson-Crick base pairing with the same number of contiguous residues in another, antiparallel nucleic acid sequence. In some embodiments, the complementarity can be at least 70%, 80%, 90%, 100% or a number or a range between any two of these values. In some embodiments, the complementarity is perfect, i.e. 100%. For example, the complementary candidate sequence segment is perfectly complementary to the candidate sequence segment, whose sequence can be deducted from the candidate sequence segment using the Watson-Crick base pairing rules.

The term “vector” as used herein, can refer to a vehicle for carrying or transferring a nucleic acid. Non-limiting examples of vectors include plasmids, bacteria, and viruses (for example, Agrobacterium tumefaciens Ti vectors).

The term “construct,” as used herein, can refer to a recombinant nucleic acid that has been generated for the purpose of the expression of a specific nucleotide sequence(s), or that is to be used in the construction of other recombinant nucleotide sequences. As used herein, the term “plasmid” can refer to a nucleic acid that can be used to replicate recombinant DNA sequences within a host organism. The sequence can be a double stranded DNA.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, “sequence identity” or “identity” in the context of two nucleic acid or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

Described herein are systems, compositions, and methods, for insertion of a cargo sequence into a target double-stranded DNA in a plant cell. Described herein is a CRISPR-associated transposases (CAST) system in plants for programmable and high efficiency DNA integration, merging CRISPR RNA-guided targeting with high insertion efficiency of transposases. CAST systems were first engineered into a powerful RNA-guided DNA-insertion tool in E. coli with nearly 100% efficiency upon selection, obviating the need for DSB in the target DNA or homology arms in the donor DNA. In 2023, two CASTs derived from Vibrio cholerae and Pseudoalteromonas, designated as Vch CAST and Pse CAST, have demonstrated ability in catalyzing the insertion of large DNA sequences in a targeted manner without inducing DSBs in mammalian cells.

Such biotechnology for plants will enable basic discoveries in plant genomics, such as the identification of essential genes and screening of ideal locus for exogenous gene insertion and expression. It will also allow improved capabilities, such as building developmental or metabolic pathways to provide biotic and abiotic stress tolerance, battle new plant epidemics and adverse effects of climate change, and enable scalable and affordable biosynthesis of valuable products in plants.

Disclosed herein are novel methodologies for targeted DNA integration in plants through the utilization of an engineered CAST system. This work creates an advanced genetic engineering toolbox, presenting opportunities for researchers engaged in the exploration of fundamental plant biology, as well as in the engineering of plants to gain desired traits and to facilitate molecular farming.

The disclosure covers the establishment of a CAST-mediated DNA integration technique in plants, along with the validation of its functionality through the integration of fluorescent cargo in Arabidopsis thaliana protoplasts and Nicotiana benthamiana leaves. Additionally, the disclosure delineates the proposed procedure to engineer a stable plant and employ it for safe harbor loci screening.

CRISPR-associated transposons or CASTs are mobile genetic elements (MGEs) that have evolved to make use of minimal CRISPR systems for RNA-guided transposition of their DNA. Unlike traditional CRISPR systems that contain interference mechanisms to degrade targeted DNA, CASTs lack proteins and/or protein domains responsible for DNA cleavage. Specialized transposon machinery, similar to that of Tn7 transposon, complexes with the CRISPR RNA (crRNA) and associated Cas proteins for transposition. CAST systems have been characterized in a wide range of bacteria and make use of variable CRISPR configurations including Type I-F, Type I-B, Type I-C, Type I-D, Type I-E, Type IV, and Type V-K.

Many CRISPR-associated transposons are similar to the Tn7 transposon which functions with a cut and paste mechanism. It contains a heteromeric transposase consisting of TnsA and TnsB proteins, and a regulator protein TnsC. Structural analysis has shown binding of the TnsB protein and sequence specific motifs on the ends of the transposon which allows for excision and mobility. Targeting for integration is done by the TnsD or TnsE proteins which preferentially target safe sites within the host chromosome or mobile elements (plasmids or bacteriophages), respectively. TnsE is not found in CASTs but a TnsD homolog, TniQ, is present and functions to bridge the gap between the transposase and CRISPR-Cas. Multiple CRISPR types have been found to associate with transposons with two of the most studied being Type I-F, which makes use of a multi-subunit effector, and Type V-K, which makes use of a single Cas12k effector. In both cases, Tn7 transposons have evolved to make use of these effectors to create R loops for site-specific integration. While TnsA is present in Type I-F systems, it is notably absent in Type V-K systems which showed higher off-target integrations during initial characterization.

A Type IF-3 CAST (Tn6677) was initially identified in Vibrio Cholerae and has been extensively studied. This system contains proteins TnsA, TnsB, and TnsC that complex with Cas6, Cas7, and a Cas5-Cas8 fusion through interactions with TniQ. Initial integration steps include TniQ complexed with Cas proteins, which binds at the target site, and TnsA and TnsB excision of the transposon, which is followed by TnsC binding to TniQ and transposase binding to TnsC. There can be off-targeting prior to this final step, but TnsB and TnsC binding leads to a final proofreading step to maintain a high on-target percentage. Tn6677 integration has been validated at near 100% on-target efficiency at site specific locations in multiple points in the host genome. Other systems have also been characterized and validated in this class with varying ranges of efficiency, and include orthogonal systems for multiplexed insertions up to 10 kb.

A Type V-K system was originally characterized from a cyanobacteria, Scytonema hofmanni, and contains a single Cas effector, Cas12k, that functions with a tracrRNA. This system functions similarly to Tn7 but does not have a TnsA protein which can result in off-targeting and chimera formation during over-expression. The Cas12k and tracrRNA complex bind to the target site and TnsC is polymerized directly adjacent prior to TniQ attachment and TnsB recognition and integration. While these systems use traditional tracrRNA characteristic of Type II CRISPR systems, they can also target with short crRNA located adjacent to the transposon end. Type V-K spacers preferentially target locations near tRNA genes, but other sites have been observed in these short crRNA guides.

Disclosed herein include systems for integration of a nucleic acid sequence into a double-stranded target sequence of a genome of a plant cell or a target plasmid in a plant cell. In some embodiments, the system comprises: i) an RNA-guided DNA binding complex or one or more first helper polynucleotides each comprising a sequence encoding a component of the RNA-guided DNA binding complex, wherein the RNA-guided DNA binding complex comprises one or more Cas proteins, a transposase, a crRNA, or any combination thereof, optionally the system comprises one or more helper accessory proteins or one or more helper accessory polynucleotides each comprising a sequence encoding at least one of the one or more helper accessory proteins, wherein the one or more helper accessory proteins comprise ClpX and ClpP; ii) a transposition complex or one or more second helper polynucleotides each comprising a sequence encoding a component of the transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

Disclosed herein include nucleic acid compositions for integration of a nucleic acid sequence into a double-stranded target sequence of a genome of a plant cell or a target plasmid in a plant cell. In some embodiments, the nucleic acid composition comprises: i) one or more first helper polynucleotides each comprising a sequence encoding a component of an RNA-guided DNA binding complex, wherein the RNA-guided DNA binding complex comprises one or more Cas proteins, a transposase, a crRNA, or any combination thereof, optionally the nucleic acid composition comprises one or more helper accessory polynucleotides each comprising a sequence encoding at least one of the one or more helper accessory proteins, wherein the one or more helper accessory proteins comprise ClpX and ClpP; ii) one or more second helper polynucleotides each comprising a sequence encoding a component of a transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

### Autonomous Replicons

In some embodiments of the systems and nucleic acid composition of the disclosure: a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon. At least one of the one or more first helper polynucleotides and/or at least one of the one or more second helper polynucleotides can be comprised within a second autonomous replicon. The first autonomous replicon, the second autonomous replicon, or both can be derived from a geminivirus. The geminivirus can comprise cabbage leaf curl virus, tomato golden mosaic virus, bean yellow dwarf virus, African cassava mosaic virus, wheat dwarf virus, miscanthus streak mastrevirus, tobacco yellow dwarf virus, tomato yellow leaf curl virus, bean golden mosaic virus, beet curly top virus, maize streak virus, or tomato pseudo-curly top virus.

Geminiviruses replicate through a rolling circle replication (RCR) cycle, and consequently, viral replicons can achieve high copy number, increasing the transient expression of, e.g., a donor polynucleotide and/or one or more first and/or second helper polynucleotides. For example, a deconstructed version of bean yellow dwarf virus (BeYDV), was used to deliver ZFNs and a repair template to tobacco cells to achieve gene targeting at an integrated reporter gene (Baltes et al., Plant Cell 2014, 26:151-163); BeYDV also have been used for targeted knock-in of a strong promoter upstream of a tomato gene that regulates anthocyanin synthesis (Cermik et al., Genome Biol 16:232, 2015). WDV is a ssDNA virus (Mastrevirus) that infects a variety of grasses, including most cereals. WDV-derived replicons can be used to express foreign proteins in cells from plants such as wheat and maize cells (Ugaki et al., supra; Matzeit et al., Plant Cell 1991, 3:247-258; and Suarez-Lopez and Gutierrez, Virology 1997, 227:389-399). Tomato leaf curl virus (ToLCV) also is a ssDNA virus (Begomovirus), and although its natural hosts are normally Solanaceous species, ToLCV-derived replicons can efficiently replicate and express GFP in rice (Pandey et al., Virol J 2009, 6:152).

Gemini virus-based replicons can be particularly useful. Geminiviruses are a large family of plant viruses that contain circular, single-stranded DNA genomes. Examples of geminiviruses include the cabbage leaf curl virus, tomato golden mosaic virus, bean yellow dwarf virus (BeYDV; also referred to as chickpea chlorotic dwarf virus), African cassava mosaic virus, wheat dwarf virus (WDV), miscanthus streak mastrevirus, tobacco yellow dwarf virus, tomato yellow leaf curl virus, bean golden mosaic virus, beet curly top virus, maize streak virus, and tomato pseudo-curly top virus.

The engineered replicon can be generated by, for example, replacing non-essential geminivirus nucleotide sequence (e.g., CP sequence) with a desired cargo sequence. Other methods for adding sequence to viral vectors include, without limitation, those discussed in Peretz et al. (Plant Physiol., 145:1251-1263, 2007).

In one example of the autonomous replicons of the disclosure, the LIR (long intergenic region) region initiates transcription of the cargo sequence, while the SIR (short intergenic region) terminates transcription. Geminivirus-derived vectors can be sent to cells in two different pathways: the cis or autonomous and trans or tethered route wherein cis employs the Rep in its native position to the LIR, driven by a C-sense promoter, and can give rise to thousands of copies of the replicons and trans employs persistent production of Rep protein through stable integration to drive production of the replicon.

In some embodiments, the donor polynucleotide comprised within the first autonomous replicon comprises, from 5′ to 3′: a first long intergenic region (LIR), the RE, the cargo sequence, the LE, a short intergenic region (SIR), a sequence encoding RepA, and a second LTR. In some embodiments, the at least one of the one or more first helper polynucleotides and/or the at least one of the one or more second helper polynucleotides comprised within the second autonomous replicon comprises, from 5′ to 3′: a first long intergenic region (LIR), the first helper polynucleotide or the second helper polynucleotide, a short intergenic region (SIR), a sequence encoding RepA, and a second LIR. The first and/or second LIR can comprise or consist of the sequence of SEQ ID NO: 1 or a sequence having one, two, or three mismatches relative to the sequence of SEQ ID NO: 1. The SIR can comprise or consist of the sequence of SEQ ID NO: 2 or a sequence having one, two, or three mismatches relative to the sequence of SEQ ID NO: 2. The sequence encoding RepA can comprise or consist of the sequence of SEQ ID NO: 3 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 3.

The amount of the donor polynucleotide in the plant cell can be capable of increasing by at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) following the onset of autonomous replication. The amount of the donor polynucleotide in the plant cell can be capable of increasing by at least 10-fold following the onset of autonomous replication.

The amount of a gene product encoded by the donor polynucleotide in the plant cell can be capable of increasing by at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) following the onset of autonomous replication. The amount of the gene product encoded by the donor polynucleotide in the plant cell can be capable of increasing by at least 10-fold following the onset of autonomous replication.

### Donor Polynucleotide

A donor polynucleotide can comprise a cargo sequence flanked by a first transposon end sequence (RE) on the 5′ end of the cargo sequence and a second transposon end sequence (LE) on the 3′ end of the cargo sequence. Cargo sequence flanked by an RE and LE is thus a transposable element (e.g., transposon) capable of being inserted into a double-stranded target sequence of a genome of a plant cell or a target plasmid in a plant cell using the systems, nucleic compositions, and methods disclosed herein.

In some embodiments, the LE comprises the sequence of any one of SEQ ID NOs: 4-5 or a sequence having one, two or three mismatches relative to the sequence of any one of SEQ ID NOs: 4-5 and wherein the RE comprises the sequence of any one of SEQ ID NOs: 6-7 or a sequence having one, two or three mismatches relative to the sequence of any one of SEQ ID NOs: 6-7. In some embodiments, the LE comprises or consists of the sequence of any one of SEQ ID NOs: 4-5 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of any one of SEQ ID NOs: 4-5. In some embodiments, the RE comprises or consists of the sequence of any one of SEQ ID NOs: 6-7 or a that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of any one of SEQ ID NOs: 6-7.

### Cargo Sequence

The length of the cargo sequence can vary. The cargo sequence can be 0.2 to 1000 kilobase pairs (kb) (e.g., 0.2 kb, 0.5 kb, 0.75 kb, 1.0 kb, 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 110 kb, 120 kb, 130 kb, 140 kb, 150 kb, 160 kb, 170 kb, 180 kb, 190 kb, 200 kb, 300 kb, 400 kb, 500 kb, 600 kb, 700 kb, 800 kb, 900 kb, 1000 kb, or a number or a range between any two of these values) in length. The cargo sequence can be 200 to 1200 base pairs (bp) (e.g., 200 bp, 210 bp, 220 bp, 230 bp, 240 bp, 250 bp, 260 bp, 270 bp, 280 bp, 290 bp, 300 bp, 310 bp, 320 bp, 330 bp, 340 bp, 350 bp, 360 bp, 370 bp, 380 bp, 390 bp, 400 bp, 410 bp, 420 bp, 430 bp, 440 bp, 450 bp, 460 bp, 470 bp, 480 bp, 490 bp, 500 bp, 510 bp, 520 bp, 530 bp, 540 bp, 550 bp, 560 bp, 570 bp, 580 bp, 590 bp, 600 bp, 610 bp, 620 bp, 630 bp, 640 bp, 650 bp, 660 bp, 670 bp, 680 bp, 690 bp, 700 bp, 710 bp, 720 bp, 730 bp, 740 bp, 750 bp, 760 bp, 770 bp, 780 bp, 790 bp, 800 bp, 810 bp, 820 bp, 830 bp, 840 bp, 850 bp, 860 bp, 870 bp, 880 bp, 890 bp, 900 bp, 910 bp, 920 bp, 930 bp, 940 bp, 950 bp, 960 bp, 970 bp, 980 bp, 990 bp, 1000 bp, 1025 bp, 1050 bp, 1075 bp, 1100 bp, 1125 bp, 1150 bp, 1175 bp, 1200 bp or a number or a range between any two of these values) in length.

Any cargo sequence contemplated by the skilled artisan can be used according to the systems, compositions, and methods of the disclosure. In some embodiments, the cargo sequence comprises one or more exogenous sequences which when introduced into plants will alter the phenotype of the plant, a plant organ, plant tissue, or portion of the plant. Exemplary exogenous sequences encode polypeptides involved in one or more important biological properties in plants. Other exemplary exogenous sequences can alter expression of exogenous or endogenous genes, either increasing or decreasing expression, optionally in response to a specific signal or stimulus.

As used herein, the term “trait” can refer either to the altered phenotype of interest or the nucleic acid which causes the altered phenotype of interest. One of the major purposes of transformation of crop plants is to add some commercially desirable, agronomically important traits to the plant. Such traits include, but are not limited to, herbicide resistance or tolerance; insect (pest) resistance or tolerance; disease resistance or tolerance (viral, bacterial, fungal, nematode or other pathogens); stress tolerance and/or resistance, as exemplified by resistance or tolerance to drought, heat, chilling, freezing, excessive moisture, salt stress, mechanical stress, extreme acidity, alkalinity, toxins, UV light, ionizing radiation or oxidative stress; increased yields, whether in quantity or quality; enhanced or altered nutrient acquisition and enhanced or altered metabolic efficiency; enhanced or altered nutritional content and makeup of plant tissues used for food, feed, fiber or processing; physical appearance; male sterility; drydown; standability; prolificacy; starch quantity and quality; oil quantity and quality; protein quality and quantity; amino acid composition; modified chemical production; altered pharmaceutical or nutraceutical properties; altered bioremediation properties; increased biomass; altered growth rate; altered fitness; altered biodegradability; altered CO₂ fixation; presence of bioindicator activity; altered digestibility by humans or animals; altered allergenicity; altered mating characteristics; altered pollen dispersal; improved environmental impact; altered nitrogen fixation capability; the production of a pharmaceutically active protein; the production of a small molecule with medicinal properties; the production of a chemical including those with industrial utility; the production of nutraceuticals, food additives, carbohydrates, RNAs, lipids, fuels, dyes, pigments, vitamins, scents, flavors, vaccines, antibodies, hormones, and the like; and alterations in plant architecture or development, including changes in developmental timing, photosynthesis, signal transduction, cell growth, reproduction, or differentiation. Additionally one could create a library of an entire genome from any organism or organelle including mammals, plants, microbes, fungi, or bacteria, represented on one or more donor polynucleotides.

In some embodiments, the modified plant may exhibit increased or decreased expression or accumulation of a product of the plant, which may be a natural product of the plant or a new or altered product of the plant. Exemplary products include an enzyme, an RNA molecule, a nutritional protein, a structural protein, an amino acid, a lipid, a fatty acid, a polysaccharide, a sugar, an alcohol, an alkaloid, a carotenoid, a propanoid, a phenylpropanoid, or terpenoid, a steroid, a flavonoid, a phenolic compound, an anthocyanin, a pigment, a vitamin or a plant hormone. In some embodiments, the modified plant has enhanced or diminished requirements for light, water, nitrogen, or trace elements. In some embodiments, the modified plant has an enhance ability to capture or fix nitrogen from its environment. In some embodiments, the modified plant is enriched for an essential amino acid as a proportion of a protein fraction of the plant. The protein fraction may be, for example, total seed protein, soluble protein, insoluble protein, water-extractable protein, and lipid-associated protein. The modification can include, e.g., overexpression, underexpression, antisense modulation, sense suppression, inducible expression, inducible repression, or inducible modulation of a gene.

A brief summary of exemplary improved properties and polypeptides of interest for either increased or decreased expression is provided below.

### Herbicide Resistance

A herbicide resistance (or tolerance) trait is a characteristic of a modified plant that is resistant to dosages of an herbicide that is typically lethal to a non-modified plant. Exemplary herbicides for which resistance is useful in a plant include glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and glufosinate herbicides. Other herbicides would be useful as would combinations of herbicide genes.

The genes encoding phosphinothricin acetyltransferase (bar), glyphosate tolerant EPSP synthase genes, glyphosate acetyltransferase, the glyphosate degradative enzyme gene gox encoding glyphosate oxidoreductase, deh (encoding a dehalogenase enzyme that inactivates dalapon), herbicide resistant (e.g., sulfonylurea and imidazolinone) acetolactate synthase, and bxn genes (encoding a nitrilase enzyme that degrades bromoxynil) are good examples of herbicide resistant genes for use in transformation. The bar gene codes for an enzyme, phosphinothricin acetyltransferase (PAT), which inactivates the herbicide phosphinothricin and prevents this compound from inhibiting glutamine synthetase enzymes. The enzyme 5 enolpyruvylshikimate 3 phosphate synthase (EPSP Synthase), is normally inhibited by the herbicide N (phosphonomethyl)glycine (glyphosate). However, genes are known that encode glyphosate resistant EPSP synthase enzymes. These genes are particularly contemplated for use in plant transformation. The deh gene encodes the enzyme dalapon dehalogenase and confers resistance to the herbicide dalapon. The bxn gene codes for a specific nitrilase enzyme that converts bromoxynil to a non herbicidal degradation product. The glyphosate acetyl transferase gene inactivates the herbicide glyphosate and prevents this compound from inhibiting EPSP synthase. Polypeptides that may produce plants having tolerance to plant herbicides include polypeptides involved in the shikimate pathway, which are of interest for providing glyphosate tolerant plants. Such polypeptides include polypeptides involved in biosynthesis of chorismate, phenylalanine, tyrosine and tryptophan.

### Insect Resistance

Potential insect resistance (or tolerance) genes that can be introduced include Bacillus thuringiensis toxin genes or Bt genes (Watrud et al., In: Engineered Organisms and the Environment, 1985). Bt genes may provide resistance to lepidopteran or coleopteran pests such as European Corn Borer (ECB). Preferred Bt toxin genes for use in such embodiments include the CryIA(b) and CryIA(c) genes. Endotoxin genes from other species of B. thuringiensis which affect insect growth or development also may be employed in this regard. It is contemplated that preferred Bt genes for use according to the present disclosure will be those in which the coding sequence has been modified to effect increased expression in plants, and for example, in monocot plants. Means for preparing synthetic genes are well known in the art and are disclosed in, for example, U.S. Pat. Nos. 5,500,365 and 5,689,052, each of the disclosures of which are specifically incorporated herein by reference in their entirety. Examples of such modified Bt toxin genes include a synthetic Bt CryIA(b) gene (Perlak et al., Proc. Natl. Acad. Sci. USA, 88:3324-3328, 1991), and the synthetic CryIA(c) gene termed 1800b (PCT Application WO 95/06128).

Protease inhibitors also may provide insect resistance (Johnson et al., Proc Natl Acad Sci USA. 1989 December; 86(24): 9871-9875.), and will thus have utility in plant transformation. The use of a protease inhibitor II gene, pinII, from tomato or potato is envisioned to be particularly useful. Even more advantageous is the use of a pinII gene in combination with a Bt toxin gene, the combined effect of which has been discovered to produce synergistic insecticidal activity. Other genes which encode inhibitors of the insect's digestive system, or those that encode enzymes or co factors that facilitate the production of inhibitors, also may be useful. This group may be exemplified by oryzacystatin and amylase inhibitors such as those from wheat and barley.

Amylase inhibitors are found in various plant species and are used to ward off insect predation via inhibition of the digestive amylases of attacking insects. Several amylase inhibitor genes have been isolated from plants and some have been introduced as exogenous nucleic acids, conferring an insect resistant phenotype that is potentially useful (“Plants, Genes, and Crop Biotechnology” by Maarten J. Chrispeels and David E. Sadava (2003) Jones and Bartlett Press).

Genes encoding lectins may confer additional or alternative insecticide properties. Lectins are multivalent carbohydrate binding proteins which have the ability to agglutinate red blood cells from a range of species. Lectins have been identified recently as insecticidal agents with activity against weevils, ECB and rootworm (Murdock et al., Phytochemistry, 29:85-89, 1990, Czapla & Lang, J. Econ. Entomol., 83:2480-2485, 1990). Lectin genes contemplated to be useful include, for example, barley and wheat germ agglutinin (WGA) and rice lectins (Gatehouse et al., J. Sci. Food. Agric, 35:373-380, 1984), with WGA being preferred. Genes controlling the production of large or small polypeptides active against insects when introduced into the insect pests, such as, e.g., lytic peptides, peptide hormones and toxins and venoms, form another aspect of the disclosure. For example, it is contemplated that the expression of juvenile hormone esterase, directed towards specific insect pests, also may result in insecticidal activity, or perhaps cause cessation of metamorphosis (Hammock et al., Nature, 344:458-461, 1990).

Genes which encode enzymes that affect the integrity of the insect cuticle form yet another aspect of the disclosure. Such genes include those encoding, e.g., chitinase, proteases, lipases and also genes for the production of nikkomycin, a compound that inhibits chitin synthesis, the introduction of any of which is contemplated to produce insect resistant plants. Genes that code for activities that affect insect molting, such as those affecting the production of ecdysteroid UDP glucosyl transferase, also fall within the scope of the useful exogenous nucleic acids of the present disclosure. Genes that code for enzymes that facilitate the production of compounds that reduce the nutritional quality of the host plant to insect pests also are encompassed by the present disclosure. In some embodiments, insecticidal activity can be conferred on a plant by altering its sterol composition. Sterols are obtained by insects from their diet and are used for hormone synthesis and membrane stability. Therefore alterations in plant sterol composition by expression of novel genes, e.g., those that directly promote the production of undesirable sterols or those that convert desirable sterols into undesirable forms, can, in some embodiments, have a negative effect on insect growth and/or development and hence endow the plant with insecticidal activity. Lipoxygenases are naturally occurring plant enzymes that have been shown to exhibit anti nutritional effects on insects and to reduce the nutritional quality of their diet. Therefore, further embodiments of the disclosure concern modified plants with enhanced lipoxygenase activity which may be resistant to insect feeding.

Tripsacum dactyloides is a species of grass that is resistant to certain insects, including corn root worm. It is anticipated that genes encoding proteins that are toxic to insects or are involved in the biosynthesis of compounds toxic to insects will be isolated from Tripsacum and that these novel genes will be useful in conferring resistance to insects. It is known that the basis of insect resistance in Tripsacum is genetic, because said resistance has been transferred to Zea mays via sexual crosses (Branson and Guss, Proceedings North Central Branch Entomological Society of America, 27:91-95, 1972). It is further anticipated that other cereal, monocot or dicot plant species may have genes encoding proteins that are toxic to insects which would be useful for producing insect resistant plants.

Further genes encoding proteins characterized as having potential insecticidal activity also may be used as exogenous nucleic acids in accordance herewith. Such genes include, for example, the cowpea trypsin inhibitor (CpTI; Hilder et al., Nature, 330:160-163, 1987) which may be used as a rootworm deterrent; genes encoding avermectin (Avermectin and Abamectin., Campbell, W. C., Ed., 1989; Ikeda et al., J. Bacteriol., 169:5615-5621, 1987) which may prove particularly useful as a corn rootworm deterrent; ribosome inactivating protein genes; and even genes that regulate plant structures. Modified plants including anti insect antibody genes and genes that code for enzymes that can convert a non toxic insecticide (pro insecticide) applied to the outside of the plant into an insecticide inside the plant also are contemplated.

Polypeptides that may improve plant tolerance to the effects of plant pests or pathogens include proteases, polypeptides involved in anthocyanin biosynthesis, polypeptides involved in cell wall metabolism, including cellulases, glucosidases, pectin methylesterase, pectinase, polygalacturonase, chitinase, chitosanase, and cellulose synthase, and polypeptides involved in biosynthesis of terpenoids or indole for production of bioactive metabolites to provide defense against herbivorous insects. Vegetative Insecticidal Proteins (VIP) are a class of proteins originally found to be produced in the vegetative growth phase of the bacterium, Bacillus cereus, but do have a spectrum of insect lethality similar to the insecticidal genes found in strains of Bacillus thuriengensis. Both the vipla and vip3 A genes have been isolated and have demonstrated insect toxicity. It is anticipated that such genes may be used in modified plants to confer insect resistance (“Plants, Genes, and Crop Biotechnology” by Maarten J. Chrispeels and David E. Sadava (2003) Jones and Bartlett Press).

### Environment or Stress Resistance

Improvement of a plant's ability to tolerate various environmental stresses such as, but not limited to, drought, excess moisture, chilling, freezing, high temperature, salt, and oxidative stress, also can be effected through expression of novel genes. Benefits may be realized in terms of increased resistance to freezing temperatures through the introduction of an “antifreeze” protein such as that of the Winter Flounder (Cutler et al, J. Plant Physiol., 135:351-354, 1989) or synthetic gene derivatives thereof. Improved chilling tolerance also may be conferred through increased expression of glycerol 3 phosphate acetyltransferase in chloroplasts (Wolter et al., The EMBO J., 4685-4692, 1992). Resistance to oxidative stress (often exacerbated by conditions such as chilling temperatures in combination with high light intensities) can be conferred by expression of superoxide dismutase (Gupta et al., 1993), and may be improved by glutathione reductase (Bowler et al., Ann Rev. Plant Physiol., 43:83-116, 1992). Such strategies can allow for tolerance to freezing in newly emerged fields as well as extending later maturity higher yielding varieties to earlier relative maturity zones.

It is contemplated that the expression of novel genes that favorably affect plant water content, total water potential, osmotic potential, or turgor will enhance the ability of the plant to tolerate drought. As used herein, the terms “drought resistance” and “drought tolerance” are used to refer to a plant's increased resistance or tolerance to stress induced by a reduction in water availability, as compared to normal circumstances, and the ability of the plant to function and survive in lower water environments. Expression of genes encoding for the biosynthesis of osmotically active solutes, such as polyol compounds, may impart protection against drought. Within this class are genes encoding for mannitol L phosphate dehydrogenase (Lee and Saier, 1982) and trehalose 6 phosphate synthase (Kaasen et al., J. Bacteriology, 174:889-898, 1992). Through the subsequent action of native phosphatases in the cell or by the introduction and coexpression of a specific phosphatase, these introduced genes will result in the accumulation of either mannitol or trehalose, respectively, both of which have been well documented as protective compounds able to mitigate the effects of stress. Mannitol accumulation in transgenic tobacco has been verified and preliminary results indicate that plants expressing high levels of this metabolite are able to tolerate an applied osmotic stress (Tarczynski et al., Science, 259:508-510, 1993, Tarczynski et al Proc. Natl. Acad. Sci. USA, 89:1-5, 1993).

Similarly, the efficacy of other metabolites in protecting either enzyme function (e.g., alanopine or propionic acid) or membrane integrity (e.g., alanopine) has been documented (Loomis et al., J. Expt. Zoology, 252:9-15, 1989), and therefore expression of genes encoding for the biosynthesis of these compounds might confer drought resistance in a manner similar to or complimentary to mannitol. Other examples of naturally occurring metabolites that are osmotically active and/or provide some direct protective effect during drought and/or desiccation include fructose, erythritol (Coxson et al., Biotropica, 24:121-133, 1992), sorbitol, dulcitol (Karsten et al., Botanica Marina, 35:11-19, 1992), glucosylglycerol (Reed et al., J. Gen. Microbiology, 130:1-4, 1984; Erdmann et al., J. Gen. Microbiology, 138:363-368, 1992), sucrose, stachyose (Koster and Leopold, Plant Physiol., 88:829-832, 1988; Blackman et al., Plant Physiol., 100:225-230, 1992), raffmose (Bernal Lugo and Leopold, Plant Physiol., 98:1207-1210, 1992), proline (Rensburg et al., J. Plant Physiol., 141:188-194, 1993), glycine betaine, ononitol and pinitol (Vernon and Bohnert, The EMBO J., 11:2077-2085, 1992). Continued canopy growth and increased reproductive fitness during times of stress will be augmented by introduction and expression of genes such as those controlling the osmotically active compounds discussed above and other such compounds. Currently preferred genes which promote the synthesis of an osmotically active polyol compound are genes which encode the enzymes mannitol 1 phosphate dehydrogenase, trehalose 6 phosphate synthase and myo-inositol O-methyltransferase.

It is contemplated that the expression of specific proteins also may increase drought tolerance. Three classes of Late Embryo genie Abundant (LEA) Proteins have been assigned based on structural similarities (see Dure et al., Plant Molecular Biology, 12:475-486, 1989). All three classes of LEAs have been demonstrated in maturing (e.g. desiccating) seeds. Within these 3 types of LEA proteins, the Type II (dehydrin type) have generally been implicated in drought and/or desiccation tolerance in vegetative plant parts (e.g. Mundy and Chua, The EMBO J., 7:2279-2286, 1988; Piatkowski et al., Plant Physiol., 94:1682-1688, 1990; Yamaguchi Shinozaki et al., Plant Cell Physiol., 33:217-224, 1992). Expression of a Type III LEA (HVA 1) in tobacco was found to influence plant height, maturity and drought tolerance (Fitzpatrick, Gen. Engineering News, 22:7, 1993). In rice, expression of the HVA 1 gene influenced tolerance to water deficit and salinity (Xu et al., Plant Physiol., 110:249-257, 1996). Expression of structural genes from any of the three LEA groups may therefore confer drought tolerance. Other types of proteins induced during water stress include thiol proteases, aldolases or transmembrane transporters (Guerrero et al., Plant Molecular Biology, 15:11-26, 1990), which may confer various protective and/or repair type functions during drought stress. It also is contemplated that genes that effect lipid biosynthesis and hence membrane composition might also be useful in conferring drought resistance on the plant. Many of these genes for improving drought resistance have complementary modes of action. Thus, it is envisaged that combinations of these genes might have additive and/or synergistic effects in improving drought resistance in plants. Many of these genes also improve freezing tolerance (or resistance); the physical stresses incurred during freezing and drought are similar in nature and may be mitigated in similar fashion. Benefit may be conferred via constitutive expression of these genes, but the preferred means of expressing these novel genes may be through the use of a turgor induced promoter (such as the promoters for the turgor induced genes described in Guerrero et al., Plant Molecular Biology, 15:11-26, 1990 and Shagan et al., Plant Physiol., 101:1397-1398, 1993 which are incorporated herein by reference). Spatial and temporal expression patterns of these genes may enable plants to better withstand stress.

Expression of genes that are involved with specific morphological traits that allow for increased water extractions from drying soil can be of benefit. For example, introduction and expression of genes that alter root characteristics may enhance water uptake. It also is contemplated that expression of genes that enhance reproductive fitness during times of stress would be of significant value. For example, expression of genes that improve the synchrony of pollen shed and receptiveness of the female flower parts, e.g., silks, would be of benefit. In addition, expression of genes that minimize kernel abortion during times of stress may increase the amount of grain to be harvested and hence be of value. Given the overall role of water in determining yield, it is contemplated that enabling plants to utilize water more efficiently, through the introduction and expression of novel genes, will improve overall performance even when soil water availability is not limiting. By introducing genes that improve the ability of plants to maximize water usage across a full range of stresses relating to water availability, yield stability or consistency of yield performance may be realized.

Polypeptides that may improve stress tolerance under a variety of stress conditions include polypeptides involved in gene regulation, such as serine/threonine-protein kinases, MAP kinases, MAP kinase kinases, and MAP kinase kinase kinases; polypeptides that act as receptors for signal transduction and regulation, such as receptor protein kinases; intracellular signaling proteins, such as protein phosphatases, GTP binding proteins, and phospholipid signaling proteins; polypeptides involved in arginine biosynthesis; polypeptides involved in ATP metabolism, including for example ATPase, adenylate transporters, and polypeptides involved in ATP synthesis and transport; polypeptides involved in glycine betaine, jasmonic acid, flavonoid or steroid biosynthesis; and hemoglobin. Enhanced or reduced activity of such polypeptides in modified plants will provide changes in the ability of a plant to respond to a variety of environmental stresses, such as chemical stress, drought stress and pest stress. Other polypeptides that can improve plant tolerance to cold or freezing temperatures include polypeptides involved in biosynthesis of trehalose or raffinose, polypeptides encoded by cold induced genes, fatty acyl desaturases and other polypeptides involved in glycerolipid or membrane lipid biosynthesis, which find use in modification of membrane fatty acid composition, alternative oxidase, calcium-dependent protein kinases, LEA proteins or uncoupling protein.

Other polypeptides for improvement of plant tolerance to heat include polypeptides involved in biosynthesis of trehalose, polypeptides involved in glycerolipid biosynthesis or membrane lipid metabolism (for altering membrane fatty acid composition), heat shock proteins or mitochondrial NDK. Other polypeptides that may improve tolerance to extreme osmotic conditions include polypeptides involved in proline biosynthesis. Other polypeptides for improvement of plant tolerance to drought conditions include aquaporins, polypeptides involved in biosynthesis of trehalose or wax, LEA proteins or invertase.

### Disease Resistance

Increased resistance (or tolerance) to diseases can, in some embodiments, be realized through introduction of genes into plants, for example, into monocotyledonous plants such as maize. It is possible to produce resistance to diseases caused by viruses, viroids, bacteria, fungi and nematodes. It also is contemplated that control of mycotoxin producing organisms may be realized through expression of introduced genes. Resistance can be affected through suppression of endogenous factors that encourage disease-causing interactions, expression of exogenous factors that are toxic to or otherwise provide protection from pathogens, or expression of factors that enhance the plant's own defense responses.

Resistance to viruses can be produced through expression of novel genes. For example, it has been demonstrated that expression of a viral coat protein in a modified plant can impart resistance to infection of the plant by that virus and perhaps other closely related viruses (Cuozzo et al, Bio/Technology, 6:549-553, 1988, Hemenway et al., The EMBO J., 7:1273-1280, 1988, Abel et al., Science, 232:738-743, 1986). It is contemplated that expression of antisense genes targeted at essential viral functions can also impart resistance to viruses. For example, an antisense gene targeted at the gene responsible for replication of viral nucleic acid may inhibit replication and lead to resistance to the virus. Interference with other viral functions through the use of antisense genes also may increase resistance to viruses.

Increased resistance to diseases caused by bacteria and fungi may be realized through introduction of novel genes. It is contemplated that genes encoding so called “peptide antibiotics,” pathogenesis related (PR) proteins, toxin resistance, or proteins affecting host pathogen interactions such as morphological characteristics will be useful. Peptide antibiotics are polypeptide sequences which are inhibitory to growth of bacteria and other microorganisms. For example, the classes of peptides referred to as cecropins and magainins inhibit growth of many species of bacteria and fungi. It is proposed that expression of PR proteins in plants, for example, monocots such as maize, may be useful in conferring resistance to bacterial disease. These genes are induced following pathogen attack on a host plant and have been divided into at least five classes of proteins (Bol, Linthorst, and Cornelissen, 1990). Included amongst the PR proteins are beta 1, 3 glucanases, chitinases, and osmotin and other proteins that are believed to function in plant resistance to disease organisms. Other genes have been identified that have antifungal properties, e.g., UDA (stinging nettle lectin), or herein (Broakaert et al., 1989; Barkai Golan et al., 1978). It is known that certain plant diseases are caused by the production of phytotoxins. Resistance to these diseases can, in some embodiments, be achieved through expression of a novel gene that encodes an enzyme capable of degrading or otherwise inactivating the phytotoxin. It also is contemplated that expression of novel genes that alter the interactions between the host plant and pathogen may be useful in reducing the ability of the disease organism to invade the tissues of the host plant,—e.g., an increase in the waxiness of the leaf cuticle or other morphological characteristics.

Polypeptides useful for imparting improved disease responses to plants include polypeptides encoded by cercosporin induced genes, antifungal proteins and proteins encoded by R-genes or SAR genes. Agronomically important diseases caused by fungal phytopathogens include: glume or leaf blotch, late blight, stalk/head rot, rice blast, leaf blight and spot, corn smut, wilt, sheath blight, stem canker, root rot, blackleg or kernel rot.

Exemplary plant viruses include tobacco or cucumber mosaic virus, ringspot virus, necrosis virus, maize dwarf mosaic virus, etc. Specific fungal, bacterial and viral pathogens of major crops include, but are not limited to:

RICE: rice brown spot fungus (Cochliobolus miyabeanus), rice blast fungus-Magnaporthe grisea (Pyricularia grisea), Magnaporthe salvinii (Sclerotium oryzae), Xanthomonas oryzae pv. oryzae, Xanthomonas oryzae pv. oryzicola, Rhizoctonia spp. (including but not limited to Rhizoctonia solani, Rhizoctonia oryzae and Rhizoctonia oryzae-sativae), Pseudomonas spp. (including but not limited to Pseudomonas plantarii, Pseudomonas avenae, Pseudomonas glumae, Pseudomonas fuscovaginae, Pseudomonas alboprecipitans, Pseudomonas syringae pv. panici, Pseudomonas syringae pv. syringae, Pseudomonas syringae pv. oryzae and Pseudomonas syringae pv. aptata), Erwinia spp. (including but not limited to Erwinia herbicola, Erwinia amylovaora, Erwinia chrysanthemi and Erwinia carotovora), Achyla spp. (including but not limited to Achyla conspicua and Achyia klebsiana), Pythium spp. (including but not limited to Pythium dissotocum, Pythium irregulare, Pythium arrhenomanes, Pythium myriotylum, Pythium catenulatum, Pythium graminicola and Pythium spinosum), Saprolegnia spp., Dictyuchus spp., Pythiogeton spp., Phytophthora spp., Alternaria padwickii, Cochliobolus miyabeanus, Curvularia spp. (including but not limited to Curvularia lunata, Curvularia affmis, Curvularia clavata, Curvularia eragrostidis, Curvularia fallax, Curvularia geniculata, Curvularia inaequalis, Curvularia intermedia, Curvularia oryzae, Curvularia oryzae-sativae, Curvularia pallescens, Curvularia senegalensis, Curvularia tuberculata, Curvularia uncinata and Curvularia verruculosa), Sarocladium oryzae, Gerlachia oryzae, Fusarium spp. (including but not limited Fusarium graminearum, Fusarium nivale and to different pathovars of Fusarium monoliforme, including pvs. fujikuroi and zeae), Sclerotium rolfsii, Phoma exigua, Mucor fragilis, Trichoderma viride, Rhizopus spp., Cercospora oryzae, Entyloma oryzae, Dreschlera gigantean, Scierophthora macrospora, Mycovellosiella oryzae, Phomopsis oryzae-sativae, Puccinia graminis, Uromyces coronatus, Cylindrocladium scoparium, Sarocladium oryzae, Gaeumannomyces graminis pv. graminis, Myrothecium verrucaria, Pyrenochaeta oryzae, Ustilaginoidea virens, Neovossia spp. (including but not limited to Neovossia horrida), Tilletia spp., Balansia oryzae-sativae, Phoma spp. (including but not limited to Phoma sorghina, Phoma insidiosa, Phoma glumarum, Phoma glumicola and Phoma oryzina), Nigrospora spp. (including but not limited to Nigrospora oryzae, Nigrospora sphaerica, Nigrospora panici and Nigrospora padwickii), Epiococcum nigrum, Phyllostica spp., Wolkia decolorans, Monascus purpureus, Aspergillus spp., Penicillium spp., Absidia spp., Mucor spp., Chaetomium spp., Dematium spp., Monilia spp., Streptomyces spp., Syncephalastrum spp., Verticillium spp., Nematospora coryli, Nakataea sigmoidea, Cladosporium spp., Bipolaris spp., Coniothyrium spp., Diplodia oryzae, Exserophilum rostratum, Helococera oryzae, Melanomma glumarum, Metashaeria spp., Mycosphaerella spp., Oidium spp., Pestalotia spp., Phaeoseptoria spp., Sphaeropsis spp., Trematosphaerella spp., rice black-streaked dwarf virus, rice dwarf virus, rice gall dwarf virus, barley yellow dwarf virus, rice grassy stunt virus, rice hoja blanca virus, rice necrosis mosaic virus, rice ragged stunt virus, rice stripe virus, rice stripe necrosis virus, rice transitory yellowing virus, rice tungro bacilliform virus, rice tungro spherical virus, rice yellow mottle virus, rice tarsonemid mite virus, Echinochloa hoja blanca virus, Echinochloa ragged stunt virus, orange leaf mycoplasma-like organism, yellow dwarf mycoplasma-like organism, Aphelenchoides besseyi, Ditylenchus angustus, Hirschmanniella spp., Criconemella spp., Meloidogyne spp., Heterodera spp., Pratylenchus spp., Hoplolaimus indicus. SOYBEANS: Phytophthora sojae, Fusarium solani f. sp. Glycines, Macrophomina phaseolina, Fusarium, Pythium, Rhizoctonia, Phialophora gregata, Sclerotinia sclerotiorum, Diaporthe phaseolorum var. sojae, Colletotrichum truncatum, Phomopsis longicolla, Cercospora kikuchii, Diaporthe phaseolonum var. meridionalis (and var. caulivora), Phakopsora pachyrhyzi, Fusarium solani, Microsphaera diffusa, Septoria glycines, Cercospora kikuchii, Macrophomina phaseolina, Sclerotinia sclerotiorum, Corynespora cassiicola, Rhizoctonia solani, Cercospora sojina, Phytophthora megasperma fsp. glycinea, Macrophomina phaseolina, Fusarium oxysporum, Diapothe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Phyllosticta sojicola, Alternaria alternata, Pseudomonas syringae p.v. glycinea, Xanthomonas campestris p.v. phaseoli, Microspaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium dearyanum, Tomato spotted wilted virus, Heterodera glycines, Fusarium solani, Soybean cyst and root knot nematodes.

CORN: Fusarium moniliforme var. subglutinans, Erwinia stewartii, Fusarium moniliforme, Gibberella zeae (Fusarium Graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (cochliobolus heterostrophus), Helminthosporium carbonum I, II, and III (Cochliobolus carborium), Exserohilum turcicum I, II and III, Helminthosporium pedicellatum, Physoderma maydis, Phyllosticta maydis, Kabatie-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganese subsp. Nebraskense, Trichoderma viride, Maize dwarf Mosaic Virus A and B, Wheat Streak Mosaic Virus, Maize Chlorotic Dwarf Virus, Claviceps sorghi, Pseudonomas avenae, Erwinia chrysantemi p.v. Zea, Erwinia corotovora, Cornstun spiroplasma, Diplodia macrospora, Sclerophthora macrospora, Peronosclerospora sorghi, Peronoscherospora philippinesis, Peronosclerospora maydis, Peronosclerospora sacchari, Spacelotheca reiliana, Physopella zea, Cephalosporium maydis, Caphalosporium acremonium, Maize Chlorotic Mottle Virus, High Plains Virus, Maize Mosaic Virus, Maize Rayado Fino Virus, Maize Streak Virus, Maize Stripe Virus, Maize Rought Dwarf Virus:

WHEAT: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium cuhnorum, Ustilago tritici, Ascochyta tritici, Cephalosporium gramineum, Collotetrichum graminicola, Erysiphe graminis f. sp. Tritici, Puccinia graminis f. sp. Tritici, Puccinia recondite f. sp. tritici, puccinia striiformis, Pyrenophora triticirepentis, Septoria nodorum, Septoria tritici, Spetoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Virus, Wheat Spindle Streak Virus, American Wheat Striate Virus, Claviceps purpurea, Tilletia tritici, Tilletia laeyis, Pstilago tritici, Tilletia indica, Rhizoctonia solani, Pythium arrhenomannes, Pythium gramicola, Pythium aphanidermatum, High Plains Virus, European Wheat Striate Virus:

CANOLA: Albugo Candida, Alternaria brassicae, Leptosharia maculans, Rhizoctonia solani, Sclerotinia sclerotiorum, Mycospaerella brassiccola, Pythium ultimum, Peronospora parasitica, Fusarium roseum, Fusarium oxysporum, Tilletia foetida, Tilletia caries, Alternaria alternata: SUNFLOWER: Plasmophora halstedii, Scherotinia sclerotiorum, Aster Yellows, Septoria helianthi, Phomopsis helianthi, Altemaria helianthi, Alternaria zinniae, Botrytis cinera, Phoma macdonaldii, Macrophomina phaseolina, Erysiphe cichoracearum, Phizopus oryzae, Rhizopus arrhizus, Rhizopus stolonifer, Puccinia helianthi, Verticillium Dahliae, Erwinia carotovoram p.v. carotovora, Cephalosporium acremonium, Phytophthora cryptogea, Albugo tragopogonis.

SORGHUM: Exserohilum turcicum, Colletotrichum graminicola (Glomerella graminicola), Cercospora sorghi, Gloeocercospora sorghi, Ascochyta sorghi, Pseudomonas syringae p.v. syringae, Xanthomonas campestris p.v. holcicola, Pseudomonas andropogonis, Puccinia purpurea, Macrophomina phaseolina, Periconia circinata, Fusarium moniliforme, Alternaria alternate, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari Sporisorium relianum (Sphacelotheca reliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium Oxysporum, Pythium arrhenomanes, Pythium graminicola. ALFALFA: Clavibater michiganensis subsp. Insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Rhizoctonia solani, Uromyces striatus, Colletotrichum trifolii race 1 and race 2, Leptosphaerulina briosiana, Stemphylium botryosum, Stagonospora meliloti, Sclerotinia trifoliorum, Alfalfa Mosaic Virus, Verticillium albo-atrum, Xanthomonas campestris p.v. alfalfae, Aphanomyces euteiches, Stemphylium herbarum, Stemphylium alfalfae.

### Plant Agronomic Characteristics

Two of the factors determining where crop plants can be grown are the average daily temperature during the growing season and the length of time between frosts. Within the areas where it is possible to grow a particular crop, there are varying limitations on the maximal time it is allowed to grow to maturity and be harvested. For example, a variety to be grown in a particular area is selected for its ability to mature and dry down to harvestable moisture content within the required period of time with maximum possible yield. Therefore, crops of varying maturities are developed for different growing locations. Apart from the need to dry down sufficiently to permit harvest, it is desirable to have maximal drying take place in the field to minimize the amount of energy required for additional drying post harvest. Also, the more readily a product such as grain can dry down, the more time there is available for growth and kernel fill. Genes that influence maturity and/or dry down can be identified and introduced into plant lines using transformation techniques to create new varieties adapted to different growing locations or the same growing location, but having improved yield to moisture ratio at harvest. Expression of genes that are involved in regulation of plant development can be useful. It is contemplated that genes can be introduced into plants that would improve standability and other plant growth characteristics. Expression of novel genes in plants which confer stronger stalks, improved root systems, or prevent or reduce ear droppage or shattering would be of great value to the farmer. Introduction and expression of genes that increase the total amount of photoassimilate available by, for example, increasing light distribution and/or interception would be advantageous. In addition, the expression of genes that increase the efficiency of photosynthesis and/or the leaf canopy would further increase gains in productivity. It is contemplated that expression of a phytochrome gene in crop plants can be advantageous. Expression of such a gene may reduce apical dominance, confer semidwarfism on a plant, or increase shade tolerance (U.S. Pat. No. 5,268,526). Such approaches would allow for increased plant populations in the field.

### Nutrient Utilization

The ability to utilize available nutrients may be a limiting factor in growth of crop plants. In some embodiments, one can alter nutrient uptake, tolerate pH extremes, mobilization through the plant, storage pools, and availability for metabolic activities by the introduction of novel genes. These modifications would allow a plant, for example, maize to more efficiently utilize available nutrients. It is contemplated that an increase in the activity of, for example, an enzyme that is normally present in the plant and involved in nutrient utilization would increase the availability of a nutrient or decrease the availability of an anti-nutritive factor. An example of such an enzyme would be phytase. It is further contemplated that enhanced nitrogen utilization by a plant is desirable. Expression of a glutamate dehydrogenase gene in plants, e.g., E. coli gdhA genes, may lead to increased fixation of nitrogen in organic compounds. Furthermore, expression of gdhA in plants may lead to enhanced resistance to the herbicide glufosinate by incorporation of excess ammonia into glutamate, thereby detoxifying the ammonia. It also is contemplated that expression of a novel gene may make a nutrient source available that was previously not accessible, e.g., an enzyme that releases a component of nutrient value from a more complex molecule, perhaps a macromolecule.

Polypeptides useful for improving nitrogen flow, sensing, uptake, storage and/or transport include those involved in aspartate, glutamine or glutamate biosynthesis, polypeptides involved in aspartate, glutamine or glutamate transport, polypeptides associated with the TOR (Target of Rapamycin) pathway, nitrate transporters, nitrate reductases, amino transferases, ammonium transporters, chlorate transporters or polypeptides involved in tetrapyrrole biosynthesis. Polypeptides useful for increasing the rate of photosynthesis include phytochrome, ribulose bisphosphate carboxylase-oxygenase, Rubisco activase, photosystem I and II proteins, electron carriers, ATP synthase, NADH dehydrogenase or cytochrome oxidase.

Polypeptides useful for increasing phosphorus uptake, transport or utilization include phosphatases or phosphate transporters.

### Male Sterility

Male sterility is useful in the production of hybrid seed. Male sterility may be produced through expression of novel genes. For example, it has been shown that expression of genes that encode proteins, RNAs, or peptides that interfere with development of the male inflorescence and/or gametophyte result in male sterility. Chimeric ribonuclease genes that express in the anthers of transgenic tobacco and oilseed rape have been demonstrated to lead to male sterility (Mariani et al, Nature, 347:737-741, 1990).

A number of mutations were discovered in maize that confer cytoplasmic male sterility. One mutation in particular, referred to as T cytoplasm, also correlates with sensitivity to Southern corn leaf blight. A DNA sequence, designated TURF 13 (Levings, Science, 250:942-947, 1990), was identified that correlates with T cytoplasm. Therefore, in some embodiments, it would be possible through the introduction of TURF 13 via transformation, to separate male sterility from disease sensitivity. As it is necessary to be able to restore male fertility for breeding purposes and for grain production, genes encoding restoration of male fertility also may be introduced.

### Altered Nutritional Content

Genes may be introduced into plants to improve or alter the nutrient quality or content of a particular crop. Introduction of genes that alter the nutrient composition of a crop may greatly enhance the feed or food value. For example, the protein of many grains is suboptimal for feed and food purposes, especially when fed to pigs, poultry, and humans. The protein is deficient in several amino acids that are essential in the diet of these species, requiring the addition of supplements to the grain. Limiting essential amino acids may include lysine, methionine, tryptophan, threonine, valine, arginine, and histidine. Some amino acids become limiting only after corn is supplemented with other inputs for feed formulations. The levels of these essential amino acids in seeds and grain may be elevated by mechanisms which include, but are not limited to, the introduction of genes to increase the biosynthesis of the amino acids, decrease the degradation of the amino acids, increase the storage of the amino acids in proteins, or increase transport of the amino acids to the seeds or grain.

Polypeptides useful for providing increased seed protein quantity and/or quality include polypeptides involved in the metabolism of amino acids in plants, particularly polypeptides involved in biosynthesis of methionine/cysteine and lysine, amino acid transporters, amino acid efflux carriers, seed storage proteins, proteases, or polypeptides involved in phytic acid metabolism. The protein composition of a crop may be altered to improve the balance of amino acids in a variety of ways including elevating expression of native proteins, decreasing expression of those with poor composition, changing the composition of native proteins, or introducing genes encoding entirely new proteins.

The introduction of genes that alter the oil content of a crop plant may also be of value. Increases in oil content may result in increases in metabolizable-energy-content and density of the seeds for use in feed and food. The introduced genes may encode enzymes that remove or reduce rate-limitations or regulated steps in fatty acid or lipid biosynthesis. Such genes may include, but are not limited to, those that encode acetyl-CoA carboxylase, ACP-acyltransferase, alpha-ketoacyl-ACP synthase, or other well known fatty acid biosynthetic activities. Other possibilities are genes that encode proteins that do not possess enzymatic activity such as acyl carrier protein. Genes may be introduced that alter the balance of fatty acids present in the 5 oil providing a more healthful or nutritive feedstuff. The introduced DNA also may encode sequences that block expression of enzymes involved in fatty acid biosynthesis, altering the proportions of fatty acids present in crops.

Genes may be introduced that enhance the nutritive value of crops, or of foods derived from crops by increasing the level of naturally occurring phytosterols, or by encoding for proteins to enable the synthesis of phytosterols in crops. The phytosterols from these crops can be processed directly into foods, or extracted and used to manufacture food products.

Genes may be introduced that enhance the nutritive value of the starch component of crops, for example by increasing the degree of branching, resulting in 5 improved utilization of the starch in livestock by delaying its metabolism. Additionally, other major constituents of a crop may be altered, including genes that affect a variety of other nutritive, processing, or other quality aspects. For example, pigmentation may be increased or decreased.

Carbohydrate metabolism may be altered, for example by increased sucrose production and/or transport. Polypeptides useful for affecting on carbohydrate metabolism include polypeptides involved in sucrose or starch metabolism, carbon assimilation or carbohydrate transport, including, for example sucrose transporters or glucose/hexose transporters, enzymes involved in glycolysis/gluconeogenesis, the pentose phosphate cycle, or raffinose biosynthesis, or polypeptides involved in glucose signaling, such as SNFI complex proteins.

Feed or food crops may also possess sub-optimal quantities of vitamins, antioxidants or other nutraceuticals, requiring supplementation to provide adequate nutritive value and ideal health value. Introduction of genes that enhance vitamin biosynthesis may be envisioned including, for example, vitamins A, E, B 12, choline, or the like. Mineral content may also be sub-optimal. Thus genes that affect the accumulation or availability of compounds containing phosphorus, sulfur, calcium, manganese, zinc, or iron among others would be valuable.

Numerous other examples of improvements of crops can be used according to the systems, nucleic acid compositions, and methods of the disclosure. The improvements may not necessarily involve grain, but may, for example, improve the value of a crop for silage. Introduction of DNA to accomplish this might include sequences that alter lignin production such as those that result in the “brown midrib” phenotype associated with superior feed value for cattle. Other genes may encode for enzymes that alter the structure of extracellular carbohydrates in the stover, or that facilitate the degradation of the carbohydrates in the non-grain portion of the crop so that it can be efficiently fermented into ethanol or other useful carbohydrates. It may be desirable to modify the nutritional content of plants by reducing undesirable components such as fats, starches, etc. This may be done, for example, by the use of exogenous nucleic acids that encode enzymes which increase plant use or metabolism of such components so that they are present at lower quantities. Alternatively, it may be done by use of exogenous nucleic acids that reduce expression levels or activity of native plant enzymes that synthesize such components.

Likewise the elimination of certain undesirable traits may improve the food or feed value of the crop. Many undesirable traits must currently be eliminated by special post-harvest processing steps and the degree to which these can be engineered into the plant prior to harvest and processing would provide significant value. Examples of such traits are the elimination of anti-nutritionals such as phytates and phenolic compounds which are commonly found in many crop species. Also, the reduction of fats, carbohydrates and certain phytohormones may be valuable for the food and feed industries as they may allow a more efficient mechanism to meet specific dietary requirements.

In addition to direct improvements in feed or food value, genes also may be introduced which improve the processing of crops and improve the value of the products resulting from the processing. One use of crops is via wetmilling. Thus novel genes that increase the efficiency and reduce the cost of such processing, for example by decreasing steeping time, may also find use. Improving the value of wetmilling products may include altering the quantity or quality of starch, oil, corn gluten meal, or the components of gluten feed. Elevation of starch may be achieved through the identification and elimination of rate limiting steps in starch biosynthesis by expressing increased amounts of enzymes involved in biosynthesis or by decreasing levels of the other components of crops resulting in proportional increases in starch. Oil is another product of wetmilling, the value of which may be improved by introduction and expression of genes. Oil properties may be altered to improve its performance in the production and use of cooking oil, shortenings, lubricants or other oil-derived products or improvement of its health attributes when used in the food-related applications. Novel fatty acids also may be synthesized which upon extraction can serve as starting materials for chemical syntheses. The changes in oil properties may be achieved by altering the type, level, or lipid arrangement of the fatty acids present in the oil. This in turn may be accomplished by the addition of genes that encode enzymes that catalyze the synthesis of novel fatty acids (e.g. fatty acid elongases, desaturases) and the lipids possessing them or by increasing levels of native fatty acids while, in some embodiments, reducing levels of precursors or breakdown products. Alternatively, DNA sequences may be introduced which slow or block steps in fatty acid biosynthesis resulting in the increase in precursor fatty acid intermediates. Genes that might be added include desaturases, epoxidases, hydratases, dehydratases, or other enzymes that catalyze reactions involving fatty acid intermediates. Representative examples of catalytic steps that might be blocked include the desaturations from stearic to oleic acid or oleic to linolenic acid resulting in the respective accumulations of stearic and oleic acids. Another example is the blockage of elongation steps resulting in the accumulation of C8 to C12 saturated fatty acids.

Polypeptides useful for providing increased seed oil quantity and/or quality include polypeptides involved in fatty acid and glycerolipid biosynthesis, beta-oxidation enzymes, enzymes involved in biosynthesis of nutritional compounds, such as carotenoids and tocopherols, or polypeptides that increase embryo size or number or thickness of aleurone.

Polypeptides involved in production of galactomannans or arabinogalactans are of interest for providing plants having increased and/or modified reserve polysaccharides for use in food, pharmaceutical, cosmetic, paper and paint industries.

Polypeptides involved in modification of flavonoid/isoflavonoid metabolism in plants include cinnamate-4-hydroxylase, chalcone synthase or flavones synthase. Enhanced or reduced activity of such polypeptides in modified plants will provide changes in the quantity and/or speed of flavonoid metabolism in plants and may improve disease resistance by enhancing synthesis of protective secondary metabolites or improving signaling pathways governing disease resistance.

Polypeptides involved in lignin biosynthesis are of interest for increasing plants' resistance to lodging and for increasing the usefulness of plant materials as biofuels.

### Production or Assimilation of Chemicals or Biologicals

It may further be considered that a modified plant prepared in accordance with the disclosure may be used for the production or manufacturing of useful biological compounds that were either not produced at all, or not produced at the same level, in the plant previously. Alternatively, plants produced in accordance with the disclosure may be made to metabolize or absorb and concentrate certain compounds, such as hazardous wastes, thereby allowing bioremediation of these compounds.

The novel plants producing these compounds are made possible by the introduction and expression of one or potentially many genes with the constructs provided by the disclosure. The vast array of possibilities include but are not limited to any biological compound which is presently produced by any organism such as proteins, nucleic acids, primary and intermediary metabolites, carbohydrate polymers, enzymes for uses in bioremediation, enzymes for modifying pathways that produce secondary plant metabolites such as falconoid or vitamins, enzymes that can produce pharmaceuticals, and for introducing enzymes that may produce compounds of interest to the manufacturing industry such as specialty chemicals and plastics. The compounds may be produced by the plant, extracted upon harvest and/or processing, and used for any presently recognized useful purpose such as pharmaceuticals, fragrances, and industrial enzymes to name a few.

### Other Characteristics

Cell cycle modification: Polypeptides encoding cell cycle enzymes and regulators of the cell cycle pathway are useful for manipulating growth rate in plants to provide early vigor and accelerated maturation. Improvements in quality traits, such as seed oil content, may also be obtained by expression of cell cycle enzymes and cell cycle regulators. Polypeptides of interest for modification of cell cycle pathway include cycling and EIF5a pathway proteins, polypeptides involved in polyamine metabolism, polypeptides which act as regulators of the cell cycle pathway, including cyclin-dependent kinases (CDKs), CDK-activating kinases, cell cycle-dependent phosphatases, CDK-inhibitors, Rb and Rb-binding proteins, or transcription factors that activate genes involved in cell proliferation and division, such as the E2F family of transcription factors, proteins involved in degradation of cyclins, such as cullins, and plant homologs of tumor suppressor polypeptides.

Plant growth regulators: Polypeptides involved in production of substances that regulate the growth of various plant tissues are of interest in the present disclosure and may be used to provide modified plants having altered morphologies and improved plant growth and development profiles leading to improvements in yield and stress response. Of particular interest are polypeptides involved in the biosynthesis, or degradation of plant growth hormones, such as gibberellins, brassinosteroids, cytokinins, auxins, ethylene or abscisic acid, and other proteins involved in the activity, uptake and/or transport of such polypeptides, including for example, cytokinin oxidase, cytokinin/purine permeases, F-box proteins, G-proteins or phytosulfokines.

Transcription factors in plants: Transcription factors play a key role in plant growth and development by controlling the expression of one or more genes in temporal, spatial and physiological specific patterns. Enhanced or reduced activity of such polypeptides in modified plants will provide significant changes in gene transcription patterns and provide a variety of beneficial effects in plant growth, development and response to environmental conditions. Transcription factors of interest include, but are not limited to myb transcription factors, including helix-turn-helix proteins, homeodomain transcription factors, leucine zipper transcription factors, MADS transcription factors, transcription factors having AP2 domains, zinc finger transcription factors, CCAAT binding transcription factors, ethylene responsive transcription factors, transcription initiation factors or UV damaged DNA binding proteins.

Homologous recombination: Increasing the rate of homologous recombination in plants is useful for accelerating the introgression of trans genes into breeding varieties by backcrossing, and to enhance the conventional breeding process by allowing rare recombinants between closely linked genes in phase repulsion to be identified more easily. Polypeptides useful for expression in plants to provide increased homologous recombination include polypeptides involved in mitosis and/or meiosis, DNA replication, nucleic acid metabolism, DNA repair pathways or homologous recombination pathways including for example, recombinases, nucleases, proteins binding to DNA double-strand breaks, single-strand DNA binding proteins, strand-exchange proteins, resolvases, ligases, helicases and polypeptide members of the RAD52 epistasis group.

Non-Protein-Expressing Exogenous Nucleic Acids Plants with decreased expression of a gene of interest can also be achieved, for example, by expression of antisense nucleic acids, dsRNA or RNAi, catalytic RNA such as ribozymes, sense expression constructs that exhibit cosuppression effects, aptamers or zinc finger proteins.

Antisense RNA reduces production of the polypeptide product of the target messenger RNA, for example by blocking translation through formation of RNA:RNA duplexes or by inducing degradation of the target mRNA. Antisense approaches are a way of preventing or reducing gene function by targeting the genetic material as disclosed in U.S. Pat. Nos. 4,801,540; 5,107,065; 5,759,829; 5,910,444; 6,184,439; and 6,198,026, all of which are incorporated herein by reference. In one approach, an antisense gene sequence is introduced that is transcribed into antisense RNA that is complementary to the target mRNA. For example, part or all of the normal gene sequences are placed under a promoter in inverted orientation so that the ‘wrong’ or complementary strand is transcribed into a non-protein expressing antisense RNA. The promoter used for the antisense gene may influence the level, timing, tissue, specificity, or inducibility of the antisense inhibition.

RNAi gene suppression in plants by transcription of a dsRNA is described in U.S. Pat. No. 6,506,559, U.S. patent application Publication No. 2002/0168707, WO 98/53083, WO 99/53050 and WO 99/61631, all of which are incorporated herein by reference. The double-stranded RNA or RNAi constructs can trigger the sequence-specific degradation of the target messenger RNA. Suppression of a gene by RNAi can be achieved using a recombinant DNA construct having a promoter operably linked to a DNA element comprising a sense and anti-sense element of a segment of genomic DNA of the gene, e.g., a segment of at least about 23 nucleotides, more preferably about 50 to 200 nucleotides where the sense and anti-sense DNA components can be directly linked or joined by an intron or artificial DNA segment that can form a loop when the transcribed RNA hybridizes to form a hairpin structure. Catalytic RNA molecules or ribozymes can also be used to inhibit expression of the target gene or genes or facilitate molecular reactions. Ribozymes are targeted to a given sequence by hybridization of sequences within the ribozyme to the target mRNA. Two stretches of homology are required for this targeting, and these stretches of homologous sequences flank the catalytic ribozyme structure. It is possible to design ribozymes that specifically pair with virtually any target mRNA and cleave the target mRNA at a specific location, thereby inactivating it. A number of classes of ribozymes have been identified. One class of ribozymes is derived from a number of small circular RNAs that are capable of self-cleavage and replication in plants. The RNAs replicate either alone (viroid RNAs) or with a helper virus (satellite RNAs). Examples include Tobacco Ringspot Virus (Prody et ah, Science, 231:1577-1580, 1986), Avocado Sunblotch Viroid (Palukaitis et ah, Virology, 99:145-151, 1979; Symons, Nuch Acids Res., 9:6527-6537, 1981), and Lucerne Transient Streak Virus (Forster and Symons, Cell, 49:211-220, 1987), and the satellite RNAs from velvet tobacco mottle virus, Solanum nodiflorum mottle virus and subterranean clover mottle virus. The design and use of target RNA-specific ribozymes is described in Haseloff, et al., Nature 334:585-591 (1988). Several different ribozyme motifs have been described with RNA cleavage activity (Symons, Annu. Rev. Biochem., 61:641-671, 1992). Other suitable ribozymes include sequences from RNase P with RNA cleavage activity (Yuan et ah, Proc. Natl. Acad. Sd. USA, 89:8006-8010, 1992; Yuan and Altman, Science, 263:1269-1273, 1994; U.S. Pat. Nos. 5,168,053 and 5,624,824), hairpin ribozyme structures (Berzal-Herranz et ah, Genes and Deveh, 6:129-134, 1992; Chowrira et ah, J. Biol. Chem., 269:25856-25864, 1994) and Hepatitis Delta virus based ribozymes (U.S. Pat. No. 5,625,047). The general design and optimization of ribozyme directed RNA cleavage activity has been discussed in detail (Haseloff and Gerlach, 1988, Nature. 1988 Aug. 18; 334(6183):585-91, Chowrira et al., J. Biol. Chem., 269:25856-25864, 1994).

Another method of reducing protein expression utilizes the phenomenon of cosuppression or gene silencing (for example, U.S. Pat. Nos. 6,063,947; 5,686,649; or 5,283,184; each of which is incorporated herein by reference). Cosuppression of an endogenous gene using a full-length cDNA sequence as well as a partial cDNA sequence are known (for example, Napoli et al., Plant Cell 2:279-289 [1990]; van der Krol et al., Plant Cell 2:291-299 [1990]; Smith et al., MoI. Gen. Genetics 224:477-481 [1990]). The phenomenon of cosuppression has also been used to inhibit plant target genes in a tissue-specific manner. In some embodiments, nucleic acids from one species of plant are expressed in another species of plant to effect cosuppression of a homologous gene. The introduced sequence generally will be substantially identical to the endogenous sequence intended to be repressed, for example, about 65%, 80%, 85%, 90%, or preferably 95% or greater identical. Higher identity may result in a more effective repression of expression of the endogenous sequence. A higher identity in a shorter than full length sequence compensates for a longer, less identical sequence. Furthermore, the introduced sequence need not have the same intron or exon pattern, and identity of non-coding segments will be equally effective. Generally, where inhibition of expression is desired, some transcription of the introduced sequence occurs. The effect may occur where the introduced sequence contains no coding sequence per se, but only intron or untranslated sequences homologous to sequences present in the primary transcript of the endogenous sequence.

Yet another method of reducing protein activity is by expressing nucleic acid ligands, so-called aptamers, which specifically bind to the protein. Aptamers may be obtained by the SELEX (Systematic Evolution of Ligands by Exponential Enrichment) method. See U.S. Pat. No. 5,270,163, incorporated herein by reference. In the SELEX method, a candidate mixture of single stranded nucleic acids having regions of randomized sequence is contacted with the protein and those nucleic acids having an increased affinity to the target are selected and amplified. After several iterations a nucleic acid with optimal affinity to the polypeptide is obtained and is used for expression in modified plants.

A zinc finger protein that binds a polypeptide-encoding sequence or its regulatory region is also used to alter expression of the nucleotide sequence. Transcription of the nucleotide sequence may be reduced or increased. Zinc finger proteins are, for example, described in Beerli et al. (1998) PNAS 95:14628-14633., or in WO 95/19431, WO 98/54311, or WO 96/06166, all incorporated herein by reference.

Other examples of non-protein expressing sequences specifically envisioned for use with the disclosure include tRNA sequences, for example, to alter codon usage, and rRNA variants, for example, which may confer resistance to various agents such as antibiotics. It is contemplated that unexpressed DNA sequences, including novel synthetic sequences, may be introduced into cells as proprietary “labels” of those cells and plants and seeds thereof. It would not be necessary for a label DNA element to disrupt the function of a gene endogenous to the host organism, as the sole function of this DNA would be to identify the origin of the organism. For example, one can introduce a unique DNA sequence into a plant and this DNA element would identify all cells, plants, and progeny of these cells as having arisen from that labeled source. Inclusion of label DNAs would enable one to distinguish proprietary germplasm or germplasm derived from such, from unlabelled germplasm.

In some embodiments, the cargo sequence comprises a detectable protein, for example a protein tag or a fluorescent protein. The tag can be an epitope tag. The epitope tag can comprise a myc tag, a FLAG tag, a polyHistidine tag, a HiBiT tag, HA tag, S-peptide tag, calmodulin-binding peptide (CBP), glutathione S-transferase (GST), maltose binding protein (MBP), or any combination thereof. The cargo sequence can comprise an exogenous sequence encoding a fluorescent protein. The fluorescent protein can comprise mPlum, mCherry, DsRed, FASTred, mOrange, EYFP, VENUS, YPet, GFP, EGFP, EmGFP, mCFP, Cerulean, CyPet, Kaede, or any combination thereof.

### Rna-Guided DNA Binding Complex and Transposition Complex

As provided above, CRISPR-associated transposons or CASTs are mobile genetic elements (MGEs) that have evolved to make use of minimal CRISPR systems for RNA-guided transposition of their DNA. Unlike traditional CRISPR systems that contain interference mechanisms to degrade targeted DNA, CASTs lack proteins and/or protein domains responsible for DNA cleavage. Specialized transposon machinery, similar to that of Tn7 transposon, complexes with the CRISPR RNA (crRNA) and associated Cas proteins for transposition. CAST systems have been characterized in a wide range of bacteria and make use of variable CRISPR configurations including Type I-F, Type I-B, Type I-C, Type I-D, Type I-E, Type IV, and Type V-K.

CRISPR-associated transposons are, in some instances, similar to the Tn7 transposon which functions with a cut and paste mechanism. It contains a heteromeric transposase consisting of TnsA and TnsB proteins, and a regulator protein TnsC. Structural analysis has shown binding of the TnsB protein and sequence specific motifs on the ends of the transposon which allows for excision and mobility. Targeting for integration is done by the TnsD or TnsE proteins which preferentially target safe sites within the host chromosome or mobile elements (plasmids or bacteriophages), respectively. TnsE is not found in CASTs but a TnsD homolog, TniQ, is present and functions to bridge the gap between the transposase and CRISPR-Cas. Multiple CRISPR types have been found to associate with transposons with two of the most studied being Type I-F, which makes use of a multi-subunit effector, and Type V-K, which makes use of a single Cas12k effector. In both cases, Tn7 transposons have evolved to make use of these effectors to create R loops for site-specific integration. While TnsA is present in Type I-F systems, it is notably absent in Type V-K systems which showed higher off-target integrations during initial characterization.

Provided herein are systems comprising an RNA-guided DNA binding complex or one or more first helper polynucleotides each comprising a sequence encoding a component of the RNA-guided DNA binding complex, wherein the RNA-guided DNA binding complex comprises one or more Cas proteins, a transposase, a crRNA, or any combination thereof. In some embodiments, there is provided nucleic acid compositions comprising one or more first helper polynucleotides each comprising a sequence encoding a component of an RNA-guided DNA binding complex, wherein the RNA-guided DNA binding complex comprises one or more Cas proteins, a transposase, a crRNA, or any combination thereof. The system, the RNA-guided DNA binding complex and/or the transposition complex can be derived from a Type I-B, Type I-D, TypeI-F, or Type V-K Crispr-associated transposase system of a bacteria. The bacteria can comprise Vibrio cholera (Vch), Pseudoalteromonas (Pse), or Scytonema hofmanni (Sho).

The one or more Cas proteins can comprise a Cas6 protein. The one or more Cas proteins can comprise a Cas7 protein. The one or more Cas proteins can comprise a Cas8 protein. The one or more Cas proteins can comprise a Cas6 protein, a Cas7 protein, and a Cas8 protein.

The Cas6 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 8-9. The Cas7 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 10-11. The Cas8 protein can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 12-13. The transposase of the RNA-guided DNA binding complex can comprise a TniQ protein. The TniQ protein can comprise an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 14-15.

The one or more transposases of the transposition complex can comprise a TnsA transposase. The one or more transposases of the transposition complex can comprise a TnsB transposase. The one or more transposases of the transposition complex can comprise a TnsC protein. The one or more transposases of the transposition complex can comprise a TnsA transposase, a TnsB transposase, and a TnsC protein. The one or more transposases of the transposition complex can comprise a TnsAB fusion protein and a TnsC protein. The TnsAB fusion protein can comprise an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 16-17. The TnsC protein can comprise an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 18-19.

In some embodiments, the system comprises one or more helper accessory proteins or one or more helper accessory polynucleotides each comprising a sequence encoding at least one of the one or more helper accessory proteins, wherein the one or more helper accessory proteins comprise ClpX and/or ClpP. ClpX (e.g., ClpX AAA+ATPase) and ClpP (e.g., ClpP peptidase) are bacterial proteins that can promote disassembly of post-transposition complex (PTC) in bacterial Tn7 and Mu transposon systems. In some embodiments, the activity of ClpX and/or ClpP can enhance the transposition efficiency, compared to methods and systems that lack said proteins.

The ClpX can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 21. The ClpP can comprise or consist of an amino acid sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 20.

crRNAs

The RNA-guided DNA-binding complex can comprise a crRNA. The terms “gRNA,” “guide RNA”, “CRISPR guide sequence” or “crRNA” may be used interchangeably throughout and refer to a nucleic acid comprising a sequence that determines the binding specificity of the RNA-guided DNA-binding complex. A crRNA hybridizes to (complementary to, partially or completely) a double-stranded target sequence (e.g., in the genome) in a cell, e.g., a plant cell. The crRNA can comprise a spacer that hybridizes to a search target sequence on a first strand of the double stranded target sequence (a target site). The spacer may be between 15-35 nucleotides, 18-33 nucleotides, or 19-35 nucleotides in length. In some embodiments, the crRNA (e.g., gRNA) sequence that hybridizes to the target site is 15, 16, 17, 18, 19, 20, 21, 22, 23, 24, 25 26, 27, 28, 29, 30, 31, 32, or 33 nucleotides in length. In some embodiments, the crRNA sequence that hybridizes to the target site is between 10-30, or between 15-25, or 25-35 nucleotides in length.

To facilitate crRNA design, many computational tools have been developed (See Prykhozhij et al (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2); 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of crRNA(s); including but not limited to, Genscript Interactive CRISPR gRNA Design Tool, WU-CRISPR, and Broad Institute GPP sgRNA Designer. There are also publicly available pre-designed gRNA sequences to target many genes and locations within the genomes of many species (human, mouse, rat, zebrafish, C. elegans), including but not limited to, IDT DNA Predesigned Alt-R CRISPR-Cas9 guide RNAs, Addgene Validated gRNA Target Sequences, and GenScript Genome-wide gRNA databases.

In addition to a sequence that binds to a target nucleic acid (e.g., a spacer), in some embodiments, the gRNA may also comprise a scaffold sequence. In some embodiments, such a gRNA may be referred to as a single guide RNA (sgRNA) or a crRNA. Exemplary scaffold sequences will be evident to one of skill in the art and can be found, for example, in Jinek, et al. Science (2012) 337(6096):816-821, and Ran, et al. Nature Protocols (2013) 8:2281-2308. crRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer).

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence. The crRNA can comprise a [repeat scaffold]-[spacer]-[repeat scaffold] structure. The first strand of the double stranded target sequence can be the sense strand.

In some embodiments, the spacer sequence of the crRNA is at least 50%, 55%, 60%, 65%, 70%, 75%, 80%, 85%, 90%, 95%, 96%, 97%, 98%, 99%, or at least 100% complementary to a search target sequence on a first strand of the double stranded target sequence. In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the search target sequence on a first strand of the double stranded target sequence (e.g., the last 5, 6, 7, 8, 9, or 10 nucleotides of the 3′ end of the target sequence). The crRNA can comprise a spacer that is complementary to a search target sequence on a first strand of the double stranded target sequence. The first strand of the double stranded target sequence can be the sense strand.

The target sequence may be flanked by a protospacer adjacent motif (PAM). A PAM site is a nucleotide sequence in proximity to a target sequence. For example, a PAM may be a DNA sequence immediately following the DNA sequence targeted by the RNA-guided DNA binding complex.

The target sequence may or may not be flanked by a protospacer adjacent motif (PAM) sequence. In certain embodiments, a nucleic acid-guided nuclease can only bind a target sequence if an appropriate PAM is present, see, for example Doudna et al., Science, 2014, 346(6213): 1258096, incorporated herein by reference. A PAM can be 5′ or 3′ of a target sequence. A PAM can be upstream or downstream of a target sequence. In some embodiments, the target sequence is immediately flanked on the 3′ end by a PAM sequence. A PAM can be 1, 2, 3, 4, 5, 6, 7, 8, 9, 10 or more nucleotides in length. In certain embodiments, a PAM is between 2-6 nucleotides in length. The target sequence may or may not be located adjacent to a PAM sequence (e.g., PAM sequence located immediately 3′ of the target sequence) (e.g., for Type I CRISPR/Cas systems and Type II CRISPR/Cas systems). In some embodiments, e.g., Type I systems, the PAM is on the alternate side of the protospacer (the 5′ end). Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (201 5)).

Non-limiting examples of the PAM sequences include: CC, CA, AG, GT, TA, AC, CA, GC, CG, GG, CT, TG, GA, AGG, TGG, T-rich PAMs (such as ITT, TTG, TTC, TTTT, NGG, NGA, NAG, NGGNG and NNAGAAW (W=A or T), NNNNGATT, NAAR (R=A or G), NNGRR (R=A or G), NNAGAA and NAAAAC, where “N” is any nucleotide. In some embodiments, e.g., for Type I-F CAST systems and the systems, nucleic acid compositions and methods described herein, the PAM sequence can be 5′-CN-3′, where “N” is any nucleotide.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization. There may be mismatches distal from the PAM.

The cargo sequence can be capable of being integrated at an integration site following binding of the RNA-guided DNA binding complex to the search target sequence. The integration site can be about 48 to 52 base pairs (e.g., 48, 49, 50, 51, or 52 base pairs) downstream of the double stranded target sequence. The double stranded target sequence can be situated within a selectable marker gene of the genome of the plant cell. The selectable marker gene can comprise a fluorescent protein coding gene, a phytoene desaturase (PDS) gene, a codA gene, a diphtheria toxin a subunits (DT-A) gene, an exotoxin A gene, a ricin toxin A gene, a cytochrome P-450 gene, an RNase T1 gene, or a barnase gene. The double stranded target sequence can be situated within a safe harbor locus of the genome of the plant cell.

### Polynucleotides and Nucleic Acid Compositions

Disclosed herein include systems for integration of a nucleic acid sequence into a double-stranded target sequence of a genome of a plant cell or a target plasmid in a plant cell. In some embodiments, the system comprises: i) an RNA-guided DNA binding complex or one or more first helper polynucleotides each comprising a sequence encoding a component of the RNA-guided DNA binding complex, wherein the RNA-guided DNA binding complex comprises one or more Cas proteins, a transposase, a crRNA, or any combination thereof, optionally the system comprises one or more helper accessory proteins or one or more helper accessory polynucleotides each comprising a sequence encoding at least one of the one or more helper accessory proteins, wherein the one or more helper accessory proteins comprise ClpX and ClpP; ii) a transposition complex or one or more second helper polynucleotides each comprising a sequence encoding a component of the transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

Disclosed herein include nucleic acid compositions for integration of a nucleic acid sequence into a double-stranded target sequence of a genome of a plant cell or a target plasmid in a plant cell. In some embodiments, the nucleic acid composition comprises: i) one or more first helper polynucleotides each comprising a sequence encoding a component of an RNA-guided DNA binding complex, wherein the RNA-guided DNA binding complex comprises one or more Cas proteins, a transposase, a crRNA, or any combination thereof, optionally the nucleic acid composition comprises one or more helper accessory polynucleotides each comprising a sequence encoding at least one of the one or more helper accessory proteins, wherein the one or more helper accessory proteins comprise ClpX and ClpP; ii) one or more second helper polynucleotides each comprising a sequence encoding a component of a transposition complex, wherein the transposition complex comprises one or more transposases and one or more transposons; and iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

Each of the one or more first and/or second helper polynucleotides, the one or more helper accessory polynucleotides or both, can be operably linked to one omore expression control elements (e.g., a promoter and/or a transcriptional terminator). Each of the one or more first helper polynucleotides can comprise a first promoter operably linked to the sequence encoding the component of the RNA-guided DNA binding complex. Each of the one or more second helper polynucleotides can comprise a second promoter operably linked to the sequence encoding the component of the transposition complex. Each of the one or more helper accessory polynucleotides can comprise a third promoter operably linked to the sequence encoding at least one of the one or more helper accessory proteins.

The first, second, and/or third promoters can be the same or different. The first, second, and/or third promoters can comprise a ubiquitous promoter, a constitutive promoter, a cell-type specific promoter, a tissue-specific promoter, an inducible promoter, or any combination thereof. In some embodiments, the constitutive promoter is selected from the group comprising: pCmYLCV911 (pCmY), pU6, pU3, pU6, pAct2, pAct-1, pUBQ10, pUBQ4, pUbi1, and PUbi2; the tissue-specific promoter is selected from the group comprising: pSIREO, pNAC10, pPAT21, phspr, pPFn2, pPEPC, PLhcb, pTA29, pLat52, pZml3, pOleosin, pGlutenin, pD-hordein, and pE8; the inducible promoter is selected from the group comprising: pAdh-1, pwunl, pGBSS, pHSP18.2, pRd29, pSR2, pCCA1, pUGT71C5, pGSE, pwin3.12, pR2329, pBs3, pCaPrx, p4xM1.1, p4xM2.3, pIFS2, pSAG12, pSEOF1, pEm, pRd29, pSAUR15A, and pChn48.

Each of the one or more first helper polynucleotides can comprise a first transcription terminator operably linked to the sequence encoding the component of the RNA-guided DNA binding complex. Each of the one or more second helper polynucleotides can comprise a second transcription terminator operably linked to the sequence encoding the component of the transposition complex; and/or Each of the one or more helper accessory polynucleotides can comprise a third transcription terminator operably linked to the sequence encoding at least one of the one or more helper accessory proteins.

The first, second, and/or third transcription terminators can be the same or different. The first, second, and/or third transcription terminators can comprise AtHSP18.2 (tHSP), tU6, tACT3, tACT3-tRb7MAR, tACT3-tTM6MAR, tEU, tEU-tTM6MAR, tEU (intronless), tEU (intronless)-tACT3-tRB7MAR, tHSP18-tEU-tRb7MAR, tHSP18-tACT3, tHSP18-tACT3-tRb7, tHSP18-tPINII-tRb7MAR, tHSP18-tPINII-tTM6MAR, tHSP18-tRb7MAR, tProteinase inhibitor II (tPINII), trbcS, or any combination thereof.

The system or the nucleic acid composition can comprise: at least three first helper polynucleotides each comprising a sequence encoding a Cas protein, wherein the sequence encoding the Cas protein is operably linked to a pCmY promoter and a tHSP terminator; a first helper polynucleotide comprising a sequence encoding a transposase protein, wherein the sequence encoding the transposase protein is operably linked to a pCmY promoter and a tHSP terminator; a first helper polynucleotide comprising a sequence encoding a crRNA, wherein the sequence encoding the crRNA is operably linked to a pU6 promoter and a tU6 terminator; and at least two second helper polynucleotides each comprising a sequence encoding a transposase, wherein the sequence encoding a transposase is operably linked to a pCmY promoter and a tHSP terminator. The system or the nucleic acid composition can comprise at least two helper accessory polynucleotides, wherein the sequence encoding at least one of the one or more helper accessory proteins is operably linked to a pCmY promoter and a tHSP terminator.

Further examples of promoters and terminators that can be used systems, compositions, and methods of the disclosure are describe below.

Exemplary classes of plant promoters are described below. Constitutive Expression promoters: Exemplary constitutive expression promoters include the ubiquitin promoter (e.g., sunflower-Binet et al. Plant Science 79: 87-94 (1991); maize-Christensen et al. Plant Molec. Biol. 12: 619-632 (1989); and Arabidopsis-Callis et al., J. Biol. Chem. 265: 12486-12493 (1990) and Norris et al., Plant Mol. Biol. 21: 895-906 (1993)); the CaMV 35S promoter (U.S. Pat. Nos. 5,858,742 and 5,322,938); or the actin promoter (e.g., rice—U.S. Pat. No. 5,641,876; McElroy et al. Plant Cell 2: 163-171 (1990), McElroy et al. Mol. Gen. Genet. 231: 150-160 (1991), and Chibbar et al. Plant Cell Rep. 12: 506-509 (1993)). Inducible Expression promoters: Exemplary inducible expression promoters include the chemically regulatable tobacco PR-I promoter (e.g., tobacco—U.S. Pat. No. 5,614,395; Arabidopsis-Lebel et al., Plant J. 16: 223-233 (1998); maize—U.S. Pat. No. 6,429,362). Various chemical regulators may be employed to induce expression, including the benzothiadiazole, isonicotinic acid, and salicylic acid compounds disclosed in U.S. Pat. Nos. 5,523,311 and 5,614,395. Other promoters inducible by certain alcohols or ketones, such as ethanol, include, for example, the alcA gene promoter from Aspergillus nidulans (Caddick et al. (1998) Nat. Biotechnol 16:177-180). A glucocorticoid-mediated induction system is described in Aoyama and Chua (1997) The Plant Journal 11: 605-612 wherein gene expression is induced by application of a glucocorticoid, for example a dexamethasone. Another class of useful promoters are water-deficit-inducible promoters, e.g. promoters which are derived from the 5′ regulatory region of genes identified as a heat shock protein 17.5 gene (HSP 17.5), an HVA22 gene (HVA22), and a cinnamic acid 4-hydroxylase (CA4H) gene of Zea mays. Another water-deficit-inducible promoter is derived from the rab-17 promoter as disclosed by Vilardell et al., Plant Molecular Biology, 17(5):985-993, 1990. See also U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, and U.S. Pat. No. 6,175,060 which discloses phosphorus deficiency inducible promoters.

As another example, numerous wound-inducible promoters have been described (e.g. Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), Warner et al. Plant J. 3: 191-201 (1993)). Logemann describe 5′ upstream sequences of the potato wunl gene. Xu et al. show that a wound-inducible promoter from the dicotyledon potato (pin2) is active in the monocotyledon rice. Rohrmeier & Lehle describe maize Wipl cDNA which is wound induced and which can be used to isolate the cognate promoter. Firek et al. and Warner et al. have described a wound-induced gene from the monocotyledon Asparagus officinalis, which is expressed at local wound and pathogen invasion sites.

Tissue-Specific Promoters: Exemplary promoters that express genes only in certain tissues are useful according to the present disclosure. For example, root specific expression can be attained using the promoter of the maize metallothionein-like (MTL) gene described by de Framond (FEBS 290: 103-106 (1991)) and also in U.S. Pat. No. 5,466,785, incorporated herein by reference. U.S. Pat. No. 5,837,848 discloses a root specific promoter. Another exemplary promoter confers pith-preferred expression (see PCT Pub. No. WO 93/07278, herein incorporated by reference, which describes the maize trpA gene and promoter that is preferentially expressed in pith cells). Leaf-specific expression may be attained, for example, by using the promoter for a maize gene encoding phosphoenol carboxylase (PEPC) (see Hudspeth & Grula, Plant Molec Biol 12: 579-589 (1989)). Pollen-specific expression may be conferred by the promoter for the maize calcium-dependent protein kinase (CDPK) gene which is expressed in pollen cells (WO 93/07278). U.S. Pat. Appl. Pub. No. 20040016025 describes tissue-specific promoters. Pollen-specific expression may be conferred by the tomato LAT52 pollen-specific promoter (Bate et. al., Plant Mol Biol. 1998 July; 37(5):859-69). See also U.S. Pat. No. 6,437,217 which discloses a root-specific maize RS81 promoter, U.S. Pat. No. 6,426,446 which discloses a root specific maize RS324 promoter, U.S. Pat. No. 6,232,526 which discloses a constitutive maize A3 promoter, U.S. Pat. No. 6,177,611 which discloses constitutive maize promoters, U.S. Pat. No. 6,433,252 which discloses a maize L3 oleosin promoter that are aleurone and seed coat-specific promoters, U.S. Pat. No. 6,429,357 which discloses a constitutive rice actin 2 promoter and intron, U.S. patent application Pub. No. 20040216189 which discloses an inducible constitutive leaf specific maize chloroplast aldolase promoter.

Optionally a plant transcriptional terminator can be used in place of the plant-expressed gene native transcriptional terminator. Exemplary transcriptional terminators are those that are known to function in plants and include the CaMV 35S terminator, the tml terminator, the nopaline synthase terminator and the pea rbcS E9 terminator. These can be used. in both monocotyledons and dicotyledons.

Various intron sequences have been shown to enhance expression, particularly in monocotyledonous cells. For example, the introns of the maize Adhl gene have been found to significantly enhance expression. Intron 1 was found to be particularly effective and enhanced expression in fusion constructs with the chloramphenicol acetyltransferase gene (Callis et al., Genes Develop. 1: 1183-1200 (1987)). The intron from the maize bronzel gene also enhances expression. Intron sequences have been routinely incorporated into plant transformation vectors, typically within the non-translated leader. U.S. Patent Application Publication 2002/0192813 discloses 5′, 3′ and intron elements useful in the design of effective plant expression vectors.

A number of non-translated leader sequences derived from viruses are also known to enhance expression, and these are particularly effective in dicotyledonous cells. Specifically, leader sequences from Tobacco Mosaic Virus (TMV, the “omega-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Sarnow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie et al., Molecular Biology of RNA, pages 237-256 (1989); or Maize Chlorotic Mottle Virus leader (MCMV) (Lommel et al., Virology 81:382-385 (1991). See also, Della-Cioppa et al., Plant Physiology 84:965-968 (1987). A minimal promoter may also be incorporated. Such a promoter has low background activity in plants when there is no transactivator present or when enhancer or response element binding sites are absent. One exemplary minimal promoter is the BzI minimal promoter, which is obtained from the bronzel gene of maize. Roth et al., Plant Cell 3: 317 (1991). A minimal promoter may also be created by use of a synthetic TATA element. The TATA element allows recognition of the promoter by RNA. polymerase factors and confers a basal level of gene expression in the absence of activation (see generally, Mukumoto (1993) Plant Mol Biol 23: 995-1003; Green (2000) Trends Biochem Sci 25: 59-63).

Sequences controlling the targeting of gene products also may be included. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). These signal sequences can be fused to heterologous gene products to effect the import of heterologous products into the chloroplast (van den Broeck, et al. Nature 313: 358-363 (1985)). DNA encoding for appropriate signal sequences can be isolated from the 5′ end of the cDNAs encoding the RUBISCO protein, the CAB protein, the EPSP synthase enzyme, the GS2 protein or many other proteins which are known to be chloroplast localized. Other gene products are localized to other organelles such as the mitochondrion and the peroxisome (e.g. Unger et al. Plant Molec. Biol. 13: 411-418 (1989)). Examples of sequences that target to such organelles are the nuclear-encoded ATPases or specific aspartate amino transferase isoforms for mitochondria. Targeting cellular protein bodies has been described by Rogers et al. (Proc. Natl. Acad. Sci. USA 82: 6512-6516 (1985)). In addition, amino terminal and carboxy-terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)).

Another element which may be introduced is a matrix attachment region element (MAR), such as the chicken lysozyme A element (Stief, 1989), which can be positioned around an expressible gene of interest to effect an increase in overall expression of the gene and diminish position dependent effects upon incorporation into the plant genome (Stief et al., Nature, 341:343, 1989; Phi-Van et al., MoI. Cell. Biol., 10:2302-2307.1990).

Use of non-plant promoter regions isolated from Drosophila melanogaster and Saccharomyces cerevisiae can be used to express genes in plants. The promoter can be derived from plant or non-plant species. In some embodiments, the nucleotide sequence of the promoter is derived from non-plant species for the expression of genes in plant cells, including but not limited to dicotyledon plant cells such as tobacco, tomato, potato, soybean, canola, sunflower, alfalfa, cotton and Arabidopsis, or monocotyledonous plant cell, such as wheat, maize, rye, rice, turf grass, oat, barley, sorghum, millet, and sugarcane. In some embodiments, the non-plant promoters are constitutive or inducible promoters derived from insect, e.g., Drosophila melanogaster or yeast, e.g., Saccharomyces cerevisiae. Promoters derived from any animal, protist, or fungi are also contemplated. These non-plant promoters can be operably linked to nucleic acid sequences encoding polypeptides or non-protein-expressing sequences including, but not limited to, antisense RNA and ribozymes, to form nucleic acid constructs, vectors, and host cells (prokaryotic or eukaryotic), comprising the promoters.

The sequence encoding the component of the RNA-guided DNA binding complex, the sequence encoding the component of the transposition complex, the sequence encoding at least one of the one or more helper accessory proteins, or any combination thereof, can be codon optimized for expression in the plant cell.

In some embodiments, the sequence encoding the component of the RNA-guided DNA binding complex encodes a Cas6 protein. The sequence encoding the Cas6 protein can comprise or consist of the nucleotide sequence of any one of SEQ ID NOs: 24-25 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 24-25. In some embodiments, the sequence encoding the component of the RNA-guided DNA binding complex encodes a Cas7 protein. The sequence encoding the Cas7 protein can comprise or consist of the nucleotide sequence of any one of SEQ ID NOs: 26-27 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 26-27. In some embodiments, the sequence encoding the component of the RNA-guided DNA binding complex encodes a Cas8 protein. The sequence encoding the Cas8 protein can comprise or consist of the nucleotide sequence of any one of SEQ ID NOs: 28-29 or a sequence that is at least 80%, 85%, 90%, 95%, 98%, 99%, or 100% identical to any one of SEQ ID NOs: 28-29. In some embodiments, the sequence encoding the component of the RNA-guided DNA binding complex encodes a TniQ protein. The sequence encoding the TniQ protein can comprise or consist of the nucleotide sequence of any one of SEQ ID NOs: 30-31 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 30-31.

In some embodiments, the sequence encoding the component of the transposition complex encodes a TnsAB fusion protein. The sequence encoding the TnsAB fusion protein can comprise or consist of the nucleotide sequence of any one of SEQ ID NOs: 32-33 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 32-33. In some embodiments, the sequence encoding the component of the transposition complex encodes a TnsC protein. The sequence encoding the TnsC protein can comprise or consist of the nucleotide sequence of any one of SEQ ID NOs: 34-35 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 34-35.

The sequence encoding ClpX can comprise or consist of the nucleotide sequence of SEQ ID NO: 37 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 37 and the sequence encoding ClpP can comprise or consist of the nucleotide sequence of SEQ ID NO: 36 or a sequence that is at least 80%, 85%, 90%, 95%, 98%, 99%, or 100% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 36.

The component of the RNA-guided DNA binding complex, the component of the transposition complex, or both, can comprise an N-terminal or a C-terminal tag. The tag can be an epitope tag. The epitope tag can comprise a myc tag, a FLAG tag, a polyHistidine tag, a HiBiT tag, HA tag, S-peptide tag, calmodulin-binding peptide (CBP), glutathione S-transferase (GST), maltose binding protein (MBP), or any combination thereof. The tag can comprise a fluorescent protein. The fluorescent protein can comprise mPlum, mCherry, DsRed, FASTred, mOrange, EYFP, VENUS, YPet, GFP, EGFP, EmGFP, mCFP, Cerulean, CyPet, Kaede, or any combination thereof.

The one or more first helper polynucleotides the one or more second helper polynucleotides, and/or the donor polynucleotide can be situated on the same nucleic acid or different nucleic acids. The one or more first helper polynucleotides, the one or more second helper polynucleotides, and/or the donor polynucleotide can be comprised within one or more vectors. The one or more vectors can comprise an RNA viral vector, a DNA viral vector, a plasmid vector, an artificial chromosome, or any combination thereof. The one or more vectors can comprise an Agrobacterium tumefaciens Ti vector. The one or more first helper polynucleotides, the one or more second helper polynucleotides, and/or the donor polynucleotide can be comprised within a T-DNA region of the Agrobacterium tumefaciens Ti vector.

The T-DNA region comprising the one or more first helper polynucleotides, the one or more second helper polynucleotides, and/or the donor polynucleotide can comprise the sequence of any one of SEQ ID NOs: 38-46. The T-DNA region comprising the one or more first helper polynucleotides, the one or more second helper polynucleotides, and/or the donor polynucleotide can comprise the sequence of any one of SEQ ID NOs: 38-46 or a sequence that is at least 80% identical (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to any one of SEQ ID NOs: 38-46.

### Plant Cell Transformation and Delivery

Any method known in the art can be used to deliver a system or nucleic acid composition of the disclosure to a plant cell. Agrobacterium (e.g., Agrobacterium tumefaciens)-mediated transformation is one method for introducing a desired genetic element into a plant. Several Agrobacterium species mediate the transfer of a specific DNA known as “T-DNA” that can be genetically engineered to carry a desired piece of DNA into many plant species. Plasmids used for delivery contain the T-DNA flanking the nucleic acid to be inserted into the plant. The major events marking the process of T-DNA mediated pathogenesis are induction of virulence genes, processing and transfer of T-DNA.

There are several methods known in the art to transform plant cells with Agrobacterium. One method is co-cultivation of Agrobacterium with cultured isolated protoplasts. This method requires an established culture system that allows culturing protoplasts and plant regeneration from cultured protoplasts. A second method is transformation of cells or tissues with Agrobacterium. This method requires (a) that the plant cells or tissues can be modified by Agrobacterium and (b) that the modified cells or tissues can be induced to regenerate into whole plants. A third method is transformation of seeds, apices or meristems with Agrobacterium. This method requires exposure of the meristematic cells of these tissues to Agrobacterium and micropropagation of the shoots or plan organs arising from these meristematic cells. Those of skill in the art are familiar with procedures for growth and suitable culture conditions for Agrobacterium as well as subsequent inoculation procedures. Liquid or semi-solid culture media can be used. The density of the Agrobacterium culture used for inoculation and the ratio of Agrobacterium cells to explant can vary from one system to the next, as can media, growth procedures, timing and lighting conditions.

Transformation of dicotyledons using Agrobacterium has long been known in the art, and transformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis. Exemplary strains include Agrobacterium tumefaciens strain C58, a nopaline-type strain that is used to mediate the transfer of DNA into a plant cell, octopine-type strains such as LBA4404 or succinamopine-type strains, e.g., EHAlOl or EHAl 05. The use of these strains for plant transformation has been reported and the methods are familiar to those of skill in the art.

U.S. Application No. 20040244075 published Dec. 2, 2004 describes improved methods of Agrobacterium-mediated transformation. The efficiency of transformation by Agrobacterium may be enhanced by using a number of methods known in the art. For example, the inclusion of a natural wound response molecule such as acetosyringone (AS) to the Agrobacterium culture has been shown to enhance transformation efficiency with Agrobacterium tumefaciens (Shahla et al., (1987) Plant Molec. Biol. 8:291-298). Alternatively, transformation efficiency may be enhanced by wounding the target tissue to be modified or transformed. Wounding of plant tissue may be achieved, for example, by punching, maceration, bombardment with microprojectiles, etc. (See e.g., Bidney et al., (1992) Plant Molec. Biol. 18:301-313).

In addition, a method described by Broothaerts, et. al. (Nature 433: 629-633, 2005) expands the bacterial genera that can be used to transfer genes into plants. This work involved the transfer of a disarmed Ti plasmid without T-DNA and another vector with T-DNA containing the marker enzyme beta-glucuronidase, into three different bacteria. Gene transfer was successful and this method significantly expands the tools available for gene delivery into plants.

Another widely used technique to genetically transform plants involves the use of microprojectile bombardment, in this process, a nucleic acid containing the desired genetic elements to be introduced into the plant is deposited on or in small dense particles, e.g., tungsten, platinum, or preferably 1 micron gold particles, which are then delivered at a high velocity into the plant tissue or plant cells using a specialized biolistics device. Many such devices have been designed and constructed; one in particular, the PDS 1000/He sold by BioRad, is the instrument most commonly used for biolistics of plant cells. The advantage of this method is that no specialized sequences need to be present on the nucleic acid molecule to be delivered into plant cells; delivery of any nucleic acid sequence is possible.

For the bombardment, cells in suspension are concentrated on filters or solid culture medium. Alternatively, immature embryos, seedling explants, or any plant tissue or target cells may be arranged on solid culture medium. The cells to be bombarded are positioned at an appropriate distance below the microprojectile stopping plate.

Various biolistics protocols have been described that differ in the type of particle or the manner in which DNA is coated onto the particle. Any technique for coating microprojectiles that allows for delivery of transforming DNA to the target cells may be used. For example, particles may be prepared by functionalizing the surface of a gold oxide particle by providing free amine groups. DNA, having a strong negative charge, will then bind to the functionalized particles. Parameters such as the concentration of DNA used to coat microprojectiles may influence the recovery of transformants containing a single copy of the transgene. For example, a lower concentration of DNA may not necessarily change, the efficiency of the transformation but may instead increase the proportion of single copy insertion events. In this regard, ranges of approximately 1 ng to approximately 10 g (10,000 ng), approximately 5 ng to 8 g or approximately 20 ng, 50 ng, 100 ng, 200 ng, 500 ng, 1 g, 2 g, 5 g, or 7 g of transforming DNA may be used per each 1.0-2.0 mg of starting 1.0 micron gold particles.

Other physical and biological parameters may be varied, such as manipulation of the DNA/microprojectile precipitate, factors that affect the flight and velocity of the projectiles, manipulation of the cells before and immediately after bombardment (including osmotic state, tissue hydration and the subculture stage or cell cycle of the recipient cells), the orientation of an immature embryo or other target tissue relative to the particle trajectory, and also the nature of the transforming DNA, such as linearized DNA or intact supercoiled plasmids. One may particularly wish to adjust physical parameters such as DNA concentration, gap distance, flight distance, tissue distance, and helium pressure. The particles delivered via biolistics can be “dry” or “wet.” In the “dry” method, the coated particles such as gold are applied onto a macrocarrier (such as a metal plate, or a carrier sheet made of a fragile material such as mylar) and dried. The gas discharge then accelerates the macrocarrier into a stopping screen, which halts the macrocarrier but allows the particles to pass through; the particles then continue their trajectory until they impact the tissue being bombarded. For the “wet” method, the droplet containing the coated particles is applied to the bottom part of a filter holder, which is attached to a base which is itself attached to a rupture disk holder used to hold the rupture disk to the helium egress tube for bombardment. The gas discharge directly displaces the DNA/gold droplet from the filter holder and accelerates the particles and their DNA cargo into the tissue being bombarded. The wet biolistics method has been described in detail elsewhere but has not previously been applied in the context of plants (Mialhe et al., MoI Mar Biol Biotechnol. 4(4):275-831995). The concentrations of the various components for coating particles and the physical parameters for delivery can be optimized using procedures known in the art.

More recently, nanomaterial mediated delivery can be used to transform and/or deliver biomolecules (e.g., nucleic acids and/or proteins) or particles comprising said biomolecules (e.g., an LNP), to a plant cell. In some embodiments, the one or more Cas proteins and the transposase of the RNA-guided DNA binding complex can be pre-complexed with the crRNA prior to the contacting (e.g., as a ribonucleoprotein particle or RNP). Any component of the systems or nucleic acid compositions described herein, or, e.g., an RNP can be formulated into a nanoparticle for delivery. For instance, the nanomaterial-mediated delivery can comprise: clay nanosheets, carbon nanotubes, carbon nanodots, self-assembled protein nanoparticles, peptides, DNA nanostructures, quantum dots, or any combination thereof. Methods for nanomaterial-mediated transformation of plant cells can also be found in U.S. Pat. No. 11,661,606, the content of which is hereby incorporated by reference in its entirety.

A variety of plant cells/tissues are suitable for transformation, including immature embryos, scutellar tissue, suspension cell cultures, immature inflorescence, shoot meristem, epithelial peels, nodal explants, callus tissue, hypocotyl tissue, cotyledons, roots, and leaves, meristem cells, and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspore-derived embryos, roots, hypocotyls, cotyledons and the like. Those cells which are capable of proliferating as callus also are recipient cells for genetic transformation.

Any suitable plant culture medium can be used. Examples of suitable media would include but are not limited to MS-based media (Murashige and Skoog, Physiol. Plant, 15:473-497, 1962) or N6-based media (Chu et al., Scientia Sinica 18:659, 1975) supplemented with additional plant growth regulators including but not limited to auxins such as picloram (4-amino-3,5,6-trichloropicolinic acid), 2,4-D (2,4-dichlorophenoxyacetic acid), naphalene-acetic acid (NAA) and dicamba (3,6-dichloroanisic acid), cytokinins such as BAP (6-benzylaminopurine) and kinetin, and gibberellins. Other media additives can include but are not limited to amino acids, macroelements, iron, microelements, vitamins and organics, carbohydrates, undefined media components such as casein hydrolysates, an appropriate gelling agent such as a form of agar, a low melting point agarose or Gelrite if desired. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Examples of such media would include but are not limited to Murashige and Skoog (Mursahige and Skoog, Physiol. Plant, 15:473-497, 1962), N6 (Chu et al., Scientia Sinica 18:659, 1975), Linsmaier and Skoog (Linsmaier and Skoog, Physio. Plant., 18:100, 1965), Uchimiya and Murashige (Uchimiya and Murashige, Plant Physiol. 15:473, 1962), Gamborg's B5 media (Gamborg et al., Exp. Cell Res., 50:151, 1968), D medium (Duncan et al., Planta, 165:322-332, 1985), Mc-Cown's Woody plant media (McCown and Lloyd,—HortScience 6:453, 1981), Nitsch and Nitsch (Nitsch and Nitsch, Science 163:85-87, 1969), and Schenk and Hildebrandt (Schenk and Hildebrandt, Can. J. Bot. 50:199-204, 1972) or derivations of these media supplemented accordingly. Those of skill in the art are aware that media and media supplements such as nutrients and growth regulators for use in transformation and regeneration and other culture conditions such as light intensity during incubation, pH, and incubation temperatures can be varied.

Those of skill in the art are aware of the numerous modifications in selective regimes, media, and growth conditions that can be varied depending on the plant system and the selective agent. Typical selective agents include but are not limited to antibiotics such as geneticin (G418), kanamycin, paromomycin or other chemicals such as glyphosate or other herbicides. Consequently, such media and culture conditions disclosed in the present disclosure can be modified or substituted with nutritionally equivalent components, or similar processes for selection and recovery of transgenic events, and fall within the scope of the present disclosure.

### Methods

Disclosed herein include methods for integration of a nucleic acid sequence into double-stranded target sequence of a genome of a plant cell or a target plasmid in a plant cell. In some embodiments, the method comprises: contacting the plant cell with a system or the nucleic acid composition of the disclosure, wherein the cargo sequence is integrated at an integration site in the genome of the plant cell or at a target site of the target plasmid upon expression of the RNA-guided DNA binding complex and the transposition complex in the plant cell.

In some embodiments, the integration site is about 48 to 52 base pairs (e.g., 48, 49, 50, 51 or 52 base pairs) downstream of the double stranded target sequence. In some embodiments, the one or more Cas proteins and the transposase of the RNA-guided DNA binding complex can be pre-complexed with the crRNA prior to the contacting.

The plant cell can be comprised within a plant. The plant cell can be comprised within a flower, a leaf, a stem, a root, terminal bud, a seed, or any other tissue of the plant. The plant cell can be a monocot plant cell or a eudicot plant cell.

In some embodiments, the integration of the cargo sequence confers i) a change in one or more of the following traits to the plant: grain number, grain size, grain weight, panicle size, tiller number, fragrance, nutritional value, shelf life, lycopene content, starch content and/or ii) lower gluten content, reduced levels of a toxin, reduced levels of steroidal glycoalkaloids, a substitution of mitosis for meiosis, asexual propagation, improved haploid breeding, and/or shortened growth time. In some embodiments, the integration of the cargo sequence confers one or more of the following traits to the plant cell and/or the plant: herbicide tolerance, drought tolerance, male sterility, insect resistance, abiotic stress tolerance, modified fatty acid metabolism, modified carbohydrate metabolism, modified seed yield, modified oil percent, modified protein percent, resistance to bacterial disease, resistance to fungal disease, and resistance to viral disease.

The system or the nucleic acid composition can be introduced into the plant cell by a technique comprising: pollen tube pathway, polyethylene glycol (PEG)-mediated gene transfer, electroporation, microinjection, microparticle bombardment, nanomaterial-mediated delivery, Agrobacterium tumefaciens-mediated transformation, or any combination thereof. The nanomaterial-mediated delivery can comprise: clay nanosheets, carbon nanotubes, carbon nanodots, self-assembled protein nanoparticles, peptides, DNA nanostructures, quantum dots, or any combination thereof. The one or more vectors can be introduced into the plant cell via Agrobacterium tumefaciens-mediated transformation of the plant cell.

Exemplary monocotyledonous plants (e.g., monocots) include, without limitation, wheat, maize, rice, orchids, onion, aloe, true lilies, grasses (e.g., Setaria), woody shrubs and trees (e.g., palms and bamboo), and food plants such as pineapple and sugar cane. Exemplary dicotyledonous plants (e.g., eudicots or dicots) include, without limitation, tomato, cassava, soybean, tobacco, potato, Arabidopsis, N. benthamiana, rose, pansy, sunflower, grape, strawberry, squash, bean, pea, and peanut.

In some embodiments, the methods described herein can include screening the plant, plant tissue, or plant cell to determine if an insertion has occurred at or near the sequence targeted by the crRNA. Any method known in the art can be used to detect insertions, such as PCR (endpoint PCR or quantitative PCR) and next generation sequences methods.

In addition, in some embodiments in which a plant part or plant cell is used, the methods provided herein can include regenerating a plant from the plant part or plant cell. The methods also can include breeding the plant (e.g., the plant into which the nucleic acids were introduced, or the plant obtained after regeneration of the plant part or plant cell used as a starting material) to obtain a genetically desired plant lineage. Methods for regenerating and breeding plants are well established in the art.

Disclosed herein include methods for screening for safe harbor loci in plants. In some embodiments, the method comprises: (a) generating a genome-wide crRNA library; (b) contacting a plant cell comprised within a plant with a system or the nucleic acid composition of disclosure, wherein: the system comprises pooled single or combinatorial crRNAs generated in step (a); or the one or more first helper polynucleotides comprise pooled single or combinatorial crRNAs generated in step a), wherein the cargo sequence is integrated into one or more double-stranded targets sites in the genome of the plant cell upon expression of the RNA-guided DNA binding complex and the transposition complex in the plant cell; (c) identifying integrants by expression of a gene product encoded by the cargo sequence; (d) subjecting the integrants to next-generation sequencing; and (e) performing bioinformatics analysis, a high-throughput phenotypic assay, or both to identify a safe harbor locus.

The plant can be a monocot plant or a eudicot plant. In some embodiments, integration of the cargo sequence at the identified safe harbor locus does not affect the growth, lifespan, health, gene expression profile, or any combination thereof, of the plant. The system or the nucleic acid composition can be introduced into the plant cell by a technique comprising: pollen tube pathway, polyethylene glycol (PEG)-mediated gene transfer, electroporation, microinjection, microparticle bombardment, nanomaterial-mediated delivery, Agrobacterium tumefaciens-mediated transformation, or any combination thereof. The nanomaterial-mediated delivery can comprise: clay nanosheets, carbon nanotubes, carbon nanodots, self-assembled protein nanoparticles, peptides, DNA nanostructures, quantum dots, or any combination thereof. In some embodiments, the one or more vectors are introduced into the plant cell via Agrobacterium tumefaciens-mediated transformation of the plant cell.

In some embodiments, the T-DNA region of the Ti vector comprises a bi-directional selection marker comprising a positive selection marker and a negative selection marker, wherein the identifying of step c) comprises: (i) generating a first filial generation (F1) plant comprising the T-DNA of the Ti vector by positive selection; and (ii) generating a second filial generation (F2) plant that does not comprise the T-DNA of the Ti vector from the first filial generation plant comprising the T-DNA of the Ti vector, by negative selection.

### Kits

Disclosed herein include kits comprising a system or nucleic acid composition described herein, and a set of instructions for use. For example, the kit can comprise a nucleic acid composition comprising one or more polynucleotides of the disclosure (e.g., the first and/or second helper polynucleotide, the one or more helper accessory polynucleotides, and/or the donor polynucleotide).

In addition to above-mentioned components, a kit can further include instructions for using the components of the kit to practice the methods. The instructions for practicing the methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

Some aspects of the embodiments discussed above are disclosed in further detail in the following examples, which are not in any way intended to limit the scope of the present disclosure.

### Example 1

### Targeted DNA Integration in Plants by CRISPR-Transposases

Provided in this example are methods and compositions of the disclosure related to targeted genome integration in plants using CRISPR-Transposases.

### Establish CAST-Mediated Targeted DNA Integration in Plants

### Construct Design of CAST Component

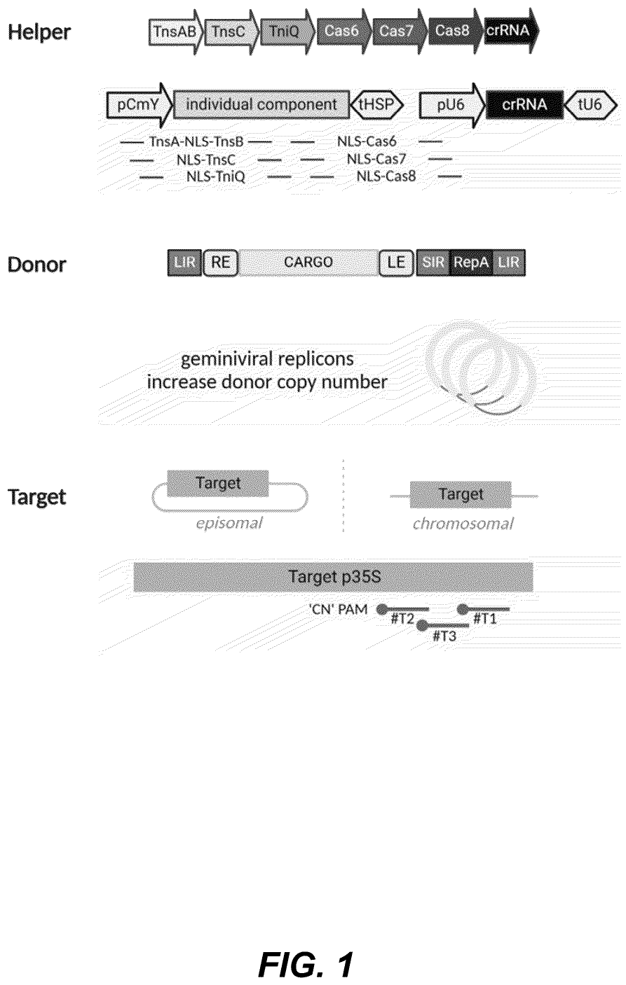

Vch and Pse CAST system was separated into three parts for modular reconstitution, as described below (FIG. 1).

Helper plasmid (pHelper) contains all essential CAST proteins and guide RNA in monocistronic cassettes. The type and position of the nuclear localization signal (NLS) was designed based on the CAST expression system in the mammalian study. pCmYLCV911 (pCmY) promoter and AtHSP18.2t (tHSP) terminator were used for all protein expression, which is experimentally the strongest promoter-terminator pair in plants. U6 promoter and terminator were used for crRNA expression.

Donor plasmid (pDonor) contains the donor DNA, which is the cargo sequence flanked by transposon left and right ends (LE and RE). The donor DNA is incorporated in a geminivirus-derived replication vector, which after entering the plant cell will initiate autonomous replication. This approach is designed to increase the donor DNA copy number in the cells to increase the insertion efficiency.

Target plasmid (pTarget) contains the intended target site for integration, which is only used in the episomal integration experiments. In the chromosomal experiments, target sites are selected from native genomic sequences. In experiments discussed below, 35S promoter is selected as the target site as it is present in a widely used GFP-expressing transgenic N. benthamiana line (16c) to drive the expression of GFP. Three target sequences on this 35S promoter were chosen with a CC PAM. Adhering to the generic construct design described here, all experiments were done in parallel using the Vch and Pse CAST with crRNA targeting three different locations on the 35S promoter labeled as T1, T2, and T3.

### Helper Protein Expression

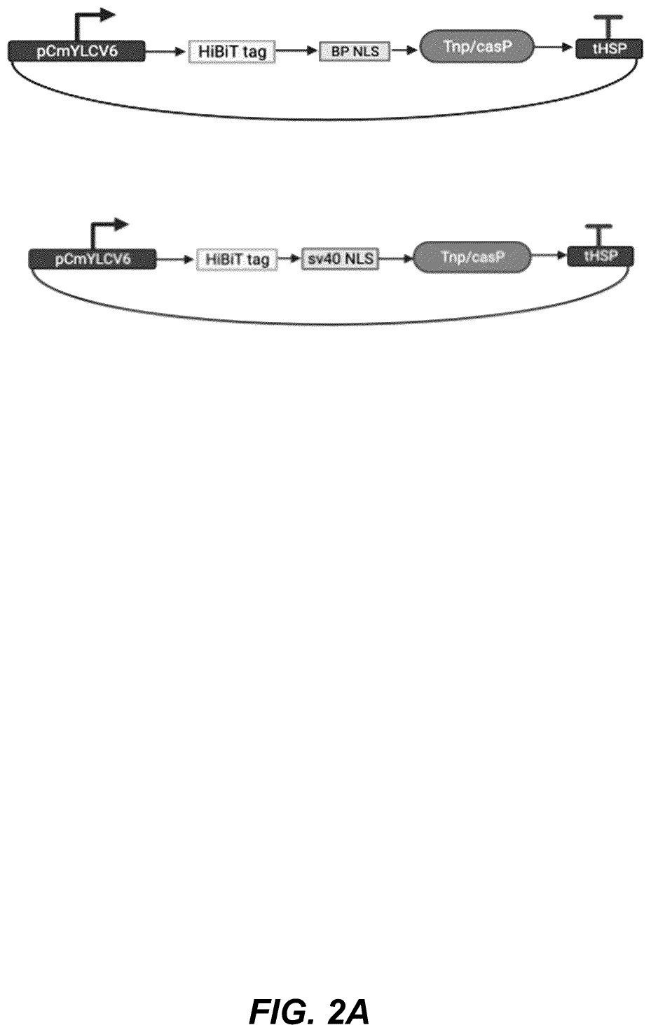

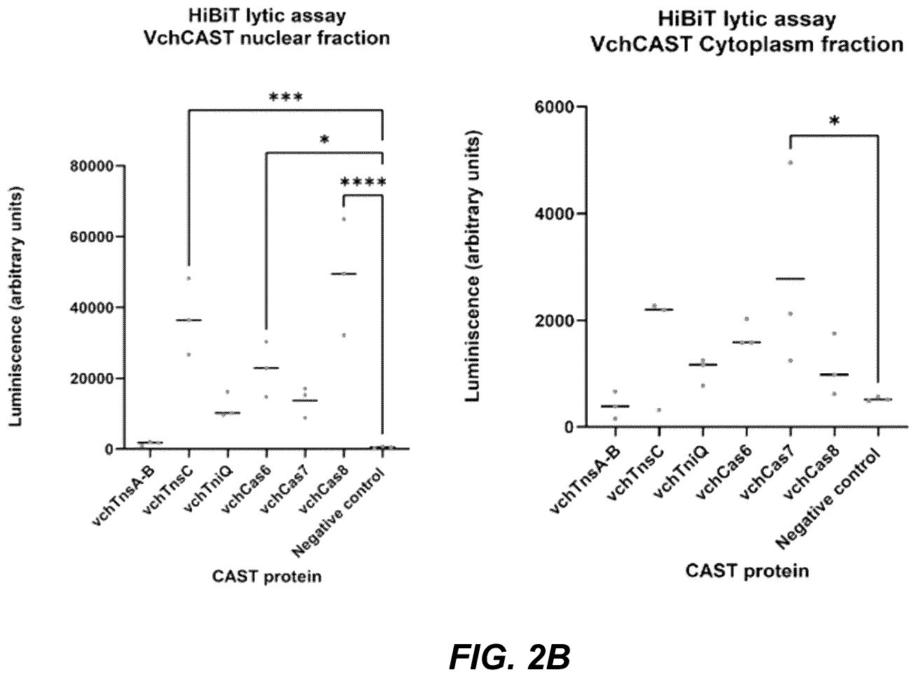

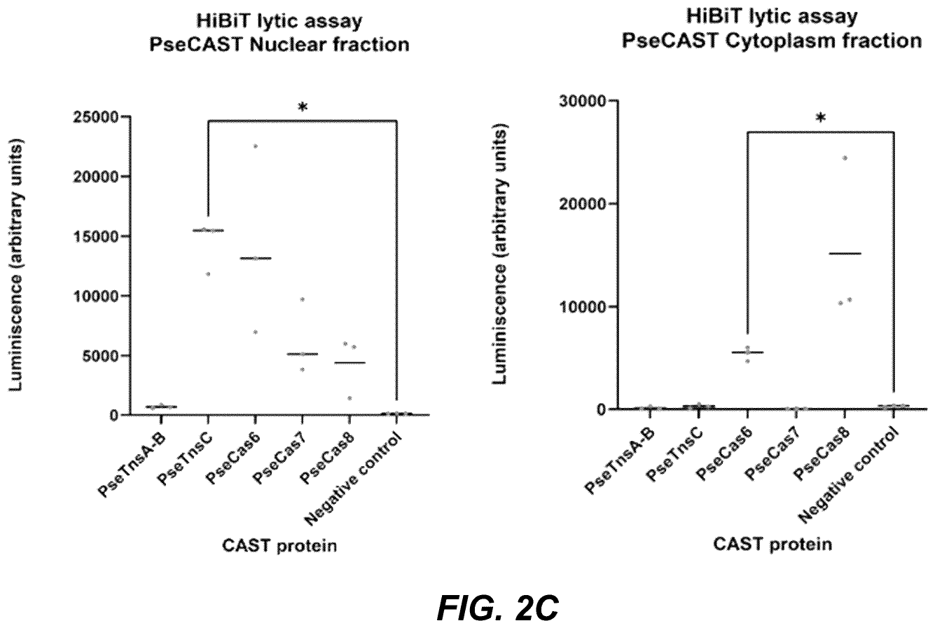

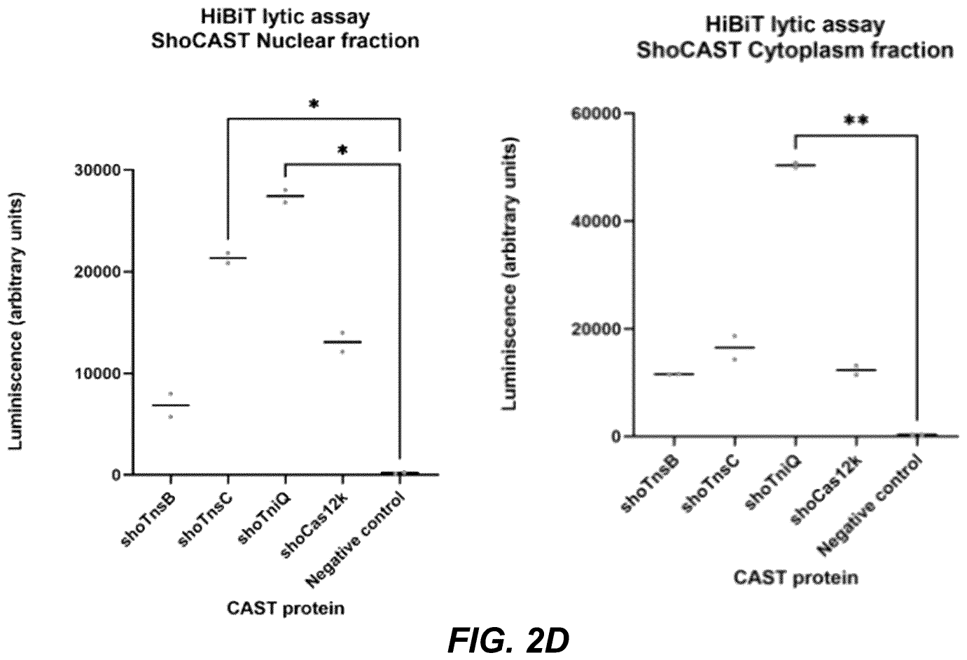

To validate CAST helper protein expression, the HiBiT lytic assay was employed which requires tagging the protein of interest with the 1.3 kDa HiBiT peptide tag. The HiBiT peptide complexes with the larger LgBiT subunit to produce a complete luciferase enzyme. The resulting complex generates a bright luciferase signal in the presence of the furimazine substrate. CAST helper proteins from the following bacterial species, Vibrio cholerae (VchCAST), Pseudoalteromonas (PseCAST) and Scytonema hofmanni (ShoCAST), were tagged with the HiBiT tag (FIG. 2A). HiBiT tagged pHelper proteins were transiently expressed using the Agrobacterium mediated T-DNA insertion. Each protein was expressed individually in plants. Initial attempts to detect protein expression failed because we were unable to lyse the plant nuclei and retrieve nuclear localized proteins. A modified protocol was devised to extract nuclei and detect the nuclear proteins separately which led to us detecting the majority of the CAST proteins (FIG. 2B-FIG. 2D).

A protocol for HiBiT lytic CAST protein detection includes the following steps: (1) Generate HiBiT tagged recombinant CAST proteins as shown in FIG. 2A; (2) Transform construct into Agrobacterium; (3) Inoculate 5 mL cultures of transformed Agrobacterium overnight at 30° C. while shaking; (4) Centrifuge cultures at 4000 g, 4° C. for 10 minutes; (5) Resuspend Agrobacterium in induction media (10 mM MES pH 5.6, 10 mM MgCl₂, 100 μM acetosyringone) and incubate at room temperature while shaking for 4 hours; (6) Infiltrate Agrobacterium into 4 week old Nicotiana benthamiana plants; (7) 3 days post infiltration, grind 3 medium size infiltrated leaves to fine powder; (8) Extract the cytoplasmic protein fraction using lysis buffer (20 mM Tris-HCl, pH 7.4, 25% Glycerol, 20 mM KCl, 2 mM EDTA; 2.5 mM MgCl₂ 2.5 ml, 250 mM Sucrose, 0.1% PMSF); (9) Centrifuge at 1500 g to pellet intact nuclei; (10) Wash nuclei up to 5 times (Wash buffer: 20 mM Tris-HCl, pH 7.4, 25% Glycerol, 2.5 mM MgCl₂, 0.2% Triton X-100); (11) Lyse nuclei with a high salt buffer (20 mM HEPES-KOH pH 7.9, 2.5 mM MgCl₂, 100 mM NaCl, 20% (v/v) Glycerol, 0.2 mM EDTA, 0.5 mM DTT, cOmplete™, Mini, EDTA-free Protease Inhibitor Cocktail (1 tablet per 10 mL volume)); (12) Combine 30 μL of Furimazine and LgBiT with 30 μL of protein sample and detect luminescence signal using an integration time of 1000 ms.

### Cast-Mediated Episomal DNA Integration in Protoplast Cells

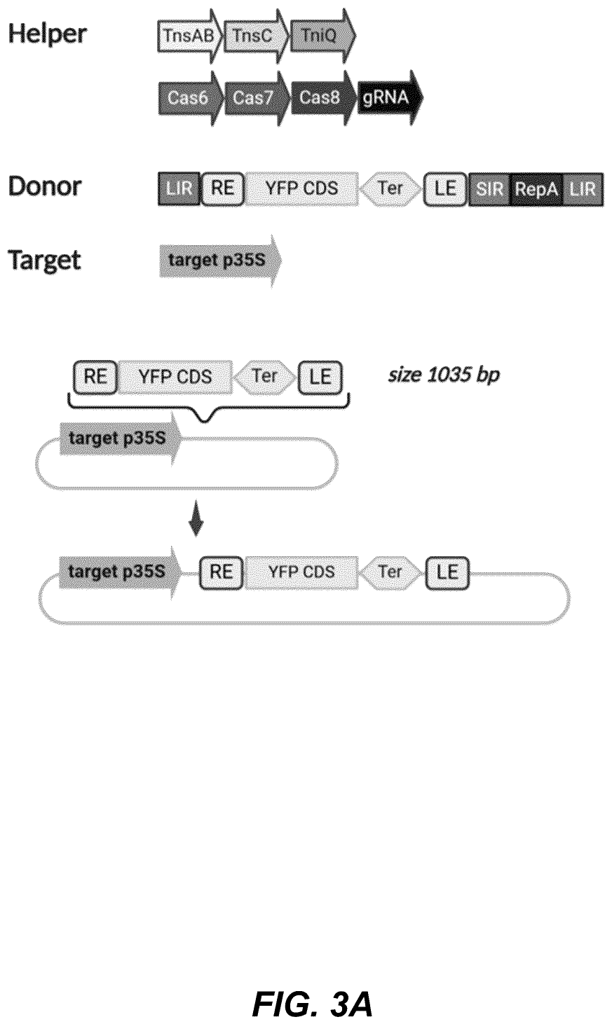

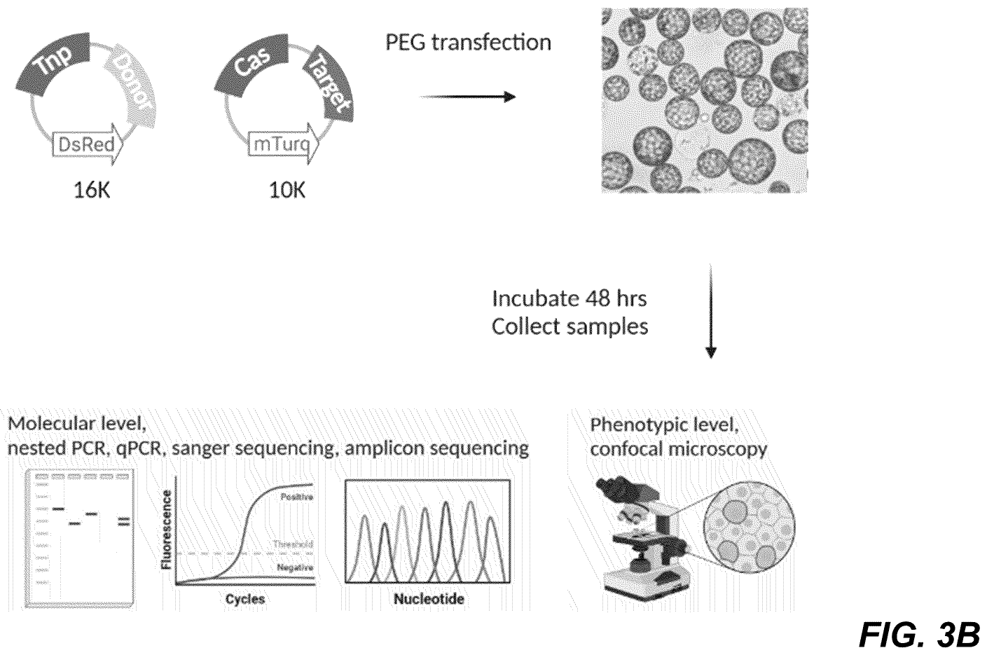

Integration in episomal DNA is more efficient than chromosomal DNA, mainly attributed to the lack of complex chromatin structure. PEG-mediated plasmid transfection of protoplasts is the only practical approach for episomal experiments. Cargo DNA was designed to be a promoter-less YFP, ensuring exclusive YFP transcription and translation from the target plasmid 35S promoter only if the intended integration occurs at the target site. In consideration of plasmid size constraints in protoplast transfection, pHelper was separated into transposases and Cas effectors to either combine with the pDonor or pTarget, respectively. Fluorescent marker (dsRed or mTurq) was added to verify the transfection efficiency (FIG. 3A).

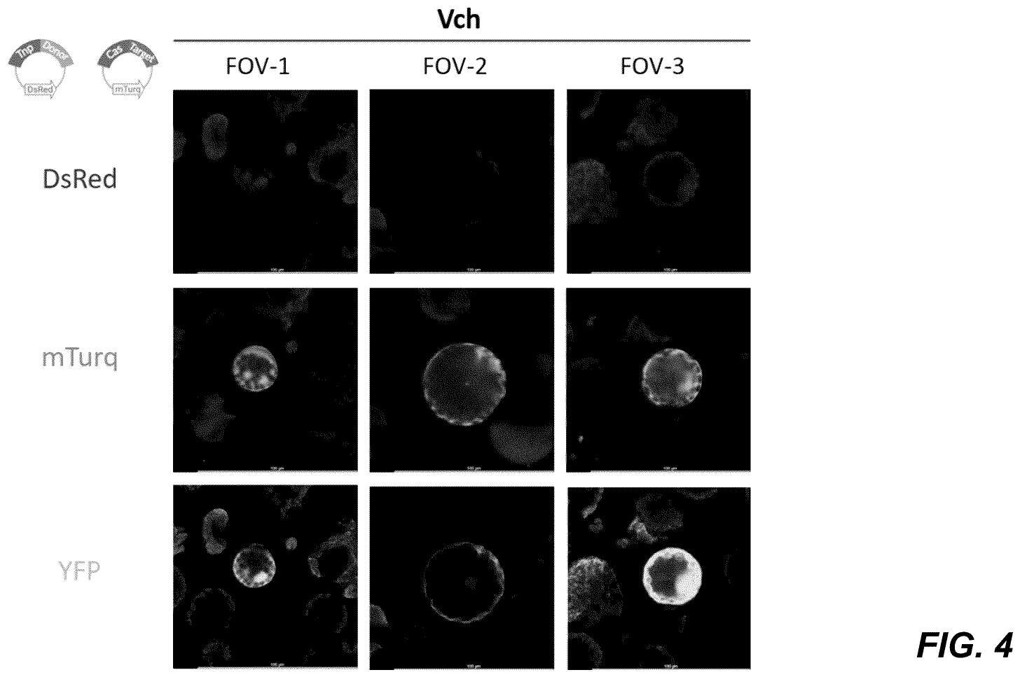

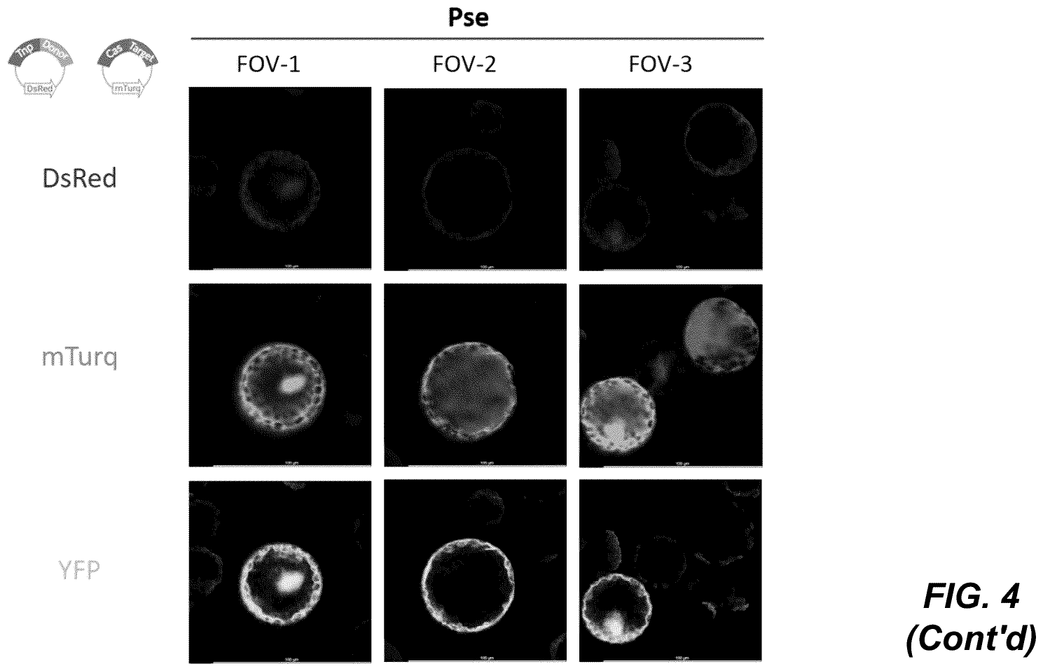

At 48 hours post-transfection, DsRed and mTurq signal confirmed co-transfection into A. thaliana protoplasts. Of primary interest is the YFP signal, and a few instances were observed among all crRNA sequences tested (FIG. 4). It should be noted that cells expressing YFP represent only a subset of cells with successful integration, as it requires the absence of premature stop codons in the sequence between the 35S promoter and the YFP start codon, which cannot be guaranteed due to the presence of transposon ends and the variable integration distance.

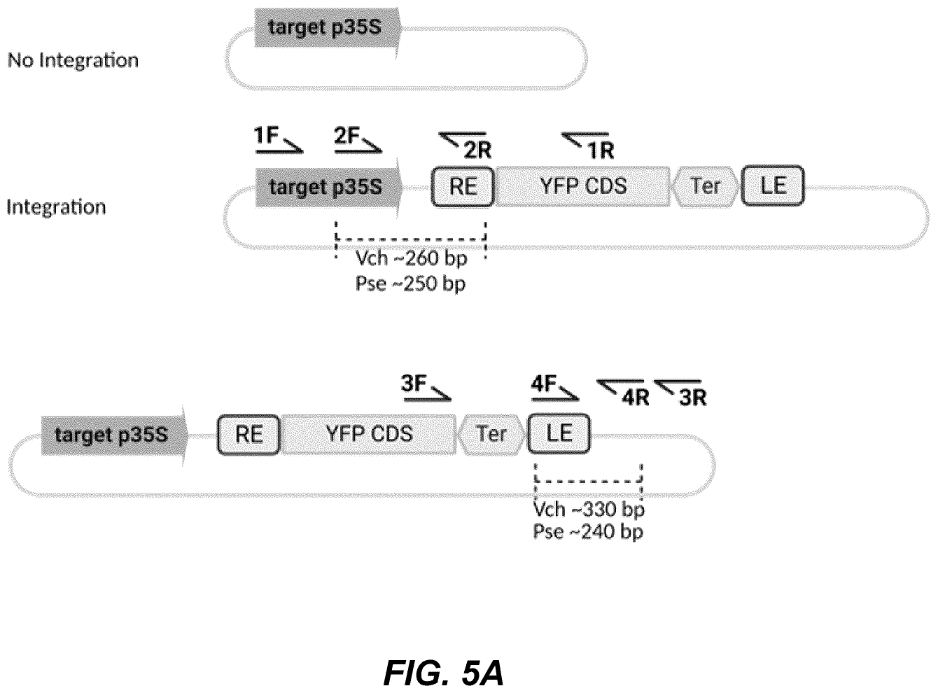

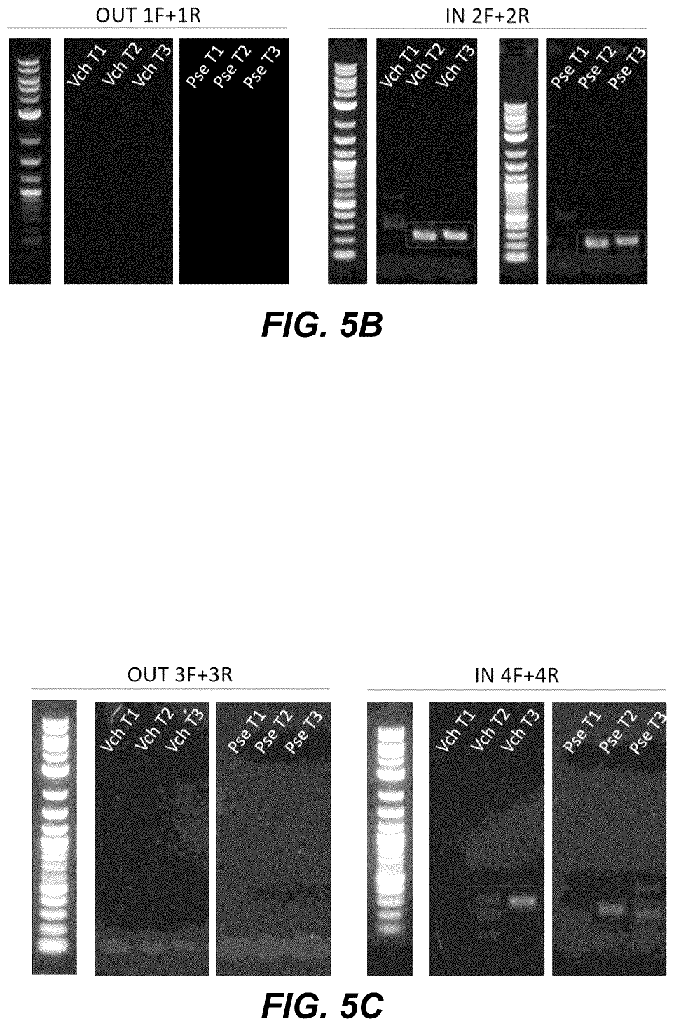

Molecular level analysis was performed next using nested PCR, where two consecutive PCR reactions are performed using two sets of primers to enhance the specificity and sensitivity of DNA amplification. To detect the integration junctions of the favored orientation Target-RE-Cargo-LE, OUT PCR reaction was done with 1F primer binding to p35S and 1R primer binding to YFP; followed by IN PCR reaction that was done with 2F primer binding to p35S and 2R primer binding to RE to specifically amplify the right end junction (FIG. 5A). Simultaneously, OUT PCR reaction was done with 3F primer binding to YFP and 3R primer binding to plasmid backbone; followed by IN PCR reaction with 4F primer binding to RE and 4R primer binding to plasmid backbone to specifically amplify the left end junction (FIG. 5A). The first OUT reaction typically yields no bands on the gel due to the low template amount. After the second round of amplification, the correct bands were detected for two out of three target choices in both Vch and Pse systems (FIG. 5B). Similar results were observed both at the RE and LE junction (FIG. 5C). The less-favored orientation is anticipated to have lower efficiency.

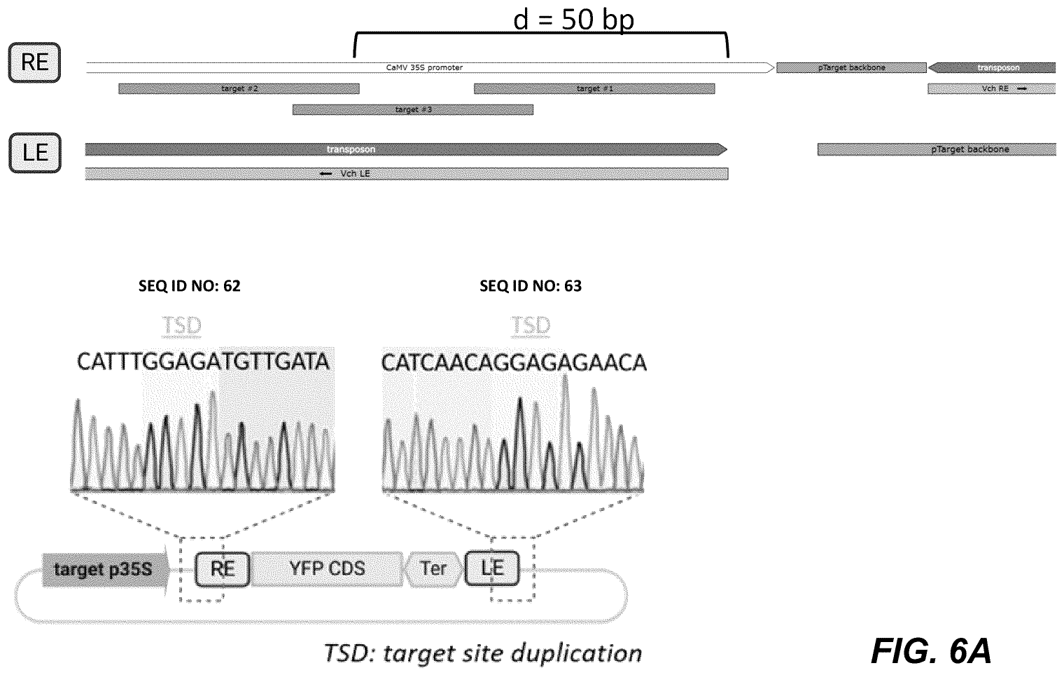

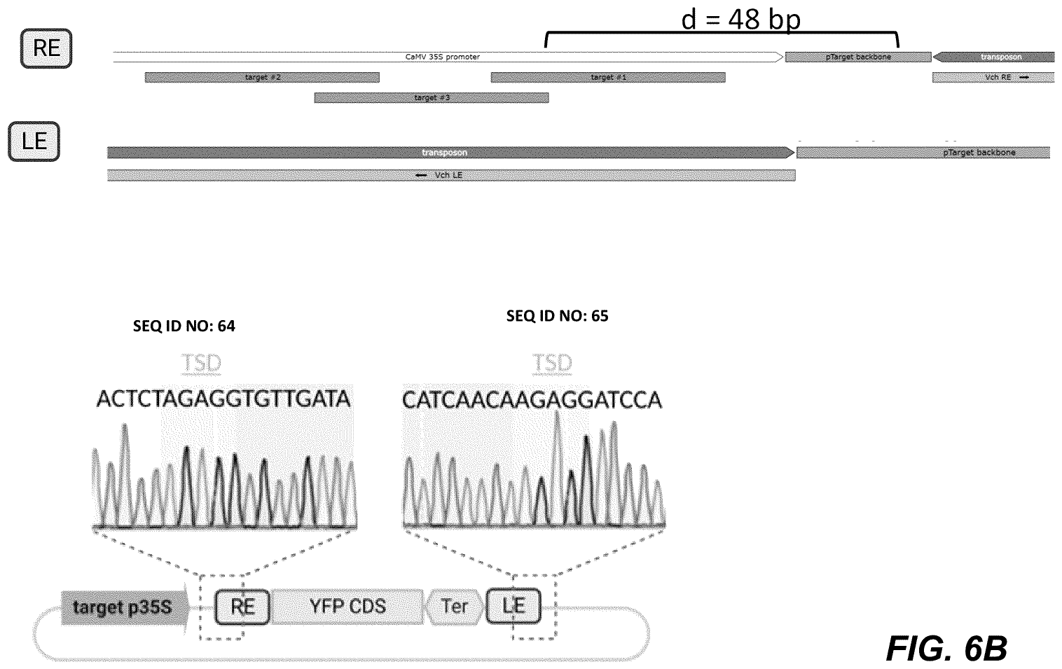

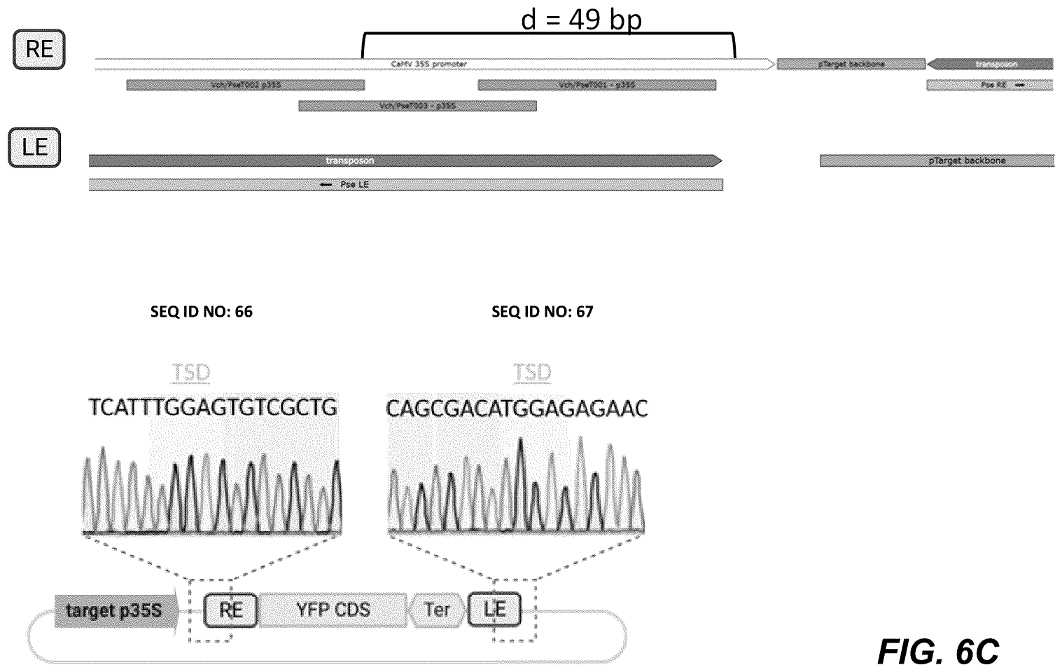

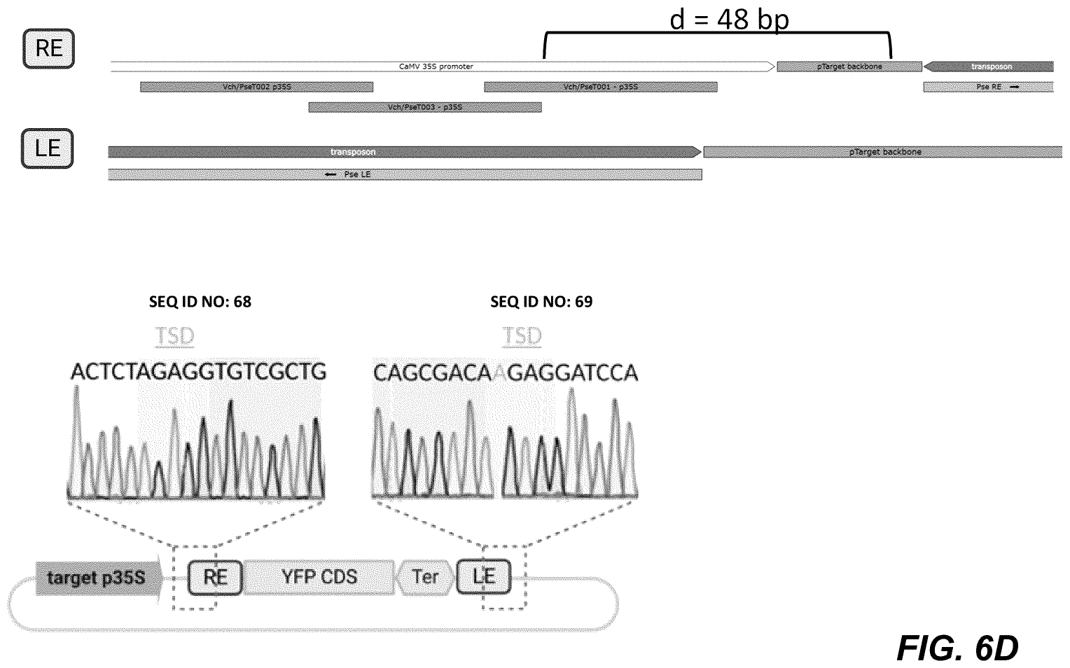

Sanger sequencing of the purified PCR bands confirmed correct integration junction products for Vch T2 & T3 and Pse T2 & T3. However, there were mixed peaks in the sequencing results originating from the variable insertion distances, given that the integration events happened around 50 bps downstream of the target site. TA-cloning was performed and single colonies selected from E. coli transformation to selectively sequence individual PCR products with set integration distance. Four colonies from Vch T2 RE exhibited different integration distances of 50, 51, 52 bp. Two colonies from Vch T2 LE exhibited an integration distance of 50 bp. These sequences also highlighted the creation of the hallmark 5-bp target site duplication (TSD) (FIG. 6A). Integration distance of ˜50 bp and TSDs were also observed for Vch T3, Pse T2, and Pse T3. (FIG. 6B-FIG. 6D). This provides the first demonstration of CAST functionality in plant cells.

Described below is an exemplary protocol for protoplast integration assays: (1) Arabidopsis protoplasts were collected and resuspended in W5 solution according to the tape method known in the art; (2) Change the buffer to MMG solution. Target for a protoplast concentration around 5×10⁵ cell/mL; (3) Use round bottom culture tubes for protoplast transfection. Add total 50 μg of plasmid into the microcentrifuge tube. Then add 100 μL of protoplasts. Mix by tapping. Add equal volume of PEG solution. Mix by tapping again; (4) Incubate the mixture at room temperature for 15 min; (5) Add 2 mL W5 solution to the culture tubes and centrifuge at 200 g for 2 minutes with acceleration and deceleration set to minimum. Remove as much as supernatant without disturbing the pellet and repeat one time; (6) Cover the well plate with 0.1% BSA for several minutes and remove fully to prevent protoplast from sticking to the bottom of the plate; (7) Add 500 μl WI solution to the protoplasts and transfer to a 12 well-plate. Gently swirl the well-plate after transferring all the protoplasts samples; (8) Incubate at room temperature at dark for around 6 hours to overnight, the check the expression with fluorescence microscopy for reporter plasmids; (9) After a 48-hour incubation period, samples are subjected either to confocal imaging or DNA isolation.

Described below is an exemplary protocol for Crude DNA isolation and nested PCR to detect integration junctions: (1) Transfer appropriate volume of protoplasts into a 1.5 ml Eppendorf tube. Centrifuge at 200 g for 2 minutes and remove the supernatant without disturbing the pellet; (2) Add 1/10 volume TE buffer relative to the original protoplast volume. Disrupt the cells by pipetting vigorously for at least 10 times and vortexing vigorously for at least 30 seconds; (3) Before any downstream analysis, centrifuge at 12000 g for 1 minute and use the supernatant as the crude DNA solution; (4) Nested PCR is carried out by two consecutive PCR reactions using NEB Phusion® High-Fidelity PCR Master Mix with HF Buffer. The cycling number is 20 for OUT PCR and 30 for IN PCR. Annealing temperature and extension time is selected based on specific reaction, and the template for the IN nested PCR is prepared by a 50-fold dilution of the OUT nested PCR product; (5) Visualize the IN nested PCR product via electrophoresis using agarose gel.

Described below is an exemplary protocol for TA cloning and Sanger sequencing to confirm integration junctions: (1) Adding an A-overhang to the 3′-end of the purified PCR amplicon using Taq DNA polymerase and dATP; (2) Follow the instructions from pGEM®-T Easy Vector Systems—Promega kit for ligation reaction; (3) Transform the reaction mixture into competent E. coli cells using standard heat shock method and blue-white screening. Incubate the plates overnight at 37° C.; (4) Pick individual white bacterial colonies and inoculate them into LB media with the suitable antibiotic. Plasmids are isolated using QIAprep Spin Miniprep Kit; (5) Submit the purified plasmid DNA with appropriate primers for Sanger sequencing (Laragen®).

Cast-Mediated DNA Integration into the Plant Genome

Described below is RNA-guided DNA integration into the plant genome. The ability of genomic insertion is crucial for many fundamental and applied studies.

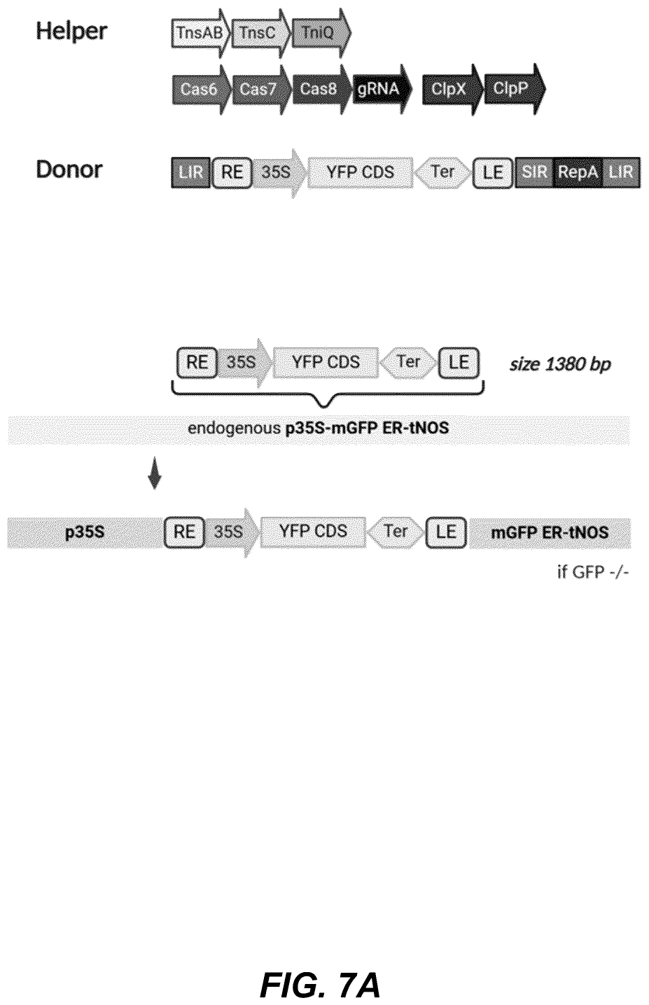

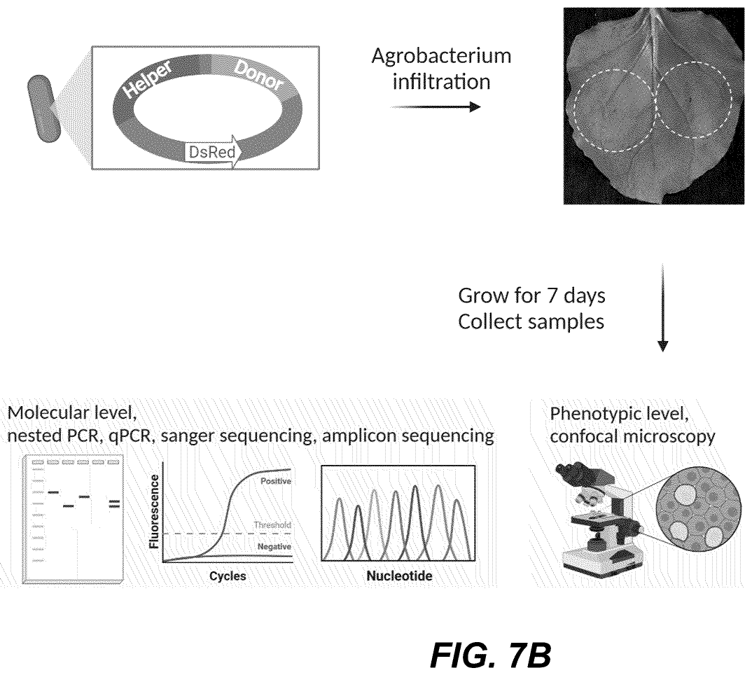

Two accessory proteins, ClpX and ClpP, were incorporated to helper plasmid because of the necessity for transpososome dissociation to initiate DNA repair. Cargo DNA was a full YFP expressed with a 35S promoter, thus, in this case, there was no positive phenotypic signal detection, as YFP can be expressed from the Agrobacterium inserted T-DNA. This can also complicate the primer design due to the presence of duplicated 35S elements (FIG. 7A). The CAST machinery is designed to integrate the full YFP cargo into the endogenous 35S promoter within the GFP cassette of the transgenic N. benthamiana. Disrupting both alleles of GFP is expected to lead to the loss of GFP expression. All components were cloned into a single plasmid, transformed into Agrobacterium, and infiltrated into N. benthamiana leaves. Plants were grown for 7 days, and DNA samples were harvested from leaves for subsequent phenotypic and molecular analysis (FIG. 7B).

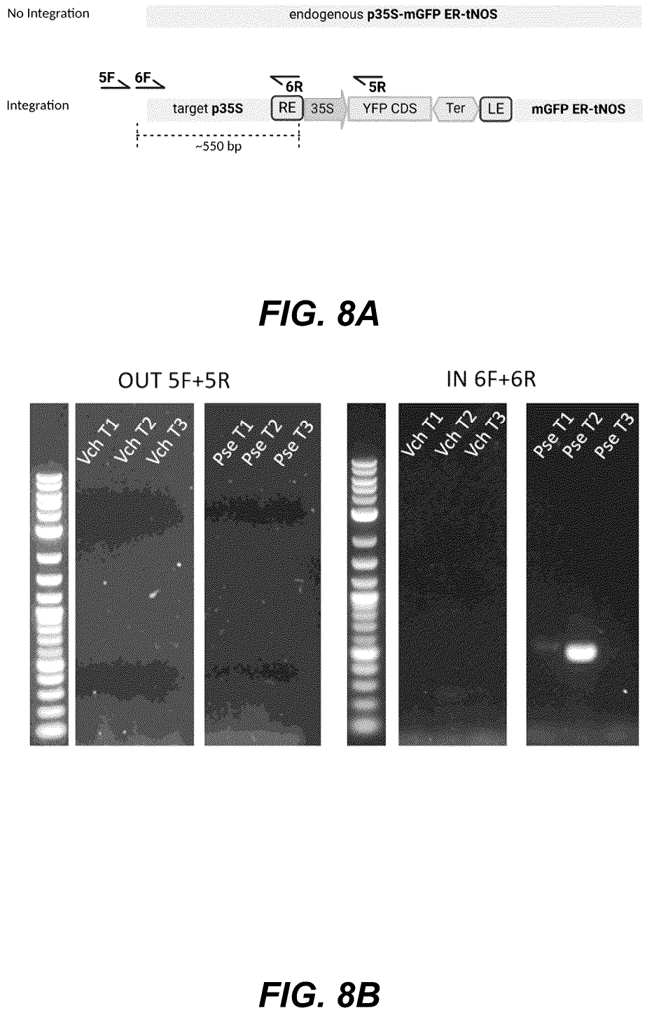

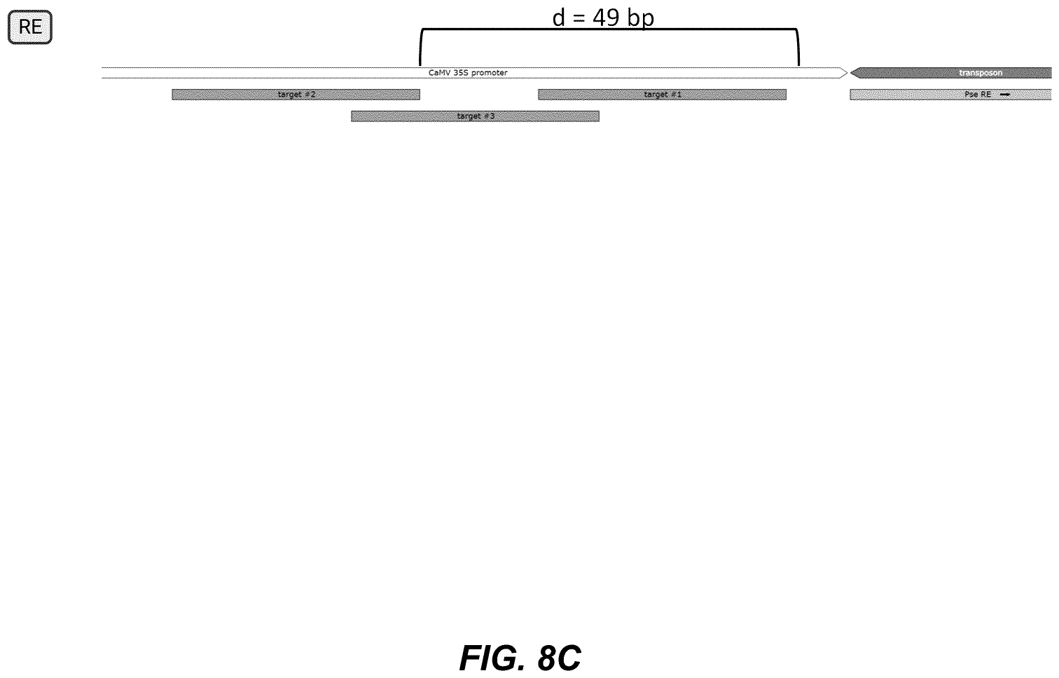

Molecular analysis was performed using the nested PCR. To minimize off-target amplification from the 35S promoter, the 5F and 6F primers specifically target the unique 5′ sequence upstream of the p35S target site, although at the expense of the long amplicon length. OUT PCR reaction was done with 5F primer and 5R primer binding to YFP; IN PCR reaction was done with 6F primer and 6R primer binding to RE to specifically amplify the right end junction (FIG. 8A). The first OUT reaction typically yields no bands due to the low template amount; after the second round of amplification, the correct bands were detected for one out of three target sites in the Pse system (FIG. 8B).

Then, the TA-cloning method was followed and individual PCR products from the Pse T2 RE PCR product were selectively sequenced. Two different integration distances of 49 and 50 bp were identified (FIG. 8C).

Described below is an exemplary protocol for Leaf integration assays: (1) Inoculate 5 mL cultures of Agrobacterium harboring the construct, and incubate at 30° C. shaking overnight; (2) Centrifuge cultures down at 4000 g for 10 minutes at room temperature, and discard supernatant and resuspend pellet in MMA media (10 mM MES pH 5.6, 10 mM MgCl₂, 100 uM acetosyringone); (3) Dilute the resuspension in more MMA to reach an OD600 of 0.35-0.40; (4) Let the inoculated MMA media sit at room temperature for 2-4 hours; (5) Infiltrate leaf with syringe and mark the infiltrated area. N. benthamiana plants are kept in growth chamber for 7 days under a 16-h-light/8-h-dark cycle; (6) Plant DNA is harvested by following the DNeasy Plant Mini Kit instruction, and after elution with water, DNA concentration is adjusted to approximately 50 ng/μl for following molecular analysis.

TABLE 1
NESTED PCR PRIMERS - PROTOPLAST EXPERIMENT

| | | | SEQ |
|---|---|---|---|
| Short | | Long | ID |
| Identifier | Sequence | Identifier | NO: |
| 1F | gccgacagtggtcccaaagatggac | F p35S-2 | 47 |
| 1R | CTTGCCGGTGGTGCAGAG | R YPET-4 | 48 |
| 2F | ccacccacgaggagcatcgtg | F p35S-3 | 49 |
| 2R | CTGAAGTTTAGACCATGAAGAGG | R2 Vch RE | 50 |
| (Vch) | C | amplicon | |
| 2R (Pse) | AGGGGCTTTATGTCAGAGTATGA | R2 Pse RE | 51 |
| | C | amplicon | |
| 3F | GACTGCTGCTGGTATTACCGAGG | F YPET-2 | 52 |
| | G | | |
| 3R | GCGGATCTAACTAATAAAGTGTC | R for LE- | 53 |
| | TACcattagtatac | dummy-1 | |
| 4F (Vch) | TCCGTGACTGATGCAAGGTTATC | F2 Vch LE | 54 |
| | GG | amplicon | |
| 4F (Pse) | AAACCAGTCTTATATCCGGGCTG | F2 Pse LE | 55 |
| | G | amplicon | |
| 4R | GCGGATCTAACTAATAAAGTGTC | R for LE- | 56 |
| | TACcattagtatac | dummy-1 | |

TABLE 2
NESTED PCR PRIMERS - LEAF EXPERIMENT

| | | | SEQ |
|---|---|---|---|
| Short | | Long | ID |
| Identifier | Sequence | Identifier | NO: |
| 5F | tggcgaacagttcatacagagtctc | F pTarget | 57 |
| | | p35S-1 | |
| 5R | TCCAGCTCGACCAGGATGG | R YPET-1 | 58 |
| 6F | tcttcgtcaacatggtggagcacg | F pTarget | 59 |
| | | p35S-2 | |
| 6R | CTGAAGTTTAGACCATGAAGAGG | R2 Vch RE | 60 |
| (Vch) | C | amplicon | |
| 6R (Pse) | AGGGGCTTTATGTCAGAGTATGA | R2 Pse RE | 61 |
| | C | amplicon | |

TABLE 3
PLASMID SUMMARY - PROTOPLAST EXPERIMENT

| Plasmid ID | Plasmid components |
|---|---|
| pGYW5CH0111 | L5 pUAP4 pCk3 Vch pTarget 35S (A) + spc (B) + |
| | Helper Cas (C) + mTurq (E) |
| pGYW5CH0112 | L5 pUAP4 pCk3 Pse pTarget 35S (A) + spc (B) + |
| | Helper Cas (C) + mTurq(E) |
| pGYW5CH0115 | L5 pUAP4 pCk2 Vch Donor YFP-spc-T001 (A) + |
| T001 | Helper Tnp (B) + spc (C) + DsRed (E) |
| pGYW5CH0115 | L5 pUAP4 pCk2 Vch Donor YFP-spc-T002 (A) + |
| T002 | Helper Tnp (B) + spc (C) + DsRed (E) |
| pGYW5CH0115 | L5 pUAP4 pCk2 Vch Donor YFP-spc-T003 (A) + |
| T003 | Helper Tnp (B) + spc (C) + DsRed (E) |
| pGYW5CH0116 | L5 pUAP4 pCk2 Pse Donor YFP-spc-T001 (A) + |
| T001 | Helper Tnp (B) + spc (C) + DsRed (E) |
| pGYW5CH0116 | L5 pUAP4 pCk2 Pse Donor YFP-spc-T002 (A) + |
| T002 | Helper Tnp (B) + spc (C) + DsRed (E) |
| pGYW5CH0116 | L5 pUAP4 pCk2 Pse Donor YFP-spc-T003 (A) + |
| T003 | Helper Tnp (B) + spc (C) + DsRed (E) |

TABLE 4
PLASMID SUMMARY - LEAF EXPERIMENT

| | | SEQ ID NO: |
|---|---|---|
| | | (T-DNA |
| Plasmid ID | Plasmid detailed name | sequence) |
| pGYW5KN0107 | L5 pCk2 Vch Donor pYFP-ClpXP-T001 (A) + | 38 |
| T001 | Helper Tnp (B) + Helper Cas (C) + DsRed (E) | |
| pGYW5KN0107 | L5 pCk2 Vch Donor pYFP-ClpXP-T002 (A) + | 39 |
| T002 | Helper Tnp (B) + Helper Cas (C) + DsRed(E) | |
| pGYW5KN0107 | L5 pCk2 Vch Donor pYFP-ClpXP-T003 (A) + | 40 |
| T003 | Helper Tnp (B) + Helper Cas (C) + DsRed(E) | |
| pGYW5KN0108 | L5 pCk2 Pse Donor pYFP-ClpXP-T001 (A) + | 41 |
| T001 | Helper Tnp (B) + Helper Cas (C) + DsRed(E) | |
| pGYW5KN0108 | L5 pCk2 Pse Donor pYFP-ClpXP-T002 (A) + | 42 |
| T002 | Helper Tnp (B) + Helper Cas (C) + DsRed(E) | |
| pGYW5KN0108 | L5 pCk2 Pse Donor pYFP-ClpXP-T003 (A) + | 43 |
| T003 | Helper Tnp (B) + Helper Cas (C) + DsRed(E) | |

TABLE 5A
EXPRESSION DATA - VCH CAST (NUCLEAR FRACTION)*

| | Rep1 | Rep2 | Rep3 |
|---|---|---|---|
| vchTnsA-B | 958.182929 | 1793.462299 | 2113.894623 |
| vchTnsC | 36484.94021 | 26769.81795 | 48279.7851 |
| vchTniQ | 9606.525542 | 10173.125 | 16238.03773 |
| vchCas6 | 22933.97474 | 30378.44578 | 14815.99063 |
| vchCas7 | 17120.45531 | 8802.426611 | 15315.90506 |
| vchCas8 | 32197.54667 | 49562.4393 | 64975.69638 |
| Negative control | 416.912023 | 783.9432465 | 599.6773796 |

*relative luminescence units normalized to protein concentration

TABLE 5B
EXPRESSION DATA - VCH CAST
(CYTOPLASMIC FRACTION)*

| | Rep1 | Rep2 | Rep3 |
|---|---|---|---|
| vchTnsA-B | 670.2429549 | 156.3945564 | 390.0156227 |
| vchTnsC | 2275.723375 | 319.8470487 | 2200.177778 |
| vchTniQ | 778.8978805 | 1166.675387 | 1252.972136 |
| vchCas6 | 1588.564004 | 1583.864819 | 2028.979321 |
| vchCas7 | 1249.630374 | 2128.845234 | 4952.360336 |
| vchCas8 | 625.5878633 | 982.0480473 | 1759.015744 |
| Negative control | 573.7433598 | 518.012268 | 492.6125331 |

*relative luminescence units normalized to protein concentration

TABLE 6A
EXPRESSION DATA - PSE CAST (NUCLEAR FRACTION)*

| | Rep1 | Rep2 | Rep3 |
|---|---|---|---|
| vchTnsA-B | 693.509481 | 865.4245229 | 579.5452139 |
| vchTnsC | 11840.68353 | 15471.35966 | 15593.2671 |
| vchTniQ | 15334.67665 | 17890.94761 | 20774.59751 |
| vchCas6 | 6986.819874 | 13141.22572 | 22538.12285 |
| vchCas7 | 5130.733098 | 9718.885697 | 3828.325613 |
| vchCas8 | 1427.339806 | 6009.792569 | 5744.788468 |
| Negative control | 127.1174404 | 147.6658962 | 172.6738303 |

*relative luminescence units normalized to protein concentration

TABLE 6B
EXPRESSION DATA - PSE CAST
(CYTOPLASMIC FRACTION)*

| | Rep1 | Rep2 | Rep3 |
|---|---|---|---|
| vchTnsA-B | 91.82135127 | 294.0918765 | 102.4193156 |
| vchTnsC | 515.7902571 | 140.7168447 | 266.8300106 |
| vchTniQ | 5334.676646 | 3890.947609 | 3774.597515 |
| vchCas6 | 4697.56809 | 6026.43677 | 5558.893581 |
| vchCas7 | 40.2196634 | 21.6954773 | 81.07759961 |
| vchCas8 | 24435.59284 | 10686.66526 | 10335.26801 |
| Negative control | 210.1881088 | 359.0901504 | 329.3213838 |

*relative luminescence units normalized to protein concentration

TABLE 7A
EXPRESSION DATA - SHO CAST (NUCLEAR FRACTION)*

| | Rep1 | Rep2 |
|---|---|---|
| shoTnsB | 7998.311228 | 5687.729904 |
| shoTnsC | 21827.61629 | 20830.12271 |
| shoTniQ | 26810.86344 | 28024.77568 |
| shoCas12k | 13997.4974 | 12136.24733 |
| Negative control | 155.8554124 | 250.2784176 |

*relative luminescence units normalized to protein concentration

TABLE 7B
EXPRESSION DATA - SHO CAST
(CYTOPLASMIC FRACTION)*

| | Rep1 | Rep2 |
|---|---|---|
| shoTnsB | 11553.97761 | 11636.99973 |
| shoTnsC | 18673.85708 | 14342.24978 |
| shoTniQ | 50803.55048 | 49965.17347 |
| shoCas12k | 13216.10215 | 11452.42927 |
| Negative control | 355.2587132 | 321.1234335 |

*relative luminescence units normalized to protein concentration

### Example 2

### Selection and Screening

Described in this Example are exemplary use cases for the CAST methods of the disclosure.

Engineering a Stable Transgenic Line with CAST and Proper Selection

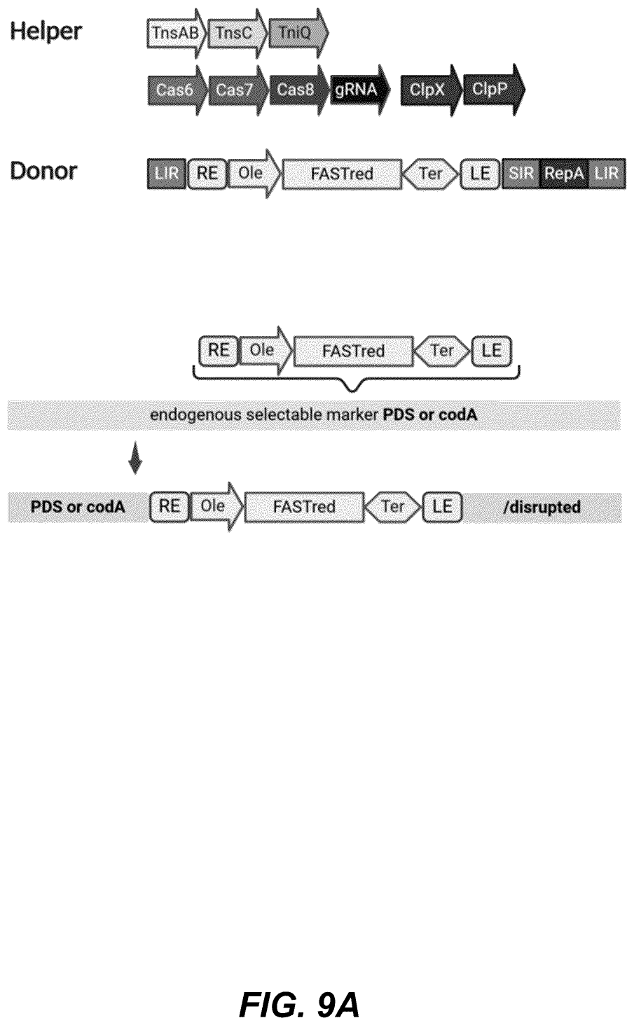

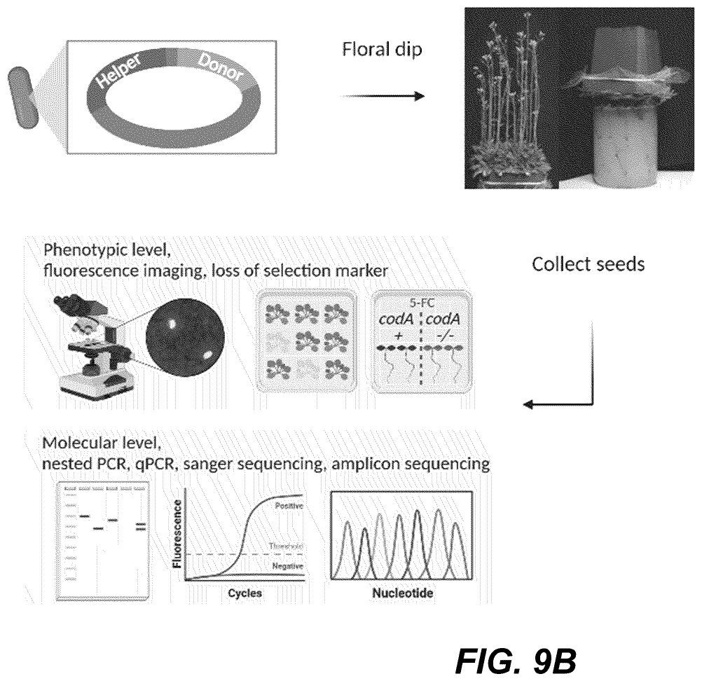

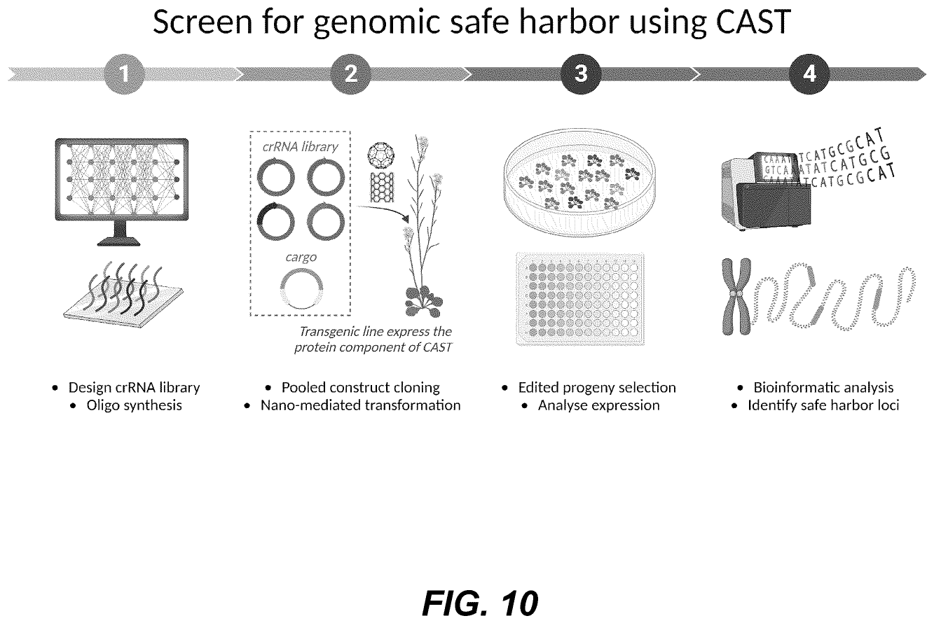

Described below is engineering a stable transgenic line with the developed CAST tool and selection. The cargo DNA is a complete FASTred cassette, expressing red fluorescence specifically in the seed coat, which simultaneously serves as the transformation marker. To select for CAST-mediated targeted integration events, a transgenic line harboring a selectable marker as the target site is employed. The options include 1) phytoene desaturase (PDS) gene, where knockout Arabidopsis exhibits an albino or mosaic phenotype corresponding to homozygous or heterozygous mutations, due to impaired chlorophyll and carotenoid synthesis, 2) codA gene, a cytosine deaminase that converts non-toxic 5-Fluorocytosine (5-FC) to toxic 5-FU causing cell death (FIG. 9A).

All components are cloned into a single plasmid and transformed into Agrobacterium. Flowering Arabidopsis plants are dipped in Agrobacterium solution for transformation of genetic materials. Subsequently, seeds are collected and germinated on selection plates to observe the albino phenotype from the loss of PDS; or on 5-FC selection plates to enrich for the loss of codA. Germinated progeny are subjected to further molecular analysis (FIG. 9B).

### Apply CAST to Screen for Genomic Safe Harbor Loci

Genomic safe harbors (GSHs) are chromosomal regions that can accommodate transgenes without adverse effects on the host organism and possess high gene expression levels. In the pursuit of advancing precise breeding and molecular farming, reliable sites for gene integration need to be identified. However, to date, only two studies reported candidate GSH in rice via mutagenesis. Identifying ideal loci for transgene expression will be beneficial for conducting more rational and reproducible studies.

After establishing CAST as a potent targeted DNA insertion tool in plants, genome-wide screens are performed to identify much-needed genomic safe harbor sites. Initially, a crRNA library via oligo synthesis is designed. Then pooled crRNA construct cloning, and Agrobacterium transformation is performed to introduce crRNA and donor construct into a transgenic line expressing the transposition enzymes and CRISPR-Cas effectors. Importantly, T-DNA should be eliminated so that the phenotype is the pure result from CAST-mediated target integration. Two major strategies have been developed to remove or prevent the integration of T-DNA: 1) genetic segregation to eliminate transgenic sequences; 2) transient expression from DNA vectors or pre-assembled protein-guide RNA ribonucleoproteins. In the case of adapting the first approach, transformation experiments are carried out in Arabidopsis via floral dip. One strategy involves incorporating the T-DNA region with a bi-functional marker consisting of the negative selection marker CodA fused to the positive selection marker NPTII. In the first generation, transgenic mutant plants are selected by kanamycin, subsequently, transgene-free mutants will be selected by 5-FC in the next generation. In the case of adapting the second approach, experiments are carried out in N. benthamiana leaves using nanotechnology-enabled DNA delivery for transient expression. Afterwards, the edited plants are subjected to barcoded NGS and quantitative analysis of cargo gene expression. In the final phase, a bioinformatic analysis is undertaken to systematically identify safe harbor loci.

### Example 3

### Modifications and Assessments

Described in this Examples are various modifications and assessments of the systems, nucleic acid compositions, and methods described above in Example 1.

### Verification of CAST Protein Expression in Plants

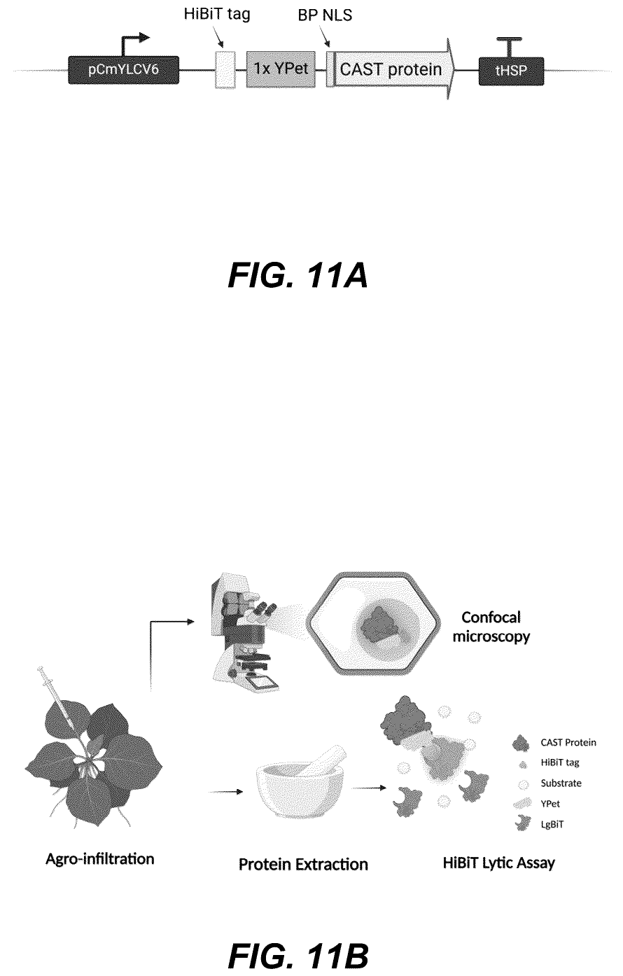

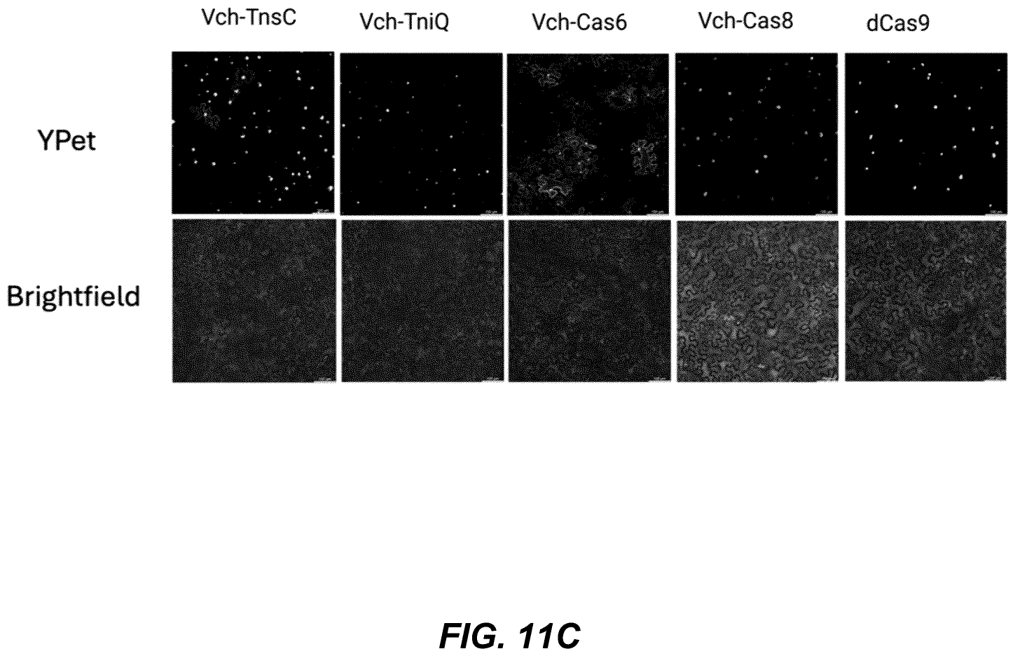

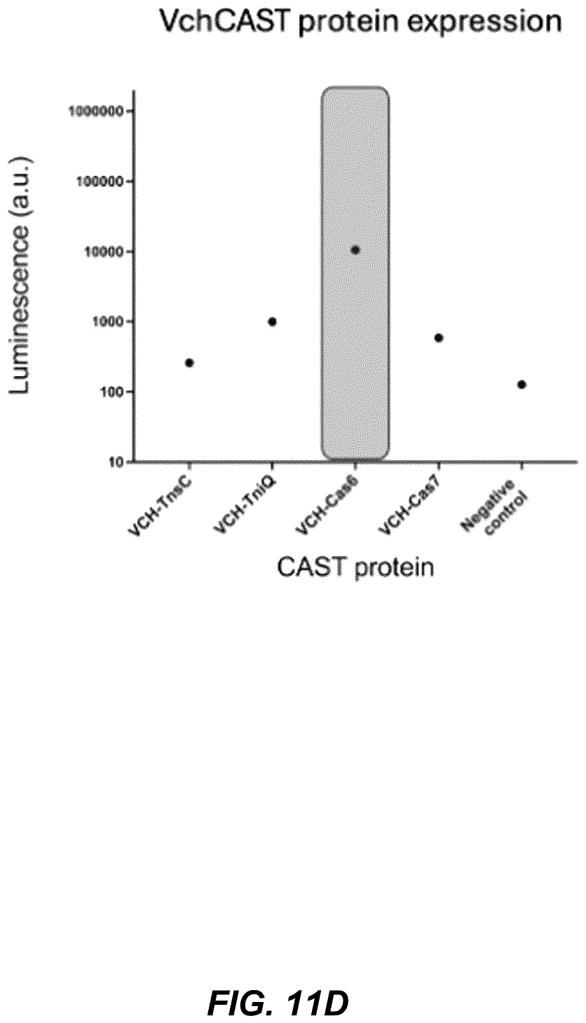

In the designs described above to express CAST proteins, they were expressed as monocistronic cassettes driven by the pCmYLCV911 (pCmY) promoter and the AtHSP18.2t (tHSP) terminator. The transposase proteins were combined into a fusion protein (TnsA-B), and a bipartite nuclear localization sequence was added (FIG. 11A). To detect protein expression, the Nano-Glo HiBiT lytic assay was used, which enables the detection of recombinant proteins tagged with a HiBiT tag as a luminescence signal. The HiBiT tag forms a complex with LgBiT (the larger part of the luciferase enzyme) to reconstitute a complete luciferase enzyme, producing a luminescent signal in the presence of the substrate furimazine. To further validate protein localization in the nucleus, a yellow fluorescent protein reporter (Ypet) at the N-terminus, downstream of the HiBiT tag was included (FIG. 11A). Constructs expressing individual CAST proteins were delivered as single plasmids for each protein using Agrobacterium tumefaciens into Nicotiana benthamiana plant leaves (FIG. 11B).

A subset of proteins from the VchCAST system, including TnsC, TniQ, Cas6, Cas7, and Cas8, was selected for the initial expression detection experiments, with dCas9 serving as a positive control and non-infiltrated leaves serving as a negative control. Most CAST proteins, while detectable using confocal microscopy (FIG. 11C), were initially undetectable using the HiBiT lytic detection assay on whole-cell lysates (FIG. 11D). Out of all proteins, VchCas6—the smallest CAST protein with a size of 24.531 kDa, showed the highest expression levels in the HiBiT assay and was the only protein that showed cytoplasmic localization in confocal images. This finding allowed for the conclusion that undetected proteins were retained in the nucleus during the protein extraction step due to the bipartite nuclear localization peptide.

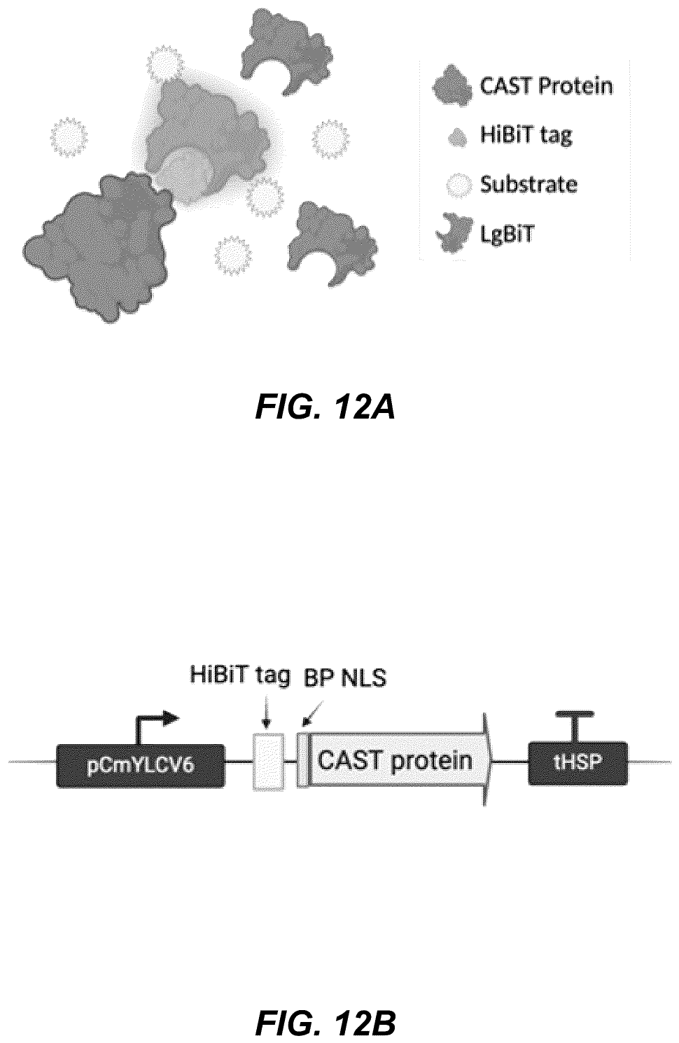

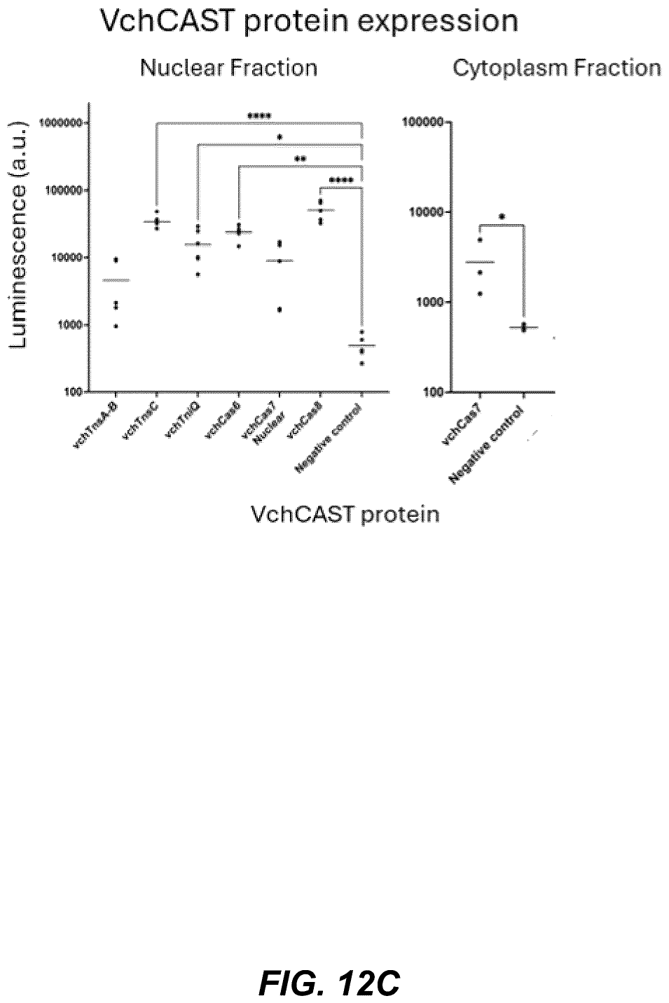

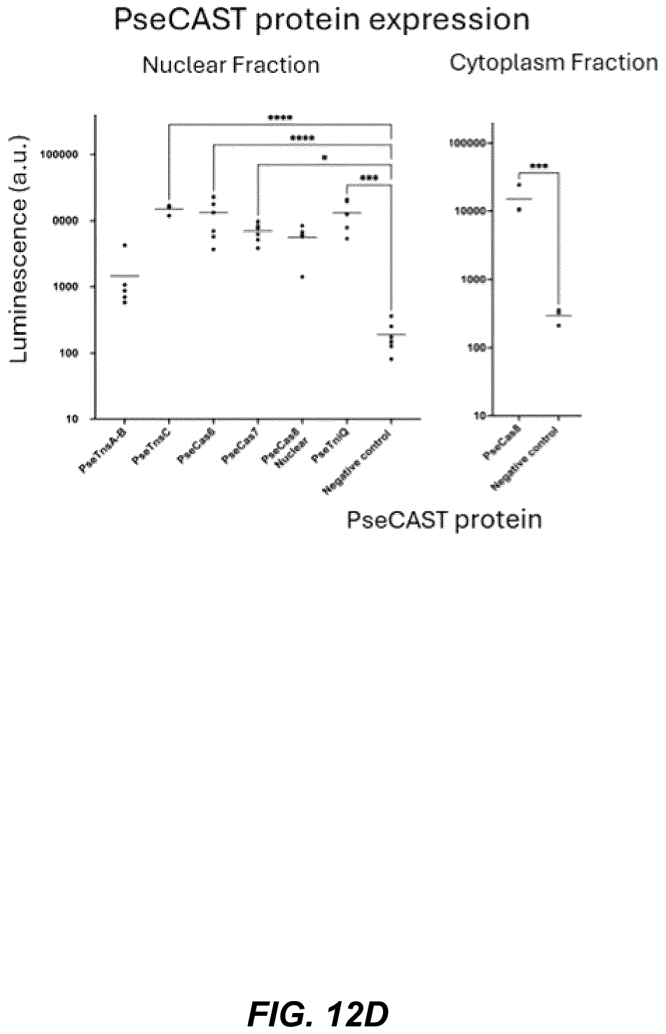

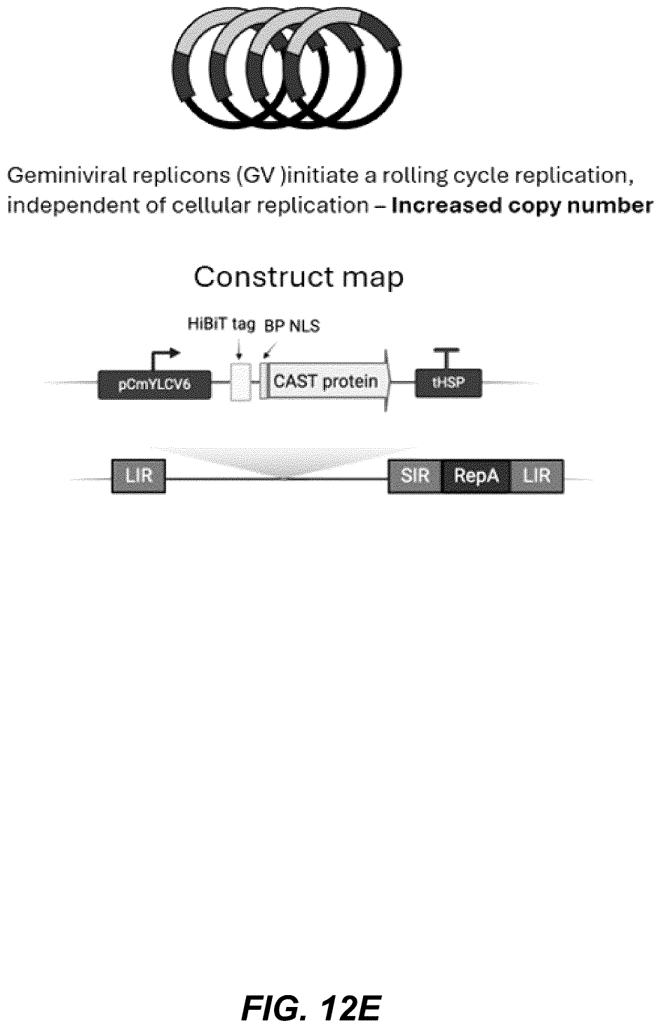

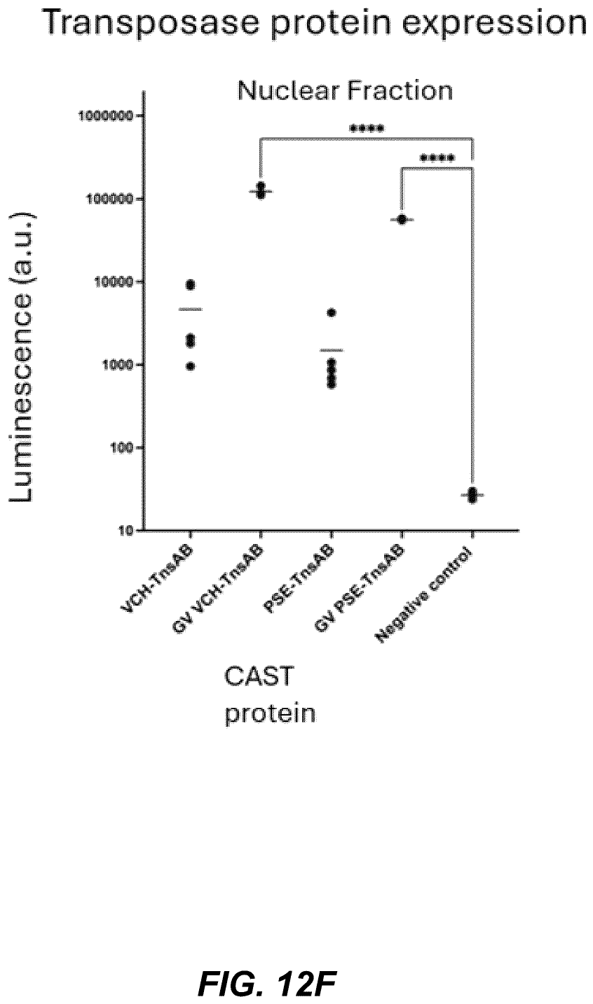

To address this, a protocol was developed to separate plant nuclei from the cytoplasmic fraction before nuclear lysis and protein detection. The Nano-Glo HiBiT Lytic Detection protocol was applied following the nuclear isolation and lysis (FIG. 12A-FIG. 12B). Using this protocol, the successful expression of all VchCAST and PseCAST proteins was detected, as indicated by the luminescent signal (FIG. 12C-FIG. 12D). Transposase proteins (TnsA-B fusion proteins) from both VchCAST and PseCAST exhibited significantly lower expression levels compared to the other CAST components (FIG. 12C-FIG. 12D). To enhance the heterologous expression of transposase proteins, geminivirus-derived replicons were incorporated into the constructs (FIG. 12E). These elements initiate autonomous DNA replication within plant cells, thereby increasing DNA copy numbers and raising the transcriptional output of transposase proteins. The inclusion of geminiviral replicons resulted in at least a 10-fold increase in transposase protein expression compared to constructs without geminiviral elements (FIG. 12F).

### Cast-Mediated Episomal DNA Integration in Plant Protoplast Cells: Molecular Analysis Across Time Points

After the episomal DNA integration experiments described above, the temporal profile of integration, integration efficiency, and integration distance was further characterized, focusing on the PseCAST derived from Pseudoalteromonas.

For the following experiments, two different cargo DNAs were tested: a random 200 bp sequence and a 1200 bp promoterless mCherry coding sequence. Given plasmid size constraints in protoplast transfection, helper plasmid was separated into transposases and Cas effectors to either combine with the pDonor or pTarget, respectively. A fluorescent marker (mTurq or YPET) was added to verify the transfection efficiency. The integration target site was a 35S plant promoter and two crRNAs (T2 and T3) were designed and tested to target the 3′ region in the 35S promoter.

Identifier and details of the PseCAST constructs are: #145, pTarget 35S-bds+Helper Cas+mTurq; #150, Helper Tnp+RE-200 bp-LE-crRNA T3+YPET; #151, Helper Tnp+RE-proless mCherry-LE-crRNA T3+YPET; #152, Helper Tnp+RE-p35S mCherry-LE-crRNA T3+YPET (positive control).

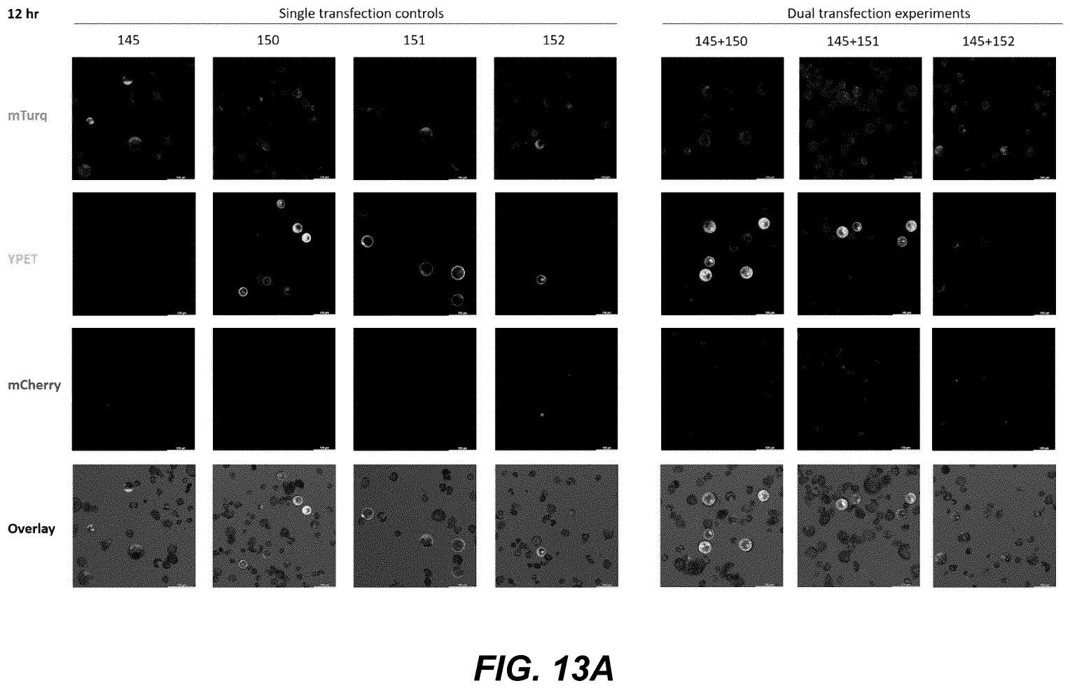

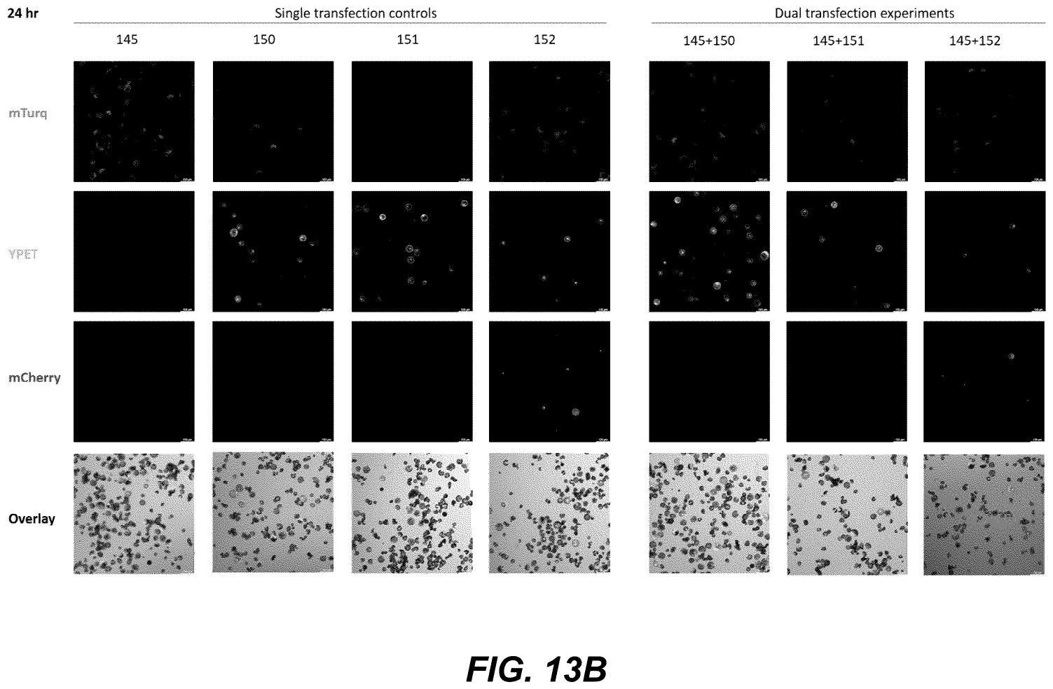

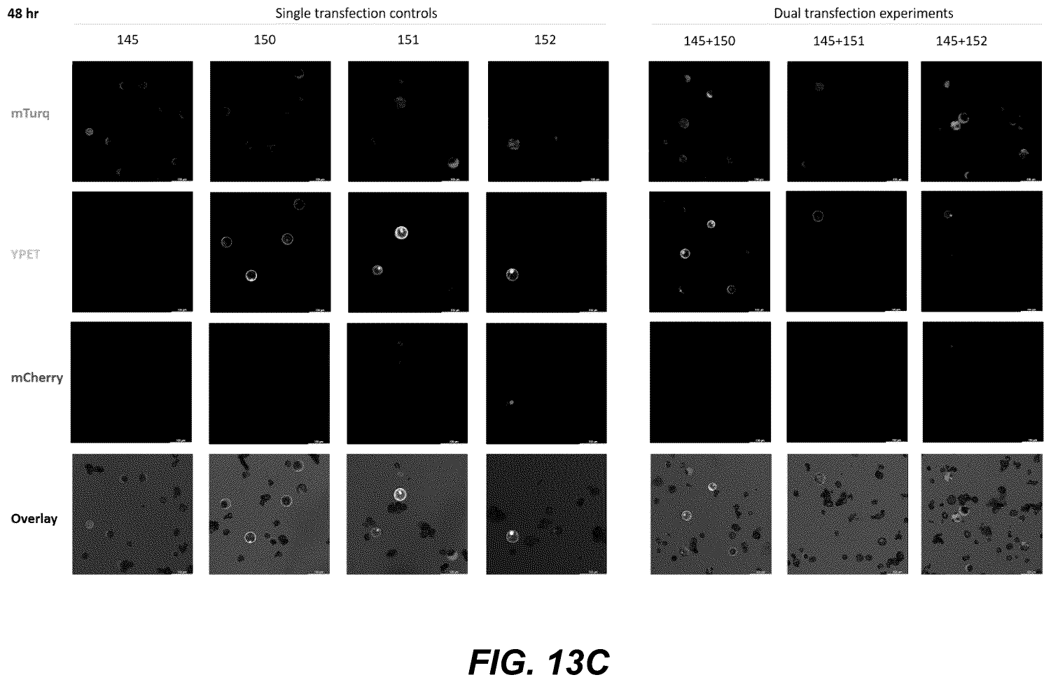

Samples were analyzed at 12-, 24-, and 48-hours post-transfection. 200 bp random cargo results were not expected to generate any fluorescence signal from targeted insertion, and were hence characterized at the molecular level. For the promoterless mCherry cargo, confocal images showed that at 12 and 24 hours, single transfection control groups did not show mCherry (as expected); and only the experimental dual transfection groups showed the gain of mCherry (FIG. 13A-FIG. 13B). However, at 48 hours, both the negative control and experiment groups showed mCherry expression (FIG. 13C). This unexpected outcome could be explained by plasmid recombination events or long-distance interaction of the promoters present upstream of mCherry coding sequence that take place when long incubation periods are used. Full mCherry cassette delivery demonstrated expression as expected.

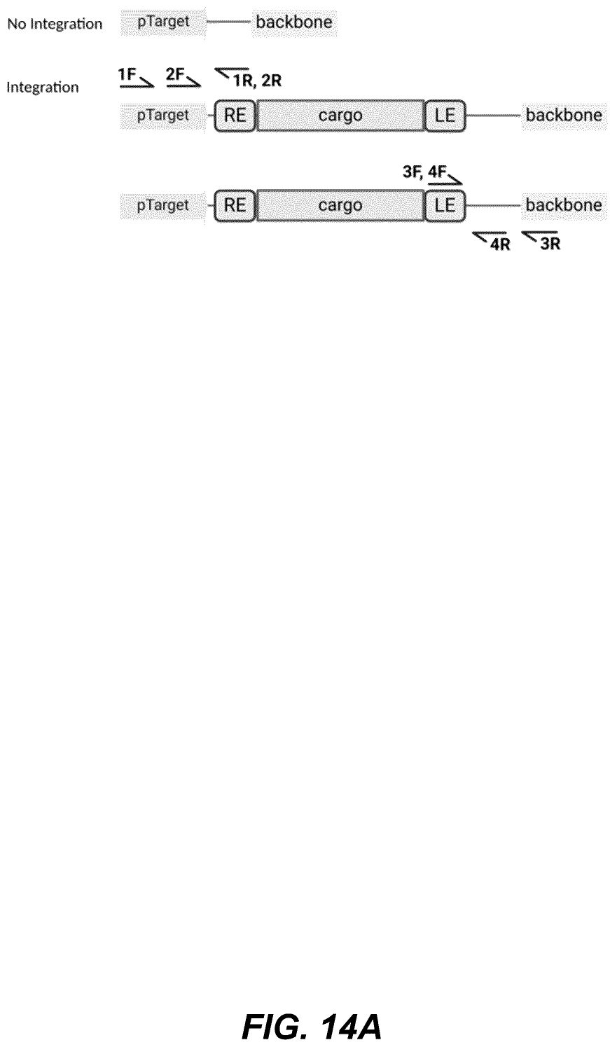

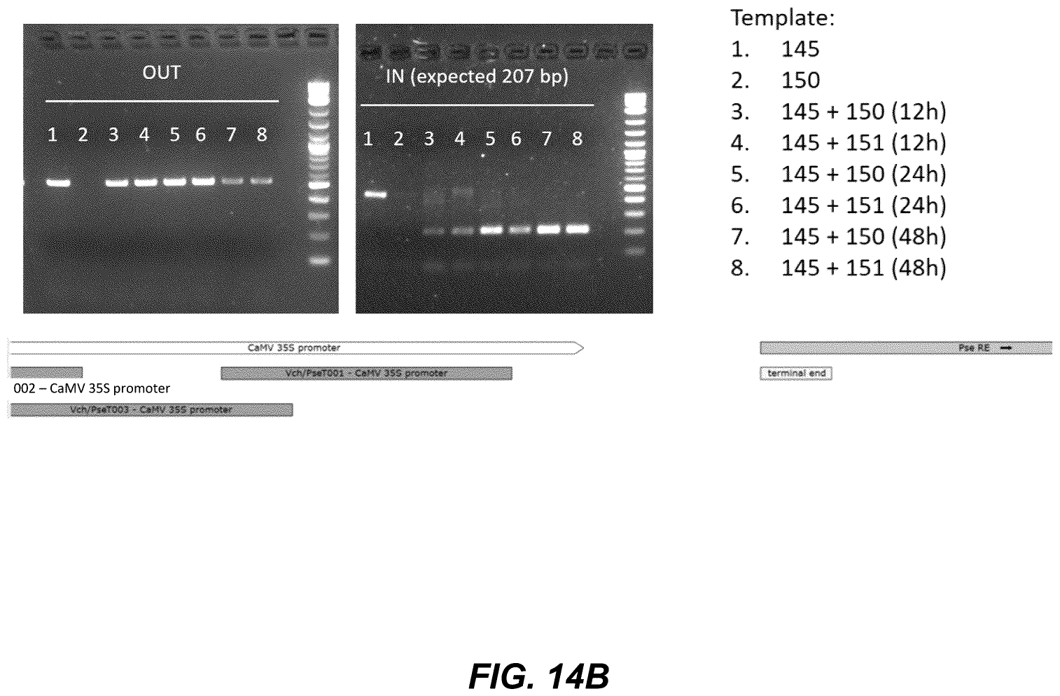

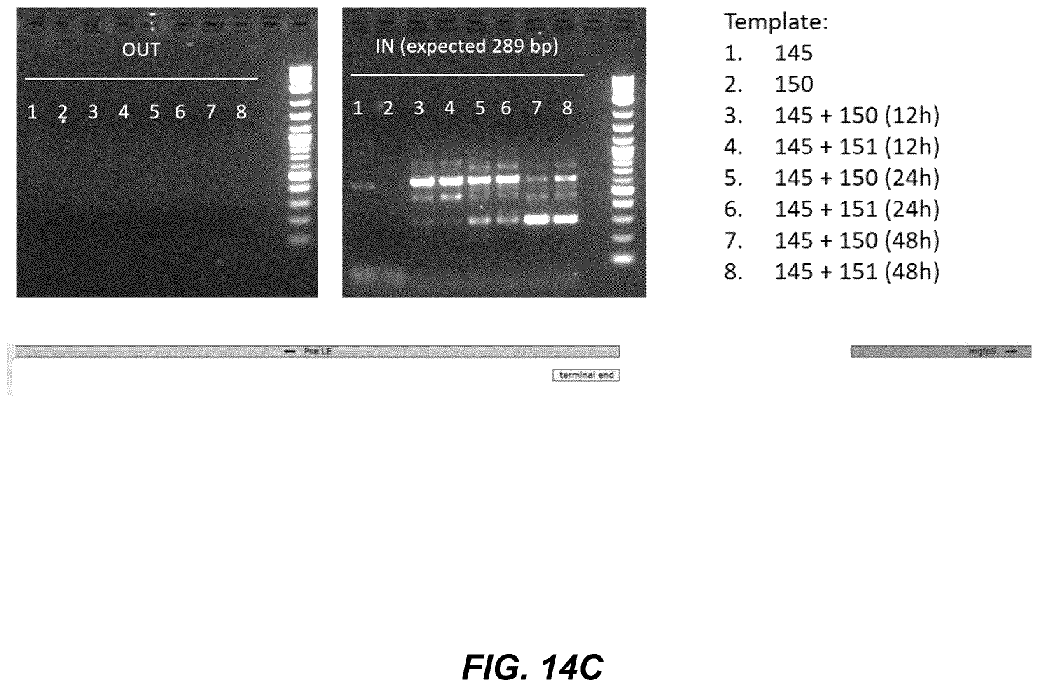

For all of the samples, molecular-level validation of targeted integration events was also conducted, in addition to the fluorescence imaging assays. One of the molecular-level analyses is junction PCR and sequencing, as correct junction sequences can confirm the CAST-mediated targeted insertion events. The samples were analyzed using nested PCR with primers 1F, 1R, 2F, 2R for the RE junction detection and 3F, 3R, 4F, 4R for the LE junction detection (FIG. 14A). Positive nested PCR results were obtained for both RE and LE junctions, showing a correct increase in band intensity over time. After gel-purification, bulk Sanger sequencing followed by TA-cloning confirmed the presence of correct RE & LE junction sequences. Results showed that CAST-mediated episomal DNA integration can occur as early as 12 hours post-protoplast transfection (FIG. 14B-FIG. 14C). Extending the incubation period while maintaining cell viability results in the observation of additional integration events.

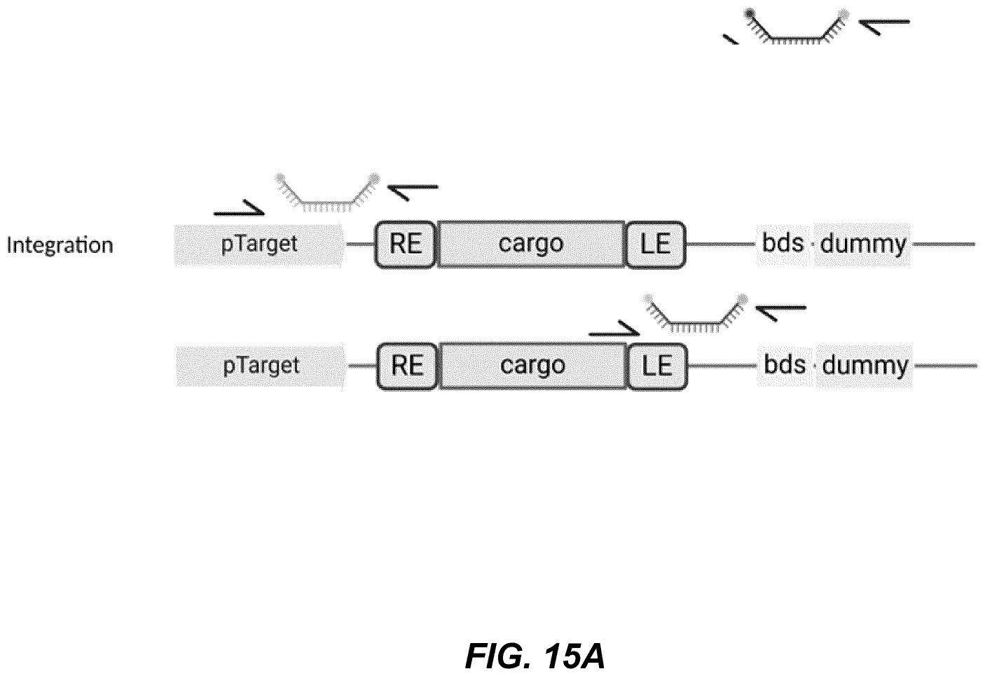

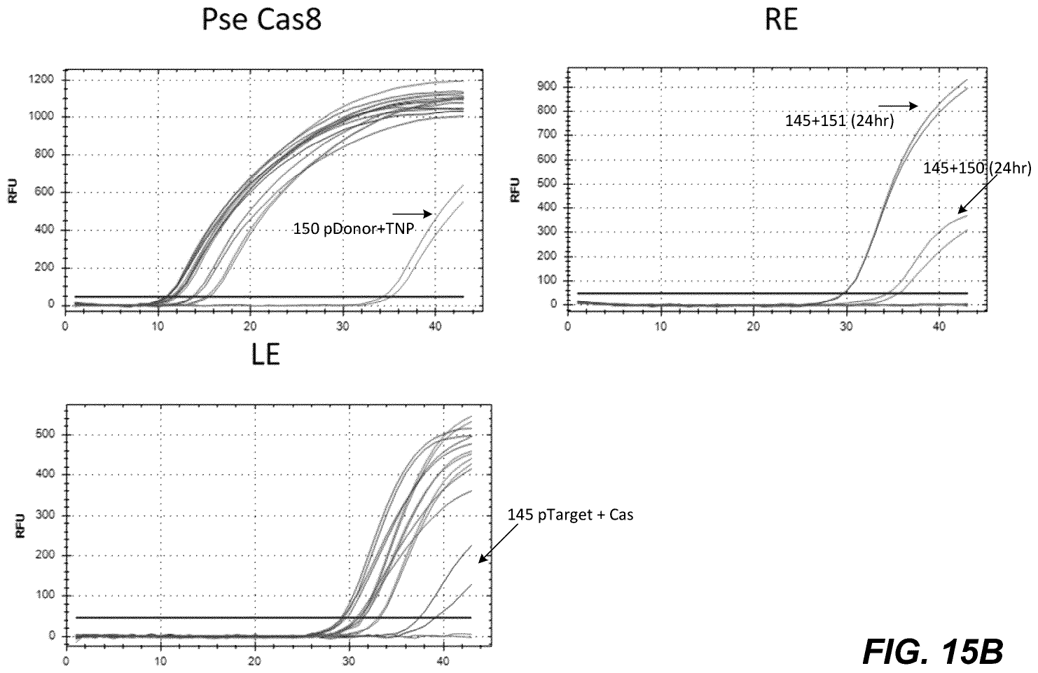

To obtain quantitative results, TaqMan probes and primers were designed to target the RE and LE junctions and the Cas8 reference gene (FIG. 15A). All probes were labeled with FAM to allow for easy threshold adjustment if needed. A detectable threshold cycle number (Ct) of approximately 30 for both RE and LE junctions was observed, distinct from the negative controls with a high Ct of more than 38 or not detectable (FIG. 15B, Table 8).

TABLE 8
THRESHOLD CYCLE (CT) VALUES

| Template | Cas8-FAM | Pse RE-FAM | Pse LE-FAM |
|---|---|---|---|
| 145 pTarget + CAS | 11.1 | NA | 38.1 |
| 150 pDonor + TNP | 34.6 | NA | NA |
| 145 + 150 (12 h) | 11.4 | Not tested | 31.1 |
| 145 + 151 (12 h) | 10.9 | Not tested | 30.8 |
| 145 + 150 (24 h) | 11.8 | 35.3 | 31.3 |
| 145 + 151 (24 h) | 11.1 | 30.1 | 29.4 |
| 145 + 150 (48 h) | 15.3 | Not tested | 33.1 |
| 145 + 151 (48 h) | 13.7 | Not tested | 29.2 |

An exemplary Taqman protocol is described below: (1) Design probes and primers using the IDT online tool; (2) Follow the protocol of Luna® Universal Probe qPCR Master Mix, and per 10 μl reaction, add 50˜100 ng extracted DNA; (3) Use the cycling program: an initial denaturation at 95° C. for 60 seconds, followed by 40 cycles of denaturation at 95° C. for 15 seconds and extension at 60° C. for 30 seconds with a plate read during the extension step.

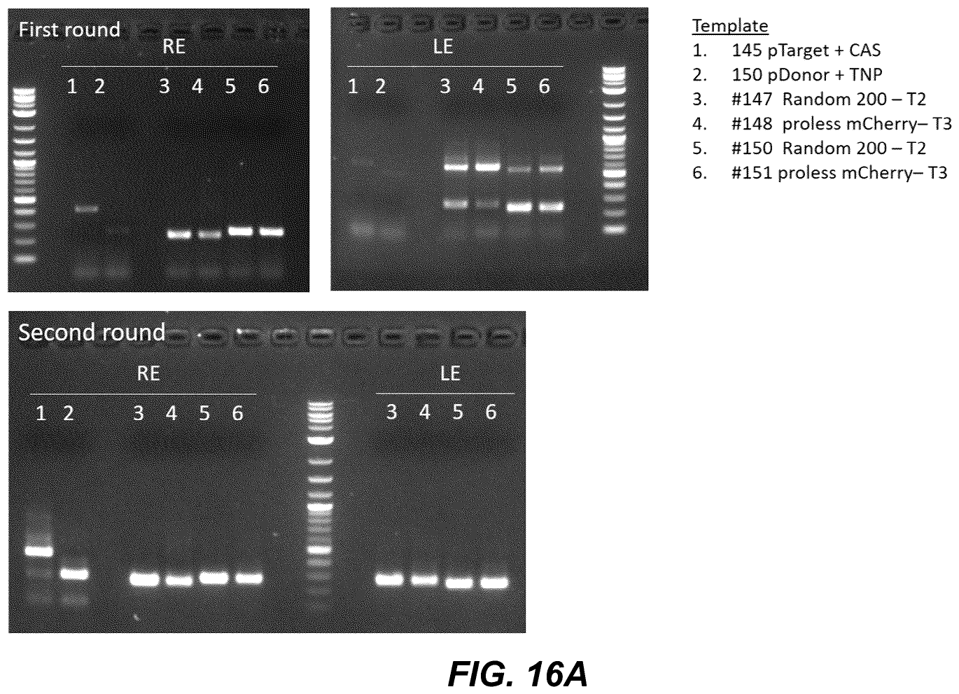

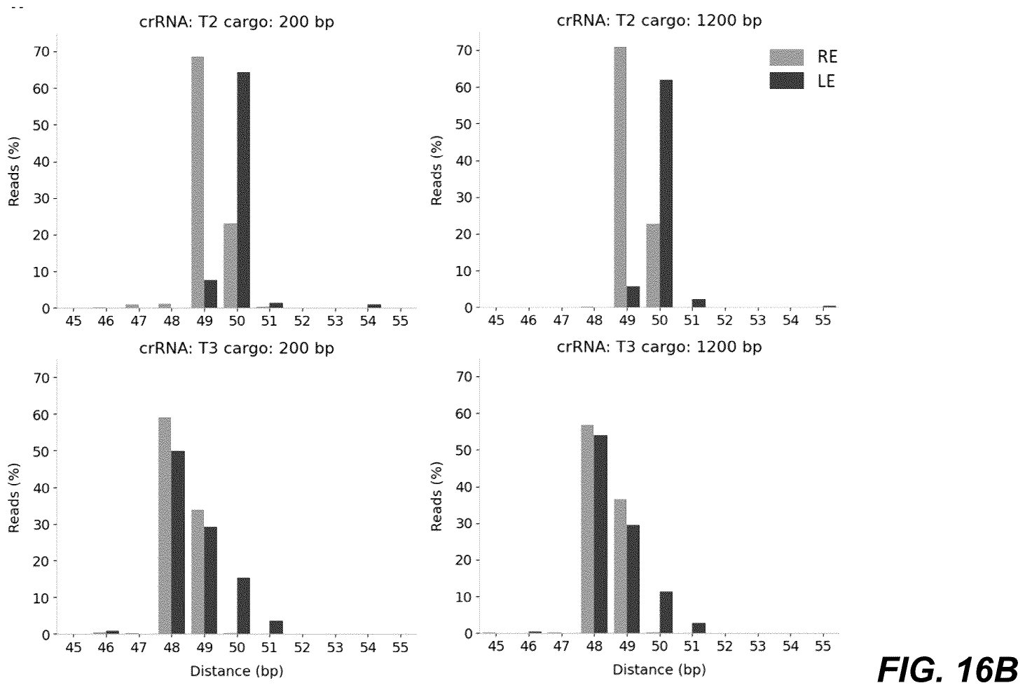

As the CAST system integrates cargo at varied distances downstream from the target site, it was next sought to determine the integration distance profile for the different crRNAs and cargo lengths tested. Primers were designed for pooled NGS sequencing, with the expected amplicon sizes as follows: RE: 198 bp (T2), 221 bp (T3); LE: 223 bp (T2), 196 bp (T3). The first round of PCR amplifies the target region while adding primer tags, and the second round introduces barcodes to the amplicons (FIG. 16A).

Results show expected integration distance profiles ranging from 48 to 51 bp away from the target site (FIG. 16B). Interestingly, variation of integration distance was observed between different crRNAs, but different cargo lengths did not affect this profile (FIG. 16B).

An exemplary protocol for amplicon sequencing is described below: (1) PCR is carried out by two consecutive PCR reactions using NEB Phusion® High-Fidelity PCR Master Mix with HF Buffer. The cycling number is 35. Annealing temperature and extension time is selected based on specific reaction; (2) Purify and measure the DNA amplicon product from the first round, and normalize their amount and dilute 10 times, then use it for the second round of PCR; (3) Purify and measure the DNA amplicon product from the second round, and then combine all samples at this point to reach 35 μl at 40 ng/μl; (4) Submit for MGH CCIB CRISPR Sequencing service.

Cast-Mediated DNA Integration into the Plant Chromosome in Leaves

Described below is a focus on RNA-guided DNA integration into the plant genome, as the ability to precisely engineer genomic loci is essential for achieving heritable genetic edits. Successful genomic integration is critical for both fundamental research and applied studies.

Cargo DNA was designed as a 200 bp random sequence at this stage. The crRNA was designed to target the cargo into the endogenous 35S promoter within the GFP cassette of the transgenic 16C N. benthamiana. Components were designed that are either cloned into a single plasmid, where cloning is more cumbersome requiring the assembly of large constructs exceeding 30 kb with repetitive sequences, or split into two plasmids. These constructs were transformed into Agrobacterium tumefaciens, and infiltrated into N. benthamiana leaves. The plants were kept in a growth chamber for 7 days, with overnight 30° C. heat shock treatment applied during the last three days. DNA samples were then harvested from leaves for subsequent phenotypic and molecular analysis.

Identifier and details of the constructs are: #120, T2/T3 LIR-RE-200 bp-LE-SIR+ClpX/ClpP+crRNA T2/3+Tnp+Cas+DsRed (T-DNA, SEQ ID NO: 44); #158, Tnp+Cas+pVmV mTurq (T-DNA, SEQ ID NO: 45); #427 T2/T3 LIR-RE-200 bp-LE-SIR+ClpX+ClpP+crRNA T002/3 (T-DNA, SEQ ID NO: 46).

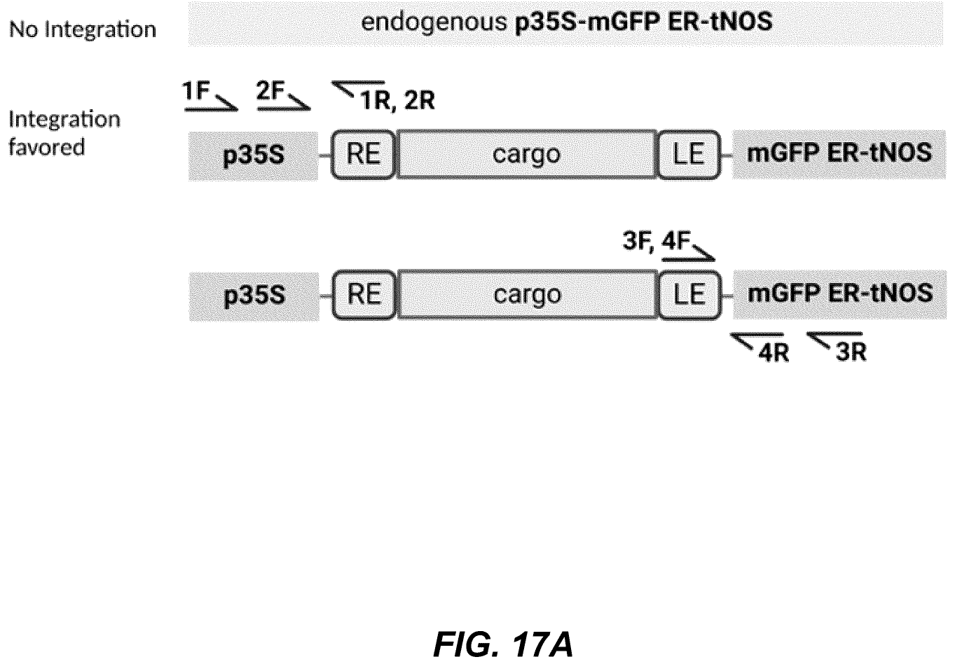

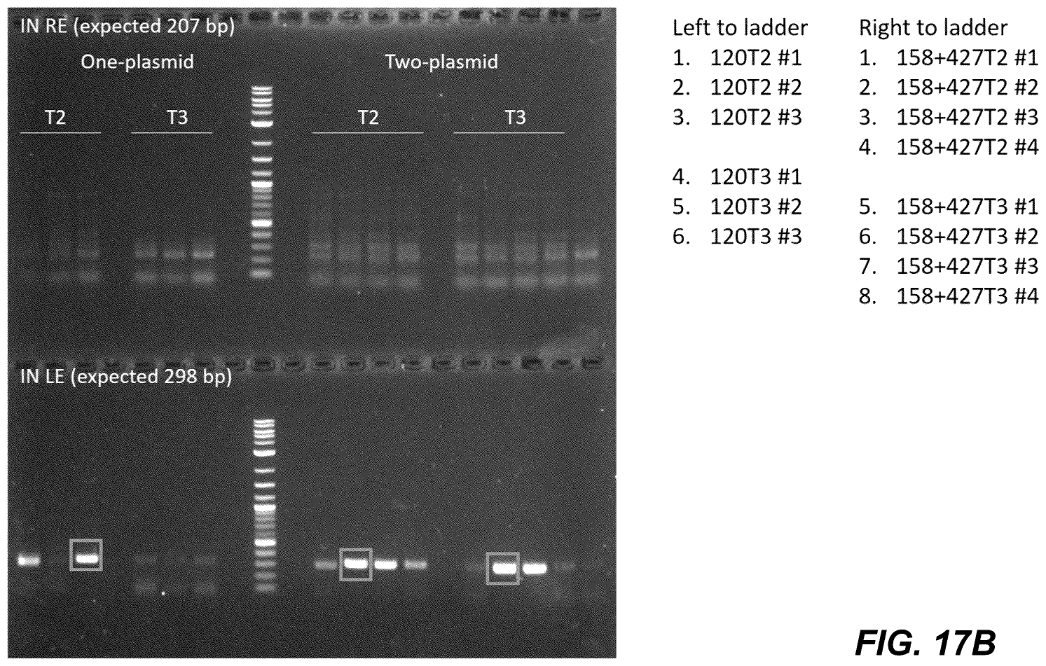

Multiple biological replicates (#1 to #4) were analyzed using nested PCR with primers 1F, 1R, 2F, 2R for the RE junction detection and 3F, 3R, 4F, 4R for the LE junction detection (FIG. 17A). The first OUT reaction typically yields no bands due to the low template amount. After the second round of amplification, the correct bands were detected for LE junctions (FIG. 17B). Bulk Sanger sequencing confirmed the LE junctions, with integration lengths of 48 to 50 bp, consistent with previous study in humans.

Improving CAST-Mediated DNA Integration into the Plant Genome in Protoplasts

To maximize integration efficiency, the CAST system was re-engineered where TnsA-B was included in the geminivirus-derived replicon to achieve high expression levels; and two accessory proteins, ClpX and ClpP, were incorporated to helper plasmid because of the necessity for transpososome dissociation to initiate DNA repair. This system was tested in protoplasts.

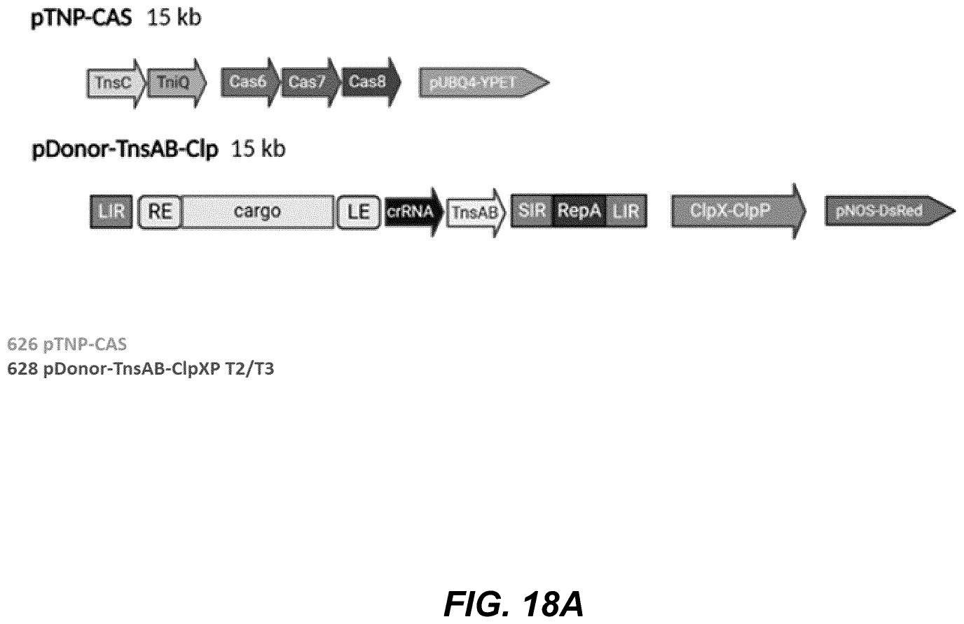

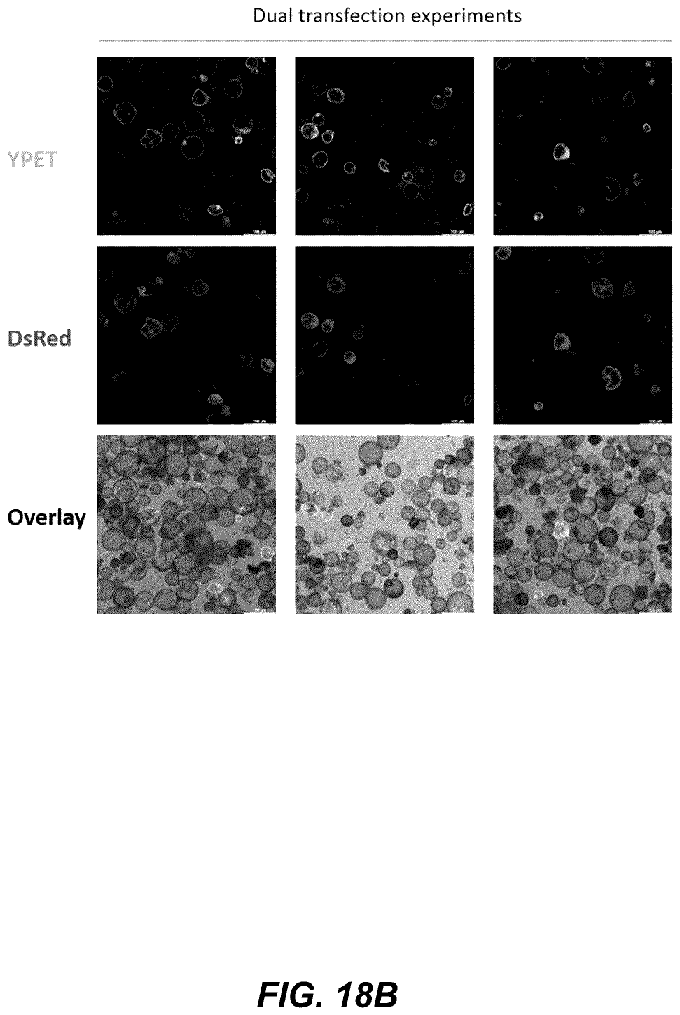

Cargo DNA was designed as a 200 bp random sequence at this stage. The crRNA was designed to target the cargo into the endogenous 35S promoter within the GFP cassette of the transgenic 16C N. benthamiana. All components were cloned into two separate plasmids, transfected into protoplasts and incubated for 72 hrs in dark and DNA samples were harvested for subsequent molecular analysis (FIG. 18A).

Identifier and details of the constructs are: #626, Tnp+Cas+pUBQ4-YPET; #628, T2/T3GV-200 bp-crRNA T2/T3-TnsAB+ClpXP+pNOS-DsRed.

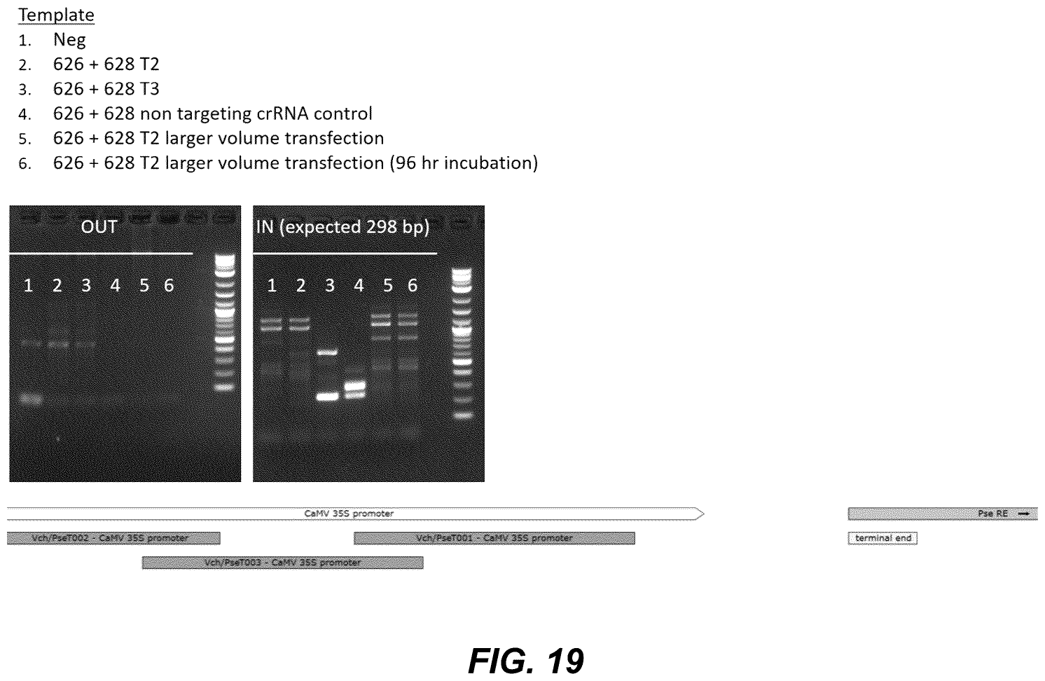

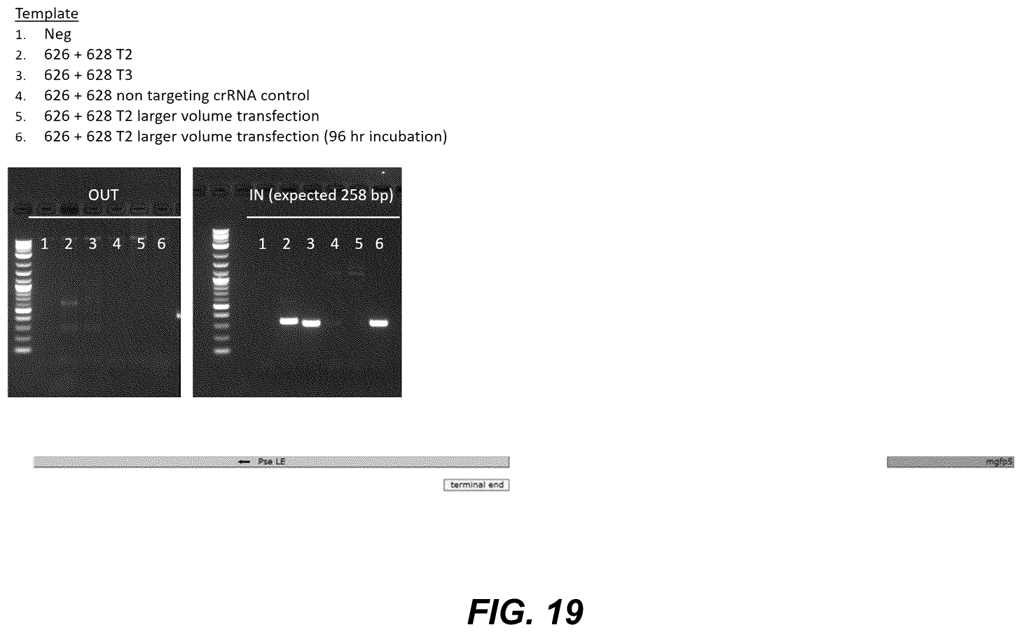

The samples were analyzed using nested PCR with primers 1F, 1R, 2F, 2R for the RE junction detection and 3F, 3R, 4F, 4R for the LE junction detection. Positive results for the RE junction were consistent with targeting by crRNA T2. The LE junction demonstrated integration distances of 49 or 50 bp as anticipated (FIG. 19).

Disclosed herein is CAST-mediated targeted gene insertion in plants for the first time using Vibrio cholera and Pseudoalteromonas CAST. Successful expression of all CAST proteins is shown in plant leaves. Episomal integration of 200 bp and 1200 bp DNA cargo is achieved in Arabidopsis thaliana protoplast cells. This was validated using nested PCR, TaqMan probe-based qPCR, and next-generation sequencing of RE and LE integration junctions.

Chromosomal integration of 200 bp DNA cargo into the genome of N. benthamiana protoplast was achieved. This was validated using nested PCR followed by Sanger sequencing of RE and LE integration junctions. Chromosomal integration of 200 bp DNA cargo into the genome Nicotiana benthamiana leaves was achieved. This was validated using nested PCR followed by Sanger sequencing of LE junction.

Shown below in Table 9-Table 10 are exemplary sequences of the disclosure.

TABLE 9
HELPER PROTEIN AND NUCLEOTIDE SEQUENCES

| | | SEQ |
|---|---|---|
| | | ID |
| Description | Sequence | NO: |
| VchCas6 | MVKWYYKTITFLPELCNNESLAAKCLRVLHGFNYQYETRNIGVSFPLWCDATV | 8 |
| amino acid | GKKISFVSKNKIELDLLLKQHYFVQMEQLQYFHISNTVLVPEDCTYVSFRRCQSI | |
| sequence | DKLTAAGLARKIRRLEKRALSRGEQFDPSSFAQKEHTAIAHYHSLGESSKQTNR | |
| | NFRLNIRMLSEQPREGNSIFSSYGLSNSENSFQPVPLI* | |
| PseCas6 | MQRYYFTVHFLPKQANLALLTGRCISIMHGFILKHNIEGMGVTFPAWSDSSIGN | 9 |
| amino acid | EIAFVYTDKEILNTLKDQAYFVDMQDCGFFKVSQVLAVPDSCEEVRFIRNQAV | |
| sequence | AKIFTGESRRRLKRLQKRALARGEDFNPKKIEAPREIDIFHRVAMTSKSSQEDYI | |
| | LHIQKQDVDCQAEPYFSNYGLASNEKFKGTVPDLSPSIDRN* | |
| VchCas7 | MKLPTNLAYERSIDPSDVCFFVVWPDDRKTPLTYNSRTLLGQMEAASLAYDVS | 10 |
| amino acid | GQPIKSATAEALAQGNPHQVDFCHVPYGASHIECSFSVSFSSELRQPYKCNSSK | |
| sequence | VKQTLVQLVELYETKIGWTELATRYLMNICNGKWLWKNTRKAYCWNIVLTP | |
| | WPWNGEKVGFEDIRTNYTSRQDFKNNKNWSAIVEMIKTAFSSTDGLAIFEVRA | |
| | TLHLPTNAMVRPSQVFTEKESGSKSKSKTQNSRVFQSTTIDGERSPILGAFKTGA | |
| | AIATIDDWYPEATEPLRVGRFGVHREDVTCYRHPSTGKDFFSILQQAEHYIEVLS | |
| | ANKTPAQETINDMHFLMANLIKGGMFQHKGD* | |
| PseCas7 | MELCNILKYDRSLYPGKAVFFYKTADSDFVPLEADINKIRGPKSGFTEAFTPQFS | 11 |
| amino acid | PKNISPQDLTHNNILTLEECYVPPNVEHIFCRFSLRVQANSLVPSGCSDPEVFSLL | |
| sequence | KELAETFKECGGYKELAVRYCRNILIGTWLWRNQNTGNTQIEIKTSKGSCYLID | |
| | NTRKLAWESKWASDDLKVLEELSNEIESALTDPNVFWSADITAKIEASFCQEIY | |
| | PSQILNDKVKQGEASKQFVKAKCADGRYAVSFNSVKIGAALQSIDDWWDEDA | |
| | SKRLRVHEFGADKEIGVARRPPDSEQNFYSIFKNTEWYLSALKNCITNKNEKIDP | |
| | AIYYLFSVLIKGGMFQKKAEAKKA* | |
| VchCas8 | MQTLKELIASNPDDLTTELKRAFRPLTPHIAIDGNELDALTILVNLTDKTDDQKD | 12 |
| amino acid | LLDRAKCKQKLRDEKWWASCINCVNYRQSHNPKFPDIRSEGVIRTQALGELPSF | |
| sequence | LLSSSKIPPYHWSYSHDSKYVNKSAFLTNEFCWDGEISCLGELLKDADHPLWNT | |
| | LKKLGCSQKTCKAMAKQLADITLTTINVTLAPNYLTQISLPDSDTSYISLSPVAS | |
| | LSMQSHFHQRLQDENRHSAITRFSRTTNMGVTAMTCGGAFRMLKSGAKFSSPP | |
| | HHRLNSKRSWLTSEHVQSLKQYQRLNKSLIPENSRIALRRKYKIELQNMVRSWF | |
| | AMQDHTLDSNILIQHLNHDLSYLGATKRFAYDPAMTKLFTELLKRELSNSINNG | |
| | EQHTNGSFLVLPNIRVCGATALSSPVTVGIPSLTAFFGFVHAFERNINRTTSSFRV | |
| | ESFAICVHQLHVEKRGLTAEFVEKGDGTISAPATRDDWQCDVVFSLILNTNFAQ | |
| | HIDQDTLVTSLPKRLARGSAKIAIDDFKHINSFSTLETAIESLPIEAGRWLSLYAQ | |
| | SNNNLSDLLAAMTEDHQLMASCVGYHLLEEPKDKPNSLRGYKHAIAECIIGLIN | |
| | SITFSSETDPNTIFWSLKNYQNYLVVQPRSINDETTDKSSL* | |
| PseCas8 | MHLKELLEITDTTERDRSLRRAFSPYTAMIDITGSEAVALIILLNLTYRKNQVDD | 13 |
| amino acid | LLDKKLAKQALKSEDHINKCIKEIAWFHTHNLKYPDIRVSKQNLAVEPPTLHSY | |
| sequence | VLSSANYPKAYGWSHNSAKVNFAKLFVSYFKWQNQVSWLAQVLATNSDNWK | |
| | SAFTSLGLSVKAFKSLCVTVKNSLPEEAIPDSVDRYSRQIRMPYHDGYLAVTPVI | |
| | SHVVQSKIQQAAIDKRARFSNVEFTRPAAVSMLAASLGGVINVLNYPPYIRSKY | |
| | HGLSNSRAFKLNNGQTVFNVEALLKPELIKALEGIIFSNNALALKQRRQQKVKN | |
| | IKELRNTLLEWFSPVFEWRLDAIENGYDLEQLESASERLEYKILSLPDNELPSLTI | |
| | PLFRLLNEMLGGVSMTQRYAFHPKLMSPLKAALQWLLVNLTDQKHVLIEEDD | |
| | EHYRYLHLSGIRVFDAQALSNPYCSGIPSLTAVWGMIHSYQRKLNEALGTNVRF | |
| | TSFSWFIRNYSAVAGKKLPELSLQGAQQSRLKRPGIIDGKYCDLVFDLIIHIDGY | |
| | EDDLQAVDSKPDILKAHFPSNFAGGVMHQPELNSNINWCCLYSNENQLFEKLR | |
| | RLPLSGCWVMPTEHKIQDLDELLLLLNSDSKLSPSMMGYMLLTEPMARVGSLE | |
| | RLHCYAEPAIGVVKYEAATSVRLKGIGNYFNSAFWMLDAQEKFMLMKKV* | |
| VchTniQ | MFLQRPKPYSDESLESFFIRVANKNGYGDVHRFLEATKRFLQDIDHNGYQTFPT | 14 |
| amino acid | DITRINPYSAKNSSSARTASFLKLAQLTFNEPPELLGLAINRTNMKYSPSTSAVV | |
| sequence | RGAEVFPRSLLRTHSIPCCPLCLRENGYASYLWHFQGYEYCHSHNVPLITTCSC | |
| | GKEFDYRVSGLKGICCKCKEPITLTSRENGHEAACTVSNWLAGHESKPLPNLPK | |
| | SYRWGLVHWWMGIKDSEFDHFSFVQFFSNWPRSFHSIIEDEVEFNLEHAVVSTS | |
| | ELRLKDLLGRLFFGSIRLPERNLQHNIILGELLCYLENRLWQDKGLIANLKMNA | |
| | LEATVMLNCSLDQIASMVEQRILKPNRKSKPNSPLDVTDYLFHFGDIFCLWLAE | |
| | FQSDEFNRSFYVSRW* | |
| PseTniQ | MAFLFSPKARAFSDESLESYLLRVVSENFFDSYEGLSLAIREELHELDFEAHGAF | 15 |
| amino acid | PVDLKRLNVYHAKHNSHFRMRALGLLETLLDLPRYELQKLALLKSDIKFNSSV | |
| sequence | ALYNNGVDIPLRFIRHHAEEAVDSIPVCSQCLAEEAYIKQSWHIKWVNACTKHQ | |
| | CALLHNCPECYAPINYIENESITHCSCGFELSCASTSPVNTLSIEHLNKLLDKGER | |
| | NDSNPLFNNMTLTERFAALLWYQERYSQTDNFCLNDAVNYFSKWPAVFNTEL | |
| | DELSKNAEMKLIDLFNKTEFKFIFGDAILACPSTQKQSESHFIYRALLDYLVTLV | |
| | ESNPKTKKPNAADLLVSVLEAATLLGTSVEQVYRLYQNGILQTAFRHKMNQRI | |
| | NPYKGAFFLRHVIEYKTSFGNDKARMYLSAWTG | |
| VchTnsAB | MATSLPTPSAITTSALEYAFHTPARNLTKSRGKNIHRYVSVKMSKRITVESTLEC | 16 |
| fusion | DACYHFDFEPSIVRFCAQPIRFLYYLNGQSHSYVPDFLVQFDTNEFVLYEVKSA | |
| amino acid | YAKNKPDFDVEWEAKVKAATELGLELELVEESDIRDTVVLNNLKRMHRYASK | |
| sequence | DELNNVHNSLLKIIKYNGAQSARCLGEQLGLKGRTVLPILCDLLSRCLLDTRLD | |
| | KPLSLESRFELASYGGSGSGKRTADGSEFESPKKKRKVGSGSGGAKKGFSSFHR | |
| | KAVSSQDTLESIELVSSANCLESVTYQDISAFPETIAVEINFRLSILRFLARKCETI | |
| | VAKSIEPHRVELQQNYSRKIPSAITIYRWWLAFRKSDYNPISLAPNIKDRGNRET | |
| | KVSTVVDSIMEQAVERVISGRKVNVSSAYKRVRRKVRQYNLTHGTKYTYPKY | |
| | ESVRKRVKKKTPFELLAAGKGERVAKREFRRMGKKILTSSVLERVEIDHTVVD | |
| | LFAVHEEYRIPLGRPWLTQLVDCYSKAVIGFYLGFEPPSYVSVSLALKNAIQRK | |
| | DDLISSYESIENEWLCYGIPDLLVTDNGKEFLSKAFDQACESLLINVHQNKVETP | |
| | DNKPHVERNYGTINTSLLDDLPGKSFSQYLQREGYDSVGEATLTLNEIREIYLIW | |
| | LVDIYHKKPNQRGTNCPNVAWKKGCQEWEPEEFSGSKDELDFKFAIVDYKQLT | |
| | KVGITVYKELSYSNDRLAEYRGKKGNHKVQFKYNPECMAVIWVLDEDMNEYF | |
| | TVNAIDYEYASRVSLWQHKYNMKYQAELNSAEYDEDKEIDAEIKIEEIADRSIV | |
| | KTNKIRARRRGARHQENSARAKSISNANPASIQKHEDEIVSADNDDWDIDYV* | |
| PseTnsAB | MYIRNLRKPSPNKNVFKFASTKVSSVVMCESSLEFDACFHHEYNDLIESFGSQP | 17 |
| fusion | EGFKYEFMGKSLPYTPDALISYTDKTQKYHEYKPYSKIASPLFRAEFAAKRAAS | |
| amino acid | LKLGIDLVLVTDRQIRVNPILNNLKLLHRYSGVYGISGIQKELLSFIHKSGVIKLN | |
| sequence | DISSQVGIPIGETRSFLFGLMHKGLVKADLGCDDLTNNPTLWATPGSGSGKRTA | |
| | DGSEFESPKKKRKVGSGSGGMTDFFNEFDESLVPLKPQTPTQYVKLDDANLIQR | |
| | DLDTFSDTFKNQALQRYKLISTIDKKLSRGWTQRNLDPILDELFKGGDVVRPN | |
| | WRTVARWRKKYIESNGDIASLADKNHKMGNRTNRIKGDDKFFDKALERFLDA | |
| | KRPTIATAYQYYKDLIVIENESIVEGKIPIISYNAFNKRIKAIPPYAVAVARHGKF | |
| | KADQWFAYCAAHVPPTRILERVEIDHTPLDLILLDDELLIPIGRPYLTLLIDVFSG | |
| | CVLGFHLSYKSPSYVSAAKAITHAIKPKSLDALNIELQNDWPCFGKFENLVVDN | |
| | GAEFWSKNLEHACQSAGINIQYNPVRKPWLKPFIERFFGVMNEYFLPELPGKTF | |
| | SNILEKEEYKPEKDAIMRFSTFVEEFHRWIADVYHQDSNSRETRIPIKRWQQGFD | |
| | AYPPLTMNEEEETRFSMLMRISDSRTLTRNGFKYQELMYDSTALADYRKHYPQ | |
| | TKETVKKLIKVDPDDISKIYVYLEELESYLEVPCTDPTGYTDGLSIYEHKTIKKIN | |
| | REVIRESKDSLGLAKARMAIHERVKQEQEVFIESKTKAKITAVKKQAQIADVSN | |
| | TGTSTIKVSEESAAPVQKHISNDNSDDWDDDLEAFE* | |
| VchTnsC | MSETREARISRAKRAFVSTPSVRKILSYMDRCRDLSDLESEPTCMMVYGASGV | 18 |
| amino acid | GKTTVIKKYLNQNRRESEAGGDIIPVLHIELPDNAKPVDAARELLVEMGDPLAL | |
| sequence | YETDLARLTKRLTELIPAVGVKLIIIDEFQHLVEERSNRVLTQVGNWLKMILNKT | |
| | KCPIVIFGMPYSKVVLQANSQLHGRFSIQVELRPFSYQGGRGVFKTFLEYLDKA | |
| | LPFEKQAGLANESLQKKLYAFSQGNMRSLRNLIYQASIEAIDNQHETITEEDFVF | |
| | ASKLTSGDKPNSWKNPFEEGVEVTEDMLRPPPKDIGWEDYLRHSTPRVSKPGR | |
| | NKNFFE* | |
| PseTnsC | MGNALTEIQIEKLRNFSDCIVMHPQIKTIFNDFDELRLNRKFQSDQQCMLLIGDT | 19 |
| amino acid | GVGKSHTINHYKKRVLATQNYSRNTMPVLVSRISRGKGLDATLVQMLADLELF | |
| sequence | GSSQIKKRGYKTDLTKKLVESLIKAQVELLIINEFQELIEFKSVQERQQIANGLKF | |
| | ISEEAKVPIVLVGMPWAAKIAEEPQWASRLVRKRKLEYFSLKNDSKYFRQYLM | |
| | GLAKKMPFDVPPKLESKNTTIALFAACRGENRALKHLLLEALKLALSCNEYLE | |
| | NKHFITAYDKFDFFNDKEKLKSKNPFKQDIKDIEIYEVIKNSSYNPNALDPEDML | |
| | TDRVFAIVK | |
| ClpP | MSYSGERDNFAPHMALVPMVIEQTSRGERSFDIYSRLLKERVIFLTGQVEDHMA | 20 |
| amino acid | NLIVAQMLFLEAENPEKDIYLYINSPGGVITAGMSIYDTMQFIKPDVSTICMGQA | |
| sequence | ASMGAFLLTAGAKGKRFCLPNSRVMIHQPLGGYQGQATDIEIHAREILKVKGR | |
| | MNELMALHTGQSLEQIERDTERDRFLSAPEAVEYGLVDSILTHRN* | |
| ClpX | MTDKRKDGSGKLLYCSFCGKSQHEVRKLIAGPSVYICDECVDLCNDIIREEIKE | 21 |
| amino acid | VAPHRERSALPTPHEIRNHLDDYVIGQEQAKKVLAVAVYNHYKRLRNGDTSN | |
| sequence | GVELGKSNILLIGPTGSGKTLLAETLARLLDVPFTMADATTLTEAGYVGEDVEN | |
| | IIQKLLQKCDYDVQKAQRGIVYIDEIDKISRKSDNPSITRDVSGEGVQQALLKLIE | |
| | GTVAAVPPQGGRKHPQQEFLQVDTSKILFICGGAFAGLDKVISHRVETGSGIGF | |
| | GATVKAKSDKASEGELLAQVEPEDLIKFGLIPEFIGRLPVVATLNELSEEALIQIL | |
| | KEPKNALTKQYQALFNLEGVDLEFRDEALDAIAKKAMARKTGARGLRSIVEAA | |
| | LLDTMYDLPSMEDVEKVVIDESVIDGQSKPLLIYGKPEAQQASGE* | |
| SV40 | CCAAAGAAGAAAAGAAAAGTT | 22 |
| Nuclear | | |
| Localization | | |
| Signal (NLS) | | |
| nucleotide | | |
| sequence | | |
| Bipartite | ATGGGAAAGCGTACTGCTGATGGTTCAGAGTTTGAAAGTCCAAAGAAGAAA | 23 |
| NLS | AGAAAAGTT | |
| nucleotide | | |
| VchCas6 | ATGGTGAAATGGTACTACAAAACAATAACATTTCTACCTGAGCTCTGCAAC | 24 |
| nucleotide | AATGAATCACTAGCAGCGAAATGTCTGCGTGTACTTCATGGATTCAACTACC | |
| sequence | AATATGAAACAAGAAACATTGGTGTTAGCTTCCCTCTTTGGTGTGACGCAAC | |
| | TGTCGGAAAGAAGATATCCTTTGTGTCCAAGAACAAGATTGAGCTTGACTT | |
| | GCTTCTCAAACAACATTACTTTGTTCAAATGGAGCAGCTTCAGTATTTCCAC | |
| | ATCTCAAATACTGTGCTGGTTCCAGAAGATTGCACTTATGTCAGTTTCAGGA | |
| | GATGTCAAAGTATTGATAAGCTTACTGCTGCTGGTTTAGCAAGAAAGATCC | |
| | GTCGATTGGAGAAGAGAGCTTTGTCAAGAGGAGAACAGTTTGATCCCTCAA | |
| | GTTTTGCTCAGAAAGAACACACCGCCATTGCTCATTATCATTCTCTCGGTGA | |
| | ATCTTCAAAGCAAACAAATAGGAATTTTCGGTTAAATATAAGGATGTTGAG | |
| | TGAACAACCAAGAGAAGGAAATTCCATCTTCTCTTCTTATGGGCTATCAAAT | |
| | TCTGAGAATTCTTTTCAGCCTGTTCCGCTAATCTGA | |
| PseCas6 | ATGCAGAGATATTATTTTACCGTTCATTTTCTTCCTAAACAAGCTAATTTGG | 25 |
| nucleotide | CCCTTCTCACCGGAAGATGCATATCTATCATGCATGGATTTATCTTGAAGCA | |
| sequence | TAACATTGAAGGAATGGGAGTAACATTTCCCGCATGGTCTGATTCAAGCAT | |
| | TGGAAACGAGATTGCTTTTGTTTACACTGATAAAGAAATCCTAAACACATTG | |
| | AAAGATCAGGCTTACTTTGTGGATATGCAAGACTGTGGTTTCTTCAAGGTTT | |
| | CTCAGGTGTTGGCTGTTCCTGATTCTTGCGAGGAAGTCAGGTTCATCCGCAA | |
| | TCAAGCCGTCGCAAAGATTTTCACTGGCGAATCGAGGAGGAGACTTAAACG | |
| | GTTACAAAAGAGAGCCTTAGCAAGAGGAGAAGATTTCAATCCAAAGAAGA | |
| | TAGAAGCTCCAAGAGAAATAGACATTTTTCACCGTGTAGCAATGACAAGCA | |
| | AAAGTTCTCAAGAAGACTACATTCTGCACATCCAGAAGCAAGATGTTGATT | |
| | GTCAAGCAGAGCCTTATTTTTCAAACTATGGTCTTGCGTCAAATGAGAAGTT | |
| | CAAAGGAACTGTACCAGACTTGAGTCCGTCCATTGATAGAAATTGA | |
| VchCas7 | ATGAAACTTCCAACAAATCTAGCTTACGAGAGGTCGATTGATCCTTCAGAT | 26 |
| nucleotide | GTTTGTTTCTTTGTGGTCTGGCCAGATGATCGGAAAACACCGTTGACATACA | |
| sequence | ATTCACGTACCCTCCTAGGGCAGATGGAGGCTGCATCTCTGGCGTATGATGT | |
| | AAGTGGTCAACCGATCAAGAGTGCCACAGCAGAAGCATTAGCTCAAGGAA | |
| | ATCCTCATCAAGTTGACTTTTGTCATGTTCCTTATGGAGCATCTCACATCGA | |
| | GTGCAGCTTCTCTGTTTCTTTTTCATCAGAGCTTCGCCAGCCTTACAAATGTA | |
| | ACAGCAGTAAAGTGAAGCAAACTCTCGTTCAGCTCGTAGAGTTGTATGAAA | |
| | CTAAGATTGGATGGACTGAACTAGCGACGAGATATTTGATGAACATATGCA | |
| | ATGGTAAATGGCTCTGGAAAAATACAAGGAAGGCTTATTGTTGGAATATAG | |
| | TCCTGACTCCATGGCCTTGGAACGGAGAGAAGGTTGGCTTTGAAGACATTA | |
| | GGACAAACTACACTTCAAGACAAGATTTCAAGAACAACAAGAATTGGTCTG | |
| | CCATTGTTGAGATGATTAAGACAGCTTTCAGTTCTACTGATGGCTTAGCGAT | |
| | CTTCGAGGTTCGTGCGACTCTTCACTTACCAACCAATGCTATGGTTAGACCT | |
| | TCCCAGGTATTCACAGAGAAAGAATCAGGGTCGAAATCAAAAAGCAAGAC | |
| | TCAGAATTCTCGAGTGTTTCAATCAACAACCATAGATGGTGAAAGATCTCCC | |
| | ATTCTTGGTGCATTCAAAACTGGTGCAGCTATAGCTACTATTGACGACTGGT | |
| | ATCCTGAAGCTACAGAACCATTGCGTGTTGGAAGATTTGGTGTCCATAGAG | |
| | AAGATGTGACTTGCTATAGACATCCTTCCACAGGGAAAGATTTTTTCTCCAT | |
| | ACTTCAACAAGCTGAACATTACATTGAAGTACTTTCAGCAAACAAAACCCC | |
| | AGCACAAGAAACCATTAATGATATGCATTTTCTTATGGCTAATCTCATCAAA | |
| | GGTGGCATGTTTCAGCACAAAGGAGACTGA | |
| PseCas7 | ATGGAGCTTTGCAATATACTCAAGTATGATCGAAGTCTTTACCCTGGGAAA | 27 |
| nucleotide | GCGGTTTTCTTCTACAAAACGGCGGACAGCGATTTTGTTCCTTTGGAAGCTG | |
| sequence | ACATTAATAAGATAAGAGGACCAAAATCAGGCTTTACAGAGGCTTTCACTC | |
| | CTCAATTCTCTCCGAAAAATATCTCTCCACAAGATCTAACTCACAACAACAT | |
| | TCTTACACTCGAAGAGTGTTATGTTCCACCAAATGTCGAACATATTTTCTGC | |
| | CGGTTTTCTCTGCGTGTCCAAGCTAATTCGTTAGTCCCCTCTGGTTGTTCCGA | |
| | TCCTGAGGTTTTTAGCCTTCTCAAAGAGCTTGCAGAAACCTTCAAAGAATGT | |
| | GGTGGTTACAAGGAGCTGGCAGTACGCTATTGTCGGAACATTTTGATTGGA | |
| | ACATGGCTTTGGAGAAATCAGAATACCGGAAACACACAGATTGAGATCAAG | |
| | ACTTCAAAAGGATCTTGCTACTTGATTGATAATACAAGAAAACTCGCTTGG | |
| | GAAAGCAAATGGGCTTCAGACGACTTGAAAGTTTTGGAAGAATTAAGCAAT | |
| | GAAATCGAATCTGCTTTAACAGATCCAAATGTGTTTTGGTCTGCAGATATAA | |
| | CCGCCAAGATCGAAGCATCTTTCTGTCAAGAGATATATCCTTCACAGATATT | |
| | GAATGATAAAGTGAAGCAAGGAGAAGCGTCAAAACAGTTTGTGAAAGCTA | |
| | AATGTGCTGATGGAAGGTATGCTGTGAGTTTCAACAGTGTTAAGATCGGTG | |
| | CTGCTTTGCAAAGTATTGATGATTGGTGGGATGAAGATGCTAGTAAAAGAC | |
| | TTAGAGTTCATGAATTTGGTGCAGACAAGGAAATTGGCGTAGCGAGGAGGC | |
| | CTCCCGATTCCGAACAAAACTTTTATTCAATCTTCAAGAACACTGAATGGTA | |
| | TCTTTCCGCCTTAAAGAATTGCATCACTAACAAGAATGAGAAGATTGACCC | |
| | AGCAATATACTATTTGTTCAGCGTGTTGATCAAAGGTGGAATGTTTCAGAAA | |
| | AAGGCAGAGGCAAAGAAAGCATGA | |
| VchCas8 | ATGCAGACTCTTAAAGAACTAATTGCTTCTAATCCGGATGACCTAACAACG | 28 |
| nucleotide | GAGCTCAAAAGGGCATTTCGCCCCCTGACTCCTCACATTGCCATTGACGGTA | |
| sequence | ACGAGCTTGACGCTCTCACCATTCTTGTGAATCTCACAGACAAAACTGATGA | |
| | TCAAAAGGATCTTCTCGATAGAGCTAAATGTAAACAGAAACTCAGAGATGA | |
| | GAAATGGTGGGCTTCTTGCATCAATTGCGTTAATTATAGACAGTCTCACAAT | |
| | CCTAAATTCCCTGATATCAGGTCTGAAGGAGTGATTCGCACTCAAGCTTTGG | |
| | GCGAACTTCCTTCATTTCTATTAAGCTCCAGTAAAATACCACCGTATCATTG | |
| | GTCATATTCTCATGATTCAAAATATGTTAACAAGTCAGCCTTTTTAACAAAT | |
| | GAATTCTGTTGGGACGGAGAGATAAGCTGCTTGGGGGAACTCCTCAAAGAT | |
| | GCGGATCATCCACTTTGGAACACACTAAAGAAGCTTGGCTGCTCACAGAAG | |
| | ACATGCAAAGCTATGGCGAAACAATTGGCTGATATCACATTAACAACGATT | |
| | AATGTAACACTCGCTCCGAATTACTTGACTCAGATTTCTCTTCCGGATTCTG | |
| | ATACAAGTTACATTTCTCTCTCGCCTGTCGCAAGTCTATCAATGCAAAGTCA | |
| | TTTCCACCAACGTTTGCAAGACGAAAACAGACATTCTGCTATAACAAGATT | |
| | CTCCCGAACAACTAACATGGGTGTGACTGCAATGACTTGTGGTGGAGCTTTC | |
| | AGGATGTTAAAGTCTGGTGCAAAGTTCTCCAGTCCACCTCATCATAGATTAA | |
| | ACTCGAAACGCTCTTGGTTGACCAGTGAGCATGTTCAATCATTGAAGCAGT | |
| | ATCAACGTCTGAACAAATCATTGATCCCTGAGAATAGTAGGATTGCTCTTAG | |
| | GCGCAAGTACAAGATTGAACTTCAAAATATGGTTCGATCATGGTTTGCCAT | |
| | GCAAGATCACACTCTCGATTCAAATATCCTTATTCAACATCTCAATCACGAT | |
| | TTGAGTTATCTCGGGGCGACCAAAAGATTTGCTTATGATCCAGCTATGACAA | |
| | AATTGTTCACTGAGCTGCTAAAGAGGGAGCTGAGTAATTCTATCAACAACG | |
| | GAGAACAACATACCAATGGCAGTTTTCTGGTCCTCCCAAACATACGGGTTT | |
| | GTGGAGCAACTGCGTTATCTTCCCCAGTAACCGTTGGCATCCCTAGCTTAAC | |
| | CGCCTTCTTTGGTTTTGTTCACGCCTTTGAGAGGAACATCAACAGGACCACT | |
| | TCTTCCTTTCGTGTCGAGAGCTTTGCAATATGTGTCCATCAGCTTCATGTGG | |
| | AGAAGCGTGGTTTGACAGCTGAGTTTGTAGAGAAGGGAGATGGGACAATTT | |
| | CAGCACCAGCAACCAGAGATGATTGGCAATGTGATGTTGTGTTTTCTCTAAT | |
| | TTTGAATACGAACTTCGCTCAACACATTGATCAGGACACACTGGTCACGAG | |
| | TTTACCTAAGCGGCTGGCAAGGGGTTCTGCTAAGATCGCAATAGATGATTTC | |
| | AAGCATATAAACTCATTCTCCACTCTTGAAACTGCAATTGAGTCTCTTCCCA | |
| | TCGAAGCTGGAAGATGGCTTAGTCTGTATGCACAGAGTAATAATAATCTTTC | |
| | GGACTTGTTAGCTGCTATGACCGAAGACCATCAACTTATGGCTAGTTGTGTT | |
| | GGCTACCACCTTCTAGAAGAACCAAAAGATAAGCCAAATAGCCTTAGAGGA | |
| | TACAAACATGCCATAGCTGAATGTATTATAGGATTAATTAATTCAATTACAT | |
| | TTTCCTCGGAAACCGATCCTAATACTATATTCTGGTCCTTAAAGAACTACCA | |
| | AAACTATCTTGTAGTTCAGCCCAGATCCATAAATGATGAAACAACAGACAA | |
| | GAGCAGCCTGTGA | |
| PseCas8 | ATGCACCTGAAAGAGCTGTTAGAGATAACAGATACAACTGAGAGAGATAG | 29 |
| nucleotide | ATCTCTCCGACGTGCCTTCTCGCCGTATACCGCCATGATCGACATTACAGGA | |
| sequence | TCAGAAGCAGTCGCTCTAATTATCCTACTCAATCTCACTTATAGAAAGAATC | |
| | AGGTAGATGACCTGTTGGATAAGAAACTCGCGAAACAAGCCCTCAAGAGTG | |
| | AGGATCATATCAACAAATGCATCAAGGAGATTGCTTGGTTTCATACTCACA | |
| | ACCTTAAATATCCAGACATAAGGGTGAGTAAGCAAAATCTTGCAGTAGAAC | |
| | CACCAACATTGCATTCTTATGTGCTTTCTTCTGCTAATTATCCCAAAGCTTAT | |
| | GGTTGGTCCCATAATTCAGCTAAAGTGAATTTTGCTAAGTTGTTTGTCTCAT | |
| | ACTTCAAATGGCAAAACCAAGTGAGCTGGCTTGCCCAAGTCTTGGCAACCA | |
| | ACTCTGATAACTGGAAAAGTGCATTTACTTCCCTTGGTTTATCGGTGAAAGC | |
| | CTTCAAATCTTTATGCGTTACAGTTAAGAATTCTTTACCTGAAGAGGCAATC | |
| | CCGGACTCAGTGGATAGATATAGCAGGCAGATTAGAATGCCTTATCATGAT | |
| | GGTTATCTTGCTGTCACTCCTGTGATATCTCACGTTGTTCAAAGCAAAATAC | |
| | AACAAGCTGCTATTGACAAAAGAGCTAGGTTTTCTAATGTTGAGTTTACCAG | |
| | ACCAGCTGCTGTTTCAATGCTGGCGGCAAGTCTAGGTGGTGTGATAAATGT | |
| | ACTTAATTATCCTCCATACATTCGATCAAAGTACCATGGACTATCGAATTCA | |
| | AGGGCTTTTAAGTTAAACAACGGACAGACTGTCTTCAACGTGGAAGCCCTC | |
| | CTGAAGCCTGAGCTTATAAAAGCTCTTGAAGGAATTATATTCTCTAACAATG | |
| | CATTGGCATTAAAACAGAGAAGGCAGCAGAAAGTAAAAAACATCAAAGAA | |
| | CTGAGAAATACCCTCCTTGAGTGGTTTTCCCCTGTTTTCGAATGGAGATTGG | |
| | ATGCAATTGAGAATGGTTATGATCTAGAACAATTGGAATCAGCGAGCGAAC | |
| | GGTTAGAGTACAAGATTCTGAGTTTGCCGGATAATGAACTACCTTCTTTGAC | |
| | TATACCCTTGTTTAGATTACTTAATGAGATGTTGGGAGGAGTTTCAATGACT | |
| | CAGAGGTATGCATTCCATCCAAAGTTGATGTCTCCATTAAAGGCTGCTCTCC | |
| | AATGGTTGCTCGTTAATTTAACTGATCAAAAGCATGTTTTAATTGAAGAAGA | |
| | TGATGAGCATTACAGATACCTTCACCTGTCAGGGATTAGGGTTTTTGATGCC | |
| | CAAGCATTGTCTAATCCTTACTGCTCTGGTATTCCTTCGCTTACGGCGGTTTG | |
| | GGGAATGATCCACAGTTATCAACGCAAACTAAACGAAGCTTTGGGCACGAA | |
| | CGTACGGTTCACAAGCTTTTCATGGTTCATCCGTAATTACTCTGCCGTTGCT | |
| | GGTAAAAAGCTACCAGAACTTAGCCTTCAGGGTGCACAACAGTCTAGATTG | |
| | AAAAGACCTGGGATAATAGACGGGAAGTATTGTGATTTGGTGTTTGACTTG | |
| | ATTATTCATATCGATGGCTATGAAGACGATTTGCAGGCTGTTGATTCAAAGC | |
| | CTGATATCCTCAAGGCTCATTTCCCAAGCAATTTCGCCGGCGGCGTGATGCA | |
| | TCAACCCGAGTTGAATTCTAATATTAATTGGTGTTGTCTTTACTCCAACGAA | |
| | AACCAGCTTTTTGAGAAGCTCCGTCGGCTTCCTCTTTCTGGATGTTGGGTTA | |
| | TGCCCACAGAACATAAGATTCAAGATCTTGATGAACTGTTGTTGCTGTTGAA | |
| | CTCGGATAGTAAACTAAGTCCAAGCATGATGGGATACATGCTTCTCACAGA | |
| | GCCAATGGCTCGTGTAGGATCGTTAGAGAGGCTTCACTGTTATGCAGAACC | |
| | TGCTATTGGTGTTGTCAAATACGAGGCAGCAACTTCTGTTCGATTAAAAGGT | |
| | ATCGGAAACTATTTCAATTCCGCGTTTTGGATGCTCGATGCTCAAGAAAAGT | |
| | TCATGCTCATGAAGAAAGTGTGA | |
| VchTniQ | ATGTTTCTTCAAAGACCTAAACCGTATAGCGACGAAAGTCTGGAATCTTTCT | 30 |
| nucleotide | TCATCCGTGTTGCCAACAAGAATGGTTATGGAGATGTTCACAGGTTTTTGGA | |
| sequence | AGCCACAAAGCGATTCCTTCAAGACATTGATCACAATGGATACCAAACATT | |
| | TCCCACTGATATAACGCGTATCAATCCTTATTCAGCCAAAAACTCTTCATCA | |
| | GCAAGAACAGCGAGTTTCTTGAAGCTAGCACAGCTCACCTTCAACGAACCA | |
| | CCAGAATTGCTAGGGCTTGCAATAAACAGAACCAACATGAAGTACTCTCCT | |
| | TCTACTAGTGCAGTGGTAAGAGGAGCAGAGGTTTTCCCAAGATCCCTCCTG | |
| | CGTACTCATTCCATTCCATGTTGCCCTCTCTGCTTGAGGGAAAATGGGTATG | |
| | CTAGTTACCTTTGGCATTTTCAAGGATACGAGTATTGTCACTCTCATAATGT | |
| | CCCTCTAATTACCACTTGTTCTTGTGGTAAAGAATTCGATTACAGAGTATCT | |
| | GGGTTAAAAGGAATTTGTTGTAAGTGCAAAGAGCCAATAACTTTGACATCG | |
| | AGAGAGAATGGCCATGAAGCAGCTTGTACTGTTAGTAATTGGCTTGCCGGT | |
| | CACGAGTCAAAACCATTGCCTAATTTACCCAAATCTTATAGATGGGGACTC | |
| | GTCCATTGGTGGATGGGAATCAAGGACTCTGAATTTGATCATTTCAGCTTTG | |
| | TGCAGTTTTTCTCAAACTGGCCTCGGTCGTTTCATAGTATCATAGAAGATGA | |
| | AGTTGAATTTAATCTTGAACATGCTGTTGTTTCTACATCTGAGCTAAGGCTC | |
| | AAAGATCTTCTTGGGAGACTGTTCTTTGGTAGCATCAGGCTTCCTGAGAGAA | |
| | ATCTGCAACATAACATTATTTTGGGTGAGCTGTTATGTTATCTAGAAAACCG | |
| | TTTGTGGCAAGATAAAGGCTTGATAGCCAATTTAAAGATGAATGCTCTTGA | |
| | GGCAACAGTGATGCTTAACTGCTCCTTGGATCAGATTGCTTCAATGGTGGAG | |
| | CAAAGAATTTTAAAGCCTAACCGAAAATCAAAGCCAAATTCACCGCTTGAT | |
| | GTGACGGATTATTTGTTTCACTTTGGTGACATTTTCTGCTTATGGTTAGCTGA | |
| | GTTTCAGTCTGATGAATTCAATCGAAGCTTTTATGTTTCTAGGTGGTGA | |
| PseTniQ | ATGGCTTTTCTTTTCTCACCAAAAGCAAGAGCGTTTTCCGATGAATCGCTTG | 31 |
| nucleotide | AAAGTTATTTACTTCGAGTTGTCTCAGAAAACTTCTTCGATTCATATGAAGG | |
| sequence | ACTATCTTTGGCCATCAGAGAAGAACTTCATGAGTTGGACTTCGAGGCTCAT | |
| | GGAGCATTTCCCGTAGATTTAAAGAGACTTAATGTTTACCATGCAAAACAC | |
| | AACTCACACTTTAGGATGAGAGCTCTTGGCCTATTAGAAACTCTTTTAGATC | |
| | TTCCTCGTTATGAGCTTCAAAAGCTAGCACTCCTGAAGTCAGACATTAAATT | |
| | CAATTCTTCCGTTGCCCTCTACAACAATGGTGTTGATATTCCTTTGAGATTCA | |
| | TTAGACATCATGCTGAGGAAGCTGTTGACTCCATTCCGGTTTGTTCTCAATG | |
| | CTTGGCTGAAGAAGCTTATATAAAGCAATCTTGGCATATCAAATGGGTTAA | |
| | TGCTTGCACCAAACATCAATGTGCTTTGCTTCACAATTGTCCTGAATGTTAT | |
| | GCACCGATCAATTACATTGAGAATGAATCAATCACTCATTGCTCTTGTGGTT | |
| | TTGAATTGAGCTGCGCCTCTACAAGTCCTGTGAACACACTCTCGATTGAACA | |
| | TTTGAACAAACTCCTGGACAAAGGAGAACGGAATGACTCAAATCCATTGTT | |
| | TAACAACATGACGTTGACAGAAAGGTTTGCTGCTTTATTATGGTATCAAGA | |
| | GAGGTATTCACAGACTGATAATTTCTGTTTGAATGATGCTGTGAACTACTTC | |
| | AGTAAGTGGCCAGCAGTGTTTAATACTGAGCTAGATGAGCTCTCAAAGAAT | |
| | GCCGAGATGAAGCTGATTGATCTCTTCAACAAGACAGAGTTTAAGTTTATCT | |
| | TTGGAGATGCAATTCTTGCTTGTCCAAGTACTCAGAAGCAGAGTGAATCTCA | |
| | TTTCATCTACCGGGCATTACTTGATTACCTTGTCACTTTAGTGGAGAGCAAT | |
| | CCCAAAACAAAGAAGCCAAACGCAGCTGATTTGCTTGTTTCTGTCCTGGAA | |
| | GCAGCAACTTTGTTGGGGACGAGTGTAGAACAAGTTTATAGGCTCTATCAG | |
| | AATGGAATACTGCAAACCGCCTTTCGGCACAAGATGAATCAGAGAATAAAC | |
| | CCTTACAAGGGTGCTTTCTTTCTCCGTCATGTGATAGAGTATAAAACCAGCT | |
| | TTGGTAACGATAAAGCTAGAATGTATCTCTCAGCTTGGTG | |
| VchTnsAB | ATGGCGACATCCCTTCCAACGCCATCAGCTATAACTACTTCGGCACTAGAGT | 32 |
| nucleotide | ATGCTTTCCATACTCCGGCTCGCAATTTGACGAAGTCTAGGGGGAAGAACA | |
| sequence | TTCATCGATATGTTAGTGTCAAGATGTCTAAAAGAATTACTGTAGAATCAAC | |
| | ACTAGAATGCGATGCTTGCTATCATTTTGACTTCGAGCCTTCTATCGTGAGA | |
| | TTTTGTGCTCAGCCTATTCGCTTTCTGTATTATTTAAATGGGCAGTCACACTC | |
| | CTATGTCCCAGATTTTCTTGTGCAATTCGACACAAACGAGTTTGTCTTGTAT | |
| | GAAGTTAAAAGCGCTTATGCGAAAAACAAACCTGATTTCGATGTTGAGTGG | |
| | GAAGCAAAGGTGAAGGCCGCAACAGAGCTGGGTTTGGAACTTGAACTTGTT | |
| | GAGGAGAGCGATATAAGAGATACCGTTGTTTTGAACAATCTTAAGAGGATG | |
| | CATAGATATGCGAGTAAGGACGAGCTTAATAACGTACACAATTCCCTACTT | |
| | AAGATCATAAAGTATAACGGAGCTCAATCCGCCCGATGTTTGGGTGAACAG | |
| | CTAGGGTTGAAAGGTCGTACGGTACTTCCTATTCTGTGTGATCTTCTTTCAA | |
| | GATGCTTGCTCGACACCAGACTTGATAAGCCCCTCTCTCTGGAAAGTCGTTT | |
| | TGAACTCGCCTCATATGGAGGATCAGGATCTGGCAAAAGAACCGCAGATGG | |
| | TTCTGAGTTCGAAAGCCCAAAGAAAAAGAGAAAGGTGGGTTCCGGTTCTGG | |
| | AGGAGCCAAGAAGGGTTTTTCTAGCTTTCACAGGAAGGCAGTTTCCTCACA | |
| | GGACACTTTAGAGAGTATAGAACTAGTTTCCAGTGCGAATTGCCTCGAATC | |
| | AGTCACTTATCAAGATATATCTGCATTTCCTGAAACCATTGCAGTGGAGATC | |
| | AACTTCAGATTGTCTATTCTCCGGTTTCTCGCAAGAAAATGTGAGACTATCG | |
| | TTGCTAAATCTATTGAGCCCCATAGAGTTGAGCTTCAACAGAATTATTCTCG | |
| | GAAGATTCCATCTGCAATAACAATATACAGATGGTGGTTGGCCTTTCGGAA | |
| | GTCAGATTACAACCCGATTAGTCTGGCTCCTAATATCAAAGACAGAGGGAA | |
| | CAGAGAAACTAAAGTATCTACTGTGGTTGACTCCATCATGGAACAAGCCGT | |
| | TGAAAGGGTTATCTCTGGTCGTAAAGTCAATGTTTCTAGCGCGTACAAACG | |
| | AGTCCGGAGGAAAGTTAGACAATATAATCTTACTCATGGAACAAAGTACAC | |
| | ATATCCAAAATACGAGAGCGTACGAAAGCGCGTAAAGAAGAAAACACCGT | |
| | TTGAGTTATTAGCTGCTGGTAAGGGAGAGAGGGTCGCCAAGCGTGAATTTA | |
| | GGAGAATGGGAAAGAAGATTCTGACGTCAAGTGTTCTCGAGAGGGTGGAG | |
| | ATAGATCACACTGTTGTGGATCTGTTCGCTGTTCATGAGGAATATAGGATTC | |
| | CTCTCGGGAGGCCATGGCTGACTCAGTTAGTTGATTGTTACAGCAAAGCAG | |
| | TCATTGGTTTCTACCTTGGCTTTGAACCTCCTAGTTATGTCTCCGTATCACTA | |
| | GCTTTAAAGAATGCCATTCAACGCAAAGATGATTTGATTTCCTCTTACGAGT | |
| | CAATAGAAAATGAATGGTTATGTTATGGTATACCAGATCTCCTTGTTACAGA | |
| | CAATGGAAAAGAATTTTTGTCCAAAGCTTTTGATCAAGCTTGTGAGAGCTTA | |
| | TTAATTAACGTGCATCAGAATAAGGTAGAAACCCCAGATAACAAGCCTCAT | |
| | GTTGAAAGAAACTACGGGACAATAAATACTTCCTTATTGGACGATTTACCT | |
| | GGTAAAAGTTTCTCTCAGTACTTGCAGAGGGAAGGATATGACTCTGTGGGA | |
| | GAAGCTACATTGACTCTCAATGAGATCAGAGAAATCTACTTGATTTGGCTTG | |
| | TCGACATATATCACAAGAAACCGAATCAAAGAGGAACTAACTGTCCAAATG | |
| | TTGCTTGGAAAAAAGGTTGCCAAGAATGGGAGCCAGAAGAATTCTCCGGGT | |
| | CAAAAGATGAGCTTGATTTTAAATTCGCAATTGTTGATTATAAACAACTTAC | |
| | AAAAGTAGGAATCACTGTTTACAAGGAACTAAGCTACTCAAACGATCGTTT | |
| | GGCAGAGTATAGAGGCAAAAAAGGAAACCACAAAGTTCAGTTCAAGTACA | |
| | ATCCGGAATGTATGGCTGTGATATGGGTTCTTGATGAAGATATGAACGAAT | |
| | ATTTCACAGTTAATGCTATCGACTATGAATACGCATCTAGGGTGTCACTGTG | |
| | GCAACATAAATACAACATGAAGTATCAAGCAGAGCTAAATTCTGCGGAATA | |
| | TGACGAAGATAAAGAGATTGATGCAGAAATTAAAATCGAGGAAATTGCTG | |
| | ATAGATCTATAGTGAAAACCAATAAAATTAGAGCTCGTCGTCGTGGTGCTA | |
| | GGCATCAAGAGAATAGTGCACGAGCTAAATCAATCTCTAATGCAAATCCTG | |
| | CAAGTATTCAGAAGCATGAAGACGAAATAGTCAGTGCTGATAATGATGATT | |
| | GGGATATCGATTACGTGTGA | |
| PseTnsAB | ATGTACATTAGGAACTTGAGAAAGCCTTCTCCAAATAAGAATGTTTTTAAGT | 33 |
| nucleotide | TCGCATCTACCAAGGTCAGCTCTGTGGTCATGTGTGAATCCAGCCTTGAGTT | |
| sequence | TGACGCTTGCTTTCATCATGAATATAACGACTTGATTGAGAGTTTTGGTTCT | |
| | CAGCCAGAAGGTTTCAAATACGAATTCATGGGTAAATCTCTCCCTTACACTC | |
| | CAGATGCTTTGATCTCCTACACGGACAAAACTCAAAAGTATCACGAGTATA | |
| | AACCATATTCAAAAATCGCATCACCTCTATTTAGAGCAGAGTTTGCTGCAAA | |
| | GAGGGCTGCAAGTCTTAAGTTGGGTATTGATTTGGTACTCGTCACAGACAG | |
| | ACAGATTAGAGTAAACCCAATACTCAACAATCTGAAGCTCCTTCATAGATA | |
| | CTCGGGCGTTTATGGTATATCTGGTATCCAGAAGGAACTACTTAGCTTTATT | |
| | CACAAAAGTGGCGTTATCAAACTCAATGATATCTCATCACAGGTTGGGATC | |
| | CCCATTGGAGAAACTCGGTCATTTCTTTTTGGACTGATGCATAAGGGACTTG | |
| | TAAAGGCTGACCTAGGTTGTGATGACCTGACAAACAACCCTACTCTTTGGG | |
| | CTACTCCTGGAAGTGGCTCAGGCAAACGTACTGCTGACGGAAGTGAGTTCG | |
| | AGTCCCCAAAGAAAAAAAGAAAAGTTGGTAGTGGATCTGGTGGAATGACT | |
| | GATTTCTTCAATGAGTTTGATGAATCGCTTGTACCACTGAAACCACAAACTC | |
| | CAACGCAGTATGTTAAGCTGGACGATGCTAATTTAATTCAGAGAGATTTAG | |
| | ATACATTCTCAGATACTTTCAAGAACCAAGCCCTCCAAAGATATAAGCTAA | |
| | TTTCTACAATTGATAAAAAGTTAAGCCGCGGATGGACTCAAAGAAATCTCG | |
| | ACCCGATCCTCGACGAACTCTTCAAAGGCGGTGATGTAGTGAGGCCAAACT | |
| | GGAGAACTGTGGCAAGATGGAGGAAGAAATATATAGAAAGCAATGGTGAC | |
| | ATCGCTTCTCTTGCTGATAAAAACCACAAAATGGGAAACAGAACAAATCGG | |
| | ATAAAAGGAGATGACAAATTTTTTGACAAGGCCTTGGAACGTTTCCTTGAC | |
| | GCGAAAAGACCTACAATAGCAACAGCTTATCAATATTACAAGGACTTGATA | |
| | GTGATTGAAAACGAGTCAATTGTTGAAGGGAAGATCCCCATCATCTCTTAT | |
| | AATGCATTTAACAAACGAATTAAAGCCATTCCTCCTTATGCTGTTGCAGTCG | |
| | CTAGGCATGGGAAATTCAAAGCTGATCAATGGTTTGCATATTGTGCAGCTC | |
| | ACGTTCCACCAACGCGTATACTTGAACGAGTGGAGATTGATCATACTCCGTT | |
| | GGATCTAATATTATTGGATGATGAGCTTCTGATTCCCATTGGCCGGCCATAT | |
| | CTTACACTTCTCATAGATGTGTTTTCAGGTTGCGTTCTGGGATTCCATTTGTC | |
| | ATACAAGAGCCCTTCTTATGTCTCTGCAGCAAAAGCAATCACTCACGCAAT | |
| | AAAACCCAAAAGCTTAGATGCGCTTAATATTGAGCTTCAGAATGACTGGCC | |
| | CTGTTTCGGGAAGTTTGAGAATTTAGTAGTTGACAATGGAGCTGAATTTTGG | |
| | TCAAAAAATTTGGAACATGCTTGTCAATCTGCTGGTATAAATATTCAATACA | |
| | ATCCGGTGCGTAAGCCATGGCTCAAGCCTTTTATAGAGAGGTTCTTTGGGGT | |
| | GATGAATGAATACTTCTTGCCGGAACTCCCAGGAAAAACATTTTCTAACATT | |
| | CTTGAGAAAGAAGAATACAAACCGGAGAAGGATGCAATAATGAGGTTCAG | |
| | CACCTTTGTTGAGGAGTTCCACCGTTGGATTGCCGACGTGTATCATCAAGAT | |
| | AGTAATAGCCGTGAAACGCGTATTCCTATTAAGCGATGGCAACAAGGATTC | |
| | GATGCTTATCCTCCTTTAACAATGAACGAAGAAGAGGAGACACGGTTTTCT | |
| | ATGCTCATGAGAATCTCTGACTCTAGGACGCTAACCAGGAATGGGTTTAAG | |
| | TACCAAGAATTGATGTATGATTCTACCGCCTTAGCAGATTACCGTAAGCATT | |
| | ACCCTCAGACAAAAGAGACTGTGAAGAAGCTAATAAAGGTCGATCCAGAT | |
| | GATATCTCCAAGATTTACGTTTATCTGGAAGAGTTGGAAAGTTACTTGGAGG | |
| | TTCCTTGCACAGATCCAACTGGATACACTGATGGTTTATCCATATATGAGCA | |
| | TAAAACCATTAAGAAGATCAACCGAGAAGTTATTAGAGAATCAAAAGATA | |
| | GCCTTGGGTTGGCGAAAGCGAGAATGGCTATTCATGAGAGAGTGAAACAAG | |
| | AGCAAGAAGTATTCATCGAAAGTAAGACCAAAGCTAAGATTACTGCAGTCA | |
| | AGAAGCAGGCTCAGATCGCGGATGTTAGTAATACAGGCACATCAACAATCA | |
| | AGGTGTCCGAGGAGTCTGCGGCTCCTGTCCAGAAACACATCTCAAATGATA | |
| | ACAGTGATGATTGGGATGATGATCTGGAAGCTTTTGAATGA | |
| VchTnsC | ATGAGTGAAACACGAGAGGCTAGGATAAGCCGGGCCAAACGGGCTTTTGTT | 34 |
| nucleotide | TCTACACCGTCTGTACGCAAGATCCTGAGCTACATGGATAGATGTAGGGAC | |
| sequence | TTATCAGATCTCGAATCAGAGCCAACTTGTATGATGGTTTATGGTGCCTCTG | |
| | GTGTGGGAAAAACCACGGTCATCAAGAAGTATTTGAATCAAAACAGAAGA | |
| | GAATCTGAGGCGGGTGGTGATATTATCCCAGTGTTACACATTGAGCTCCCTG | |
| | ATAATGCAAAGCCTGTTGATGCCGCACGGGAGCTGTTGGTTGAGATGGGGG | |
| | ATCCACTGGCCCTCTATGAAACTGATTTAGCAAGACTTACTAAGAGATTGAC | |
| | TGAGCTGATCCCTGCTGTTGGGGTGAAGCTTATAATCATTGATGAATTTCAG | |
| | CATCTTGTGGAAGAGAGATCCAATAGAGTGTTAACTCAAGTTGGAAACTGG | |
| | CTTAAGATGATCTTAAACAAAACCAAATGCCCTATTGTAATATTTGGAATGC | |
| | CTTACTCAAAGGTTGTTCTTCAAGCCAATTCGCAGCTTCACGGAAGGTTTTC | |
| | AATACAAGTCGAACTACGTCCCTTCTCTTACCAAGGAGGAAGAGGAGTTTT | |
| | CAAGACATTCTTGGAGTATCTTGATAAAGCTTTACCTTTCGAGAAGCAAGCA | |
| | GGCTTAGCAAATGAAAGTCTTCAGAAAAAACTGTATGCTTTCTCTCAGGGC | |
| | AACATGAGATCCTTAAGGAATCTCATATATCAGGCAAGTATTGAAGCTATT | |
| | GACAACCAACATGAAACCATTACAGAGGAGGATTTTGTGTTTGCTTCAAAA | |
| | TTGACAAGCGGAGACAAACCAAATTCATGGAAAAATCCTTTTGAAGAAGGT | |
| | GTTGAAGTGACAGAAGACATGCTTCGTCCACCACCCAAAGATATTGGATGG | |
| | GAAGATTACCTAAGGCATTCTACTCCCCGTGTATCCAAGCCGGGCAGAAAC | |
| | AAGAATTTCTTCGAATGA | |
| PseTnsC | ATGGGAAATGCTCTTACAGAGATTCAAATAGAGAAGTTACGGAATTTCAGT | 35 |
| nucleotide | GATTGCATCGTGATGCATCCTCAGATAAAGACCATTTTCAATGATTTTGATG | |
| sequence | AACTAAGGCTTAATAGAAAGTTTCAGAGTGATCAGCAATGTATGCTTCTTAT | |
| | AGGAGATACTGGTGTTGGCAAATCACACACCATCAACCATTACAAGAAAAG | |
| | AGTCCTCGCAACACAAAACTATTCAAGAAACACAATGCCGGTCCTTGTTTCT | |
| | CGCATATCACGTGGGAAAGGTTTGGATGCTACTTTGGTGCAGATGCTGGCG | |
| | GATCTAGAGCTGTTTGGTTCTTCTCAGATCAAGAAGCGGGGCTACAAAACA | |
| | GACCTTACCAAAAAATTAGTTGAGAGTTTGATCAAAGCTCAAGTGGAGCTG | |
| | TTGATTATCAATGAATTCCAAGAGCTTATTGAGTTTAAAAGTGTTCAAGAAA | |
| | GACAACAGATTGCAAATGGTCTAAAGTTTATTAGTGAAGAAGCCAAGGTTC | |
| | CCATTGTTTTGGTTGGAATGCCATGGGCAGCAAAGATAGCTGAGGAACCAC | |
| | AATGGGCTTCTCGACTCGTAAGAAAACGAAAGCTAGAATATTTTTCACTCA | |
| | AGAATGACAGCAAGTACTTTAGGCAGTATTTGATGGGTCTTGCCAAAAAGA | |
| | TGCCTTTCGACGTTCCTCCTAAATTAGAATCGAAGAACACTACGATTGCGCT | |
| | ATTTGCAGCTTGTAGAGGGGAAAACAGAGCTTTGAAGCACCTTCTTTTAGA | |
| | AGCACTGAAGCTCGCCCTTTCATGCAATGAGTATCTTGAGAACAAGCATTTC | |
| | ATCACAGCTTATGACAAATTCGATTTCTTCAACGACAAGGAGAAACTGAAA | |
| | TCCAAAAACCCTTTCAAGCAAGATATAAAAGATATTGAAATTTATGAAGTT | |
| | ATTAAAAATTCTTCCTACAATCCAAATGCACTTGATCCAGAAGATATGTTGA | |
| | CTGATCGAGTATTTGCTATTGTGAAA | |
| ClpP | ATGAGTTATTCTGGAGAACGGGATAATTTTGCACCTCACATGGCATTAGTTC | 36 |
| nucleotide | CAATGGTGATTGAACAAACAAGTAGAGGGGAGCGAAGCTTTGACATTTACT | |
| sequence | CTCGTCTATTAAAAGAAAGGGTCATCTTCTTAACCGGGCAAGTTGAAGATC | |
| | ATATGGCAAATCTGATTGTAGCTCAGATGCTTTTTCTTGAAGCAGAGAATCC | |
| | TGAGAAAGATATCTATCTCTACATTAACTCTCCTGGTGGTGTAATAACTGCT | |
| | GGAATGTCTATATATGATACAATGCAGTTTATCAAACCTGATGTTTCAACAA | |
| | TATGTATGGGTCAGGCTGCGTCAATGGGAGCTTTTCTTTTGACGGCTGGCGC | |
| | AAAAGGAAAGCGGTTCTGCCTACCGAACTCAAGGGTGATGATCCATCAACC | |
| | ACTTGGAGGATACCAAGGTCAAGCTACCGATATAGAGATTCATGCTAGAGA | |
| | AATTTTGAAGGTGAAGGGTAGAATGAATGAACTGATGGCCCTTCACACTGG | |
| | ACAATCGCTCGAGCAGATTGAGCGTGACACAGAGAGAGACAGGTTTTTATC | |
| | CGCACCAGAAGCTGTTGAGTATGGTTTGGTTGATTCTATTCTTACTCATAGA | |
| | AACTGA | |
| ClpX | ATGACTGATAAACGGAAAGATGGTTCCGGCAAGCTTCTTTACTGCAGCTTTT | 37 |
| nucleotide | GCGGGAAATCTCAACATGAAGTTAGGAAACTTATTGCAGGGCCTTCTGTTT | |
| sequence | ATATCTGTGACGAATGTGTTGATCTCTGCAATGATATTATTCGAGAAGAGAT | |
| | CAAAGAAGTGGCTCCTCACAGAGAGAGGAGTGCGTTGCCGACACCACACG | |
| | AGATTAGAAATCATCTTGATGATTATGTCATTGGTCAAGAACAAGCAAAGA | |
| | AGGTCCTTGCTGTTGCAGTGTACAACCATTACAAGAGGCTCAGAAACGGAG | |
| | ATACTTCTAATGGTGTGGAACTTGGCAAATCAAACATCCTGCTCATTGGACC | |
| | CACTGGAAGTGGAAAGACACTCCTAGCAGAGACATTGGCAAGGCTCTTAGA | |
| | CGTTCCTTTCACTATGGCTGATGCAACTACTCTTACTGAGGCTGGTTATGTT | |
| | GGAGAAGATGTGGAGAATATTATTCAAAAGTTATTGCAGAAATGTGATTAC | |
| | GATGTCCAGAAAGCTCAGAGAGGGATAGTCTACATAGATGAGATAGACAA | |
| | GATCTCTAGGAAGAGTGATAATCCCTCGATAACACGCGACGTTTCTGGCGA | |
| | AGGTGTTCAACAAGCTTTGTTGAAGCTCATTGAAGGCACCGTAGCGGCAGT | |
| | TCCACCACAAGGAGGAAGAAAGCATCCTCAGCAAGAGTTTCTCCAAGTGGA | |
| | TACCTCAAAAATCTTATTTATATGTGGTGGTGCCTTCGCTGGGCTTGACAAA | |
| | GTTATCTCACACCGTGTAGAAACTGGTAGCGGGATTGGGTTTGGTGCAACG | |
| | GTGAAGGCAAAATCTGATAAGGCTTCTGAAGGAGAGCTTCTGGCTCAAGTT | |
| | GAACCAGAGGATCTTATCAAGTTTGGACTAATTCCTGAGTTCATCGGTAGAC | |
| | TTCCTGTCGTTGCTACGTTGAACGAGTTAAGTGAGGAAGCCCTGATTCAAAT | |
| | CTTGAAAGAGCCAAAGAATGCTCTGACAAAGCAATATCAGGCTCTTTTCAA | |
| | TCTTGAGGGAGTTGACTTGGAATTTCGTGATGAAGCGCTGGATGCTATAGCT | |
| | AAGAAAGCAATGGCGAGGAAAACCGGAGCTCGTGGTCTAAGATCGATTGTT | |
| | GAAGCCGCTTTATTGGATACAATGTATGATTTGCCCTCCATGGAAGATGTTG | |
| | AGAAAGTGGTCATCGACGAGTCTGTGATTGATGGACAGTCAAAGCCATTGC | |
| | TCATATATGGTAAACCTGAAGCACAGCAGGCATCAGGAGAATGA | |

TABLE 10
DONOR CONSTRUCT SEQUENCES

| | | SEQ |
|---|---|---|
| | | ID |
| Description | Sequence | NO: |
| Long | tagcagaaggcatgttgttgtgactccgag | 1 |
| intergenic | gggttgcctcaaactctatcttataaccgg | |
| region | cgtggaggcatggaggcaggggtattttgg | |
| (LIR) | tcattttaatagatagtggaaaatgacgtg | |
| | gaatttacttaaagacgaagtctttgcgac | |
| | aagggggggcccacgccgaatttgttaaag | |
| | gtgttcacactataaaagcatatacgatgt | |
| | gatggtatttgatggagcgtatattgtatc | |
| | aggtatttccgttggatacgaataatatta | |
| | ccggcgtggcccccccttatcgcgagtgct | |
| | ttagcacgagcggtccagatttaaagtaga | |
| | aaatttcccgcccactaggtattcgtacga | |
| | ccctc | |
| Short | aataaaatgattattttatgaatatatttc | 2 |
| intergenic | attgtgcaagtagatagaaattacatatgt | |
| region | tacataacacacgaaataaacaaaaaaaga | |
| (SIR) | caatccaaaaacaaacaccccaaaaaaaat | |
| | aatcactttagataaactcgtatgaggaga | |
| | ggcacgt | |
| RepA | tcagtgactcgacgattcccgagcaaaaaa | 3 |
| coding | agtctccccgtcacacatgtagtgggtgac | |
| sequence | gcaattatctttaaagtaatccttctgttg | |
| | acttgtcattgataacatccagtcttcgtc | |
| | aggattgcaaagaattatagaagggatccc | |
| | accttttattttcttcttttttccatattt | |
| | agggttgacagtgaaatcagactggcaacc | |
| | tattaattgcttccacaatgggacgaactt | |
| | gaaggggatgtcgtcgatgatattataggt | |
| | ggcgtgttcatcgtagttggtgaaatcgat | |
| | ggtaccgttccaatagttgtgtcgtccgag | |
| | acttctagcccaggtggtctttccggtacg | |
| | agttggtccgcagatgtagaggctggggtg | |
| | tcggattccattccttccattgtccttgtt | |
| | aaatcggccatccattcaaggtcagattga | |
| | gcttgttggtatgagacaggatgtatgtaa | |
| | gtataagcgtctatgcttacatggtataga | |
| | tgggtttccctccaggagtgtagatcttcg | |
| | tggcagcgaagatctgattctgtgaagggc | |
| | gacacatacggttcaggttgtggagggaat | |
| | aatttgttggctgaatattccagccattga | |
| | agctttgttgcccattcatgagggaattct | |
| | tccttgatcatgtcaagatattcctcctta | |
| | gacgttgcagtctggataatagttctccat | |
| | cgtgcgtcagatttgcgaggagaaacctta | |
| | tgatctcggaaatctcctctggttttaata | |
| | tctccgtcctttgatatgtaatcaaggact | |
| | tgtttagagtttctagctggctggatatta | |
| | gggtgatttccttcaaaatcgaaaaaagaa | |
| | ggatccctaatacaaggttttttatcaagc | |
| | tggagGagagcatgatagtgggtagtgcca | |
| | tcttgatgaagctcagaagcaacaccaagg | |
| | aagaaaataagaaaaggtgtgagtttctcc | |
| | cagagaaactggaataaatcatctctttga | |
| | gatgagcacttgggataggtaaggaaaaca | |
| | tatttagattggagtctgaagttcttacta | |
| | gcagaaggcat | |
| 2^{nd} | CCGTATAGGCAGCTGAAGATAAAAACAATC | 4 |
| trans- | GATTCGATGTTTAAGTTAATGTAATTCTGC | |
| posonend | CGAAAAGGCAGTGAGTAGTAATTGAAAAAA | |
| sequence | CCAGTCTTATATCCGGGCTGGTTTTTTTGT | |
| (LE) for | TTTTAGCGTTATAACTTTTATTTCACTATG | |
| Pse | TCACTGTATGGTTGCATTTTATGTCAGTGA | |
| | ATGGTTGCAGGTTTTTTGTAAGTTATTGAT | |
| | ATGCCTATCCAGTGTATGTCACGCTTTGCT | |
| | TGCAGCGACA | |
| 2^{nd} | GTGTCCGTGACTGATGCAAGGTTATCGGAA | 5 |
| trans- | CTGTAATGATTATATGTTTTCTAATTTTGA | |
| posonend | AGTGGTTCTTTTGATGAGTTTTGCCAGTTA | |
| sequence | CTGAAAGTAACTTTCTTACTGCAGTAGTTT | |
| (LE) for | TGCTGAAATACTCGATTCACAAAAATATCA | |
| Vch | ACTTATGGTTGTTTTGTGAGATATCAATAT | |
| | ATGGTTGTTTTGTGGTTAAGTTGCTGATTA | |
| | TAAATAATTATTAAATATCACTTTATGGTT | |
| | GCATCAACA | |
| 1^{st} | TGTCGCTGCAACCATAGTTTGACATAATGC | 6 |
| trans- | AACCATACCTTGACATAATTAAAGTCATAC | |
| posonend | TCTGACATAAAGCCCCTTATTAAAAATGAT | |
| sequence | GCTAAAAATAAATTCCATTATAATCAATTA | |
| (RE) for | TTTAAGAATTAATTAAACCATAGTGTGACA | |
| Pse | TAGTGGCTTTTTATTTGTATAGTATGTGTA | |
| | CTCAAATAATAAAAATTTACTTATGTATAT | |
| | ACGCAACTTAAGAAAGCCATCTCCAAATAA | |
| | AAATGTATTT | |
| 1^{st} | TGTTGATACAACCATAAAATGATAATTACA | 7 |
| trans- | CCCATAAATTGATAATTATCACACCCATAA | |
| posonend | ATTGATATTGCCTCTTCATGGTCTAAACTT | |
| sequence | CAGTAAGTTTACGACATTTTCATGAAGAGG | |
| (RE) for | TCATTTCATGACAAGCTTACCTACGCCCTC | |
| Pse | AGCAATTACGACTTCGGCGTTAGAGTATGC | |
| | ATTCCATACTCCCGCTCGCAATCTAACGAA | |
| | ATCTCGCGGAAAAAATATTCATCGTTATGT | |
| | CAGTGTAAAG | |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system for integration of a nucleic acid sequence into a double-stranded target sequence of a genome of a plant cell or a target plasmid in a plant cell, comprising:
i) an RNA-guided DNA binding complex or one or more first helper polynucleotides each comprising a sequence encoding a component of the RNA-guided DNA binding complex, wherein the RNA-guided DNA binding complex comprises one or more Cas proteins, a transposase, a crRNA, or any combination thereof, optionally the system comprises one or more helper accessory proteins or one or more helper accessory polynucleotides each comprising a sequence encoding at least one of the one or more helper accessory proteins, wherein the one or more helper accessory proteins comprise ClpX and ClpP;
ii) a transposition complex or one or more second helper polynucleotides each comprising a sequence encoding a component of the transposition complex, wherein the transposition complex comprises one or more transposases and one or more transposons; and
iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

2. A nucleic acid composition for integration of a nucleic acid sequence into a double-stranded target sequence of a genome of a plant cell or a target plasmid in a plant cell, comprising:
i) one or more first helper polynucleotides each comprising a sequence encoding a component of an RNA-guided DNA binding complex, wherein the RNA-guided DNA binding complex comprises one or more Cas proteins, a transposase, a crRNA, or any combination thereof, optionally the nucleic acid composition comprises one or more helper accessory polynucleotides each comprising a sequence encoding at least one of the one or more helper accessory proteins, wherein the one or more helper accessory proteins comprise ClpX and ClpP;
ii) one or more second helper polynucleotides each comprising a sequence encoding a component of a transposition complex, wherein the transposition complex comprises one or more transposases and one or more transposons; and
iii) a donor polynucleotide comprising a cargo sequence flanked by a first transposon end sequence (RE) on the 5′ end of the cargo sequence and a second transposon end sequence (LE) on the 3′ end of the cargo sequence, wherein the donor polynucleotide is comprised within a first autonomous replicon.

3. (canceled)

4. The system of claim 1, wherein the first autonomous replicon is derived from a geminivirus.

5. (canceled)

6. The system of claim 1, wherein the donor polynucleotide comprised within the first autonomous replicon comprises, from 5′ to 3′: a first long intergenic region (LIR), the RE, the cargo sequence, the LE, a short intergenic region (SIR), a sequence encoding RepA, and a second LIR.

7. (canceled)

8. The system of claim 6, wherein:
the first and/or second LIR comprise the sequence of SEQ ID NO: 1 or a sequence having one, two, or three mismatches relative to the sequence of SEQ ID NO: 1;
the SIR comprises the sequence of SEQ ID NO: 2 or a sequence having one, two, or three mismatches relative to the sequence of SEQ ID NO: 2; and
the sequence encoding RepA comprises the sequence of SEQ ID NO: 3 or a sequence that is at least 80%, 85%, 90%, 95%, 98%, 99%, or 100% identical to SEQ ID NO: 3.

9. (canceled)

10. (canceled)

11. The system of claim 1, wherein the LE comprises the sequence of any one of SEQ ID NOs: 4-5 or a sequence having one, two or three mismatches relative to the sequence of any one of SEQ ID NOs: 4-5 and wherein the RE comprises the sequence of any one of SEQ ID NOs: 6-7 or a sequence having one, two or three mismatches relative to the sequence of any one of SEQ ID NOs: 6-7.

12. The system of claim 1, wherein the cargo sequence is 0.2 to 1000 kb in length, optionally 200 to 1200 bp in length.

13. (canceled)

14. (canceled)

15. (canceled)

16. The system of claim 1, wherein the one or more Cas proteins comprise a Cas6 protein, a Cas7 protein, and a Cas8 protein.

17. The system of claim 16, wherein:
the Cas6 protein comprises an amino acid sequence that is at least 80%, 85%, 90%, 95%, 98%, 99%, or 100% identical to any one of SEQ ID NOs: 8-9;
the Cas7 protein comprises an amino acid sequence that is at least 80%, 85%, 90%, 95%, 98%, 99%, or 100% identical to any one of SEQ ID NOs: 10-11; and/or
the Cas8 protein comprises an amino acid sequence that is at least 80%, 85%, 90%, 95%, 98%, 99%, or 100% identical to any one of SEQ ID NOs: 12-13.

18. The system of claim 1, wherein the transposase of the RNA-guided DNA binding complex comprises a TniQ protein, optionally the TniQ protein comprises an amino acid sequence that is at least 80%, 85%, 90%, 95%, 98%, 99%, or 100% identical to any one of SEQ ID NOs: 14-15.

19. The system of claim 1, wherein the one or more transposases of the transposition complex comprise: a TnsA transposase, a TnsB transposase, and a TnsC protein; or a TnsAB fusion protein and a TnsC protein.

20. (canceled)

21. The system of claim 19, wherein:
the TnsAB fusion protein comprises an amino acid sequence that is at least 80%, 85%, 90%, 95%, 98%, 99%, or 100% identical to any one of SEQ ID NOs: 16-17; and/or
the TnsC protein comprises an amino acid sequence that is at least 80%, 85%, 90%, 95%, 98%, 99%, or 100% identical to any one of SEQ ID NOs: 18-19.

22. (canceled)

23. (canceled)

24. The system of claim 1, wherein:
the ClpX comprises an amino acid sequence that is at least 80%, 85%, 90%, 95%, 98%, 99%, or 100% identical to SEQ ID NO: 21; and/or
the ClpP comprises an amino acid sequence that is at least 80%, 85%, 90%, 95%, 98%, 99%, or 100% identical to SEQ ID NO: 20.

25.-28. (canceled)

29. The system of claim 1, wherein the cargo sequence is capable of being integrated at an integration site following binding of the RNA-guided DNA binding complex to a search target sequence on a first strand of the double stranded target sequence, wherein the integration site is about 48 to 52 base pairs downstream of the double stranded target sequence.

30.-38. (canceled)

39. The system of claim 1, comprising:
at least three first helper polynucleotides each comprising a sequence encoding a Cas protein, wherein the sequence encoding the Cas protein is operably linked to a pCmY promoter and a tHSP terminator;
a first helper polynucleotide comprising a sequence encoding a transposase protein, wherein the sequence encoding the transposase protein is operably linked to a pCmY promoter and a tHSP terminator;
a first helper polynucleotide comprising a sequence encoding a crRNA, wherein the sequence encoding the crRNA is operably linked to a pU6 promoter and a tU6 terminator;
at least two second helper polynucleotides each comprising a sequence encoding a transposase, wherein the sequence encoding a transposase is operably linked to a pCmY promoter and a tHSP terminator; and
at least two helper accessory polynucleotides, wherein the sequence encoding at least one of the one or more helper accessory proteins is operably linked to a pCmY promoter and a tHSP terminator.

40. (canceled)

41. The system of claim 1, wherein the sequence encoding the component of the RNA-guided DNA binding complex, the sequence encoding the component of the transposition complex, the sequence encoding at least one of the one or more helper accessory proteins, or any combination thereof, is codon optimized for expression in the plant cell.

42. The system of claim 1, wherein:
the sequence encoding the component of the RNA-guided DNA binding complex encodes a Cas6 protein, optionally the sequence encoding the Cas6 protein comprises the nucleotide sequence of any one of SEQ ID NOs: 24-25 or a sequence that is at least 80%, 85%, 90%, 95%, 98%, 99%, or 100% identical to any one of SEQ ID NOs: 24-25;
the sequence encoding the component of the RNA-guided DNA binding complex encodes a Cas7 protein, optionally the sequence encoding the Cas7 protein comprises the nucleotide sequence of any one of SEQ ID NOs: 26-27 or a sequence that is at least 80%, 85%, 90%, 95%, 98%, 99%, or 100% identical to any one of SEQ ID NOs: 26-27;
the sequence encoding the component of the RNA-guided DNA binding complex encodes a Cas8 protein, optionally the sequence encoding the Cas8 protein comprises the nucleotide sequence of any one of SEQ ID NOs: 28-29 or a sequence that is at least 80%, 85%, 90%, 95%, 98%, 99%, or 100% identical to any one of SEQ ID NOs: 28-29;
the sequence encoding the component of the RNA-guided DNA binding complex encodes a TniQ protein, optionally the sequence encoding the TniQ protein comprises the nucleotide sequence of any one of SEQ ID NOs: 30-31 or a sequence that is at least 80%, 85%, 90%, 95%, 98%, 99%, or 100% identical to any one of SEQ ID NOs: 30-31;
the sequence encoding the component of the transposition complex encodes a TnsAB fusion protein, optionally the sequence encoding the TnsAB fusion protein comprises the nucleotide sequence of any one of SEQ ID NOs: 32-33 or a sequence that is at least 80%, 85%, 90%, 95%, 98%, 99%, or 100% identical to any one of SEQ ID NOs: 32-33;
the sequence encoding the component of the transposition complex encodes a TnsC protein, optionally the sequence encoding the TnsC protein comprises the nucleotide sequence of any one of SEQ ID NOs: 34-35 or a sequence that is at least 80%, 85%, 90%, 95%, 98%, 99%, or 100% identical to any one of SEQ ID NOs: 34-35; and/or
the sequence encoding ClpX comprises the nucleotide sequence of SEQ ID NO: 37 or a sequence that is at least 80%, 85%, 90%, 95%, 98%, 99%, or 100% identical to SEQ ID NO: 37 and the sequence encoding ClpP comprises the nucleotide sequence of SEQ ID NO: 36 or a sequence that is at least 80%, 85%, 90%, 95%, 98%, 99%, or 100% identical to SEQ ID NO: 36.

43.-52. (canceled)

53. The system of claim 1, wherein the one or more first helper polynucleotides, the one or more second helper polynucleotides, and/or the donor polynucleotide are comprised within one or more vectors, wherein the one or more vectors comprise an Agrobacterium tumefaciens Ti vector, and the one or more first helper polynucleotides, the one or more second helper polynucleotides, and/or the donor polynucleotide are comprised within a T-DNA region of the Agrobacterium tumefaciens Ti vector.

54. (canceled)

55. The system of claim 53, wherein the T-DNA region comprising the one or more first helper polynucleotides, the one or more second helper polynucleotides, and/or the donor polynucleotide comprises the sequence of any one of SEQ ID NOs: 38-46.

56. A method for integration of a nucleic acid sequence into a double-stranded target sequence of a genome of a plant cell or a target plasmid in a plant cell, comprising: contacting the plant cell with the system of claim 1, wherein the cargo sequence is integrated at an integration site in the genome of the plant cell or at a target site of the target plasmid upon expression of the RNA-guided DNA binding complex and the transposition complex in the plant cell.

57.-73. (canceled)


SEQUENCE LISTING (69 sequences)

SEQ ID NO: 1 - DNA, 365 bp, synthetic construct
  source 1..365: mol_type = other DNA
tagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctc

SEQ ID NO: 2 - DNA, 157 bp, synthetic construct
  source 1..157: mol_type = other DNA
aataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgt

SEQ ID NO: 3 - DNA, 1091 bp, synthetic construct
  source 1..1091: mol_type = other DNA
tcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcat

SEQ ID NO: 4 - DNA, 250 bp, synthetic construct
  source 1..250: mol_type = other DNA
ccgtataggcagctgaagataaaaacaatcgattcgatgtttaagttaatgtaattctgccgaaaaggcagtgagtagtaattgaaaaaaccagtcttatatccgggctggtttttttgtttttagcgttataacttttatttcactatgtcactgtatggttgcattttatgtcagtgaatggttgcaggttttttgtaagttattgatatgcctatccagtgtatgtcacgctttgcttgcagcgaca

SEQ ID NO: 5 - DNA, 249 bp, synthetic construct
  source 1..249: mol_type = other DNA
gtgtccgtgactgatgcaaggttatcggaactgtaatgattatatgttttctaattttgaagtggttcttttgatgagttttgccagttactgaaagtaactttcttactgcagtagttttgctgaaatactcgattcacaaaaatatcaacttatggttgttttgtgagatatcaatatatggttgttttgtggttaagttgctgattataaataattattaaatatcactttatggttgcatcaaca

SEQ ID NO: 6 - DNA, 250 bp, synthetic construct
  source 1..250: mol_type = other DNA
tgtcgctgcaaccatagtttgacataatgcaaccataccttgacataattaaagtcatactctgacataaagccccttattaaaaatgatgctaaaaataaattccattataatcaattatttaagaattaattaaaccatagtgtgacatagtggctttttatttgtatagtatgtgtactcaaataataaaaatttacttatgtatatacgcaacttaagaaagccatctccaaataaaaatgtattt

SEQ ID NO: 7 - DNA, 250 bp, synthetic construct
  source 1..250: mol_type = other DNA
tgttgatacaaccataaaatgataattacacccataaattgataattatcacacccataaattgatattgcctcttcatggtctaaacttcagtaagtttacgacattttcatgaagaggtcatttcatgacaagcttacctacgccctcagcaattacgacttcggcgttagagtatgcattccatactcccgctcgcaatctaacgaaatctcgcggaaaaaatattcatcgttatgtcagtgtaaag

SEQ ID NO: 8 - protein, 200 aa, synthetic construct
MVKWYYKTITFLPELCNNESLAAKCLRVLHGFNYQYETRNIGVSFPLWCDATVGKKISFVSKNKIELDLLLKQHYFVQMEQLQYFHISNTVLVPEDCTYVSFRRCQSIDKLTAAGLARKIRRLEKRALSRGEQFDPSSFAQKEHTAIAHYHSLGESSKQTNRNFRLNIRMLSEQPREGNSIFSSYGLSNSENSFQPVPLI

SEQ ID NO: 9 - protein, 203 aa, synthetic construct
MQRYYFTVHFLPKQANLALLTGRCISIMHGFILKHNIEGMGVTFPAWSDSSIGNEIAFVYTDKEILNTLKDQAYFVDMQDCGFFKVSQVLAVPDSCEEVRFIRNQAVAKIFTGESRRRLKRLQKRALARGEDFNPKKIEAPREIDIFHRVAMTSKSSQEDYILHIQKQDVDCQAEPYFSNYGLASNEKFKGTVPDLSPSIDRN

SEQ ID NO: 10 - protein, 352 aa, synthetic construct
MKLPTNLAYERSIDPSDVCFFVVWPDDRKTPLTYNSRTLLGQMEAASLAYDVSGQPIKSATAEALAQGNPHQVDFCHVPYGASHIECSFSVSFSSELRQPYKCNSSKVKQTLVQLVELYETKIGWTELATRYLMNICNGKWLWKNTRKAYCWNIVLTPWPWNGEKVGFEDIRTNYTSRQDFKNNKNWSAIVEMIKTAFSSTDGLAIFEVRATLHLPTNAMVRPSQVFTEKESGSKSKSKTQNSRVFQSTTIDGERSPILGAFKTGAAIATIDDWYPEATEPLRVGRFGVHREDVTCYRHPSTGKDFFSILQQAEHYIEVLSANKTPAQETINDMHFLMANLIKGGMFQHKGD

SEQ ID NO: 11 - protein, 350 aa, synthetic construct
MELCNILKYDRSLYPGKAVFFYKTADSDFVPLEADINKIRGPKSGFTEAFTPQFSPKNISPQDLTHNNILTLEECYVPPNVEHIFCRFSLRVQANSLVPSGCSDPEVFSLLKELAETFKECGGYKELAVRYCRNILIGTWLWRNQNTGNTQIEIKTSKGSCYLIDNTRKLAWESKWASDDLKVLEELSNEIESALTDPNVFWSADITAKIEASFCQEIYPSQILNDKVKQGEASKQFVKAKCADGRYAVSFNSVKIGAALQSIDDWWDEDASKRLRVHEFGADKEIGVARRPPDSEQNFYSIFKNTEWYLSALKNCITNKNEKIDPAIYYLFSVLIKGGMFQKKAEAKKA

SEQ ID NO: 12 - protein, 640 aa, synthetic construct
MQTLKELIASNPDDLTTELKRAFRPLTPHIAIDGNELDALTILVNLTDKTDDQKDLLDRAKCKQKLRDEKWWASCINCVNYRQSHNPKFPDIRSEGVIRTQALGELPSFLLSSSKIPPYHWSYSHDSKYVNKSAFLTNEFCWDGEISCLGELLKDADHPLWNTLKKLGCSQKTCKAMAKQLADITLTTINVTLAPNYLTQISLPDSDTSYISLSPVASLSMQSHFHQRLQDENRHSAITRFSRTTNMGVTAMTCGGAFRMLKSGAKFSSPPHHRLNSKRSWLTSEHVQSLKQYQRLNKSLIPENSRIALRRKYKIELQNMVRSWFAMQDHTLDSNILIQHLNHDLSYLGATKRFAYDPAMTKLFTELLKRELSNSINNGEQHTNGSFLVLPNIRVCGATALSSPVTVGIPSLTAFFGFVHAFERNINRTTSSFRVESFAICVHQLHVEKRGLTAEFVEKGDGTISAPATRDDWQCDVVFSLILNTNFAQHIDQDTLVTSLPKRLARGSAKIAIDDFKHINSFSTLETAIESLPIEAGRWLSLYAQSNNNLSDLLAAMTEDHQLMASCVGYHLLEEPKDKPNSLRGYKHAIAECIIGLINSITFSSETDPNTIFWSLKNYQNYLVVQPRSINDETTDKSSL

SEQ ID NO: 13 - protein, 695 aa, synthetic construct
MHLKELLEITDTTERDRSLRRAFSPYTAMIDITGSEAVALIILLNLTYRKNQVDDLLDKKLAKQALKSEDHINKCIKEIAWFHTHNLKYPDIRVSKQNLAVEPPTLHSYVLSSANYPKAYGWSHNSAKVNFAKLFVSYFKWQNQVSWLAQVLATNSDNWKSAFTSLGLSVKAFKSLCVTVKNSLPEEAIPDSVDRYSRQIRMPYHDGYLAVTPVISHVVQSKIQQAAIDKRARFSNVEFTRPAAVSMLAASLGGVINVLNYPPYIRSKYHGLSNSRAFKLNNGQTVFNVEALLKPELIKALEGIIFSNNALALKQRRQQKVKNIKELRNTLLEWFSPVFEWRLDAIENGYDLEQLESASERLEYKILSLPDNELPSLTIPLFRLLNEMLGGVSMTQRYAFHPKLMSPLKAALQWLLVNLTDQKHVLIEEDDEHYRYLHLSGIRVFDAQALSNPYCSGIPSLTAVWGMIHSYQRKLNEALGTNVRFTSFSWFIRNYSAVAGKKLPELSLQGAQQSRLKRPGIIDGKYCDLVFDLIIHIDGYEDDLQAVDSKPDILKAHFPSNFAGGVMHQPELNSNINWCCLYSNENQLFEKLRRLPLSGCWVMPTEHKIQDLDELLLLLNSDSKLSPSMMGYMLLTEPMARVGSLERLHCYAEPAIGVVKYEAATSVRLKGIGNYFNSAFWMLDAQEKFMLMKKV

SEQ ID NO: 14 - protein, 394 aa, synthetic construct
MFLQRPKPYSDESLESFFIRVANKNGYGDVHRFLEATKRFLQDIDHNGYQTFPTDITRINPYSAKNSSSARTASFLKLAQLTFNEPPELLGLAINRTNMKYSPSTSAVVRGAEVFPRSLLRTHSIPCCPLCLRENGYASYLWHFQGYEYCHSHNVPLITTCSCGKEFDYRVSGLKGICCKCKEPITLTSRENGHEAACTVSNWLAGHESKPLPNLPKSYRWGLVHWWMGIKDSEFDHFSFVQFFSNWPRSFHSIIEDEVEFNLEHAVVSTSELRLKDLLGRLFFGSIRLPERNLQHNIILGELLCYLENRLWQDKGLIANLKMNALEATVMLNCSLDQIASMVEQRILKPNRKSKPNSPLDVTDYLFHFGDIFCLWLAEFQSDEFNRSFYVSRW

SEQ ID NO: 15 - protein, 411 aa, synthetic construct
MAFLFSPKARAFSDESLESYLLRVVSENFFDSYEGLSLAIREELHELDFEAHGAFPVDLKRLNVYHAKHNSHFRMRALGLLETLLDLPRYELQKLALLKSDIKFNSSVALYNNGVDIPLRFIRHHAEEAVDSIPVCSQCLAEEAYIKQSWHIKWVNACTKHQCALLHNCPECYAPINYIENESITHCSCGFELSCASTSPVNTLSIEHLNKLLDKGERNDSNPLFNNMTLTERFAALLWYQERYSQTDNFCLNDAVNYFSKWPAVFNTELDELSKNAEMKLIDLFNKTEFKFIFGDAILACPSTQKQSESHFIYRALLDYLVTLVESNPKTKKPNAADLLVSVLEAATLLGTSVEQVYRLYQNGILQTAFRHKMNQRINPYKGAFFLRHVIEYKTSFGNDKARMYLSAWTG

SEQ ID NO: 16 - protein, 861 aa, synthetic construct
MATSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYGGSGSGKRTADGSEFESPKKKRKVGSGSGGAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 17 - protein, 851 aa, synthetic construct
MYIRNLRKPSPNKNVFKFASTKVSSVVMCESSLEFDACFHHEYNDLIESFGSQPEGFKYEFMGKSLPYTPDALISYTDKTQKYHEYKPYSKIASPLFRAEFAAKRAASLKLGIDLVLVTDRQIRVNPILNNLKLLHRYSGVYGISGIQKELLSFIHKSGVIKLNDISSQVGIPIGETRSFLFGLMHKGLVKADLGCDDLTNNPTLWATPGSGSGKRTADGSEFESPKKKRKVGSGSGGMTDFFNEFDESLVPLKPQTPTQYVKLDDANLIQRDLDTFSDTFKNQALQRYKLISTIDKKLSRGWTQRNLDPILDELFKGGDVVRPNWRTVARWRKKYIESNGDIASLADKNHKMGNRTNRIKGDDKFFDKALERFLDAKRPTIATAYQYYKDLIVIENESIVEGKIPIISYNAFNKRIKAIPPYAVAVARHGKFKADQWFAYCAAHVPPTRILERVEIDHTPLDLILLDDELLIPIGRPYLTLLIDVFSGCVLGFHLSYKSPSYVSAAKAITHAIKPKSLDALNIELQNDWPCFGKFENLVVDNGAEFWSKNLEHACQSAGINIQYNPVRKPWLKPFIERFFGVMNEYFLPELPGKTFSNILEKEEYKPEKDAIMRFSTFVEEFHRWIADVYHQDSNSRETRIPIKRWQQGFDAYPPLTMNEEEETRFSMLMRISDSRTLTRNGFKYQELMYDSTALADYRKHYPQTKETVKKLIKVDPDDISKIYVYLEELESYLEVPCTDPTGYTDGLSIYEHKTIKKINREVIRESKDSLGLAKARMAIHERVKQEQEVFIESKTKAKITAVKKQAQIADVSNTGTSTIKVSEESAAPVQKHISNDNSDDWDDDLEAFE

SEQ ID NO: 18 - protein, 330 aa, synthetic construct
MSETREARISRAKRAFVSTPSVRKILSYMDRCRDLSDLESEPTCMMVYGASGVGKTTVIKKYLNQNRRESEAGGDIIPVLHIELPDNAKPVDAARELLVEMGDPLALYETDLARLTKRLTELIPAVGVKLIIIDEFQHLVEERSNRVLTQVGNWLKMILNKTKCPIVIFGMPYSKVVLQANSQLHGRFSIQVELRPFSYQGGRGVFKTFLEYLDKALPFEKQAGLANESLQKKLYAFSQGNMRSLRNLIYQASIEAIDNQHETITEEDFVFASKLTSGDKPNSWKNPFEEGVEVTEDMLRPPPKDIGWEDYLRHSTPRVSKPGRNKNFFE

SEQ ID NO: 19 - protein, 334 aa, synthetic construct
MGNALTEIQIEKLRNFSDCIVMHPQIKTIFNDFDELRLNRKFQSDQQCMLLIGDTGVGKSHTINHYKKRVLATQNYSRNTMPVLVSRISRGKGLDATLVQMLADLELFGSSQIKKRGYKTDLTKKLVESLIKAQVELLIINEFQELIEFKSVQERQQIANGLKFISEEAKVPIVLVGMPWAAKIAEEPQWASRLVRKRKLEYFSLKNDSKYFRQYLMGLAKKMPFDVPPKLESKNTTIALFAACRGENRALKHLLLEALKLALSCNEYLENKHFITAYDKFDFFNDKEKLKSKNPFKQDIKDIEIYEVIKNSSYNPNALDPEDMLTDRVFAIVK

SEQ ID NO: 20 - protein, 207 aa, synthetic construct
MSYSGERDNFAPHMALVPMVIEQTSRGERSFDIYSRLLKERVIFLTGQVEDHMANLIVAQMLFLEAENPEKDIYLYINSPGGVITAGMSIYDTMQFIKPDVSTICMGQAASMGAFLLTAGAKGKRFCLPNSRVMIHQPLGGYQGQATDIEIHAREILKVKGRMNELMALHTGQSLEQIERDTERDRFLSAPEAVEYGLVDSILTHRN

SEQ ID NO: 21 - protein, 424 aa, synthetic construct
MTDKRKDGSGKLLYCSFCGKSQHEVRKLIAGPSVYICDECVDLCNDIIREEIKEVAPHRERSALPTPHEIRNHLDDYVIGQEQAKKVLAVAVYNHYKRLRNGDTSNGVELGKSNILLIGPTGSGKTLLAETLARLLDVPFTMADATTLTEAGYVGEDVENIIQKLLQKCDYDVQKAQRGIVYIDEIDKISRKSDNPSITRDVSGEGVQQALLKLIEGTVAAVPPQGGRKHPQQEFLQVDTSKILFICGGAFAGLDKVISHRVETGSGIGFGATVKAKSDKASEGELLAQVEPEDLIKFGLIPEFIGRLPVVATLNELSEEALIQILKEPKNALTKQYQALFNLEGVDLEFRDEALDAIAKKAMARKTGARGLRSIVEAALLDTMYDLPSMEDVEKVVIDESVIDGQSKPLLIYGKPEAQQASGE

SEQ ID NO: 22 - DNA, 21 bp, synthetic construct
  source 1..21: mol_type = other DNA
ccaaagaagaaaagaaaagtt

SEQ ID NO: 23 - DNA, 60 bp, synthetic construct
  source 1..60: mol_type = other DNA
atgggaaagcgtactgctgatggttcagagtttgaaagtccaaagaagaaaagaaaagtt

SEQ ID NO: 24 - DNA, 603 bp, synthetic construct
  source 1..603: mol_type = other DNA
atggtgaaatggtactacaaaacaataacatttctacctgagctctgcaacaatgaatcactagcagcgaaatgtctgcgtgtacttcatggattcaactaccaatatgaaacaagaaacattggtgttagcttccctctttggtgtgacgcaactgtcggaaagaagatatcctttgtgtccaagaacaagattgagcttgacttgcttctcaaacaacattactttgttcaaatggagcagcttcagtatttccacatctcaaatactgtgctggttccagaagattgcacttatgtcagtttcaggagatgtcaaagtattgataagcttactgctgctggtttagcaagaaagatccgtcgattggagaagagagctttgtcaagaggagaacagtttgatccctcaagttttgctcagaaagaacacaccgccattgctcattatcattctctcggtgaatcttcaaagcaaacaaataggaattttcggttaaatataaggatgttgagtgaacaaccaagagaaggaaattccatcttctcttcttatgggctatcaaattctgagaattcttttcagcctgttccgctaatctga

SEQ ID NO: 25 - DNA, 612 bp, synthetic construct
  source 1..612: mol_type = other DNA
atgcagagatattattttaccgttcattttcttcctaaacaagctaatttggcccttctcaccggaagatgcatatctatcatgcatggatttatcttgaagcataacattgaaggaatgggagtaacatttcccgcatggtctgattcaagcattggaaacgagattgcttttgtttacactgataaagaaatcctaaacacattgaaagatcaggcttactttgtggatatgcaagactgtggtttcttcaaggtttctcaggtgttggctgttcctgattcttgcgaggaagtcaggttcatccgcaatcaagccgtcgcaaagattttcactggcgaatcgaggaggagacttaaacggttacaaaagagagccttagcaagaggagaagatttcaatccaaagaagatagaagctccaagagaaatagacatttttcaccgtgtagcaatgacaagcaaaagttctcaagaagactacattctgcacatccagaagcaagatgttgattgtcaagcagagccttatttttcaaactatggtcttgcgtcaaatgagaagttcaaaggaactgtaccagacttgagtccgtccattgatagaaattga

SEQ ID NO: 26 - DNA, 1059 bp, synthetic construct
  source 1..1059: mol_type = other DNA
atgaaacttccaacaaatctagcttacgagaggtcgattgatccttcagatgtttgtttctttgtggtctggccagatgatcggaaaacaccgttgacatacaattcacgtaccctcctagggcagatggaggctgcatctctggcgtatgatgtaagtggtcaaccgatcaagagtgccacagcagaagcattagctcaaggaaatcctcatcaagttgacttttgtcatgttccttatggagcatctcacatcgagtgcagcttctctgtttctttttcatcagagcttcgccagccttacaaatgtaacagcagtaaagtgaagcaaactctcgttcagctcgtagagttgtatgaaactaagattggatggactgaactagcgacgagatatttgatgaacatatgcaatggtaaatggctctggaaaaatacaaggaaggcttattgttggaatatagtcctgactccatggccttggaacggagagaaggttggctttgaagacattaggacaaactacacttcaagacaagatttcaagaacaacaagaattggtctgccattgttgagatgattaagacagctttcagttctactgatggcttagcgatcttcgaggttcgtgcgactcttcacttaccaaccaatgctatggttagaccttcccaggtattcacagagaaagaatcagggtcgaaatcaaaaagcaagactcagaattctcgagtgtttcaatcaacaaccatagatggtgaaagatctcccattcttggtgcattcaaaactggtgcagctatagctactattgacgactggtatcctgaagctacagaaccattgcgtgttggaagatttggtgtccatagagaagatgtgacttgctatagacatccttccacagggaaagattttttctccatacttcaacaagctgaacattacattgaagtactttcagcaaacaaaaccccagcacaagaaaccattaatgatatgcattttcttatggctaatctcatcaaaggtggcatgtttcagcacaaaggagactga

SEQ ID NO: 27 - DNA, 1053 bp, synthetic construct
  source 1..1053: mol_type = other DNA
atggagctttgcaatatactcaagtatgatcgaagtctttaccctgggaaagcggttttcttctacaaaacggcggacagcgattttgttcctttggaagctgacattaataagataagaggaccaaaatcaggctttacagaggctttcactcctcaattctctccgaaaaatatctctccacaagatctaactcacaacaacattcttacactcgaagagtgttatgttccaccaaatgtcgaacatattttctgccggttttctctgcgtgtccaagctaattcgttagtcccctctggttgttccgatcctgaggtttttagccttctcaaagagcttgcagaaaccttcaaagaatgtggtggttacaaggagctggcagtacgctattgtcggaacattttgattggaacatggctttggagaaatcagaataccggaaacacacagattgagatcaagacttcaaaaggatcttgctacttgattgataatacaagaaaactcgcttgggaaagcaaatgggcttcagacgacttgaaagttttggaagaattaagcaatgaaatcgaatctgctttaacagatccaaatgtgttttggtctgcagatataaccgccaagatcgaagcatctttctgtcaagagatatatccttcacagatattgaatgataaagtgaagcaaggagaagcgtcaaaacagtttgtgaaagctaaatgtgctgatggaaggtatgctgtgagtttcaacagtgttaagatcggtgctgctttgcaaagtattgatgattggtgggatgaagatgctagtaaaagacttagagttcatgaatttggtgcagacaaggaaattggcgtagcgaggaggcctcccgattccgaacaaaacttttattcaatcttcaagaacactgaatggtatctttccgccttaaagaattgcatcactaacaagaatgagaagattgacccagcaatatactatttgttcagcgtgttgatcaaaggtggaatgtttcagaaaaaggcagaggcaaagaaagcatga

SEQ ID NO: 28 - DNA, 1923 bp, synthetic construct
  source 1..1923: mol_type = other DNA
atgcagactcttaaagaactaattgcttctaatccggatgacctaacaacggagctcaaaagggcatttcgccccctgactcctcacattgccattgacggtaacgagcttgacgctctcaccattcttgtgaatctcacagacaaaactgatgatcaaaaggatcttctcgatagagctaaatgtaaacagaaactcagagatgagaaatggtgggcttcttgcatcaattgcgttaattatagacagtctcacaatcctaaattccctgatatcaggtctgaaggagtgattcgcactcaagctttgggcgaacttccttcatttctattaagctccagtaaaataccaccgtatcattggtcatattctcatgattcaaaatatgttaacaagtcagcctttttaacaaatgaattctgttgggacggagagataagctgcttgggggaactcctcaaagatgcggatcatccactttggaacacactaaagaagcttggctgctcacagaagacatgcaaagctatggcgaaacaattggctgatatcacattaacaacgattaatgtaacactcgctccgaattacttgactcagatttctcttccggattctgatacaagttacatttctctctcgcctgtcgcaagtctatcaatgcaaagtcatttccaccaacgtttgcaagacgaaaacagacattctgctataacaagattctcccgaacaactaacatgggtgtgactgcaatgacttgtggtggagctttcaggatgttaaagtctggtgcaaagttctccagtccacctcatcatagattaaactcgaaacgctcttggttgaccagtgagcatgttcaatcattgaagcagtatcaacgtctgaacaaatcattgatccctgagaatagtaggattgctcttaggcgcaagtacaagattgaacttcaaaatatggttcgatcatggtttgccatgcaagatcacactctcgattcaaatatccttattcaacatctcaatcacgatttgagttatctcggggcgaccaaaagatttgcttatgatccagctatgacaaaattgttcactgagctgctaaagagggagctgagtaattctatcaacaacggagaacaacataccaatggcagttttctggtcctcccaaacatacgggtttgtggagcaactgcgttatcttccccagtaaccgttggcatccctagcttaaccgccttctttggttttgttcacgcctttgagaggaacatcaacaggaccacttcttcctttcgtgtcgagagctttgcaatatgtgtccatcagcttcatgtggagaagcgtggtttgacagctgagtttgtagagaagggagatgggacaatttcagcaccagcaaccagagatgattggcaatgtgatgttgtgttttctctaattttgaatacgaacttcgctcaacacattgatcaggacacactggtcacgagtttacctaagcggctggcaaggggttctgctaagatcgcaatagatgatttcaagcatataaactcattctccactcttgaaactgcaattgagtctcttcccatcgaagctggaagatggcttagtctgtatgcacagagtaataataatctttcggacttgttagctgctatgaccgaagaccatcaacttatggctagttgtgttggctaccaccttctagaagaaccaaaagataagccaaatagccttagaggatacaaacatgccatagctgaatgtattataggattaattaattcaattacattttcctcggaaaccgatcctaatactatattctggtccttaaagaactaccaaaactatcttgtagttcagcccagatccataaatgatgaaacaacagacaagagcagcctgtga

SEQ ID NO: 29 - DNA, 2088 bp, synthetic construct
  source 1..2088: mol_type = other DNA
atgcacctgaaagagctgttagagataacagatacaactgagagagatagatctctccgacgtgccttctcgccgtataccgccatgatcgacattacaggatcagaagcagtcgctctaattatcctactcaatctcacttatagaaagaatcaggtagatgacctgttggataagaaactcgcgaaacaagccctcaagagtgaggatcatatcaacaaatgcatcaaggagattgcttggtttcatactcacaaccttaaatatccagacataagggtgagtaagcaaaatcttgcagtagaaccaccaacattgcattcttatgtgctttcttctgctaattatcccaaagcttatggttggtcccataattcagctaaagtgaattttgctaagttgtttgtctcatacttcaaatggcaaaaccaagtgagctggcttgcccaagtcttggcaaccaactctgataactggaaaagtgcatttacttcccttggtttatcggtgaaagccttcaaatctttatgcgttacagttaagaattctttacctgaagaggcaatcccggactcagtggatagatatagcaggcagattagaatgccttatcatgatggttatcttgctgtcactcctgtgatatctcacgttgttcaaagcaaaatacaacaagctgctattgacaaaagagctaggttttctaatgttgagtttaccagaccagctgctgtttcaatgctggcggcaagtctaggtggtgtgataaatgtacttaattatcctccatacattcgatcaaagtaccatggactatcgaattcaagggcttttaagttaaacaacggacagactgtcttcaacgtggaagccctcctgaagcctgagcttataaaagctcttgaaggaattatattctctaacaatgcattggcattaaaacagagaaggcagcagaaagtaaaaaacatcaaagaactgagaaataccctccttgagtggttttcccctgttttcgaatggagattggatgcaattgagaatggttatgatctagaacaattggaatcagcgagcgaacggttagagtacaagattctgagtttgccggataatgaactaccttctttgactatacccttgtttagattacttaatgagatgttgggaggagtttcaatgactcagaggtatgcattccatccaaagttgatgtctccattaaaggctgctctccaatggttgctcgttaatttaactgatcaaaagcatgttttaattgaagaagatgatgagcattacagataccttcacctgtcagggattagggtttttgatgcccaagcattgtctaatccttactgctctggtattccttcgcttacggcggtttggggaatgatccacagttatcaacgcaaactaaacgaagctttgggcacgaacgtacggttcacaagcttttcatggttcatccgtaattactctgccgttgctggtaaaaagctaccagaacttagccttcagggtgcacaacagtctagattgaaaagacctgggataatagacgggaagtattgtgatttggtgtttgacttgattattcatatcgatggctatgaagacgatttgcaggctgttgattcaaagcctgatatcctcaaggctcatttcccaagcaatttcgccggcggcgtgatgcatcaacccgagttgaattctaatattaattggtgttgtctttactccaacgaaaaccagctttttgagaagctccgtcggcttcctctttctggatgttgggttatgcccacagaacataagattcaagatcttgatgaactgttgttgctgttgaactcggatagtaaactaagtccaagcatgatgggatacatgcttctcacagagccaatggctcgtgtaggatcgttagagaggcttcactgttatgcagaacctgctattggtgttgtcaaatacgaggcagcaacttctgttcgattaaaaggtatcggaaactatttcaattccgcgttttggatgctcgatgctcaagaaaagttcatgctcatgaagaaagtgtga

SEQ ID NO: 30 - DNA, 1185 bp, synthetic construct
  source 1..1185: mol_type = other DNA
atgtttcttcaaagacctaaaccgtatagcgacgaaagtctggaatctttcttcatccgtgttgccaacaagaatggttatggagatgttcacaggtttttggaagccacaaagcgattccttcaagacattgatcacaatggataccaaacatttcccactgatataacgcgtatcaatccttattcagccaaaaactcttcatcagcaagaacagcgagtttcttgaagctagcacagctcaccttcaacgaaccaccagaattgctagggcttgcaataaacagaaccaacatgaagtactctccttctactagtgcagtggtaagaggagcagaggttttcccaagatccctcctgcgtactcattccattccatgttgccctctctgcttgagggaaaatgggtatgctagttacctttggcattttcaaggatacgagtattgtcactctcataatgtccctctaattaccacttgttcttgtggtaaagaattcgattacagagtatctgggttaaaaggaatttgttgtaagtgcaaagagccaataactttgacatcgagagagaatggccatgaagcagcttgtactgttagtaattggcttgccggtcacgagtcaaaaccattgcctaatttacccaaatcttatagatggggactcgtccattggtggatgggaatcaaggactctgaatttgatcatttcagctttgtgcagtttttctcaaactggcctcggtcgtttcatagtatcatagaagatgaagttgaatttaatcttgaacatgctgttgtttctacatctgagctaaggctcaaagatcttcttgggagactgttctttggtagcatcaggcttcctgagagaaatctgcaacataacattattttgggtgagctgttatgttatctagaaaaccgtttgtggcaagataaaggcttgatagccaatttaaagatgaatgctcttgaggcaacagtgatgcttaactgctccttggatcagattgcttcaatggtggagcaaagaattttaaagcctaaccgaaaatcaaagccaaattcaccgcttgatgtgacggattatttgtttcactttggtgacattttctgcttatggttagctgagtttcagtctgatgaattcaatcgaagcttttatgtttctaggtggtga

SEQ ID NO: 31 - DNA, 1229 bp, synthetic construct
  source 1..1229: mol_type = other DNA
atggcttttcttttctcaccaaaagcaagagcgttttccgatgaatcgcttgaaagttatttacttcgagttgtctcagaaaacttcttcgattcatatgaaggactatctttggccatcagagaagaacttcatgagttggacttcgaggctcatggagcatttcccgtagatttaaagagacttaatgtttaccatgcaaaacacaactcacactttaggatgagagctcttggcctattagaaactcttttagatcttcctcgttatgagcttcaaaagctagcactcctgaagtcagacattaaattcaattcttccgttgccctctacaacaatggtgttgatattcctttgagattcattagacatcatgctgaggaagctgttgactccattccggtttgttctcaatgcttggctgaagaagcttatataaagcaatcttggcatatcaaatgggttaatgcttgcaccaaacatcaatgtgctttgcttcacaattgtcctgaatgttatgcaccgatcaattacattgagaatgaatcaatcactcattgctcttgtggttttgaattgagctgcgcctctacaagtcctgtgaacacactctcgattgaacatttgaacaaactcctggacaaaggagaacggaatgactcaaatccattgtttaacaacatgacgttgacagaaaggtttgctgctttattatggtatcaagagaggtattcacagactgataatttctgtttgaatgatgctgtgaactacttcagtaagtggccagcagtgtttaatactgagctagatgagctctcaaagaatgccgagatgaagctgattgatctcttcaacaagacagagtttaagtttatctttggagatgcaattcttgcttgtccaagtactcagaagcagagtgaatctcatttcatctaccgggcattacttgattaccttgtcactttagtggagagcaatcccaaaacaaagaagccaaacgcagctgatttgcttgtttctgtcctggaagcagcaactttgttggggacgagtgtagaacaagtttataggctctatcagaatggaatactgcaaaccgcctttcggcacaagatgaatcagagaataaacccttacaagggtgctttctttctccgtcatgtgatagagtataaaaccagctttggtaacgataaagctagaatgtatctctcagcttggtg

SEQ ID NO: 32 - DNA, 2586 bp, synthetic construct
  source 1..2586: mol_type = other DNA
atggcgacatcccttccaacgccatcagctataactacttcggcactagagtatgctttccatactccggctcgcaatttgacgaagtctagggggaagaacattcatcgatatgttagtgtcaagatgtctaaaagaattactgtagaatcaacactagaatgcgatgcttgctatcattttgacttcgagccttctatcgtgagattttgtgctcagcctattcgctttctgtattatttaaatgggcagtcacactcctatgtcccagattttcttgtgcaattcgacacaaacgagtttgtcttgtatgaagttaaaagcgcttatgcgaaaaacaaacctgatttcgatgttgagtgggaagcaaaggtgaaggccgcaacagagctgggtttggaacttgaacttgttgaggagagcgatataagagataccgttgttttgaacaatcttaagaggatgcatagatatgcgagtaaggacgagcttaataacgtacacaattccctacttaagatcataaagtataacggagctcaatccgcccgatgtttgggtgaacagctagggttgaaaggtcgtacggtacttcctattctgtgtgatcttctttcaagatgcttgctcgacaccagacttgataagcccctctctctggaaagtcgttttgaactcgcctcatatggaggatcaggatctggcaaaagaaccgcagatggttctgagttcgaaagcccaaagaaaaagagaaaggtgggttccggttctggaggagccaagaagggtttttctagctttcacaggaaggcagtttcctcacaggacactttagagagtatagaactagtttccagtgcgaattgcctcgaatcagtcacttatcaagatatatctgcatttcctgaaaccattgcagtggagatcaacttcagattgtctattctccggtttctcgcaagaaaatgtgagactatcgttgctaaatctattgagccccatagagttgagcttcaacagaattattctcggaagattccatctgcaataacaatatacagatggtggttggcctttcggaagtcagattacaacccgattagtctggctcctaatatcaaagacagagggaacagagaaactaaagtatctactgtggttgactccatcatggaacaagccgttgaaagggttatctctggtcgtaaagtcaatgtttctagcgcgtacaaacgagtccggaggaaagttagacaatataatcttactcatggaacaaagtacacatatccaaaatacgagagcgtacgaaagcgcgtaaagaagaaaacaccgtttgagttattagctgctggtaagggagagagggtcgccaagcgtgaatttaggagaatgggaaagaagattctgacgtcaagtgttctcgagagggtggagatagatcacactgttgtggatctgttcgctgttcatgaggaatataggattcctctcgggaggccatggctgactcagttagttgattgttacagcaaagcagtcattggtttctaccttggctttgaacctcctagttatgtctccgtatcactagctttaaagaatgccattcaacgcaaagatgatttgatttcctcttacgagtcaatagaaaatgaatggttatgttatggtataccagatctccttgttacagacaatggaaaagaatttttgtccaaagcttttgatcaagcttgtgagagcttattaattaacgtgcatcagaataaggtagaaaccccagataacaagcctcatgttgaaagaaactacgggacaataaatacttccttattggacgatttacctggtaaaagtttctctcagtacttgcagagggaaggatatgactctgtgggagaagctacattgactctcaatgagatcagagaaatctacttgatttggcttgtcgacatatatcacaagaaaccgaatcaaagaggaactaactgtccaaatgttgcttggaaaaaaggttgccaagaatgggagccagaagaattctccgggtcaaaagatgagcttgattttaaattcgcaattgttgattataaacaacttacaaaagtaggaatcactgtttacaaggaactaagctactcaaacgatcgtttggcagagtatagaggcaaaaaaggaaaccacaaagttcagttcaagtacaatccggaatgtatggctgtgatatgggttcttgatgaagatatgaacgaatatttcacagttaatgctatcgactatgaatacgcatctagggtgtcactgtggcaacataaatacaacatgaagtatcaagcagagctaaattctgcggaatatgacgaagataaagagattgatgcagaaattaaaatcgaggaaattgctgatagatctatagtgaaaaccaataaaattagagctcgtcgtcgtggtgctaggcatcaagagaatagtgcacgagctaaatcaatctctaatgcaaatcctgcaagtattcagaagcatgaagacgaaatagtcagtgctgataatgatgattgggatatcgattacgtgtga

SEQ ID NO: 33 - DNA, 2556 bp, synthetic construct
  source 1..2556: mol_type = other DNA
atgtacattaggaacttgagaaagccttctccaaataagaatgtttttaagttcgcatctaccaaggtcagctctgtggtcatgtgtgaatccagccttgagtttgacgcttgctttcatcatgaatataacgacttgattgagagttttggttctcagccagaaggtttcaaatacgaattcatgggtaaatctctcccttacactccagatgctttgatctcctacacggacaaaactcaaaagtatcacgagtataaaccatattcaaaaatcgcatcacctctatttagagcagagtttgctgcaaagagggctgcaagtcttaagttgggtattgatttggtactcgtcacagacagacagattagagtaaacccaatactcaacaatctgaagctccttcatagatactcgggcgtttatggtatatctggtatccagaaggaactacttagctttattcacaaaagtggcgttatcaaactcaatgatatctcatcacaggttgggatccccattggagaaactcggtcatttctttttggactgatgcataagggacttgtaaaggctgacctaggttgtgatgacctgacaaacaaccctactctttgggctactcctggaagtggctcaggcaaacgtactgctgacggaagtgagttcgagtccccaaagaaaaaaagaaaagttggtagtggatctggtggaatgactgatttcttcaatgagtttgatgaatcgcttgtaccactgaaaccacaaactccaacgcagtatgttaagctggacgatgctaatttaattcagagagatttagatacattctcagatactttcaagaaccaagccctccaaagatataagctaatttctacaattgataaaaagttaagccgcggatggactcaaagaaatctcgacccgatcctcgacgaactcttcaaaggcggtgatgtagtgaggccaaactggagaactgtggcaagatggaggaagaaatatatagaaagcaatggtgacatcgcttctcttgctgataaaaaccacaaaatgggaaacagaacaaatcggataaaaggagatgacaaattttttgacaaggccttggaacgtttccttgacgcgaaaagacctacaatagcaacagcttatcaatattacaaggacttgatagtgattgaaaacgagtcaattgttgaagggaagatccccatcatctcttataatgcatttaacaaacgaattaaagccattcctccttatgctgttgcagtcgctaggcatgggaaattcaaagctgatcaatggtttgcatattgtgcagctcacgttccaccaacgcgtatacttgaacgagtggagattgatcatactccgttggatctaatattattggatgatgagcttctgattcccattggccggccatatcttacacttctcatagatgtgttttcaggttgcgttctgggattccatttgtcatacaagagcccttcttatgtctctgcagcaaaagcaatcactcacgcaataaaacccaaaagcttagatgcgcttaatattgagcttcagaatgactggccctgtttcgggaagtttgagaatttagtagttgacaatggagctgaattttggtcaaaaaatttggaacatgcttgtcaatctgctggtataaatattcaatacaatccggtgcgtaagccatggctcaagccttttatagagaggttctttggggtgatgaatgaatacttcttgccggaactcccaggaaaaacattttctaacattcttgagaaagaagaatacaaaccggagaaggatgcaataatgaggttcagcacctttgttgaggagttccaccgttggattgccgacgtgtatcatcaagatagtaatagccgtgaaacgcgtattcctattaagcgatggcaacaaggattcgatgcttatcctcctttaacaatgaacgaagaagaggagacacggttttctatgctcatgagaatctctgactctaggacgctaaccaggaatgggtttaagtaccaagaattgatgtatgattctaccgccttagcagattaccgtaagcattaccctcagacaaaagagactgtgaagaagctaataaaggtcgatccagatgatatctccaagatttacgtttatctggaagagttggaaagttacttggaggttccttgcacagatccaactggatacactgatggtttatccatatatgagcataaaaccattaagaagatcaaccgagaagttattagagaatcaaaagatagccttgggttggcgaaagcgagaatggctattcatgagagagtgaaacaagagcaagaagtattcatcgaaagtaagaccaaagctaagattactgcagtcaagaagcaggctcagatcgcggatgttagtaatacaggcacatcaacaatcaaggtgtccgaggagtctgcggctcctgtccagaaacacatctcaaatgataacagtgatgattgggatgatgatctggaagcttttgaatga

SEQ ID NO: 34 - DNA, 993 bp, synthetic construct
  source 1..993: mol_type = other DNA
atgagtgaaacacgagaggctaggataagccgggccaaacgggcttttgtttctacaccgtctgtacgcaagatcctgagctacatggatagatgtagggacttatcagatctcgaatcagagccaacttgtatgatggtttatggtgcctctggtgtgggaaaaaccacggtcatcaagaagtatttgaatcaaaacagaagagaatctgaggcgggtggtgatattatcccagtgttacacattgagctccctgataatgcaaagcctgttgatgccgcacgggagctgttggttgagatgggggatccactggccctctatgaaactgatttagcaagacttactaagagattgactgagctgatccctgctgttggggtgaagcttataatcattgatgaatttcagcatcttgtggaagagagatccaatagagtgttaactcaagttggaaactggcttaagatgatcttaaacaaaaccaaatgccctattgtaatatttggaatgccttactcaaaggttgttcttcaagccaattcgcagcttcacggaaggttttcaatacaagtcgaactacgtcccttctcttaccaaggaggaagaggagttttcaagacattcttggagtatcttgataaagctttacctttcgagaagcaagcaggcttagcaaatgaaagtcttcagaaaaaactgtatgctttctctcagggcaacatgagatccttaaggaatctcatatatcaggcaagtattgaagctattgacaaccaacatgaaaccattacagaggaggattttgtgtttgcttcaaaattgacaagcggagacaaaccaaattcatggaaaaatccttttgaagaaggtgttgaagtgacagaagacatgcttcgtccaccacccaaagatattggatgggaagattacctaaggcattctactccccgtgtatccaagccgggcagaaacaagaatttcttcgaatga

SEQ ID NO: 35 - DNA, 1002 bp, synthetic construct
  source 1..1002: mol_type = other DNA
atgggaaatgctcttacagagattcaaatagagaagttacggaatttcagtgattgcatcgtgatgcatcctcagataaagaccattttcaatgattttgatgaactaaggcttaatagaaagtttcagagtgatcagcaatgtatgcttcttataggagatactggtgttggcaaatcacacaccatcaaccattacaagaaaagagtcctcgcaacacaaaactattcaagaaacacaatgccggtccttgtttctcgcatatcacgtgggaaaggtttggatgctactttggtgcagatgctggcggatctagagctgtttggttcttctcagatcaagaagcggggctacaaaacagaccttaccaaaaaattagttgagagtttgatcaaagctcaagtggagctgttgattatcaatgaattccaagagcttattgagtttaaaagtgttcaagaaagacaacagattgcaaatggtctaaagtttattagtgaagaagccaaggttcccattgttttggttggaatgccatgggcagcaaagatagctgaggaaccacaatgggcttctcgactcgtaagaaaacgaaagctagaatatttttcactcaagaatgacagcaagtactttaggcagtatttgatgggtcttgccaaaaagatgcctttcgacgttcctcctaaattagaatcgaagaacactacgattgcgctatttgcagcttgtagaggggaaaacagagctttgaagcaccttcttttagaagcactgaagctcgccctttcatgcaatgagtatcttgagaacaagcatttcatcacagcttatgacaaattcgatttcttcaacgacaaggagaaactgaaatccaaaaaccctttcaagcaagatataaaagatattgaaatttatgaagttattaaaaattcttcctacaatccaaatgcacttgatccagaagatatgttgactgatcgagtatttgctattgtgaaa

SEQ ID NO: 36 - DNA, 624 bp, synthetic construct
  source 1..624: mol_type = other DNA
atgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactga

SEQ ID NO: 37 - DNA, 1275 bp, synthetic construct
  source 1..1275: mol_type = other DNA
atgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatga

SEQ ID NO: 38 - DNA, 29807 bp, synthetic construct
  source 1..29807: mol_type = other DNA
tggcaggatatattgtggtgtaaacataacgaattgctcttcagcaaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgttgatacaaccataaaatgataattacacccataaattgataattatcacacccataaattgatattgcctcttcatggtctaaacttcagtaagtttacgacattttcatgaagaggtcatttcatgacaagcttacctacgccctcagcaattacgacttcggcgttagagtatgcattccatactcccgctcgcaatctaacgaaatctcgcggaaaaaatattcatcgttatgtcagtgtaaagcgctagcaaggagtgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacggccgcgtatttttacaacaattaccaacaacaacaaacaacaaacaacattacaattactatttacaattacagcggccgcccccttcaccaaaaaaatggctgcagccaagggcgaggagctgttcaccggggtggtgcccatcctggtcgagctggacggcgacgtaaacggccacaagttcagcgtgtccggcgagggcgagggcgatgccacctacggcaagctgaccctgaagctgctctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgttcaaagaccccaacgagaagcgcgatcacatggtcttgttagaatttttgactgctgctggtattaccgagggtatgaatgaattgtacaaaggctccggaccgaagaagaagaggaaggtttaaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagagtgtccgtgactgatgcaaggttatcggaactgtaatgattatatgttttctaattttgaagtggttcttttgatgagttttgccagttactgaaagtaactttcttactgcagtagttttgctgaaatactcgattcacaaaaatatcaacttatggttgttttgtgagatatcaatatatggttgttttgtggttaagttgctgattataaataattattaaatatcactttatggttgcatcaacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatgtcattactactgcaaagtagctgataacttcctctatataaggaagttcatttcatttggctttactgctgaataagtagataactacgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatggcgacatcccttccaacgccatcagctataactacttcggcactagagtatgctttccatactccggctcgcaatttgacgaagtctagggggaagaacattcatcgatatgttagtgtcaagatgtctaaaagaattactgtagaatcaacactagaatgcgatgcttgctatcattttgacttcgagccttctatcgtgagattttgtgctcagcctattcgctttctgtattatttaaatgggcagtcacactcctatgtcccagattttcttgtgcaattcgacacaaacgagtttgtcttgtatgaagttaaaagcgcttatgcgaaaaacaaacctgatttcgatgttgagtgggaagcaaaggtgaaggccgcaacagagctgggtttggaacttgaacttgttgaggagagcgatataagagataccgttgttttgaacaatcttaagaggatgcatagatatgcgagtaaggacgagcttaataacgtacacaattccctacttaagatcataaagtataacggagctcaatccgcccgatgtttgggtgaacagctagggttgaaaggtcgtacggtacttcctattctgtgtgatcttctttcaagatgcttgctcgacaccagacttgataagcccctctctctggaaagtcgttttgaactcgcctcatatggaggatcaggatctggcaaaagaaccgcagatggttctgagttcgaaagcccaaagaaaaagagaaaggtgggttccggttctggaggagccaagaagggtttttctagctttcacaggaaggcagtttcctcacaggacactttagagagtatagaactagtttccagtgcgaattgcctcgaatcagtcacttatcaagatatatctgcatttcctgaaaccattgcagtggagatcaacttcagattgtctattctccggtttctcgcaagaaaatgtgagactatcgttgctaaatctattgagccccatagagttgagcttcaacagaattattctcggaagattccatctgcaataacaatatacagatggtggttggcctttcggaagtcagattacaacccgattagtctggctcctaatatcaaagacagagggaacagagaaactaaagtatctactgtggttgactccatcatggaacaagccgttgaaagggttatctctggtcgtaaagtcaatgtttctagcgcgtacaaacgagtccggaggaaagttagacaatataatcttactcatggaacaaagtacacatatccaaaatacgagagcgtacgaaagcgcgtaaagaagaaaacaccgtttgagttattagctgctggtaagggagagagggtcgccaagcgtgaatttaggagaatgggaaagaagattctgacgtcaagtgttctcgagagggtggagatagatcacactgttgtggatctgttcgctgttcatgaggaatataggattcctctcgggaggccatggctgactcagttagttgattgttacagcaaagcagtcattggtttctaccttggctttgaacctcctagttatgtctccgtatcactagctttaaagaatgccattcaacgcaaagatgatttgatttcctcttacgagtcaatagaaaatgaatggttatgttatggtataccagatctccttgttacagacaatggaaaagaatttttgtccaaagcttttgatcaagcttgtgagagcttattaattaacgtgcatcagaataaggtagaaaccccagataacaagcctcatgttgaaagaaactacgggacaataaatacttccttattggacgatttacctggtaaaagtttctctcagtacttgcagagggaaggatatgactctgtgggagaagctacattgactctcaatgagatcagagaaatctacttgatttggcttgtcgacatatatcacaagaaaccgaatcaaagaggaactaactgtccaaatgttgcttggaaaaaaggttgccaagaatgggagccagaagaattctccgggtcaaaagatgagcttgattttaaattcgcaattgttgattataaacaacttacaaaagtaggaatcactgtttacaaggaactaagctactcaaacgatcgtttggcagagtatagaggcaaaaaaggaaaccacaaagttcagttcaagtacaatccggaatgtatggctgtgatatgggttcttgatgaagatatgaacgaatatttcacagttaatgctatcgactatgaatacgcatctagggtgtcactgtggcaacataaatacaacatgaagtatcaagcagagctaaattctgcggaatatgacgaagataaagagattgatgcagaaattaaaatcgaggaaattgctgatagatctatagtgaaaaccaataaaattagagctcgtcgtcgtggtgctaggcatcaagagaatagtgcacgagctaaatcaatctctaatgcaaatcctgcaagtattcagaagcatgaagacgaaatagtcagtgctgataatgatgattgggatatcgattacgtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagtgaaacacgagaggctaggataagccgggccaaacgggcttttgtttctacaccgtctgtacgcaagatcctgagctacatggatagatgtagggacttatcagatctcgaatcagagccaacttgtatgatggtttatggtgcctctggtgtgggaaaaaccacggtcatcaagaagtatttgaatcaaaacagaagagaatctgaggcgggtggtgatattatcccagtgttacacattgagctccctgataatgcaaagcctgttgatgccgcacgggagctgttggttgagatgggggatccactggccctctatgaaactgatttagcaagacttactaagagattgactgagctgatccctgctgttggggtgaagcttataatcattgatgaatttcagcatcttgtggaagagagatccaatagagtgttaactcaagttggaaactggcttaagatgatcttaaacaaaaccaaatgccctattgtaatatttggaatgccttactcaaaggttgttcttcaagccaattcgcagcttcacggaaggttttcaatacaagtcgaactacgtcccttctcttaccaaggaggaagaggagttttcaagacattcttggagtatcttgataaagctttacctttcgagaagcaagcaggcttagcaaatgaaagtcttcagaaaaaactgtatgctttctctcagggcaacatgagatccttaaggaatctcatatatcaggcaagtattgaagctattgacaaccaacatgaaaccattacagaggaggattttgtgtttgcttcaaaattgacaagcggagacaaaccaaattcatggaaaaatccttttgaagaaggtgttgaagtgacagaagacatgcttcgtccaccacccaaagatattggatgggaagattacctaaggcattctactccccgtgtatccaagccgggcagaaacaagaatttcttcgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgtttcttcaaagacctaaaccgtatagcgacgaaagtctggaatctttcttcatccgtgttgccaacaagaatggttatggagatgttcacaggtttttggaagccacaaagcgattccttcaagacattgatcacaatggataccaaacatttcccactgatataacgcgtatcaatccttattcagccaaaaactcttcatcagcaagaacagcgagtttcttgaagctagcacagctcaccttcaacgaaccaccagaattgctagggcttgcaataaacagaaccaacatgaagtactctccttctactagtgcagtggtaagaggagcagaggttttcccaagatccctcctgcgtactcattccattccatgttgccctctctgcttgagggaaaatgggtatgctagttacctttggcattttcaaggatacgagtattgtcactctcataatgtccctctaattaccacttgttcttgtggtaaagaattcgattacagagtatctgggttaaaaggaatttgttgtaagtgcaaagagccaataactttgacatcgagagagaatggccatgaagcagcttgtactgttagtaattggcttgccggtcacgagtcaaaaccattgcctaatttacccaaatcttatagatggggactcgtccattggtggatgggaatcaaggactctgaatttgatcatttcagctttgtgcagtttttctcaaactggcctcggtcgtttcatagtatcatagaagatgaagttgaatttaatcttgaacatgctgttgtttctacatctgagctaaggctcaaagatcttcttgggagactgttctttggtagcatcaggcttcctgagagaaatctgcaacataacattattttgggtgagctgttatgttatctagaaaaccgtttgtggcaagataaaggcttgatagccaatttaaagatgaatgctcttgaggcaacagtgatgcttaactgctccttggatcagattgcttcaatggtggagcaaagaattttaaagcctaaccgaaaatcaaagccaaattcaccgcttgatgtgacggattatttgtttcactttggtgacattttctgcttatggttagctgagtttcagtctgatgaattcaatcgaagcttttatgtttctaggtggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtgtgaaatggtactacaaaacaataacatttctacctgagctctgcaacaatgaatcactagcagcgaaatgtctgcgtgtacttcatggattcaactaccaatatgaaacaagaaacattggtgttagcttccctctttggtgtgacgcaactgtcggaaagaagatatcctttgtgtccaagaacaagattgagcttgacttgcttctcaaacaacattactttgttcaaatggagcagcttcagtatttccacatctcaaatactgtgctggttccagaagattgcacttatgtcagtttcaggagatgtcaaagtattgataagcttactgctgctggtttagcaagaaagatccgtcgattggagaagagagctttgtcaagaggagaacagtttgatccctcaagttttgctcagaaagaacacaccgccattgctcattatcattctctcggtgaatcttcaaagcaaacaaataggaattttcggttaaatataaggatgttgagtgaacaaccaagagaaggaaattccatcttctcttcttatgggctatcaaattctgagaattcttttcagcctgttccgctaatctgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgaaacttccaacaaatctagcttacgagaggtcgattgatccttcagatgtttgtttctttgtggtctggccagatgatcggaaaacaccgttgacatacaattcacgtaccctcctagggcagatggaggctgcatctctggcgtatgatgtaagtggtcaaccgatcaagagtgccacagcagaagcattagctcaaggaaatcctcatcaagttgacttttgtcatgttccttatggagcatctcacatcgagtgcagcttctctgtttctttttcatcagagcttcgccagccttacaaatgtaacagcagtaaagtgaagcaaactctcgttcagctcgtagagttgtatgaaactaagattggatggactgaactagcgacgagatatttgatgaacatatgcaatggtaaatggctctggaaaaatacaaggaaggcttattgttggaatatagtcctgactccatggccttggaacggagagaaggttggctttgaagacattaggacaaactacacttcaagacaagatttcaagaacaacaagaattggtctgccattgttgagatgattaagacagctttcagttctactgatggcttagcgatcttcgaggttcgtgcgactcttcacttaccaaccaatgctatggttagaccttcccaggtattcacagagaaagaatcagggtcgaaatcaaaaagcaagactcagaattctcgagtgtttcaatcaacaaccatagatggtgaaagatctcccattcttggtgcattcaaaactggtgcagctatagctactattgacgactggtatcctgaagctacagaaccattgcgtgttggaagatttggtgtccatagagaagatgtgacttgctatagacatccttccacagggaaagattttttctccatacttcaacaagctgaacattacattgaagtactttcagcaaacaaaaccccagcacaagaaaccattaatgatatgcattttcttatggctaatctcatcaaaggtggcatgtttcagcacaaaggagactgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagactcttaaagaactaattgcttctaatccggatgacctaacaacggagctcaaaagggcatttcgccccctgactcctcacattgccattgacggtaacgagcttgacgctctcaccattcttgtgaatctcacagacaaaactgatgatcaaaaggatcttctcgatagagctaaatgtaaacagaaactcagagatgagaaatggtgggcttcttgcatcaattgcgttaattatagacagtctcacaatcctaaattccctgatatcaggtctgaaggagtgattcgcactcaagctttgggcgaacttccttcatttctattaagctccagtaaaataccaccgtatcattggtcatattctcatgattcaaaatatgttaacaagtcagcctttttaacaaatgaattctgttgggacggagagataagctgcttgggggaactcctcaaagatgcggatcatccactttggaacacactaaagaagcttggctgctcacagaagacatgcaaagctatggcgaaacaattggctgatatcacattaacaacgattaatgtaacactcgctccgaattacttgactcagatttctcttccggattctgatacaagttacatttctctctcgcctgtcgcaagtctatcaatgcaaagtcatttccaccaacgtttgcaagacgaaaacagacattctgctataacaagattctcccgaacaactaacatgggtgtgactgcaatgacttgtggtggagctttcaggatgttaaagtctggtgcaaagttctccagtccacctcatcatagattaaactcgaaacgctcttggttgaccagtgagcatgttcaatcattgaagcagtatcaacgtctgaacaaatcattgatccctgagaatagtaggattgctcttaggcgcaagtacaagattgaacttcaaaatatggttcgatcatggtttgccatgcaagatcacactctcgattcaaatatccttattcaacatctcaatcacgatttgagttatctcggggcgaccaaaagatttgcttatgatccagctatgacaaaattgttcactgagctgctaaagagggagctgagtaattctatcaacaacggagaacaacataccaatggcagttttctggtcctcccaaacatacgggtttgtggagcaactgcgttatcttccccagtaaccgttggcatccctagcttaaccgccttctttggttttgttcacgcctttgagaggaacatcaacaggaccacttcttcctttcgtgtcgagagctttgcaatatgtgtccatcagcttcatgtggagaagcgtggtttgacagctgagtttgtagagaagggagatgggacaatttcagcaccagcaaccagagatgattggcaatgtgatgttgtgttttctctaattttgaatacgaacttcgctcaacacattgatcaggacacactggtcacgagtttacctaagcggctggcaaggggttctgctaagatcgcaatagatgatttcaagcatataaactcattctccactcttgaaactgcaattgagtctcttcccatcgaagctggaagatggcttagtctgtatgcacagagtaataataatctttcggacttgttagctgctatgaccgaagaccatcaacttatggctagttgtgttggctaccaccttctagaagaaccaaaagataagccaaatagccttagaggatacaaacatgccatagctgaatgtattataggattaattaattcaattacattttcctcggaaaccgatcctaatactatattctggtccttaaagaactaccaaaactatcttgtagttcagcccagatccataaatgatgaaacaacagacaagagcagcctgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgaataggcgtatcacgaggccctttcgtcttcaagaattagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcgggatcgaagatccattccaagcctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgtgtctcgcccttcagcacgccgtcgcgggggtacaggcgctcggtggaggcctcccagcccatcgtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatttcgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataccgtgactcccttaattctcatgtatggttcgctatactgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 39 - DNA, 29807 bp, synthetic construct
  source 1..29807: mol_type = other DNA
tggcaggatatattgtggtgtaaacataacgaattgctcttcagcaaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgttgatacaaccataaaatgataattacacccataaattgataattatcacacccataaattgatattgcctcttcatggtctaaacttcagtaagtttacgacattttcatgaagaggtcatttcatgacaagcttacctacgccctcagcaattacgacttcggcgttagagtatgcattccatactcccgctcgcaatctaacgaaatctcgcggaaaaaatattcatcgttatgtcagtgtaaagcgctagcaaggagtgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacggccgcgtatttttacaacaattaccaacaacaacaaacaacaaacaacattacaattactatttacaattacagcggccgcccccttcaccaaaaaaatggctgcagccaagggcgaggagctgttcaccggggtggtgcccatcctggtcgagctggacggcgacgtaaacggccacaagttcagcgtgtccggcgagggcgagggcgatgccacctacggcaagctgaccctgaagctgctctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgttcaaagaccccaacgagaagcgcgatcacatggtcttgttagaatttttgactgctgctggtattaccgagggtatgaatgaattgtacaaaggctccggaccgaagaagaagaggaaggtttaaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagagtgtccgtgactgatgcaaggttatcggaactgtaatgattatatgttttctaattttgaagtggttcttttgatgagttttgccagttactgaaagtaactttcttactgcagtagttttgctgaaatactcgattcacaaaaatatcaacttatggttgttttgtgagatatcaatatatggttgttttgtggttaagttgctgattataaataattattaaatatcactttatggttgcatcaacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatgtcattactactgcaaagtagctgataacactgacgtaagggatgacgcacaatcccactactttactgctgaataagtagataactacgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatggcgacatcccttccaacgccatcagctataactacttcggcactagagtatgctttccatactccggctcgcaatttgacgaagtctagggggaagaacattcatcgatatgttagtgtcaagatgtctaaaagaattactgtagaatcaacactagaatgcgatgcttgctatcattttgacttcgagccttctatcgtgagattttgtgctcagcctattcgctttctgtattatttaaatgggcagtcacactcctatgtcccagattttcttgtgcaattcgacacaaacgagtttgtcttgtatgaagttaaaagcgcttatgcgaaaaacaaacctgatttcgatgttgagtgggaagcaaaggtgaaggccgcaacagagctgggtttggaacttgaacttgttgaggagagcgatataagagataccgttgttttgaacaatcttaagaggatgcatagatatgcgagtaaggacgagcttaataacgtacacaattccctacttaagatcataaagtataacggagctcaatccgcccgatgtttgggtgaacagctagggttgaaaggtcgtacggtacttcctattctgtgtgatcttctttcaagatgcttgctcgacaccagacttgataagcccctctctctggaaagtcgttttgaactcgcctcatatggaggatcaggatctggcaaaagaaccgcagatggttctgagttcgaaagcccaaagaaaaagagaaaggtgggttccggttctggaggagccaagaagggtttttctagctttcacaggaaggcagtttcctcacaggacactttagagagtatagaactagtttccagtgcgaattgcctcgaatcagtcacttatcaagatatatctgcatttcctgaaaccattgcagtggagatcaacttcagattgtctattctccggtttctcgcaagaaaatgtgagactatcgttgctaaatctattgagccccatagagttgagcttcaacagaattattctcggaagattccatctgcaataacaatatacagatggtggttggcctttcggaagtcagattacaacccgattagtctggctcctaatatcaaagacagagggaacagagaaactaaagtatctactgtggttgactccatcatggaacaagccgttgaaagggttatctctggtcgtaaagtcaatgtttctagcgcgtacaaacgagtccggaggaaagttagacaatataatcttactcatggaacaaagtacacatatccaaaatacgagagcgtacgaaagcgcgtaaagaagaaaacaccgtttgagttattagctgctggtaagggagagagggtcgccaagcgtgaatttaggagaatgggaaagaagattctgacgtcaagtgttctcgagagggtggagatagatcacactgttgtggatctgttcgctgttcatgaggaatataggattcctctcgggaggccatggctgactcagttagttgattgttacagcaaagcagtcattggtttctaccttggctttgaacctcctagttatgtctccgtatcactagctttaaagaatgccattcaacgcaaagatgatttgatttcctcttacgagtcaatagaaaatgaatggttatgttatggtataccagatctccttgttacagacaatggaaaagaatttttgtccaaagcttttgatcaagcttgtgagagcttattaattaacgtgcatcagaataaggtagaaaccccagataacaagcctcatgttgaaagaaactacgggacaataaatacttccttattggacgatttacctggtaaaagtttctctcagtacttgcagagggaaggatatgactctgtgggagaagctacattgactctcaatgagatcagagaaatctacttgatttggcttgtcgacatatatcacaagaaaccgaatcaaagaggaactaactgtccaaatgttgcttggaaaaaaggttgccaagaatgggagccagaagaattctccgggtcaaaagatgagcttgattttaaattcgcaattgttgattataaacaacttacaaaagtaggaatcactgtttacaaggaactaagctactcaaacgatcgtttggcagagtatagaggcaaaaaaggaaaccacaaagttcagttcaagtacaatccggaatgtatggctgtgatatgggttcttgatgaagatatgaacgaatatttcacagttaatgctatcgactatgaatacgcatctagggtgtcactgtggcaacataaatacaacatgaagtatcaagcagagctaaattctgcggaatatgacgaagataaagagattgatgcagaaattaaaatcgaggaaattgctgatagatctatagtgaaaaccaataaaattagagctcgtcgtcgtggtgctaggcatcaagagaatagtgcacgagctaaatcaatctctaatgcaaatcctgcaagtattcagaagcatgaagacgaaatagtcagtgctgataatgatgattgggatatcgattacgtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagtgaaacacgagaggctaggataagccgggccaaacgggcttttgtttctacaccgtctgtacgcaagatcctgagctacatggatagatgtagggacttatcagatctcgaatcagagccaacttgtatgatggtttatggtgcctctggtgtgggaaaaaccacggtcatcaagaagtatttgaatcaaaacagaagagaatctgaggcgggtggtgatattatcccagtgttacacattgagctccctgataatgcaaagcctgttgatgccgcacgggagctgttggttgagatgggggatccactggccctctatgaaactgatttagcaagacttactaagagattgactgagctgatccctgctgttggggtgaagcttataatcattgatgaatttcagcatcttgtggaagagagatccaatagagtgttaactcaagttggaaactggcttaagatgatcttaaacaaaaccaaatgccctattgtaatatttggaatgccttactcaaaggttgttcttcaagccaattcgcagcttcacggaaggttttcaatacaagtcgaactacgtcccttctcttaccaaggaggaagaggagttttcaagacattcttggagtatcttgataaagctttacctttcgagaagcaagcaggcttagcaaatgaaagtcttcagaaaaaactgtatgctttctctcagggcaacatgagatccttaaggaatctcatatatcaggcaagtattgaagctattgacaaccaacatgaaaccattacagaggaggattttgtgtttgcttcaaaattgacaagcggagacaaaccaaattcatggaaaaatccttttgaagaaggtgttgaagtgacagaagacatgcttcgtccaccacccaaagatattggatgggaagattacctaaggcattctactccccgtgtatccaagccgggcagaaacaagaatttcttcgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgtttcttcaaagacctaaaccgtatagcgacgaaagtctggaatctttcttcatccgtgttgccaacaagaatggttatggagatgttcacaggtttttggaagccacaaagcgattccttcaagacattgatcacaatggataccaaacatttcccactgatataacgcgtatcaatccttattcagccaaaaactcttcatcagcaagaacagcgagtttcttgaagctagcacagctcaccttcaacgaaccaccagaattgctagggcttgcaataaacagaaccaacatgaagtactctccttctactagtgcagtggtaagaggagcagaggttttcccaagatccctcctgcgtactcattccattccatgttgccctctctgcttgagggaaaatgggtatgctagttacctttggcattttcaaggatacgagtattgtcactctcataatgtccctctaattaccacttgttcttgtggtaaagaattcgattacagagtatctgggttaaaaggaatttgttgtaagtgcaaagagccaataactttgacatcgagagagaatggccatgaagcagcttgtactgttagtaattggcttgccggtcacgagtcaaaaccattgcctaatttacccaaatcttatagatggggactcgtccattggtggatgggaatcaaggactctgaatttgatcatttcagctttgtgcagtttttctcaaactggcctcggtcgtttcatagtatcatagaagatgaagttgaatttaatcttgaacatgctgttgtttctacatctgagctaaggctcaaagatcttcttgggagactgttctttggtagcatcaggcttcctgagagaaatctgcaacataacattattttgggtgagctgttatgttatctagaaaaccgtttgtggcaagataaaggcttgatagccaatttaaagatgaatgctcttgaggcaacagtgatgcttaactgctccttggatcagattgcttcaatggtggagcaaagaattttaaagcctaaccgaaaatcaaagccaaattcaccgcttgatgtgacggattatttgtttcactttggtgacattttctgcttatggttagctgagtttcagtctgatgaattcaatcgaagcttttatgtttctaggtggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtgtgaaatggtactacaaaacaataacatttctacctgagctctgcaacaatgaatcactagcagcgaaatgtctgcgtgtacttcatggattcaactaccaatatgaaacaagaaacattggtgttagcttccctctttggtgtgacgcaactgtcggaaagaagatatcctttgtgtccaagaacaagattgagcttgacttgcttctcaaacaacattactttgttcaaatggagcagcttcagtatttccacatctcaaatactgtgctggttccagaagattgcacttatgtcagtttcaggagatgtcaaagtattgataagcttactgctgctggtttagcaagaaagatccgtcgattggagaagagagctttgtcaagaggagaacagtttgatccctcaagttttgctcagaaagaacacaccgccattgctcattatcattctctcggtgaatcttcaaagcaaacaaataggaattttcggttaaatataaggatgttgagtgaacaaccaagagaaggaaattccatcttctcttcttatgggctatcaaattctgagaattcttttcagcctgttccgctaatctgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgaaacttccaacaaatctagcttacgagaggtcgattgatccttcagatgtttgtttctttgtggtctggccagatgatcggaaaacaccgttgacatacaattcacgtaccctcctagggcagatggaggctgcatctctggcgtatgatgtaagtggtcaaccgatcaagagtgccacagcagaagcattagctcaaggaaatcctcatcaagttgacttttgtcatgttccttatggagcatctcacatcgagtgcagcttctctgtttctttttcatcagagcttcgccagccttacaaatgtaacagcagtaaagtgaagcaaactctcgttcagctcgtagagttgtatgaaactaagattggatggactgaactagcgacgagatatttgatgaacatatgcaatggtaaatggctctggaaaaatacaaggaaggcttattgttggaatatagtcctgactccatggccttggaacggagagaaggttggctttgaagacattaggacaaactacacttcaagacaagatttcaagaacaacaagaattggtctgccattgttgagatgattaagacagctttcagttctactgatggcttagcgatcttcgaggttcgtgcgactcttcacttaccaaccaatgctatggttagaccttcccaggtattcacagagaaagaatcagggtcgaaatcaaaaagcaagactcagaattctcgagtgtttcaatcaacaaccatagatggtgaaagatctcccattcttggtgcattcaaaactggtgcagctatagctactattgacgactggtatcctgaagctacagaaccattgcgtgttggaagatttggtgtccatagagaagatgtgacttgctatagacatccttccacagggaaagattttttctccatacttcaacaagctgaacattacattgaagtactttcagcaaacaaaaccccagcacaagaaaccattaatgatatgcattttcttatggctaatctcatcaaaggtggcatgtttcagcacaaaggagactgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagactcttaaagaactaattgcttctaatccggatgacctaacaacggagctcaaaagggcatttcgccccctgactcctcacattgccattgacggtaacgagcttgacgctctcaccattcttgtgaatctcacagacaaaactgatgatcaaaaggatcttctcgatagagctaaatgtaaacagaaactcagagatgagaaatggtgggcttcttgcatcaattgcgttaattatagacagtctcacaatcctaaattccctgatatcaggtctgaaggagtgattcgcactcaagctttgggcgaacttccttcatttctattaagctccagtaaaataccaccgtatcattggtcatattctcatgattcaaaatatgttaacaagtcagcctttttaacaaatgaattctgttgggacggagagataagctgcttgggggaactcctcaaagatgcggatcatccactttggaacacactaaagaagcttggctgctcacagaagacatgcaaagctatggcgaaacaattggctgatatcacattaacaacgattaatgtaacactcgctccgaattacttgactcagatttctcttccggattctgatacaagttacatttctctctcgcctgtcgcaagtctatcaatgcaaagtcatttccaccaacgtttgcaagacgaaaacagacattctgctataacaagattctcccgaacaactaacatgggtgtgactgcaatgacttgtggtggagctttcaggatgttaaagtctggtgcaaagttctccagtccacctcatcatagattaaactcgaaacgctcttggttgaccagtgagcatgttcaatcattgaagcagtatcaacgtctgaacaaatcattgatccctgagaatagtaggattgctcttaggcgcaagtacaagattgaacttcaaaatatggttcgatcatggtttgccatgcaagatcacactctcgattcaaatatccttattcaacatctcaatcacgatttgagttatctcggggcgaccaaaagatttgcttatgatccagctatgacaaaattgttcactgagctgctaaagagggagctgagtaattctatcaacaacggagaacaacataccaatggcagttttctggtcctcccaaacatacgggtttgtggagcaactgcgttatcttccccagtaaccgttggcatccctagcttaaccgccttctttggttttgttcacgcctttgagaggaacatcaacaggaccacttcttcctttcgtgtcgagagctttgcaatatgtgtccatcagcttcatgtggagaagcgtggtttgacagctgagtttgtagagaagggagatgggacaatttcagcaccagcaaccagagatgattggcaatgtgatgttgtgttttctctaattttgaatacgaacttcgctcaacacattgatcaggacacactggtcacgagtttacctaagcggctggcaaggggttctgctaagatcgcaatagatgatttcaagcatataaactcattctccactcttgaaactgcaattgagtctcttcccatcgaagctggaagatggcttagtctgtatgcacagagtaataataatctttcggacttgttagctgctatgaccgaagaccatcaacttatggctagttgtgttggctaccaccttctagaagaaccaaaagataagccaaatagccttagaggatacaaacatgccatagctgaatgtattataggattaattaattcaattacattttcctcggaaaccgatcctaatactatattctggtccttaaagaactaccaaaactatcttgtagttcagcccagatccataaatgatgaaacaacagacaagagcagcctgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgaataggcgtatcacgaggccctttcgtcttcaagaattagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcgggatcgaagatccattccaagcctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgtgtctcgcccttcagcacgccgtcgcgggggtacaggcgctcggtggaggcctcccagcccatcgtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatttcgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataccgtgactcccttaattctcatgtatggttcgctatactgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 40 - DNA, 29807 bp, synthetic construct
  source 1..29807: mol_type = other DNA
tggcaggatatattgtggtgtaaacataacgaattgctcttcagcaaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgttgatacaaccataaaatgataattacacccataaattgataattatcacacccataaattgatattgcctcttcatggtctaaacttcagtaagtttacgacattttcatgaagaggtcatttcatgacaagcttacctacgccctcagcaattacgacttcggcgttagagtatgcattccatactcccgctcgcaatctaacgaaatctcgcggaaaaaatattcatcgttatgtcagtgtaaagcgctagcaaggagtgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacggccgcgtatttttacaacaattaccaacaacaacaaacaacaaacaacattacaattactatttacaattacagcggccgcccccttcaccaaaaaaatggctgcagccaagggcgaggagctgttcaccggggtggtgcccatcctggtcgagctggacggcgacgtaaacggccacaagttcagcgtgtccggcgagggcgagggcgatgccacctacggcaagctgaccctgaagctgctctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgttcaaagaccccaacgagaagcgcgatcacatggtcttgttagaatttttgactgctgctggtattaccgagggtatgaatgaattgtacaaaggctccggaccgaagaagaagaggaaggtttaaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagagtgtccgtgactgatgcaaggttatcggaactgtaatgattatatgttttctaattttgaagtggttcttttgatgagttttgccagttactgaaagtaactttcttactgcagtagttttgctgaaatactcgattcacaaaaatatcaacttatggttgttttgtgagatatcaatatatggttgttttgtggttaagttgctgattataaataattattaaatatcactttatggttgcatcaacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatgtcattactactgcaaagtagctgataacatcccactatccttcgcaagacccttcctctactttactgctgaataagtagataactacgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatggcgacatcccttccaacgccatcagctataactacttcggcactagagtatgctttccatactccggctcgcaatttgacgaagtctagggggaagaacattcatcgatatgttagtgtcaagatgtctaaaagaattactgtagaatcaacactagaatgcgatgcttgctatcattttgacttcgagccttctatcgtgagattttgtgctcagcctattcgctttctgtattatttaaatgggcagtcacactcctatgtcccagattttcttgtgcaattcgacacaaacgagtttgtcttgtatgaagttaaaagcgcttatgcgaaaaacaaacctgatttcgatgttgagtgggaagcaaaggtgaaggccgcaacagagctgggtttggaacttgaacttgttgaggagagcgatataagagataccgttgttttgaacaatcttaagaggatgcatagatatgcgagtaaggacgagcttaataacgtacacaattccctacttaagatcataaagtataacggagctcaatccgcccgatgtttgggtgaacagctagggttgaaaggtcgtacggtacttcctattctgtgtgatcttctttcaagatgcttgctcgacaccagacttgataagcccctctctctggaaagtcgttttgaactcgcctcatatggaggatcaggatctggcaaaagaaccgcagatggttctgagttcgaaagcccaaagaaaaagagaaaggtgggttccggttctggaggagccaagaagggtttttctagctttcacaggaaggcagtttcctcacaggacactttagagagtatagaactagtttccagtgcgaattgcctcgaatcagtcacttatcaagatatatctgcatttcctgaaaccattgcagtggagatcaacttcagattgtctattctccggtttctcgcaagaaaatgtgagactatcgttgctaaatctattgagccccatagagttgagcttcaacagaattattctcggaagattccatctgcaataacaatatacagatggtggttggcctttcggaagtcagattacaacccgattagtctggctcctaatatcaaagacagagggaacagagaaactaaagtatctactgtggttgactccatcatggaacaagccgttgaaagggttatctctggtcgtaaagtcaatgtttctagcgcgtacaaacgagtccggaggaaagttagacaatataatcttactcatggaacaaagtacacatatccaaaatacgagagcgtacgaaagcgcgtaaagaagaaaacaccgtttgagttattagctgctggtaagggagagagggtcgccaagcgtgaatttaggagaatgggaaagaagattctgacgtcaagtgttctcgagagggtggagatagatcacactgttgtggatctgttcgctgttcatgaggaatataggattcctctcgggaggccatggctgactcagttagttgattgttacagcaaagcagtcattggtttctaccttggctttgaacctcctagttatgtctccgtatcactagctttaaagaatgccattcaacgcaaagatgatttgatttcctcttacgagtcaatagaaaatgaatggttatgttatggtataccagatctccttgttacagacaatggaaaagaatttttgtccaaagcttttgatcaagcttgtgagagcttattaattaacgtgcatcagaataaggtagaaaccccagataacaagcctcatgttgaaagaaactacgggacaataaatacttccttattggacgatttacctggtaaaagtttctctcagtacttgcagagggaaggatatgactctgtgggagaagctacattgactctcaatgagatcagagaaatctacttgatttggcttgtcgacatatatcacaagaaaccgaatcaaagaggaactaactgtccaaatgttgcttggaaaaaaggttgccaagaatgggagccagaagaattctccgggtcaaaagatgagcttgattttaaattcgcaattgttgattataaacaacttacaaaagtaggaatcactgtttacaaggaactaagctactcaaacgatcgtttggcagagtatagaggcaaaaaaggaaaccacaaagttcagttcaagtacaatccggaatgtatggctgtgatatgggttcttgatgaagatatgaacgaatatttcacagttaatgctatcgactatgaatacgcatctagggtgtcactgtggcaacataaatacaacatgaagtatcaagcagagctaaattctgcggaatatgacgaagataaagagattgatgcagaaattaaaatcgaggaaattgctgatagatctatagtgaaaaccaataaaattagagctcgtcgtcgtggtgctaggcatcaagagaatagtgcacgagctaaatcaatctctaatgcaaatcctgcaagtattcagaagcatgaagacgaaatagtcagtgctgataatgatgattgggatatcgattacgtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagtgaaacacgagaggctaggataagccgggccaaacgggcttttgtttctacaccgtctgtacgcaagatcctgagctacatggatagatgtagggacttatcagatctcgaatcagagccaacttgtatgatggtttatggtgcctctggtgtgggaaaaaccacggtcatcaagaagtatttgaatcaaaacagaagagaatctgaggcgggtggtgatattatcccagtgttacacattgagctccctgataatgcaaagcctgttgatgccgcacgggagctgttggttgagatgggggatccactggccctctatgaaactgatttagcaagacttactaagagattgactgagctgatccctgctgttggggtgaagcttataatcattgatgaatttcagcatcttgtggaagagagatccaatagagtgttaactcaagttggaaactggcttaagatgatcttaaacaaaaccaaatgccctattgtaatatttggaatgccttactcaaaggttgttcttcaagccaattcgcagcttcacggaaggttttcaatacaagtcgaactacgtcccttctcttaccaaggaggaagaggagttttcaagacattcttggagtatcttgataaagctttacctttcgagaagcaagcaggcttagcaaatgaaagtcttcagaaaaaactgtatgctttctctcagggcaacatgagatccttaaggaatctcatatatcaggcaagtattgaagctattgacaaccaacatgaaaccattacagaggaggattttgtgtttgcttcaaaattgacaagcggagacaaaccaaattcatggaaaaatccttttgaagaaggtgttgaagtgacagaagacatgcttcgtccaccacccaaagatattggatgggaagattacctaaggcattctactccccgtgtatccaagccgggcagaaacaagaatttcttcgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgtttcttcaaagacctaaaccgtatagcgacgaaagtctggaatctttcttcatccgtgttgccaacaagaatggttatggagatgttcacaggtttttggaagccacaaagcgattccttcaagacattgatcacaatggataccaaacatttcccactgatataacgcgtatcaatccttattcagccaaaaactcttcatcagcaagaacagcgagtttcttgaagctagcacagctcaccttcaacgaaccaccagaattgctagggcttgcaataaacagaaccaacatgaagtactctccttctactagtgcagtggtaagaggagcagaggttttcccaagatccctcctgcgtactcattccattccatgttgccctctctgcttgagggaaaatgggtatgctagttacctttggcattttcaaggatacgagtattgtcactctcataatgtccctctaattaccacttgttcttgtggtaaagaattcgattacagagtatctgggttaaaaggaatttgttgtaagtgcaaagagccaataactttgacatcgagagagaatggccatgaagcagcttgtactgttagtaattggcttgccggtcacgagtcaaaaccattgcctaatttacccaaatcttatagatggggactcgtccattggtggatgggaatcaaggactctgaatttgatcatttcagctttgtgcagtttttctcaaactggcctcggtcgtttcatagtatcatagaagatgaagttgaatttaatcttgaacatgctgttgtttctacatctgagctaaggctcaaagatcttcttgggagactgttctttggtagcatcaggcttcctgagagaaatctgcaacataacattattttgggtgagctgttatgttatctagaaaaccgtttgtggcaagataaaggcttgatagccaatttaaagatgaatgctcttgaggcaacagtgatgcttaactgctccttggatcagattgcttcaatggtggagcaaagaattttaaagcctaaccgaaaatcaaagccaaattcaccgcttgatgtgacggattatttgtttcactttggtgacattttctgcttatggttagctgagtttcagtctgatgaattcaatcgaagcttttatgtttctaggtggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtgtgaaatggtactacaaaacaataacatttctacctgagctctgcaacaatgaatcactagcagcgaaatgtctgcgtgtacttcatggattcaactaccaatatgaaacaagaaacattggtgttagcttccctctttggtgtgacgcaactgtcggaaagaagatatcctttgtgtccaagaacaagattgagcttgacttgcttctcaaacaacattactttgttcaaatggagcagcttcagtatttccacatctcaaatactgtgctggttccagaagattgcacttatgtcagtttcaggagatgtcaaagtattgataagcttactgctgctggtttagcaagaaagatccgtcgattggagaagagagctttgtcaagaggagaacagtttgatccctcaagttttgctcagaaagaacacaccgccattgctcattatcattctctcggtgaatcttcaaagcaaacaaataggaattttcggttaaatataaggatgttgagtgaacaaccaagagaaggaaattccatcttctcttcttatgggctatcaaattctgagaattcttttcagcctgttccgctaatctgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgaaacttccaacaaatctagcttacgagaggtcgattgatccttcagatgtttgtttctttgtggtctggccagatgatcggaaaacaccgttgacatacaattcacgtaccctcctagggcagatggaggctgcatctctggcgtatgatgtaagtggtcaaccgatcaagagtgccacagcagaagcattagctcaaggaaatcctcatcaagttgacttttgtcatgttccttatggagcatctcacatcgagtgcagcttctctgtttctttttcatcagagcttcgccagccttacaaatgtaacagcagtaaagtgaagcaaactctcgttcagctcgtagagttgtatgaaactaagattggatggactgaactagcgacgagatatttgatgaacatatgcaatggtaaatggctctggaaaaatacaaggaaggcttattgttggaatatagtcctgactccatggccttggaacggagagaaggttggctttgaagacattaggacaaactacacttcaagacaagatttcaagaacaacaagaattggtctgccattgttgagatgattaagacagctttcagttctactgatggcttagcgatcttcgaggttcgtgcgactcttcacttaccaaccaatgctatggttagaccttcccaggtattcacagagaaagaatcagggtcgaaatcaaaaagcaagactcagaattctcgagtgtttcaatcaacaaccatagatggtgaaagatctcccattcttggtgcattcaaaactggtgcagctatagctactattgacgactggtatcctgaagctacagaaccattgcgtgttggaagatttggtgtccatagagaagatgtgacttgctatagacatccttccacagggaaagattttttctccatacttcaacaagctgaacattacattgaagtactttcagcaaacaaaaccccagcacaagaaaccattaatgatatgcattttcttatggctaatctcatcaaaggtggcatgtttcagcacaaaggagactgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagactcttaaagaactaattgcttctaatccggatgacctaacaacggagctcaaaagggcatttcgccccctgactcctcacattgccattgacggtaacgagcttgacgctctcaccattcttgtgaatctcacagacaaaactgatgatcaaaaggatcttctcgatagagctaaatgtaaacagaaactcagagatgagaaatggtgggcttcttgcatcaattgcgttaattatagacagtctcacaatcctaaattccctgatatcaggtctgaaggagtgattcgcactcaagctttgggcgaacttccttcatttctattaagctccagtaaaataccaccgtatcattggtcatattctcatgattcaaaatatgttaacaagtcagcctttttaacaaatgaattctgttgggacggagagataagctgcttgggggaactcctcaaagatgcggatcatccactttggaacacactaaagaagcttggctgctcacagaagacatgcaaagctatggcgaaacaattggctgatatcacattaacaacgattaatgtaacactcgctccgaattacttgactcagatttctcttccggattctgatacaagttacatttctctctcgcctgtcgcaagtctatcaatgcaaagtcatttccaccaacgtttgcaagacgaaaacagacattctgctataacaagattctcccgaacaactaacatgggtgtgactgcaatgacttgtggtggagctttcaggatgttaaagtctggtgcaaagttctccagtccacctcatcatagattaaactcgaaacgctcttggttgaccagtgagcatgttcaatcattgaagcagtatcaacgtctgaacaaatcattgatccctgagaatagtaggattgctcttaggcgcaagtacaagattgaacttcaaaatatggttcgatcatggtttgccatgcaagatcacactctcgattcaaatatccttattcaacatctcaatcacgatttgagttatctcggggcgaccaaaagatttgcttatgatccagctatgacaaaattgttcactgagctgctaaagagggagctgagtaattctatcaacaacggagaacaacataccaatggcagttttctggtcctcccaaacatacgggtttgtggagcaactgcgttatcttccccagtaaccgttggcatccctagcttaaccgccttctttggttttgttcacgcctttgagaggaacatcaacaggaccacttcttcctttcgtgtcgagagctttgcaatatgtgtccatcagcttcatgtggagaagcgtggtttgacagctgagtttgtagagaagggagatgggacaatttcagcaccagcaaccagagatgattggcaatgtgatgttgtgttttctctaattttgaatacgaacttcgctcaacacattgatcaggacacactggtcacgagtttacctaagcggctggcaaggggttctgctaagatcgcaatagatgatttcaagcatataaactcattctccactcttgaaactgcaattgagtctcttcccatcgaagctggaagatggcttagtctgtatgcacagagtaataataatctttcggacttgttagctgctatgaccgaagaccatcaacttatggctagttgtgttggctaccaccttctagaagaaccaaaagataagccaaatagccttagaggatacaaacatgccatagctgaatgtattataggattaattaattcaattacattttcctcggaaaccgatcctaatactatattctggtccttaaagaactaccaaaactatcttgtagttcagcccagatccataaatgatgaaacaacagacaagagcagcctgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgaataggcgtatcacgaggccctttcgtcttcaagaattagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcgggatcgaagatccattccaagcctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgtgtctcgcccttcagcacgccgtcgcgggggtacaggcgctcggtggaggcctcccagcccatcgtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatttcgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataccgtgactcccttaattctcatgtatggttcgctatactgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 41 - DNA, 29999 bp, synthetic construct
  source 1..29999: mol_type = other DNA
tggcaggatatattgtggtgtaaacataacgaattgctcttcagcaaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgtcgctgcaaccatagtttgacataatgcaaccataccttgacataattaaagtcatactctgacataaagccccttattaaaaatgatgctaaaaataaattccattataatcaattatttaagaattaattaaaccatagtgtgacatagtggctttttatttgtatagtatgtgtactcaaataataaaaatttacttatgtatatacgcaacttaagaaagccatctccaaataaaaatgtatttcgctagcaaggagtgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacggccgcgtatttttacaacaattaccaacaacaacaaacaacaaacaacattacaattactatttacaattacagcggccgcccccttcaccaaaaaaatggctgcagccaagggcgaggagctgttcaccggggtggtgcccatcctggtcgagctggacggcgacgtaaacggccacaagttcagcgtgtccggcgagggcgagggcgatgccacctacggcaagctgaccctgaagctgctctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgttcaaagaccccaacgagaagcgcgatcacatggtcttgttagaatttttgactgctgctggtattaccgagggtatgaatgaattgtacaaaggctccggaccgaagaagaagaggaaggtttaaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagccgtataggcagctgaagataaaaacaatcgattcgatgtttaagttaatgtaattctgccgaaaaggcagtgagtagtaattgaaaaaaccagtcttatatccgggctggtttttttgtttttagcgttataacttttatttcactatgtcactgtatggttgcattttatgtcagtgaatggttgcaggttttttgtaagttattgatatgcctatccagtgtatgtcacgctttgcttgcagcgacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatggtgacctgccgtataggcagctgaagatttcctctatataaggaagttcatttcatttggtaattctgccgaaaaggcagtgagtagtgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgtacattaggaacttgagaaagccttctccaaataagaatgtttttaagttcgcatctaccaaggtcagctctgtggtcatgtgtgaatccagccttgagtttgacgcttgctttcatcatgaatataacgacttgattgagagttttggttctcagccagaaggtttcaaatacgaattcatgggtaaatctctcccttacactccagatgctttgatctcctacacggacaaaactcaaaagtatcacgagtataaaccatattcaaaaatcgcatcacctctatttagagcagagtttgctgcaaagagggctgcaagtcttaagttgggtattgatttggtactcgtcacagacagacagattagagtaaacccaatactcaacaatctgaagctccttcatagatactcgggcgtttatggtatatctggtatccagaaggaactacttagctttattcacaaaagtggcgttatcaaactcaatgatatctcatcacaggttgggatccccattggagaaactcggtcatttctttttggactgatgcataagggacttgtaaaggctgacctaggttgtgatgacctgacaaacaaccctactctttgggctactcctggaagtggctcaggcaaacgtactgctgacggaagtgagttcgagtccccaaagaaaaaaagaaaagttggtagtggatctggtggaatgactgatttcttcaatgagtttgatgaatcgcttgtaccactgaaaccacaaactccaacgcagtatgttaagctggacgatgctaatttaattcagagagatttagatacattctcagatactttcaagaaccaagccctccaaagatataagctaatttctacaattgataaaaagttaagccgcggatggactcaaagaaatctcgacccgatcctcgacgaactcttcaaaggcggtgatgtagtgaggccaaactggagaactgtggcaagatggaggaagaaatatatagaaagcaatggtgacatcgcttctcttgctgataaaaaccacaaaatgggaaacagaacaaatcggataaaaggagatgacaaattttttgacaaggccttggaacgtttccttgacgcgaaaagacctacaatagcaacagcttatcaatattacaaggacttgatagtgattgaaaacgagtcaattgttgaagggaagatccccatcatctcttataatgcatttaacaaacgaattaaagccattcctccttatgctgttgcagtcgctaggcatgggaaattcaaagctgatcaatggtttgcatattgtgcagctcacgttccaccaacgcgtatacttgaacgagtggagattgatcatactccgttggatctaatattattggatgatgagcttctgattcccattggccggccatatcttacacttctcatagatgtgttttcaggttgcgttctgggattccatttgtcatacaagagcccttcttatgtctctgcagcaaaagcaatcactcacgcaataaaacccaaaagcttagatgcgcttaatattgagcttcagaatgactggccctgtttcgggaagtttgagaatttagtagttgacaatggagctgaattttggtcaaaaaatttggaacatgcttgtcaatctgctggtataaatattcaatacaatccggtgcgtaagccatggctcaagccttttatagagaggttctttggggtgatgaatgaatacttcttgccggaactcccaggaaaaacattttctaacattcttgagaaagaagaatacaaaccggagaaggatgcaataatgaggttcagcacctttgttgaggagttccaccgttggattgccgacgtgtatcatcaagatagtaatagccgtgaaacgcgtattcctattaagcgatggcaacaaggattcgatgcttatcctcctttaacaatgaacgaagaagaggagacacggttttctatgctcatgagaatctctgactctaggacgctaaccaggaatgggtttaagtaccaagaattgatgtatgattctaccgccttagcagattaccgtaagcattaccctcagacaaaagagactgtgaagaagctaataaaggtcgatccagatgatatctccaagatttacgtttatctggaagagttggaaagttacttggaggttccttgcacagatccaactggatacactgatggtttatccatatatgagcataaaaccattaagaagatcaaccgagaagttattagagaatcaaaagatagccttgggttggcgaaagcgagaatggctattcatgagagagtgaaacaagagcaagaagtattcatcgaaagtaagaccaaagctaagattactgcagtcaagaagcaggctcagatcgcggatgttagtaatacaggcacatcaacaatcaaggtgtccgaggagtctgcggctcctgtccagaaacacatctcaaatgataacagtgatgattgggatgatgatctggaagcttttgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaatgctcttacagagattcaaatagagaagttacggaatttcagtgattgcatcgtgatgcatcctcagataaagaccattttcaatgattttgatgaactaaggcttaatagaaagtttcagagtgatcagcaatgtatgcttcttataggagatactggtgttggcaaatcacacaccatcaaccattacaagaaaagagtcctcgcaacacaaaactattcaagaaacacaatgccggtccttgtttctcgcatatcacgtgggaaaggtttggatgctactttggtgcagatgctggcggatctagagctgtttggttcttctcagatcaagaagcggggctacaaaacagaccttaccaaaaaattagttgagagtttgatcaaagctcaagtggagctgttgattatcaatgaattccaagagcttattgagtttaaaagtgttcaagaaagacaacagattgcaaatggtctaaagtttattagtgaagaagccaaggttcccattgttttggttggaatgccatgggcagcaaagatagctgaggaaccacaatgggcttctcgactcgtaagaaaacgaaagctagaatatttttcactcaagaatgacagcaagtactttaggcagtatttgatgggtcttgccaaaaagatgcctttcgacgttcctcctaaattagaatcgaagaacactacgattgcgctatttgcagcttgtagaggggaaaacagagctttgaagcaccttcttttagaagcactgaagctcgccctttcatgcaatgagtatcttgagaacaagcatttcatcacagcttatgacaaattcgatttcttcaacgacaaggagaaactgaaatccaaaaaccctttcaagcaagatataaaagatattgaaatttatgaagttattaaaaattcttcctacaatccaaatgcacttgatccagaagatatgttgactgatcgagtatttgctattgtgaaaggaagcggaaagaggactgcagatggatcagaatttgagtccccaaagaagaaaaggaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggcttttcttttctcaccaaaagcaagagcgttttccgatgaatcgcttgaaagttatttacttcgagttgtctcagaaaacttcttcgattcatatgaaggactatctttggccatcagagaagaacttcatgagttggacttcgaggctcatggagcatttcccgtagatttaaagagacttaatgtttaccatgcaaaacacaactcacactttaggatgagagctcttggcctattagaaactcttttagatcttcctcgttatgagcttcaaaagctagcactcctgaagtcagacattaaattcaattcttccgttgccctctacaacaatggtgttgatattcctttgagattcattagacatcatgctgaggaagctgttgactccattccggtttgttctcaatgcttggctgaagaagcttatataaagcaatcttggcatatcaaatgggttaatgcttgcaccaaacatcaatgtgctttgcttcacaattgtcctgaatgttatgcaccgatcaattacattgagaatgaatcaatcactcattgctcttgtggttttgaattgagctgcgcctctacaagtcctgtgaacacactctcgattgaacatttgaacaaactcctggacaaaggagaacggaatgactcaaatccattgtttaacaacatgacgttgacagaaaggtttgctgctttattatggtatcaagagaggtattcacagactgataatttctgtttgaatgatgctgtgaactacttcagtaagtggccagcagtgtttaatactgagctagatgagctctcaaagaatgccgagatgaagctgattgatctcttcaacaagacagagtttaagtttatctttggagatgcaattcttgcttgtccaagtactcagaagcagagtgaatctcatttcatctaccgggcattacttgattaccttgtcactttagtggagagcaatcccaaaacaaagaagccaaacgcagctgatttgcttgtttctgtcctggaagcagcaactttgttggggacgagtgtagaacaagtttataggctctatcagaatggaatactgcaaaccgcctttcggcacaagatgaatcagagaataaacccttacaagggtgctttctttctccgtcatgtgatagagtataaaaccagctttggtaacgataaagctagaatgtatctctcagcttggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagagatattattttaccgttcattttcttcctaaacaagctaatttggcccttctcaccggaagatgcatatctatcatgcatggatttatcttgaagcataacattgaaggaatgggagtaacatttcccgcatggtctgattcaagcattggaaacgagattgcttttgtttacactgataaagaaatcctaaacacattgaaagatcaggcttactttgtggatatgcaagactgtggtttcttcaaggtttctcaggtgttggctgttcctgattcttgcgaggaagtcaggttcatccgcaatcaagccgtcgcaaagattttcactggcgaatcgaggaggagacttaaacggttacaaaagagagccttagcaagaggagaagatttcaatccaaagaagatagaagctccaagagaaatagacatttttcaccgtgtagcaatgacaagcaaaagttctcaagaagactacattctgcacatccagaagcaagatgttgattgtcaagcagagccttatttttcaaactatggtcttgcgtcaaatgagaagttcaaaggaactgtaccagacttgagtccgtccattgatagaaattgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggagctttgcaatatactcaagtatgatcgaagtctttaccctgggaaagcggttttcttctacaaaacggcggacagcgattttgttcctttggaagctgacattaataagataagaggaccaaaatcaggctttacagaggctttcactcctcaattctctccgaaaaatatctctccacaagatctaactcacaacaacattcttacactcgaagagtgttatgttccaccaaatgtcgaacatattttctgccggttttctctgcgtgtccaagctaattcgttagtcccctctggttgttccgatcctgaggtttttagccttctcaaagagcttgcagaaaccttcaaagaatgtggtggttacaaggagctggcagtacgctattgtcggaacattttgattggaacatggctttggagaaatcagaataccggaaacacacagattgagatcaagacttcaaaaggatcttgctacttgattgataatacaagaaaactcgcttgggaaagcaaatgggcttcagacgacttgaaagttttggaagaattaagcaatgaaatcgaatctgctttaacagatccaaatgtgttttggtctgcagatataaccgccaagatcgaagcatctttctgtcaagagatatatccttcacagatattgaatgataaagtgaagcaaggagaagcgtcaaaacagtttgtgaaagctaaatgtgctgatggaaggtatgctgtgagtttcaacagtgttaagatcggtgctgctttgcaaagtattgatgattggtgggatgaagatgctagtaaaagacttagagttcatgaatttggtgcagacaaggaaattggcgtagcgaggaggcctcccgattccgaacaaaacttttattcaatcttcaagaacactgaatggtatctttccgccttaaagaattgcatcactaacaagaatgagaagattgacccagcaatatactatttgttcagcgtgttgatcaaaggtggaatgtttcagaaaaaggcagaggcaaagaaagcatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcacctgaaagagctgttagagataacagatacaactgagagagatagatctctccgacgtgccttctcgccgtataccgccatgatcgacattacaggatcagaagcagtcgctctaattatcctactcaatctcacttatagaaagaatcaggtagatgacctgttggataagaaactcgcgaaacaagccctcaagagtgaggatcatatcaacaaatgcatcaaggagattgcttggtttcatactcacaaccttaaatatccagacataagggtgagtaagcaaaatcttgcagtagaaccaccaacattgcattcttatgtgctttcttctgctaattatcccaaagcttatggttggtcccataattcagctaaagtgaattttgctaagttgtttgtctcatacttcaaatggcaaaaccaagtgagctggcttgcccaagtcttggcaaccaactctgataactggaaaagtgcatttacttcccttggtttatcggtgaaagccttcaaatctttatgcgttacagttaagaattctttacctgaagaggcaatcccggactcagtggatagatatagcaggcagattagaatgccttatcatgatggttatcttgctgtcactcctgtgatatctcacgttgttcaaagcaaaatacaacaagctgctattgacaaaagagctaggttttctaatgttgagtttaccagaccagctgctgtttcaatgctggcggcaagtctaggtggtgtgataaatgtacttaattatcctccatacattcgatcaaagtaccatggactatcgaattcaagggcttttaagttaaacaacggacagactgtcttcaacgtggaagccctcctgaagcctgagcttataaaagctcttgaaggaattatattctctaacaatgcattggcattaaaacagagaaggcagcagaaagtaaaaaacatcaaagaactgagaaataccctccttgagtggttttcccctgttttcgaatggagattggatgcaattgagaatggttatgatctagaacaattggaatcagcgagcgaacggttagagtacaagattctgagtttgccggataatgaactaccttctttgactatacccttgtttagattacttaatgagatgttgggaggagtttcaatgactcagaggtatgcattccatccaaagttgatgtctccattaaaggctgctctccaatggttgctcgttaatttaactgatcaaaagcatgttttaattgaagaagatgatgagcattacagataccttcacctgtcagggattagggtttttgatgcccaagcattgtctaatccttactgctctggtattccttcgcttacggcggtttggggaatgatccacagttatcaacgcaaactaaacgaagctttgggcacgaacgtacggttcacaagcttttcatggttcatccgtaattactctgccgttgctggtaaaaagctaccagaacttagccttcagggtgcacaacagtctagattgaaaagacctgggataatagacgggaagtattgtgatttggtgtttgacttgattattcatatcgatggctatgaagacgatttgcaggctgttgattcaaagcctgatatcctcaaggctcatttcccaagcaatttcgccggcggcgtgatgcatcaacccgagttgaattctaatattaattggtgttgtctttactccaacgaaaaccagctttttgagaagctccgtcggcttcctctttctggatgttgggttatgcccacagaacataagattcaagatcttgatgaactgttgttgctgttgaactcggatagtaaactaagtccaagcatgatgggatacatgcttctcacagagccaatggctcgtgtaggatcgttagagaggcttcactgttatgcagaacctgctattggtgttgtcaaatacgaggcagcaacttctgttcgattaaaaggtatcggaaactatttcaattccgcgttttggatgctcgatgctcaagaaaagttcatgctcatgaagaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgaataggcgtatcacgaggccctttcgtcttcaagaattagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcgggatcgaagatccattccaagcctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgtgtctcgcccttcagcacgccgtcgcgggggtacaggcgctcggtggaggcctcccagcccatcgtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatttcgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataccgtgactcccttaattctcatgtatggttcgctatactgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 42 - DNA, 29999 bp, synthetic construct
  source 1..29999: mol_type = other DNA
tggcaggatatattgtggtgtaaacataacgaattgctcttcagcaaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgtcgctgcaaccatagtttgacataatgcaaccataccttgacataattaaagtcatactctgacataaagccccttattaaaaatgatgctaaaaataaattccattataatcaattatttaagaattaattaaaccatagtgtgacatagtggctttttatttgtatagtatgtgtactcaaataataaaaatttacttatgtatatacgcaacttaagaaagccatctccaaataaaaatgtatttcgctagcaaggagtgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacggccgcgtatttttacaacaattaccaacaacaacaaacaacaaacaacattacaattactatttacaattacagcggccgcccccttcaccaaaaaaatggctgcagccaagggcgaggagctgttcaccggggtggtgcccatcctggtcgagctggacggcgacgtaaacggccacaagttcagcgtgtccggcgagggcgagggcgatgccacctacggcaagctgaccctgaagctgctctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgttcaaagaccccaacgagaagcgcgatcacatggtcttgttagaatttttgactgctgctggtattaccgagggtatgaatgaattgtacaaaggctccggaccgaagaagaagaggaaggtttaaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagccgtataggcagctgaagataaaaacaatcgattcgatgtttaagttaatgtaattctgccgaaaaggcagtgagtagtaattgaaaaaaccagtcttatatccgggctggtttttttgtttttagcgttataacttttatttcactatgtcactgtatggttgcattttatgtcagtgaatggttgcaggttttttgtaagttattgatatgcctatccagtgtatgtcacgctttgcttgcagcgacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatggtgacctgccgtataggcagctgaagatactgacgtaagggatgacgcacaatcccactataattctgccgaaaaggcagtgagtagtgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgtacattaggaacttgagaaagccttctccaaataagaatgtttttaagttcgcatctaccaaggtcagctctgtggtcatgtgtgaatccagccttgagtttgacgcttgctttcatcatgaatataacgacttgattgagagttttggttctcagccagaaggtttcaaatacgaattcatgggtaaatctctcccttacactccagatgctttgatctcctacacggacaaaactcaaaagtatcacgagtataaaccatattcaaaaatcgcatcacctctatttagagcagagtttgctgcaaagagggctgcaagtcttaagttgggtattgatttggtactcgtcacagacagacagattagagtaaacccaatactcaacaatctgaagctccttcatagatactcgggcgtttatggtatatctggtatccagaaggaactacttagctttattcacaaaagtggcgttatcaaactcaatgatatctcatcacaggttgggatccccattggagaaactcggtcatttctttttggactgatgcataagggacttgtaaaggctgacctaggttgtgatgacctgacaaacaaccctactctttgggctactcctggaagtggctcaggcaaacgtactgctgacggaagtgagttcgagtccccaaagaaaaaaagaaaagttggtagtggatctggtggaatgactgatttcttcaatgagtttgatgaatcgcttgtaccactgaaaccacaaactccaacgcagtatgttaagctggacgatgctaatttaattcagagagatttagatacattctcagatactttcaagaaccaagccctccaaagatataagctaatttctacaattgataaaaagttaagccgcggatggactcaaagaaatctcgacccgatcctcgacgaactcttcaaaggcggtgatgtagtgaggccaaactggagaactgtggcaagatggaggaagaaatatatagaaagcaatggtgacatcgcttctcttgctgataaaaaccacaaaatgggaaacagaacaaatcggataaaaggagatgacaaattttttgacaaggccttggaacgtttccttgacgcgaaaagacctacaatagcaacagcttatcaatattacaaggacttgatagtgattgaaaacgagtcaattgttgaagggaagatccccatcatctcttataatgcatttaacaaacgaattaaagccattcctccttatgctgttgcagtcgctaggcatgggaaattcaaagctgatcaatggtttgcatattgtgcagctcacgttccaccaacgcgtatacttgaacgagtggagattgatcatactccgttggatctaatattattggatgatgagcttctgattcccattggccggccatatcttacacttctcatagatgtgttttcaggttgcgttctgggattccatttgtcatacaagagcccttcttatgtctctgcagcaaaagcaatcactcacgcaataaaacccaaaagcttagatgcgcttaatattgagcttcagaatgactggccctgtttcgggaagtttgagaatttagtagttgacaatggagctgaattttggtcaaaaaatttggaacatgcttgtcaatctgctggtataaatattcaatacaatccggtgcgtaagccatggctcaagccttttatagagaggttctttggggtgatgaatgaatacttcttgccggaactcccaggaaaaacattttctaacattcttgagaaagaagaatacaaaccggagaaggatgcaataatgaggttcagcacctttgttgaggagttccaccgttggattgccgacgtgtatcatcaagatagtaatagccgtgaaacgcgtattcctattaagcgatggcaacaaggattcgatgcttatcctcctttaacaatgaacgaagaagaggagacacggttttctatgctcatgagaatctctgactctaggacgctaaccaggaatgggtttaagtaccaagaattgatgtatgattctaccgccttagcagattaccgtaagcattaccctcagacaaaagagactgtgaagaagctaataaaggtcgatccagatgatatctccaagatttacgtttatctggaagagttggaaagttacttggaggttccttgcacagatccaactggatacactgatggtttatccatatatgagcataaaaccattaagaagatcaaccgagaagttattagagaatcaaaagatagccttgggttggcgaaagcgagaatggctattcatgagagagtgaaacaagagcaagaagtattcatcgaaagtaagaccaaagctaagattactgcagtcaagaagcaggctcagatcgcggatgttagtaatacaggcacatcaacaatcaaggtgtccgaggagtctgcggctcctgtccagaaacacatctcaaatgataacagtgatgattgggatgatgatctggaagcttttgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaatgctcttacagagattcaaatagagaagttacggaatttcagtgattgcatcgtgatgcatcctcagataaagaccattttcaatgattttgatgaactaaggcttaatagaaagtttcagagtgatcagcaatgtatgcttcttataggagatactggtgttggcaaatcacacaccatcaaccattacaagaaaagagtcctcgcaacacaaaactattcaagaaacacaatgccggtccttgtttctcgcatatcacgtgggaaaggtttggatgctactttggtgcagatgctggcggatctagagctgtttggttcttctcagatcaagaagcggggctacaaaacagaccttaccaaaaaattagttgagagtttgatcaaagctcaagtggagctgttgattatcaatgaattccaagagcttattgagtttaaaagtgttcaagaaagacaacagattgcaaatggtctaaagtttattagtgaagaagccaaggttcccattgttttggttggaatgccatgggcagcaaagatagctgaggaaccacaatgggcttctcgactcgtaagaaaacgaaagctagaatatttttcactcaagaatgacagcaagtactttaggcagtatttgatgggtcttgccaaaaagatgcctttcgacgttcctcctaaattagaatcgaagaacactacgattgcgctatttgcagcttgtagaggggaaaacagagctttgaagcaccttcttttagaagcactgaagctcgccctttcatgcaatgagtatcttgagaacaagcatttcatcacagcttatgacaaattcgatttcttcaacgacaaggagaaactgaaatccaaaaaccctttcaagcaagatataaaagatattgaaatttatgaagttattaaaaattcttcctacaatccaaatgcacttgatccagaagatatgttgactgatcgagtatttgctattgtgaaaggaagcggaaagaggactgcagatggatcagaatttgagtccccaaagaagaaaaggaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggcttttcttttctcaccaaaagcaagagcgttttccgatgaatcgcttgaaagttatttacttcgagttgtctcagaaaacttcttcgattcatatgaaggactatctttggccatcagagaagaacttcatgagttggacttcgaggctcatggagcatttcccgtagatttaaagagacttaatgtttaccatgcaaaacacaactcacactttaggatgagagctcttggcctattagaaactcttttagatcttcctcgttatgagcttcaaaagctagcactcctgaagtcagacattaaattcaattcttccgttgccctctacaacaatggtgttgatattcctttgagattcattagacatcatgctgaggaagctgttgactccattccggtttgttctcaatgcttggctgaagaagcttatataaagcaatcttggcatatcaaatgggttaatgcttgcaccaaacatcaatgtgctttgcttcacaattgtcctgaatgttatgcaccgatcaattacattgagaatgaatcaatcactcattgctcttgtggttttgaattgagctgcgcctctacaagtcctgtgaacacactctcgattgaacatttgaacaaactcctggacaaaggagaacggaatgactcaaatccattgtttaacaacatgacgttgacagaaaggtttgctgctttattatggtatcaagagaggtattcacagactgataatttctgtttgaatgatgctgtgaactacttcagtaagtggccagcagtgtttaatactgagctagatgagctctcaaagaatgccgagatgaagctgattgatctcttcaacaagacagagtttaagtttatctttggagatgcaattcttgcttgtccaagtactcagaagcagagtgaatctcatttcatctaccgggcattacttgattaccttgtcactttagtggagagcaatcccaaaacaaagaagccaaacgcagctgatttgcttgtttctgtcctggaagcagcaactttgttggggacgagtgtagaacaagtttataggctctatcagaatggaatactgcaaaccgcctttcggcacaagatgaatcagagaataaacccttacaagggtgctttctttctccgtcatgtgatagagtataaaaccagctttggtaacgataaagctagaatgtatctctcagcttggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagagatattattttaccgttcattttcttcctaaacaagctaatttggcccttctcaccggaagatgcatatctatcatgcatggatttatcttgaagcataacattgaaggaatgggagtaacatttcccgcatggtctgattcaagcattggaaacgagattgcttttgtttacactgataaagaaatcctaaacacattgaaagatcaggcttactttgtggatatgcaagactgtggtttcttcaaggtttctcaggtgttggctgttcctgattcttgcgaggaagtcaggttcatccgcaatcaagccgtcgcaaagattttcactggcgaatcgaggaggagacttaaacggttacaaaagagagccttagcaagaggagaagatttcaatccaaagaagatagaagctccaagagaaatagacatttttcaccgtgtagcaatgacaagcaaaagttctcaagaagactacattctgcacatccagaagcaagatgttgattgtcaagcagagccttatttttcaaactatggtcttgcgtcaaatgagaagttcaaaggaactgtaccagacttgagtccgtccattgatagaaattgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggagctttgcaatatactcaagtatgatcgaagtctttaccctgggaaagcggttttcttctacaaaacggcggacagcgattttgttcctttggaagctgacattaataagataagaggaccaaaatcaggctttacagaggctttcactcctcaattctctccgaaaaatatctctccacaagatctaactcacaacaacattcttacactcgaagagtgttatgttccaccaaatgtcgaacatattttctgccggttttctctgcgtgtccaagctaattcgttagtcccctctggttgttccgatcctgaggtttttagccttctcaaagagcttgcagaaaccttcaaagaatgtggtggttacaaggagctggcagtacgctattgtcggaacattttgattggaacatggctttggagaaatcagaataccggaaacacacagattgagatcaagacttcaaaaggatcttgctacttgattgataatacaagaaaactcgcttgggaaagcaaatgggcttcagacgacttgaaagttttggaagaattaagcaatgaaatcgaatctgctttaacagatccaaatgtgttttggtctgcagatataaccgccaagatcgaagcatctttctgtcaagagatatatccttcacagatattgaatgataaagtgaagcaaggagaagcgtcaaaacagtttgtgaaagctaaatgtgctgatggaaggtatgctgtgagtttcaacagtgttaagatcggtgctgctttgcaaagtattgatgattggtgggatgaagatgctagtaaaagacttagagttcatgaatttggtgcagacaaggaaattggcgtagcgaggaggcctcccgattccgaacaaaacttttattcaatcttcaagaacactgaatggtatctttccgccttaaagaattgcatcactaacaagaatgagaagattgacccagcaatatactatttgttcagcgtgttgatcaaaggtggaatgtttcagaaaaaggcagaggcaaagaaagcatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcacctgaaagagctgttagagataacagatacaactgagagagatagatctctccgacgtgccttctcgccgtataccgccatgatcgacattacaggatcagaagcagtcgctctaattatcctactcaatctcacttatagaaagaatcaggtagatgacctgttggataagaaactcgcgaaacaagccctcaagagtgaggatcatatcaacaaatgcatcaaggagattgcttggtttcatactcacaaccttaaatatccagacataagggtgagtaagcaaaatcttgcagtagaaccaccaacattgcattcttatgtgctttcttctgctaattatcccaaagcttatggttggtcccataattcagctaaagtgaattttgctaagttgtttgtctcatacttcaaatggcaaaaccaagtgagctggcttgcccaagtcttggcaaccaactctgataactggaaaagtgcatttacttcccttggtttatcggtgaaagccttcaaatctttatgcgttacagttaagaattctttacctgaagaggcaatcccggactcagtggatagatatagcaggcagattagaatgccttatcatgatggttatcttgctgtcactcctgtgatatctcacgttgttcaaagcaaaatacaacaagctgctattgacaaaagagctaggttttctaatgttgagtttaccagaccagctgctgtttcaatgctggcggcaagtctaggtggtgtgataaatgtacttaattatcctccatacattcgatcaaagtaccatggactatcgaattcaagggcttttaagttaaacaacggacagactgtcttcaacgtggaagccctcctgaagcctgagcttataaaagctcttgaaggaattatattctctaacaatgcattggcattaaaacagagaaggcagcagaaagtaaaaaacatcaaagaactgagaaataccctccttgagtggttttcccctgttttcgaatggagattggatgcaattgagaatggttatgatctagaacaattggaatcagcgagcgaacggttagagtacaagattctgagtttgccggataatgaactaccttctttgactatacccttgtttagattacttaatgagatgttgggaggagtttcaatgactcagaggtatgcattccatccaaagttgatgtctccattaaaggctgctctccaatggttgctcgttaatttaactgatcaaaagcatgttttaattgaagaagatgatgagcattacagataccttcacctgtcagggattagggtttttgatgcccaagcattgtctaatccttactgctctggtattccttcgcttacggcggtttggggaatgatccacagttatcaacgcaaactaaacgaagctttgggcacgaacgtacggttcacaagcttttcatggttcatccgtaattactctgccgttgctggtaaaaagctaccagaacttagccttcagggtgcacaacagtctagattgaaaagacctgggataatagacgggaagtattgtgatttggtgtttgacttgattattcatatcgatggctatgaagacgatttgcaggctgttgattcaaagcctgatatcctcaaggctcatttcccaagcaatttcgccggcggcgtgatgcatcaacccgagttgaattctaatattaattggtgttgtctttactccaacgaaaaccagctttttgagaagctccgtcggcttcctctttctggatgttgggttatgcccacagaacataagattcaagatcttgatgaactgttgttgctgttgaactcggatagtaaactaagtccaagcatgatgggatacatgcttctcacagagccaatggctcgtgtaggatcgttagagaggcttcactgttatgcagaacctgctattggtgttgtcaaatacgaggcagcaacttctgttcgattaaaaggtatcggaaactatttcaattccgcgttttggatgctcgatgctcaagaaaagttcatgctcatgaagaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgaataggcgtatcacgaggccctttcgtcttcaagaattagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcgggatcgaagatccattccaagcctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgtgtctcgcccttcagcacgccgtcgcgggggtacaggcgctcggtggaggcctcccagcccatcgtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatttcgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataccgtgactcccttaattctcatgtatggttcgctatactgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 43 - DNA, 29999 bp, synthetic construct
  source 1..29999: mol_type = other DNA
tggcaggatatattgtggtgtaaacataacgaattgctcttcagcaaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgtcgctgcaaccatagtttgacataatgcaaccataccttgacataattaaagtcatactctgacataaagccccttattaaaaatgatgctaaaaataaattccattataatcaattatttaagaattaattaaaccatagtgtgacatagtggctttttatttgtatagtatgtgtactcaaataataaaaatttacttatgtatatacgcaacttaagaaagccatctccaaataaaaatgtatttcgctagcaaggagtgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgacggccgcgtatttttacaacaattaccaacaacaacaaacaacaaacaacattacaattactatttacaattacagcggccgcccccttcaccaaaaaaatggctgcagccaagggcgaggagctgttcaccggggtggtgcccatcctggtcgagctggacggcgacgtaaacggccacaagttcagcgtgtccggcgagggcgagggcgatgccacctacggcaagctgaccctgaagctgctctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaaactgttcaaagaccccaacgagaagcgcgatcacatggtcttgttagaatttttgactgctgctggtattaccgagggtatgaatgaattgtacaaaggctccggaccgaagaagaagaggaaggtttaaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagccgtataggcagctgaagataaaaacaatcgattcgatgtttaagttaatgtaattctgccgaaaaggcagtgagtagtaattgaaaaaaccagtcttatatccgggctggtttttttgtttttagcgttataacttttatttcactatgtcactgtatggttgcattttatgtcagtgaatggttgcaggttttttgtaagttattgatatgcctatccagtgtatgtcacgctttgcttgcagcgacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatggtgacctgccgtataggcagctgaagatatcccactatccttcgcaagacccttcctctataattctgccgaaaaggcagtgagtagtgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgtacattaggaacttgagaaagccttctccaaataagaatgtttttaagttcgcatctaccaaggtcagctctgtggtcatgtgtgaatccagccttgagtttgacgcttgctttcatcatgaatataacgacttgattgagagttttggttctcagccagaaggtttcaaatacgaattcatgggtaaatctctcccttacactccagatgctttgatctcctacacggacaaaactcaaaagtatcacgagtataaaccatattcaaaaatcgcatcacctctatttagagcagagtttgctgcaaagagggctgcaagtcttaagttgggtattgatttggtactcgtcacagacagacagattagagtaaacccaatactcaacaatctgaagctccttcatagatactcgggcgtttatggtatatctggtatccagaaggaactacttagctttattcacaaaagtggcgttatcaaactcaatgatatctcatcacaggttgggatccccattggagaaactcggtcatttctttttggactgatgcataagggacttgtaaaggctgacctaggttgtgatgacctgacaaacaaccctactctttgggctactcctggaagtggctcaggcaaacgtactgctgacggaagtgagttcgagtccccaaagaaaaaaagaaaagttggtagtggatctggtggaatgactgatttcttcaatgagtttgatgaatcgcttgtaccactgaaaccacaaactccaacgcagtatgttaagctggacgatgctaatttaattcagagagatttagatacattctcagatactttcaagaaccaagccctccaaagatataagctaatttctacaattgataaaaagttaagccgcggatggactcaaagaaatctcgacccgatcctcgacgaactcttcaaaggcggtgatgtagtgaggccaaactggagaactgtggcaagatggaggaagaaatatatagaaagcaatggtgacatcgcttctcttgctgataaaaaccacaaaatgggaaacagaacaaatcggataaaaggagatgacaaattttttgacaaggccttggaacgtttccttgacgcgaaaagacctacaatagcaacagcttatcaatattacaaggacttgatagtgattgaaaacgagtcaattgttgaagggaagatccccatcatctcttataatgcatttaacaaacgaattaaagccattcctccttatgctgttgcagtcgctaggcatgggaaattcaaagctgatcaatggtttgcatattgtgcagctcacgttccaccaacgcgtatacttgaacgagtggagattgatcatactccgttggatctaatattattggatgatgagcttctgattcccattggccggccatatcttacacttctcatagatgtgttttcaggttgcgttctgggattccatttgtcatacaagagcccttcttatgtctctgcagcaaaagcaatcactcacgcaataaaacccaaaagcttagatgcgcttaatattgagcttcagaatgactggccctgtttcgggaagtttgagaatttagtagttgacaatggagctgaattttggtcaaaaaatttggaacatgcttgtcaatctgctggtataaatattcaatacaatccggtgcgtaagccatggctcaagccttttatagagaggttctttggggtgatgaatgaatacttcttgccggaactcccaggaaaaacattttctaacattcttgagaaagaagaatacaaaccggagaaggatgcaataatgaggttcagcacctttgttgaggagttccaccgttggattgccgacgtgtatcatcaagatagtaatagccgtgaaacgcgtattcctattaagcgatggcaacaaggattcgatgcttatcctcctttaacaatgaacgaagaagaggagacacggttttctatgctcatgagaatctctgactctaggacgctaaccaggaatgggtttaagtaccaagaattgatgtatgattctaccgccttagcagattaccgtaagcattaccctcagacaaaagagactgtgaagaagctaataaaggtcgatccagatgatatctccaagatttacgtttatctggaagagttggaaagttacttggaggttccttgcacagatccaactggatacactgatggtttatccatatatgagcataaaaccattaagaagatcaaccgagaagttattagagaatcaaaagatagccttgggttggcgaaagcgagaatggctattcatgagagagtgaaacaagagcaagaagtattcatcgaaagtaagaccaaagctaagattactgcagtcaagaagcaggctcagatcgcggatgttagtaatacaggcacatcaacaatcaaggtgtccgaggagtctgcggctcctgtccagaaacacatctcaaatgataacagtgatgattgggatgatgatctggaagcttttgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaatgctcttacagagattcaaatagagaagttacggaatttcagtgattgcatcgtgatgcatcctcagataaagaccattttcaatgattttgatgaactaaggcttaatagaaagtttcagagtgatcagcaatgtatgcttcttataggagatactggtgttggcaaatcacacaccatcaaccattacaagaaaagagtcctcgcaacacaaaactattcaagaaacacaatgccggtccttgtttctcgcatatcacgtgggaaaggtttggatgctactttggtgcagatgctggcggatctagagctgtttggttcttctcagatcaagaagcggggctacaaaacagaccttaccaaaaaattagttgagagtttgatcaaagctcaagtggagctgttgattatcaatgaattccaagagcttattgagtttaaaagtgttcaagaaagacaacagattgcaaatggtctaaagtttattagtgaagaagccaaggttcccattgttttggttggaatgccatgggcagcaaagatagctgaggaaccacaatgggcttctcgactcgtaagaaaacgaaagctagaatatttttcactcaagaatgacagcaagtactttaggcagtatttgatgggtcttgccaaaaagatgcctttcgacgttcctcctaaattagaatcgaagaacactacgattgcgctatttgcagcttgtagaggggaaaacagagctttgaagcaccttcttttagaagcactgaagctcgccctttcatgcaatgagtatcttgagaacaagcatttcatcacagcttatgacaaattcgatttcttcaacgacaaggagaaactgaaatccaaaaaccctttcaagcaagatataaaagatattgaaatttatgaagttattaaaaattcttcctacaatccaaatgcacttgatccagaagatatgttgactgatcgagtatttgctattgtgaaaggaagcggaaagaggactgcagatggatcagaatttgagtccccaaagaagaaaaggaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggcttttcttttctcaccaaaagcaagagcgttttccgatgaatcgcttgaaagttatttacttcgagttgtctcagaaaacttcttcgattcatatgaaggactatctttggccatcagagaagaacttcatgagttggacttcgaggctcatggagcatttcccgtagatttaaagagacttaatgtttaccatgcaaaacacaactcacactttaggatgagagctcttggcctattagaaactcttttagatcttcctcgttatgagcttcaaaagctagcactcctgaagtcagacattaaattcaattcttccgttgccctctacaacaatggtgttgatattcctttgagattcattagacatcatgctgaggaagctgttgactccattccggtttgttctcaatgcttggctgaagaagcttatataaagcaatcttggcatatcaaatgggttaatgcttgcaccaaacatcaatgtgctttgcttcacaattgtcctgaatgttatgcaccgatcaattacattgagaatgaatcaatcactcattgctcttgtggttttgaattgagctgcgcctctacaagtcctgtgaacacactctcgattgaacatttgaacaaactcctggacaaaggagaacggaatgactcaaatccattgtttaacaacatgacgttgacagaaaggtttgctgctttattatggtatcaagagaggtattcacagactgataatttctgtttgaatgatgctgtgaactacttcagtaagtggccagcagtgtttaatactgagctagatgagctctcaaagaatgccgagatgaagctgattgatctcttcaacaagacagagtttaagtttatctttggagatgcaattcttgcttgtccaagtactcagaagcagagtgaatctcatttcatctaccgggcattacttgattaccttgtcactttagtggagagcaatcccaaaacaaagaagccaaacgcagctgatttgcttgtttctgtcctggaagcagcaactttgttggggacgagtgtagaacaagtttataggctctatcagaatggaatactgcaaaccgcctttcggcacaagatgaatcagagaataaacccttacaagggtgctttctttctccgtcatgtgatagagtataaaaccagctttggtaacgataaagctagaatgtatctctcagcttggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagagatattattttaccgttcattttcttcctaaacaagctaatttggcccttctcaccggaagatgcatatctatcatgcatggatttatcttgaagcataacattgaaggaatgggagtaacatttcccgcatggtctgattcaagcattggaaacgagattgcttttgtttacactgataaagaaatcctaaacacattgaaagatcaggcttactttgtggatatgcaagactgtggtttcttcaaggtttctcaggtgttggctgttcctgattcttgcgaggaagtcaggttcatccgcaatcaagccgtcgcaaagattttcactggcgaatcgaggaggagacttaaacggttacaaaagagagccttagcaagaggagaagatttcaatccaaagaagatagaagctccaagagaaatagacatttttcaccgtgtagcaatgacaagcaaaagttctcaagaagactacattctgcacatccagaagcaagatgttgattgtcaagcagagccttatttttcaaactatggtcttgcgtcaaatgagaagttcaaaggaactgtaccagacttgagtccgtccattgatagaaattgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggagctttgcaatatactcaagtatgatcgaagtctttaccctgggaaagcggttttcttctacaaaacggcggacagcgattttgttcctttggaagctgacattaataagataagaggaccaaaatcaggctttacagaggctttcactcctcaattctctccgaaaaatatctctccacaagatctaactcacaacaacattcttacactcgaagagtgttatgttccaccaaatgtcgaacatattttctgccggttttctctgcgtgtccaagctaattcgttagtcccctctggttgttccgatcctgaggtttttagccttctcaaagagcttgcagaaaccttcaaagaatgtggtggttacaaggagctggcagtacgctattgtcggaacattttgattggaacatggctttggagaaatcagaataccggaaacacacagattgagatcaagacttcaaaaggatcttgctacttgattgataatacaagaaaactcgcttgggaaagcaaatgggcttcagacgacttgaaagttttggaagaattaagcaatgaaatcgaatctgctttaacagatccaaatgtgttttggtctgcagatataaccgccaagatcgaagcatctttctgtcaagagatatatccttcacagatattgaatgataaagtgaagcaaggagaagcgtcaaaacagtttgtgaaagctaaatgtgctgatggaaggtatgctgtgagtttcaacagtgttaagatcggtgctgctttgcaaagtattgatgattggtgggatgaagatgctagtaaaagacttagagttcatgaatttggtgcagacaaggaaattggcgtagcgaggaggcctcccgattccgaacaaaacttttattcaatcttcaagaacactgaatggtatctttccgccttaaagaattgcatcactaacaagaatgagaagattgacccagcaatatactatttgttcagcgtgttgatcaaaggtggaatgtttcagaaaaaggcagaggcaaagaaagcatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcacctgaaagagctgttagagataacagatacaactgagagagatagatctctccgacgtgccttctcgccgtataccgccatgatcgacattacaggatcagaagcagtcgctctaattatcctactcaatctcacttatagaaagaatcaggtagatgacctgttggataagaaactcgcgaaacaagccctcaagagtgaggatcatatcaacaaatgcatcaaggagattgcttggtttcatactcacaaccttaaatatccagacataagggtgagtaagcaaaatcttgcagtagaaccaccaacattgcattcttatgtgctttcttctgctaattatcccaaagcttatggttggtcccataattcagctaaagtgaattttgctaagttgtttgtctcatacttcaaatggcaaaaccaagtgagctggcttgcccaagtcttggcaaccaactctgataactggaaaagtgcatttacttcccttggtttatcggtgaaagccttcaaatctttatgcgttacagttaagaattctttacctgaagaggcaatcccggactcagtggatagatatagcaggcagattagaatgccttatcatgatggttatcttgctgtcactcctgtgatatctcacgttgttcaaagcaaaatacaacaagctgctattgacaaaagagctaggttttctaatgttgagtttaccagaccagctgctgtttcaatgctggcggcaagtctaggtggtgtgataaatgtacttaattatcctccatacattcgatcaaagtaccatggactatcgaattcaagggcttttaagttaaacaacggacagactgtcttcaacgtggaagccctcctgaagcctgagcttataaaagctcttgaaggaattatattctctaacaatgcattggcattaaaacagagaaggcagcagaaagtaaaaaacatcaaagaactgagaaataccctccttgagtggttttcccctgttttcgaatggagattggatgcaattgagaatggttatgatctagaacaattggaatcagcgagcgaacggttagagtacaagattctgagtttgccggataatgaactaccttctttgactatacccttgtttagattacttaatgagatgttgggaggagtttcaatgactcagaggtatgcattccatccaaagttgatgtctccattaaaggctgctctccaatggttgctcgttaatttaactgatcaaaagcatgttttaattgaagaagatgatgagcattacagataccttcacctgtcagggattagggtttttgatgcccaagcattgtctaatccttactgctctggtattccttcgcttacggcggtttggggaatgatccacagttatcaacgcaaactaaacgaagctttgggcacgaacgtacggttcacaagcttttcatggttcatccgtaattactctgccgttgctggtaaaaagctaccagaacttagccttcagggtgcacaacagtctagattgaaaagacctgggataatagacgggaagtattgtgatttggtgtttgacttgattattcatatcgatggctatgaagacgatttgcaggctgttgattcaaagcctgatatcctcaaggctcatttcccaagcaatttcgccggcggcgtgatgcatcaacccgagttgaattctaatattaattggtgttgtctttactccaacgaaaaccagctttttgagaagctccgtcggcttcctctttctggatgttgggttatgcccacagaacataagattcaagatcttgatgaactgttgttgctgttgaactcggatagtaaactaagtccaagcatgatgggatacatgcttctcacagagccaatggctcgtgtaggatcgttagagaggcttcactgttatgcagaacctgctattggtgttgtcaaatacgaggcagcaacttctgttcgattaaaaggtatcggaaactatttcaattccgcgttttggatgctcgatgctcaagaaaagttcatgctcatgaagaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgaataggcgtatcacgaggccctttcgtcttcaagaattagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcgggatcgaagatccattccaagcctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgtgtctcgcccttcagcacgccgtcgcgggggtacaggcgctcggtggaggcctcccagcccatcgtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatttcgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataccgtgactcccttaattctcatgtatggttcgctatactgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 44 - DNA, 28739 bp, synthetic construct
  source 1..28739: mol_type = other DNA
tggcaggatatattgtggtgtaaacataacgaattgctcttcagcaaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgtcgctgcaaccatagtttgacataatgcaaccataccttgacataattaaagtcatactctgacataaagccccttattaaaaatgatgctaaaaataaattccattataatcaattatttaagaattaattaaaccatagtgtgacatagtggctttttatttgtatagtatgtgtactcaaataataaaaatttacttatgtatatacgcaacttaagaaagccatctccaaataaaaatgtatttctacctgttccatggccaaccgctagcaaggaggtggagatccttcctcctagatccaaaatacggtaccgtcaacaatctcactcgagagtcgacgtatgtgcaaaactaagcattccgaagccattgttagccgtatgaatagggaaactaaacccagtgataagacctgatgttttcgcttctttaattacatttggagatgcccgctatacaggagccgtataggcagctgaagataaaaacaatcgattcgatgtttaagttaatgtaattctgccgaaaaggcagtgagtagtaattgaaaaaaccagtcttatatccgggctggtttttttgtttttagcgttataacttttatttcactatgtcactgtatggttgcattttatgtcagtgaatggttgcaggttttttgtaagttattgatatgcctatccagtgtatgtcacgctttgcttgcagcgacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatggtgacctgccgtataggcagctgaagatactgacgtaagggatgacgcacaatcccactataattctgccgaaaaggcagtgagtagtgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgtacattaggaacttgagaaagccttctccaaataagaatgtttttaagttcgcatctaccaaggtcagctctgtggtcatgtgtgaatccagccttgagtttgacgcttgctttcatcatgaatataacgacttgattgagagttttggttctcagccagaaggtttcaaatacgaattcatgggtaaatctctcccttacactccagatgctttgatctcctacacggacaaaactcaaaagtatcacgagtataaaccatattcaaaaatcgcatcacctctatttagagcagagtttgctgcaaagagggctgcaagtcttaagttgggtattgatttggtactcgtcacagacagacagattagagtaaacccaatactcaacaatctgaagctccttcatagatactcgggcgtttatggtatatctggtatccagaaggaactacttagctttattcacaaaagtggcgttatcaaactcaatgatatctcatcacaggttgggatccccattggagaaactcggtcatttctttttggactgatgcataagggacttgtaaaggctgacctaggttgtgatgacctgacaaacaaccctactctttgggctactcctggaagtggctcaggcaaacgtactgctgacggaagtgagttcgagtccccaaagaaaaaaagaaaagttggtagtggatctggtggaatgactgatttcttcaatgagtttgatgaatcgcttgtaccactgaaaccacaaactccaacgcagtatgttaagctggacgatgctaatttaattcagagagatttagatacattctcagatactttcaagaaccaagccctccaaagatataagctaatttctacaattgataaaaagttaagccgcggatggactcaaagaaatctcgacccgatcctcgacgaactcttcaaaggcggtgatgtagtgaggccaaactggagaactgtggcaagatggaggaagaaatatatagaaagcaatggtgacatcgcttctcttgctgataaaaaccacaaaatgggaaacagaacaaatcggataaaaggagatgacaaattttttgacaaggccttggaacgtttccttgacgcgaaaagacctacaatagcaacagcttatcaatattacaaggacttgatagtgattgaaaacgagtcaattgttgaagggaagatccccatcatctcttataatgcatttaacaaacgaattaaagccattcctccttatgctgttgcagtcgctaggcatgggaaattcaaagctgatcaatggtttgcatattgtgcagctcacgttccaccaacgcgtatacttgaacgagtggagattgatcatactccgttggatctaatattattggatgatgagcttctgattcccattggccggccatatcttacacttctcatagatgtgttttcaggttgcgttctgggattccatttgtcatacaagagcccttcttatgtctctgcagcaaaagcaatcactcacgcaataaaacccaaaagcttagatgcgcttaatattgagcttcagaatgactggccctgtttcgggaagtttgagaatttagtagttgacaatggagctgaattttggtcaaaaaatttggaacatgcttgtcaatctgctggtataaatattcaatacaatccggtgcgtaagccatggctcaagccttttatagagaggttctttggggtgatgaatgaatacttcttgccggaactcccaggaaaaacattttctaacattcttgagaaagaagaatacaaaccggagaaggatgcaataatgaggttcagcacctttgttgaggagttccaccgttggattgccgacgtgtatcatcaagatagtaatagccgtgaaacgcgtattcctattaagcgatggcaacaaggattcgatgcttatcctcctttaacaatgaacgaagaagaggagacacggttttctatgctcatgagaatctctgactctaggacgctaaccaggaatgggtttaagtaccaagaattgatgtatgattctaccgccttagcagattaccgtaagcattaccctcagacaaaagagactgtgaagaagctaataaaggtcgatccagatgatatctccaagatttacgtttatctggaagagttggaaagttacttggaggttccttgcacagatccaactggatacactgatggtttatccatatatgagcataaaaccattaagaagatcaaccgagaagttattagagaatcaaaagatagccttgggttggcgaaagcgagaatggctattcatgagagagtgaaacaagagcaagaagtattcatcgaaagtaagaccaaagctaagattactgcagtcaagaagcaggctcagatcgcggatgttagtaatacaggcacatcaacaatcaaggtgtccgaggagtctgcggctcctgtccagaaacacatctcaaatgataacagtgatgattgggatgatgatctggaagcttttgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaatgctcttacagagattcaaatagagaagttacggaatttcagtgattgcatcgtgatgcatcctcagataaagaccattttcaatgattttgatgaactaaggcttaatagaaagtttcagagtgatcagcaatgtatgcttcttataggagatactggtgttggcaaatcacacaccatcaaccattacaagaaaagagtcctcgcaacacaaaactattcaagaaacacaatgccggtccttgtttctcgcatatcacgtgggaaaggtttggatgctactttggtgcagatgctggcggatctagagctgtttggttcttctcagatcaagaagcggggctacaaaacagaccttaccaaaaaattagttgagagtttgatcaaagctcaagtggagctgttgattatcaatgaattccaagagcttattgagtttaaaagtgttcaagaaagacaacagattgcaaatggtctaaagtttattagtgaagaagccaaggttcccattgttttggttggaatgccatgggcagcaaagatagctgaggaaccacaatgggcttctcgactcgtaagaaaacgaaagctagaatatttttcactcaagaatgacagcaagtactttaggcagtatttgatgggtcttgccaaaaagatgcctttcgacgttcctcctaaattagaatcgaagaacactacgattgcgctatttgcagcttgtagaggggaaaacagagctttgaagcaccttcttttagaagcactgaagctcgccctttcatgcaatgagtatcttgagaacaagcatttcatcacagcttatgacaaattcgatttcttcaacgacaaggagaaactgaaatccaaaaaccctttcaagcaagatataaaagatattgaaatttatgaagttattaaaaattcttcctacaatccaaatgcacttgatccagaagatatgttgactgatcgagtatttgctattgtgaaaggaagcggaaagaggactgcagatggatcagaatttgagtccccaaagaagaaaaggaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggcttttcttttctcaccaaaagcaagagcgttttccgatgaatcgcttgaaagttatttacttcgagttgtctcagaaaacttcttcgattcatatgaaggactatctttggccatcagagaagaacttcatgagttggacttcgaggctcatggagcatttcccgtagatttaaagagacttaatgtttaccatgcaaaacacaactcacactttaggatgagagctcttggcctattagaaactcttttagatcttcctcgttatgagcttcaaaagctagcactcctgaagtcagacattaaattcaattcttccgttgccctctacaacaatggtgttgatattcctttgagattcattagacatcatgctgaggaagctgttgactccattccggtttgttctcaatgcttggctgaagaagcttatataaagcaatcttggcatatcaaatgggttaatgcttgcaccaaacatcaatgtgctttgcttcacaattgtcctgaatgttatgcaccgatcaattacattgagaatgaatcaatcactcattgctcttgtggttttgaattgagctgcgcctctacaagtcctgtgaacacactctcgattgaacatttgaacaaactcctggacaaaggagaacggaatgactcaaatccattgtttaacaacatgacgttgacagaaaggtttgctgctttattatggtatcaagagaggtattcacagactgataatttctgtttgaatgatgctgtgaactacttcagtaagtggccagcagtgtttaatactgagctagatgagctctcaaagaatgccgagatgaagctgattgatctcttcaacaagacagagtttaagtttatctttggagatgcaattcttgcttgtccaagtactcagaagcagagtgaatctcatttcatctaccgggcattacttgattaccttgtcactttagtggagagcaatcccaaaacaaagaagccaaacgcagctgatttgcttgtttctgtcctggaagcagcaactttgttggggacgagtgtagaacaagtttataggctctatcagaatggaatactgcaaaccgcctttcggcacaagatgaatcagagaataaacccttacaagggtgctttctttctccgtcatgtgatagagtataaaaccagctttggtaacgataaagctagaatgtatctctcagcttggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagagatattattttaccgttcattttcttcctaaacaagctaatttggcccttctcaccggaagatgcatatctatcatgcatggatttatcttgaagcataacattgaaggaatgggagtaacatttcccgcatggtctgattcaagcattggaaacgagattgcttttgtttacactgataaagaaatcctaaacacattgaaagatcaggcttactttgtggatatgcaagactgtggtttcttcaaggtttctcaggtgttggctgttcctgattcttgcgaggaagtcaggttcatccgcaatcaagccgtcgcaaagattttcactggcgaatcgaggaggagacttaaacggttacaaaagagagccttagcaagaggagaagatttcaatccaaagaagatagaagctccaagagaaatagacatttttcaccgtgtagcaatgacaagcaaaagttctcaagaagactacattctgcacatccagaagcaagatgttgattgtcaagcagagccttatttttcaaactatggtcttgcgtcaaatgagaagttcaaaggaactgtaccagacttgagtccgtccattgatagaaattgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggagctttgcaatatactcaagtatgatcgaagtctttaccctgggaaagcggttttcttctacaaaacggcggacagcgattttgttcctttggaagctgacattaataagataagaggaccaaaatcaggctttacagaggctttcactcctcaattctctccgaaaaatatctctccacaagatctaactcacaacaacattcttacactcgaagagtgttatgttccaccaaatgtcgaacatattttctgccggttttctctgcgtgtccaagctaattcgttagtcccctctggttgttccgatcctgaggtttttagccttctcaaagagcttgcagaaaccttcaaagaatgtggtggttacaaggagctggcagtacgctattgtcggaacattttgattggaacatggctttggagaaatcagaataccggaaacacacagattgagatcaagacttcaaaaggatcttgctacttgattgataatacaagaaaactcgcttgggaaagcaaatgggcttcagacgacttgaaagttttggaagaattaagcaatgaaatcgaatctgctttaacagatccaaatgtgttttggtctgcagatataaccgccaagatcgaagcatctttctgtcaagagatatatccttcacagatattgaatgataaagtgaagcaaggagaagcgtcaaaacagtttgtgaaagctaaatgtgctgatggaaggtatgctgtgagtttcaacagtgttaagatcggtgctgctttgcaaagtattgatgattggtgggatgaagatgctagtaaaagacttagagttcatgaatttggtgcagacaaggaaattggcgtagcgaggaggcctcccgattccgaacaaaacttttattcaatcttcaagaacactgaatggtatctttccgccttaaagaattgcatcactaacaagaatgagaagattgacccagcaatatactatttgttcagcgtgttgatcaaaggtggaatgtttcagaaaaaggcagaggcaaagaaagcatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcacctgaaagagctgttagagataacagatacaactgagagagatagatctctccgacgtgccttctcgccgtataccgccatgatcgacattacaggatcagaagcagtcgctctaattatcctactcaatctcacttatagaaagaatcaggtagatgacctgttggataagaaactcgcgaaacaagccctcaagagtgaggatcatatcaacaaatgcatcaaggagattgcttggtttcatactcacaaccttaaatatccagacataagggtgagtaagcaaaatcttgcagtagaaccaccaacattgcattcttatgtgctttcttctgctaattatcccaaagcttatggttggtcccataattcagctaaagtgaattttgctaagttgtttgtctcatacttcaaatggcaaaaccaagtgagctggcttgcccaagtcttggcaaccaactctgataactggaaaagtgcatttacttcccttggtttatcggtgaaagccttcaaatctttatgcgttacagttaagaattctttacctgaagaggcaatcccggactcagtggatagatatagcaggcagattagaatgccttatcatgatggttatcttgctgtcactcctgtgatatctcacgttgttcaaagcaaaatacaacaagctgctattgacaaaagagctaggttttctaatgttgagtttaccagaccagctgctgtttcaatgctggcggcaagtctaggtggtgtgataaatgtacttaattatcctccatacattcgatcaaagtaccatggactatcgaattcaagggcttttaagttaaacaacggacagactgtcttcaacgtggaagccctcctgaagcctgagcttataaaagctcttgaaggaattatattctctaacaatgcattggcattaaaacagagaaggcagcagaaagtaaaaaacatcaaagaactgagaaataccctccttgagtggttttcccctgttttcgaatggagattggatgcaattgagaatggttatgatctagaacaattggaatcagcgagcgaacggttagagtacaagattctgagtttgccggataatgaactaccttctttgactatacccttgtttagattacttaatgagatgttgggaggagtttcaatgactcagaggtatgcattccatccaaagttgatgtctccattaaaggctgctctccaatggttgctcgttaatttaactgatcaaaagcatgttttaattgaagaagatgatgagcattacagataccttcacctgtcagggattagggtttttgatgcccaagcattgtctaatccttactgctctggtattccttcgcttacggcggtttggggaatgatccacagttatcaacgcaaactaaacgaagctttgggcacgaacgtacggttcacaagcttttcatggttcatccgtaattactctgccgttgctggtaaaaagctaccagaacttagccttcagggtgcacaacagtctagattgaaaagacctgggataatagacgggaagtattgtgatttggtgtttgacttgattattcatatcgatggctatgaagacgatttgcaggctgttgattcaaagcctgatatcctcaaggctcatttcccaagcaatttcgccggcggcgtgatgcatcaacccgagttgaattctaatattaattggtgttgtctttactccaacgaaaaccagctttttgagaagctccgtcggcttcctctttctggatgttgggttatgcccacagaacataagattcaagatcttgatgaactgttgttgctgttgaactcggatagtaaactaagtccaagcatgatgggatacatgcttctcacagagccaatggctcgtgtaggatcgttagagaggcttcactgttatgcagaacctgctattggtgttgtcaaatacgaggcagcaacttctgttcgattaaaaggtatcggaaactatttcaattccgcgttttggatgctcgatgctcaagaaaagttcatgctcatgaagaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgaataggcgtatcacgaggccctttcgtcttcaagaattagcttggcaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcgtcgcaagaccggcaacaggattcaatcttaagaaactttattgccaaatgtttgaacgatcgggatcgaagatccattccaagcctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgtgtctcgcccttcagcacgccgtcgcgggggtacaggcgctcggtggaggcctcccagcccatcgtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatttcgcctcgatcgagttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctcagtggctccttcaacgttgcggttctgtcagttccaaacgtaaaacggcttgtcccgcgtcatcggcgggggtcataccgtgactcccttaattctcatgtatggttcgctatactgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 45 - DNA, 22059 bp, synthetic construct
  source 1..22059: mol_type = other DNA
tggcaggatatattgtggtgtaaacataacgaattgctcttcacagaggagaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggtatactaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgtacattaggaacttgagaaagccttctccaaataagaatgtttttaagttcgcatctaccaaggtcagctctgtggtcatgtgtgaatccagccttgagtttgacgcttgctttcatcatgaatataacgacttgattgagagttttggttctcagccagaaggtttcaaatacgaattcatgggtaaatctctcccttacactccagatgctttgatctcctacacggacaaaactcaaaagtatcacgagtataaaccatattcaaaaatcgcatcacctctatttagagcagagtttgctgcaaagagggctgcaagtcttaagttgggtattgatttggtactcgtcacagacagacagattagagtaaacccaatactcaacaatctgaagctccttcatagatactcgggcgtttatggtatatctggtatccagaaggaactacttagctttattcacaaaagtggcgttatcaaactcaatgatatctcatcacaggttgggatccccattggagaaactcggtcatttctttttggactgatgcataagggacttgtaaaggctgacctaggttgtgatgacctgacaaacaaccctactctttgggctactcctggaagtggctcaggcaaacgtactgctgacggaagtgagttcgagtccccaaagaaaaaaagaaaagttggtagtggatctggtggaatgactgatttcttcaatgagtttgatgaatcgcttgtaccactgaaaccacaaactccaacgcagtatgttaagctggacgatgctaatttaattcagagagatttagatacattctcagatactttcaagaaccaagccctccaaagatataagctaatttctacaattgataaaaagttaagccgcggatggactcaaagaaatctcgacccgatcctcgacgaactcttcaaaggcggtgatgtagtgaggccaaactggagaactgtggcaagatggaggaagaaatatatagaaagcaatggtgacatcgcttctcttgctgataaaaaccacaaaatgggaaacagaacaaatcggataaaaggagatgacaaattttttgacaaggccttggaacgtttccttgacgcgaaaagacctacaatagcaacagcttatcaatattacaaggacttgatagtgattgaaaacgagtcaattgttgaagggaagatccccatcatctcttataatgcatttaacaaacgaattaaagccattcctccttatgctgttgcagtcgctaggcatgggaaattcaaagctgatcaatggtttgcatattgtgcagctcacgttccaccaacgcgtatacttgaacgagtggagattgatcatactccgttggatctaatattattggatgatgagcttctgattcccattggccggccatatcttacacttctcatagatgtgttttcaggttgcgttctgggattccatttgtcatacaagagcccttcttatgtctctgcagcaaaagcaatcactcacgcaataaaacccaaaagcttagatgcgcttaatattgagcttcagaatgactggccctgtttcgggaagtttgagaatttagtagttgacaatggagctgaattttggtcaaaaaatttggaacatgcttgtcaatctgctggtataaatattcaatacaatccggtgcgtaagccatggctcaagccttttatagagaggttctttggggtgatgaatgaatacttcttgccggaactcccaggaaaaacattttctaacattcttgagaaagaagaatacaaaccggagaaggatgcaataatgaggttcagcacctttgttgaggagttccaccgttggattgccgacgtgtatcatcaagatagtaatagccgtgaaacgcgtattcctattaagcgatggcaacaaggattcgatgcttatcctcctttaacaatgaacgaagaagaggagacacggttttctatgctcatgagaatctctgactctaggacgctaaccaggaatgggtttaagtaccaagaattgatgtatgattctaccgccttagcagattaccgtaagcattaccctcagacaaaagagactgtgaagaagctaataaaggtcgatccagatgatatctccaagatttacgtttatctggaagagttggaaagttacttggaggttccttgcacagatccaactggatacactgatggtttatccatatatgagcataaaaccattaagaagatcaaccgagaagttattagagaatcaaaagatagccttgggttggcgaaagcgagaatggctattcatgagagagtgaaacaagagcaagaagtattcatcgaaagtaagaccaaagctaagattactgcagtcaagaagcaggctcagatcgcggatgttagtaatacaggcacatcaacaatcaaggtgtccgaggagtctgcggctcctgtccagaaacacatctcaaatgataacagtgatgattgggatgatgatctggaagcttttgaatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaatgctcttacagagattcaaatagagaagttacggaatttcagtgattgcatcgtgatgcatcctcagataaagaccattttcaatgattttgatgaactaaggcttaatagaaagtttcagagtgatcagcaatgtatgcttcttataggagatactggtgttggcaaatcacacaccatcaaccattacaagaaaagagtcctcgcaacacaaaactattcaagaaacacaatgccggtccttgtttctcgcatatcacgtgggaaaggtttggatgctactttggtgcagatgctggcggatctagagctgtttggttcttctcagatcaagaagcggggctacaaaacagaccttaccaaaaaattagttgagagtttgatcaaagctcaagtggagctgttgattatcaatgaattccaagagcttattgagtttaaaagtgttcaagaaagacaacagattgcaaatggtctaaagtttattagtgaagaagccaaggttcccattgttttggttggaatgccatgggcagcaaagatagctgaggaaccacaatgggcttctcgactcgtaagaaaacgaaagctagaatatttttcactcaagaatgacagcaagtactttaggcagtatttgatgggtcttgccaaaaagatgcctttcgacgttcctcctaaattagaatcgaagaacactacgattgcgctatttgcagcttgtagaggggaaaacagagctttgaagcaccttcttttagaagcactgaagctcgccctttcatgcaatgagtatcttgagaacaagcatttcatcacagcttatgacaaattcgatttcttcaacgacaaggagaaactgaaatccaaaaaccctttcaagcaagatataaaagatattgaaatttatgaagttattaaaaattcttcctacaatccaaatgcacttgatccagaagatatgttgactgatcgagtatttgctattgtgaaaggaagcggaaagaggactgcagatggatcagaatttgagtccccaaagaagaaaaggaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggcttttcttttctcaccaaaagcaagagcgttttccgatgaatcgcttgaaagttatttacttcgagttgtctcagaaaacttcttcgattcatatgaaggactatctttggccatcagagaagaacttcatgagttggacttcgaggctcatggagcatttcccgtagatttaaagagacttaatgtttaccatgcaaaacacaactcacactttaggatgagagctcttggcctattagaaactcttttagatcttcctcgttatgagcttcaaaagctagcactcctgaagtcagacattaaattcaattcttccgttgccctctacaacaatggtgttgatattcctttgagattcattagacatcatgctgaggaagctgttgactccattccggtttgttctcaatgcttggctgaagaagcttatataaagcaatcttggcatatcaaatgggttaatgcttgcaccaaacatcaatgtgctttgcttcacaattgtcctgaatgttatgcaccgatcaattacattgagaatgaatcaatcactcattgctcttgtggttttgaattgagctgcgcctctacaagtcctgtgaacacactctcgattgaacatttgaacaaactcctggacaaaggagaacggaatgactcaaatccattgtttaacaacatgacgttgacagaaaggtttgctgctttattatggtatcaagagaggtattcacagactgataatttctgtttgaatgatgctgtgaactacttcagtaagtggccagcagtgtttaatactgagctagatgagctctcaaagaatgccgagatgaagctgattgatctcttcaacaagacagagtttaagtttatctttggagatgcaattcttgcttgtccaagtactcagaagcagagtgaatctcatttcatctaccgggcattacttgattaccttgtcactttagtggagagcaatcccaaaacaaagaagccaaacgcagctgatttgcttgtttctgtcctggaagcagcaactttgttggggacgagtgtagaacaagtttataggctctatcagaatggaatactgcaaaccgcctttcggcacaagatgaatcagagaataaacccttacaagggtgctttctttctccgtcatgtgatagagtataaaaccagctttggtaacgataaagctagaatgtatctctcagcttggtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatgaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcagagatattattttaccgttcattttcttcctaaacaagctaatttggcccttctcaccggaagatgcatatctatcatgcatggatttatcttgaagcataacattgaaggaatgggagtaacatttcccgcatggtctgattcaagcattggaaacgagattgcttttgtttacactgataaagaaatcctaaacacattgaaagatcaggcttactttgtggatatgcaagactgtggtttcttcaaggtttctcaggtgttggctgttcctgattcttgcgaggaagtcaggttcatccgcaatcaagccgtcgcaaagattttcactggcgaatcgaggaggagacttaaacggttacaaaagagagccttagcaagaggagaagatttcaatccaaagaagatagaagctccaagagaaatagacatttttcaccgtgtagcaatgacaagcaaaagttctcaagaagactacattctgcacatccagaagcaagatgttgattgtcaagcagagccttatttttcaaactatggtcttgcgtcaaatgagaagttcaaaggaactgtaccagacttgagtccgtccattgatagaaattgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatggagctttgcaatatactcaagtatgatcgaagtctttaccctgggaaagcggttttcttctacaaaacggcggacagcgattttgttcctttggaagctgacattaataagataagaggaccaaaatcaggctttacagaggctttcactcctcaattctctccgaaaaatatctctccacaagatctaactcacaacaacattcttacactcgaagagtgttatgttccaccaaatgtcgaacatattttctgccggttttctctgcgtgtccaagctaattcgttagtcccctctggttgttccgatcctgaggtttttagccttctcaaagagcttgcagaaaccttcaaagaatgtggtggttacaaggagctggcagtacgctattgtcggaacattttgattggaacatggctttggagaaatcagaataccggaaacacacagattgagatcaagacttcaaaaggatcttgctacttgattgataatacaagaaaactcgcttgggaaagcaaatgggcttcagacgacttgaaagttttggaagaattaagcaatgaaatcgaatctgctttaacagatccaaatgtgttttggtctgcagatataaccgccaagatcgaagcatctttctgtcaagagatatatccttcacagatattgaatgataaagtgaagcaaggagaagcgtcaaaacagtttgtgaaagctaaatgtgctgatggaaggtatgctgtgagtttcaacagtgttaagatcggtgctgctttgcaaagtattgatgattggtgggatgaagatgctagtaaaagacttagagttcatgaatttggtgcagacaaggaaattggcgtagcgaggaggcctcccgattccgaacaaaacttttattcaatcttcaagaacactgaatggtatctttccgccttaaagaattgcatcactaacaagaatgagaagattgacccagcaatatactatttgttcagcgtgttgatcaaaggtggaatgtttcagaaaaaggcagaggcaaagaaagcatgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgcacctgaaagagctgttagagataacagatacaactgagagagatagatctctccgacgtgccttctcgccgtataccgccatgatcgacattacaggatcagaagcagtcgctctaattatcctactcaatctcacttatagaaagaatcaggtagatgacctgttggataagaaactcgcgaaacaagccctcaagagtgaggatcatatcaacaaatgcatcaaggagattgcttggtttcatactcacaaccttaaatatccagacataagggtgagtaagcaaaatcttgcagtagaaccaccaacattgcattcttatgtgctttcttctgctaattatcccaaagcttatggttggtcccataattcagctaaagtgaattttgctaagttgtttgtctcatacttcaaatggcaaaaccaagtgagctggcttgcccaagtcttggcaaccaactctgataactggaaaagtgcatttacttcccttggtttatcggtgaaagccttcaaatctttatgcgttacagttaagaattctttacctgaagaggcaatcccggactcagtggatagatatagcaggcagattagaatgccttatcatgatggttatcttgctgtcactcctgtgatatctcacgttgttcaaagcaaaatacaacaagctgctattgacaaaagagctaggttttctaatgttgagtttaccagaccagctgctgtttcaatgctggcggcaagtctaggtggtgtgataaatgtacttaattatcctccatacattcgatcaaagtaccatggactatcgaattcaagggcttttaagttaaacaacggacagactgtcttcaacgtggaagccctcctgaagcctgagcttataaaagctcttgaaggaattatattctctaacaatgcattggcattaaaacagagaaggcagcagaaagtaaaaaacatcaaagaactgagaaataccctccttgagtggttttcccctgttttcgaatggagattggatgcaattgagaatggttatgatctagaacaattggaatcagcgagcgaacggttagagtacaagattctgagtttgccggataatgaactaccttctttgactatacccttgtttagattacttaatgagatgttgggaggagtttcaatgactcagaggtatgcattccatccaaagttgatgtctccattaaaggctgctctccaatggttgctcgttaatttaactgatcaaaagcatgttttaattgaagaagatgatgagcattacagataccttcacctgtcagggattagggtttttgatgcccaagcattgtctaatccttactgctctggtattccttcgcttacggcggtttggggaatgatccacagttatcaacgcaaactaaacgaagctttgggcacgaacgtacggttcacaagcttttcatggttcatccgtaattactctgccgttgctggtaaaaagctaccagaacttagccttcagggtgcacaacagtctagattgaaaagacctgggataatagacgggaagtattgtgatttggtgtttgacttgattattcatatcgatggctatgaagacgatttgcaggctgttgattcaaagcctgatatcctcaaggctcatttcccaagcaatttcgccggcggcgtgatgcatcaacccgagttgaattctaatattaattggtgttgtctttactccaacgaaaaccagctttttgagaagctccgtcggcttcctctttctggatgttgggttatgcccacagaacataagattcaagatcttgatgaactgttgttgctgttgaactcggatagtaaactaagtccaagcatgatgggatacatgcttctcacagagccaatggctcgtgtaggatcgttagagaggcttcactgttatgcagaacctgctattggtgttgtcaaatacgaggcagcaacttctgttcgattaaaaggtatcggaaactatttcaattccgcgttttggatgctcgatgctcaagaaaagttcatgctcatgaagaaagtgtgagcttatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtagcttaatgggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgccaagctaattcttgaagatgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgtcacttgtacagctcgtccatgccgagagtgatcccggcggcggtcacgaactccagcaggaccatgtgatcgcgcttctcgttggggtctttgctcagcttggactgggtgctcaggtagtggttgtcgggcagcagcacggggccgtcgccgatgggggtgttctgctggtagtggtcggcgagctgcacgccgccgtcctcgatgttgtggcggatcttgaagttggccttgatgccgttcttctgcttgtcggcggtgatatagacgttgtcgctaaagtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcacgccccaggacagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatacaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcatcaagattcttttcttcgcctggttcttctttttcaatttctacgtattcttcttcgtattctggcagtataggatcttgtatctgtacattcttcatttttgaacataggttgcatatgtgccgcatattgatctgcttcttgctgagcttacataatacttccatagtttttcccgtaaacattggattcttgatgctacatcttggataattaccttctggctccggttcgctaggttgaagagcaattctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 46 - DNA, 14022 bp, synthetic construct
  source 1..14022: mol_type = other DNA
tggcaggatatattgtggtgtaaacataacggatccggtctcaggagaatgaggagaatgtagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcggtatactaatgaggagtgtcgctgcaaccatagtttgacataatgcaaccataccttgacataattaaagtcatactctgacataaagccccttattaaaaatgatgctaaaaataaattccattataatcaattatttaagaattaattaaaccatagtgtgacatagtggctttttatttgtatagtatgtgtactcaaataataaaaatttacttatgtatatacgcaacttaagaaagccatctccaaataaaaatgtatttctacctgttccatggccaaccgctagcaaggaggtggagatccttcctcctagatccaaaatacggtaccgtcaacaatctcactcgagagtcgacgtatgtgcaaaactaagcattccgaagccattgttagccgtatgaatagggaaactaaacccagtgataagacctgatgttttcgcttctttaattacatttggagatgcccgctatacaggagccgtataggcagctgaagataaaaacaatcgattcgatgtttaagttaatgtaattctgccgaaaaggcagtgagtagtaattgaaaaaaccagtcttatatccgggctggtttttttgtttttagcgttataacttttatttcactatgtcactgtatggttgcattttatgtcagtgaatggttgcaggttttttgtaagttattgatatgcctatccagtgtatgtcacgctttgcttgcagcgacacgctacagaggagtctgagcaggacaactcgcgtagtgagagttacatgttcgttgggttcttccgacacggacctgagttggccaacgtcccacctgaggtctgtgccccggtgatgagaagtgtgcatctcgttcttgcagctcgtcagtactttcagaatcatggcgtgcatggtagaatgacccttataacggacttcgacatggcaatcgctaggtaaatgaatggaaaaagtggcaccgagtcggtgctttttttcccggggcgcgccgatatcgagctctcccggcgcgccgatatcgagctcagtgtttgatcgccggcggtaccgagtgtacttcaagtcagtgggaaatcaataaaatgattattttatgaatatatttcattgtgcaagtagatagaaattacatatgttacataacacacgaaataaacaaaaaaagacaatccaaaaacaaacaccccaaaaaaaataatcactttagataaactcgtatgaggagaggcacgttcagtgactcgacgattcccgagcaaaaaaagtctccccgtcacacatgtagtgggtgacgcaattatctttaaagtaatccttctgttgacttgtcattgataacatccagtcttcgtcaggattgcaaagaattatagaagggatcccaccttttattttcttcttttttccatatttagggttgacagtgaaatcagactggcaacctattaattgcttccacaatgggacgaacttgaaggggatgtcgtcgatgatattataggtggcgtgttcatcgtagttggtgaaatcgatggtaccgttccaatagttgtgtcgtccgagacttctagcccaggtggtctttccggtacgagttggtccgcagatgtagaggctggggtgtcggattccattccttccattgtccttgttaaatcggccatccattcaaggtcagattgagcttgttggtatgagacaggatgtatgtaagtataagcgtctatgcttacatggtatagatgggtttccctccaggagtgtagatcttcgtggcagcgaagatctgattctgtgaagggcgacacatacggttcaggttgtggagggaataatttgttggctgaatattccagccattgaagctttgttgcccattcatgagggaattcttccttgatcatgtcaagatattcctccttagacgttgcagtctggataatagttctccatcgtgcgtcagatttgcgaggagaaaccttatgatctcggaaatctcctctggttttaatatctccgtcctttgatatgtaatcaaggacttgtttagagtttctagctggctggatattagggtgatttccttcaaaatcgaaaaaagaaggatccctaatacaaggttttttatcaagctggaggagagcatgatagtgggtagtgccatcttgatgaagctcagaagcaacaccaaggaagaaaataagaaaaggtgtgagtttctcccagagaaactggaataaatcatctctttgagatgagcacttgggataggtaaggaaaacatatttagattggagtctgaagttcttactagcagaaggcatgttgttgtgactccgaggggttgcctcaaactctatcttataaccggcgtggaggcatggaggcaggggtattttggtcattttaatagatagtggaaaatgacgtggaatttacttaaagacgaagtctttgcgacaagggggggcccacgccgaatttaatattaccggcgtggcccccccttatcgcgagtgctttagcacgagcggtccagatttaaagtagaaaatttcccgcccactagggttaaaggtgttcacactataaaagcatatacgatgtgatggtatttgatggagcgtatattgtatcaggtatttccgttggatacgaattattcgtacgaccctcatagtttaaactatcagtgggtagcttaatggtagacactttattagttagatccgctgttattctgcacgattgcgcttgccggtggaaatttaaggacgagtgtagtgcactcacccttagccttcagcgtgacaagattagggtagcaccggaaacagcgattttacggacagatagtacagtctgagactcagtcactggtccctaaacgggagccggtacgtcggcggttcgctagcaaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgactgataaacggaaagatggttccggcaagcttctttactgcagcttttgcgggaaatctcaacatgaagttaggaaacttattgcagggccttctgtttatatctgtgacgaatgtgttgatctctgcaatgatattattcgagaagagatcaaagaagtggctcctcacagagagaggagtgcgttgccgacaccacacgagattagaaatcatcttgatgattatgtcattggtcaagaacaagcaaagaaggtccttgctgttgcagtgtacaaccattacaagaggctcagaaacggagatacttctaatggtgtggaacttggcaaatcaaacatcctgctcattggacccactggaagtggaaagacactcctagcagagacattggcaaggctcttagacgttcctttcactatggctgatgcaactactcttactgaggctggttatgttggagaagatgtggagaatattattcaaaagttattgcagaaatgtgattacgatgtccagaaagctcagagagggatagtctacatagatgagatagacaagatctctaggaagagtgataatccctcgataacacgcgacgtttctggcgaaggtgttcaacaagctttgttgaagctcattgaaggcaccgtagcggcagttccaccacaaggaggaagaaagcatcctcagcaagagtttctccaagtggatacctcaaaaatcttatttatatgtggtggtgccttcgctgggcttgacaaagttatctcacaccgtgtagaaactggtagcgggattgggtttggtgcaacggtgaaggcaaaatctgataaggcttctgaaggagagcttctggctcaagttgaaccagaggatcttatcaagtttggactaattcctgagttcatcggtagacttcctgtcgttgctacgttgaacgagttaagtgaggaagccctgattcaaatcttgaaagagccaaagaatgctctgacaaagcaatatcaggctcttttcaatcttgagggagttgacttggaatttcgtgatgaagcgctggatgctatagctaagaaagcaatggcgaggaaaaccggagctcgtggtctaagatcgattgttgaagccgctttattggatacaatgtatgatttgccctccatggaagatgttgagaaagtggtcatcgacgagtctgtgattgatggacagtcaaagccattgctcatatatggtaaacctgaagcacagcaggcatcaggagaatgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctatacaggagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaaagcgtactgctgatggttcagagtttgaaagtccaaagaagaaaagaaaagttggtagtggtatgagttattctggagaacgggataattttgcacctcacatggcattagttccaatggtgattgaacaaacaagtagaggggagcgaagctttgacatttactctcgtctattaaaagaaagggtcatcttcttaaccgggcaagttgaagatcatatggcaaatctgattgtagctcagatgctttttcttgaagcagagaatcctgagaaagatatctatctctacattaactctcctggtggtgtaataactgctggaatgtctatatatgatacaatgcagtttatcaaacctgatgtttcaacaatatgtatgggtcaggctgcgtcaatgggagcttttcttttgacggctggcgcaaaaggaaagcggttctgcctaccgaactcaagggtgatgatccatcaaccacttggaggataccaaggtcaagctaccgatatagagattcatgctagagaaattttgaaggtgaagggtagaatgaatgaactgatggcccttcacactggacaatcgctcgagcagattgagcgtgacacagagagagacaggtttttatccgcaccagaagctgttgagtatggtttggttgattctattcttactcatagaaactgaatatgaagatgaagatgaaatatttggtgtgtcaaataaaaaggttgtgtgcttaagtttgtgtttttttcttggcttgttgtgttatgaatttgtggctttttctaatattaaatgaatgtaacatctcattataatgaataaacaaatgtttctataatccattgtgaatgttttgttggatctcttctccagcatataactactgtatgtgctatggtatggactatggaatatgattaaagataagcgctacagaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgataatggtgacctgccgtataggcagctgaagatactgacgtaagggatgacgcacaatcccactataattctgccgaaaaggcagtgagtagtgcttttttttttgcaaaattttccagatcgatttcttcttcctctgttcttcggcgttcaatttctggggttttctcttcgttttctgtaactgaaacctaaaatttgacctaaaaaaaatctcaaataatatgattcagtggttttgtacttttcagttagttgagttttgcagttccgatgagataaaccaatacgctaggtatacttgagaccggatcctgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagtgctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcaccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctggaacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctgg

SEQ ID NO: 47 - DNA, 25 bp, synthetic construct
  source 1..25: mol_type = other DNA
gccgacagtggtcccaaagatggac

SEQ ID NO: 48 - DNA, 18 bp, synthetic construct
  source 1..18: mol_type = other DNA
cttgccggtggtgcagag

SEQ ID NO: 49 - DNA, 21 bp, synthetic construct
  source 1..21: mol_type = other DNA
ccacccacgaggagcatcgtg

SEQ ID NO: 50 - DNA, 24 bp, synthetic construct
  source 1..24: mol_type = other DNA
ctgaagtttagaccatgaagaggc

SEQ ID NO: 51 - DNA, 24 bp, synthetic construct
  source 1..24: mol_type = other DNA
aggggctttatgtcagagtatgac

SEQ ID NO: 52 - DNA, 24 bp, synthetic construct
  source 1..24: mol_type = other DNA
gactgctgctggtattaccgaggg

SEQ ID NO: 53 - DNA, 37 bp, synthetic construct
  source 1..37: mol_type = other DNA
gcggatctaactaataaagtgtctaccattagtatac

SEQ ID NO: 54 - DNA, 25 bp, synthetic construct
  source 1..25: mol_type = other DNA
tccgtgactgatgcaaggttatcgg

SEQ ID NO: 55 - DNA, 24 bp, synthetic construct
  source 1..24: mol_type = other DNA
aaaccagtcttatatccgggctgg

SEQ ID NO: 56 - DNA, 37 bp, synthetic construct; identical to SEQ ID NO: 53
  source 1..37: mol_type = other DNA

SEQ ID NO: 57 - DNA, 25 bp, synthetic construct
  source 1..25: mol_type = other DNA
tggcgaacagttcatacagagtctc

SEQ ID NO: 58 - DNA, 19 bp, synthetic construct
  source 1..19: mol_type = other DNA
tccagctcgaccaggatgg

SEQ ID NO: 59 - DNA, 24 bp, synthetic construct
  source 1..24: mol_type = other DNA
tcttcgtcaacatggtggagcacg

SEQ ID NO: 60 - DNA, 24 bp, synthetic construct
  source 1..24: mol_type = other DNA
ctgaagtttagaccatgaagaggc

SEQ ID NO: 61 - DNA, 24 bp, synthetic construct
  source 1..24: mol_type = other DNA
aggggctttatgtcagagtatgac

SEQ ID NO: 62 - DNA, 18 bp, synthetic construct
  source 1..18: mol_type = other DNA
catttggagatgttgata

SEQ ID NO: 63 - DNA, 18 bp, synthetic construct
  source 1..18: mol_type = other DNA
catcaacaggagagaaca

SEQ ID NO: 64 - DNA, 18 bp, synthetic construct
  source 1..18: mol_type = other DNA
actctagaggtgttgata

SEQ ID NO: 65 - DNA, 18 bp, synthetic construct
  source 1..18: mol_type = other DNA
catcaacaagaggatcca

SEQ ID NO: 66 - DNA, 18 bp, synthetic construct
  source 1..18: mol_type = other DNA
tcatttggagtgtcgctg

SEQ ID NO: 67 - DNA, 18 bp, synthetic construct
  source 1..18: mol_type = other DNA
cagcgacatggagagaac

SEQ ID NO: 68 - DNA, 18 bp, synthetic construct
  source 1..18: mol_type = other DNA
actctagaggtgtcgctg

SEQ ID NO: 69 - DNA, 18 bp, synthetic construct
  source 1..18: mol_type = other DNA
cagcgacaagaggatcca